(19) United States
(12) Patent Application Publication
(10) Pub. No.: US 2020/0190568 A1
(43) Pub. Date: Jun. 18, 2020

(54) METHODS FOR DETECTING THE AGE OF BIOLOGICAL SAMPLES USING METHYLATION MARKERS

(71) Applicant: OneSkin Technologies, Inc., San Francisco, CA (US)

(72) Inventors: Mariana Lima Boroni Martins, Rio de Janeiro (BR); Edgar Andres Ochoa Cruz, San Francisco, CA (US); Carolina Reis de Oliveira, San Francisco, CA (US); Alessandra Arcoverde Cavalcanti Zonari, Coimbra (PT); Juliana Lott de Carvalho, Lago Norte (BR)

(21) Appl. No.: 16/709,777

(22) Filed: Dec. 10, 2019

Related U.S. Application Data
(60) Provisional application No. 62/777,717, filed on Dec. 10, 2018.

(51) Int. Cl. C12Q 1/6869; C12Q 1/6806; G06N 20/00; C12Q 1/6883; G16B 50/10; G16B 30/00

(52) U.S. Cl. CPC C12Q 1/6869; C12Q 1/6806; G06N 20/00; G16B 50/30; G16B 50/10; G16B 30/00; C12Q 2600/154; C12Q 1/6883

(57) ABSTRACT

The disclosure relates to systems, software and methods for gerontological classification of subjects based on a detection of a plurality of epigenetic markers such as methylation status of nucleotides (e.g., CpG) in the genomic DNA.

### APPLICATION FOR CLAIM OF PRIORITY

This application claims the benefit under 35 U.S.C. § 119(e) of U.S. Provisional Application No. 62/777,717, filed Dec. 10, 2018. The disclosure of the above-identified application is incorporated herein by reference as if set forth in full.

### SEQUENCE LISTING

The instant application contains a Sequence Listing, which has been submitted electronically in ASCII format and is hereby incorporated by reference in its entirety. Said ASCII copy, created on Dec. 6, 2019, is named 104273-0025_SL.txt and is 90,688 bytes in size.

### FIELD OF THE DISCLOSURE

The disclosure generally relates to molecular biology, genomics, and informatics. Embodiments of the disclosure relate to methods and systems for detecting age of a biological specimen, e.g., human tissues, by detecting status of methylation markers in the genomic DNA.

### BACKGROUND

A wide variety of analytical techniques are devoted to characterizing biological specimen on the basis of age, which is particularly useful in forensic medicine, female reproductive biology and substance abuse (van Oorschot et al., Investigative Genetics 1:14, 2010; Thompson et al., Methods Mol Biol. 830:3-16, 2012; Binder et al., Epigenetics, 13:1-31, 2017; Kozlenkov et al., Genes (Basel), 8(6). pii: E152, 2017). Existing methods such as DNA fingerprinting and radio-dating of teeth enamel are of limited prognostic significance (Buchholz et al., Surface and Interface Analysis, 42:398, 2010). Other techniques such as telomere shortening, mitochondrial mutations, and single joint T-cell receptor excision circle rearrangements are burdened by low accuracy (Bekaert et al., Epigenetics, 10(10): 922-930, 2015).

Accurate gerontological determinations are especially useful in the field of cosmetics, wherein subjective tissue properties such as clarity, texture, elasticity, color, tone, pliability, firmness, tightness, smoothness, thickness, radiance, evenness, laxity, oiliness, and wrinkles, are still being used to categorize skin tissue as “young”/“old” or “healthy”/“unhealthy.” These tissue-typing methods are invasive, time-consuming, expensive, and also require use of sophisticated tools and devices. Above all, these analytical methods and the data derived therefrom are highly subjective and have limited reproducibility.

Recent discoveries in molecular biology have yielded new paradigms in tissue typing. For example, epigenetic changes are believed to contribute significantly to aging and related conditions such as immunodeficiency, and degenerative diseases (Pal et al., Sci Adv., 2(7): e1600584, 2016). Age-associated changes in DNA methylation have been studied. Differences in the DNA methylome in aging humans are often commonly associated with global CpG hypomethylation, especially at repetitive DNA sequences (Heyn et al., PNAS USA, 109(26), 10522-10527, 2012).

However, there seems to be some dispute in the diagnostic community with regard to the level of association between aging and gDNA methylation. Subject-independent parameters such as tissue type, disease state, and assay platform all have been postulated to affect the actual level and genomic sites of hypomethylation, thereby introducing some variability to the biometric assays.

Accordingly, there is an unmet need for sensitive, optimized, non-invasive gerontological analytical systems and methods that are capable of, accurately and probabilistically, detecting age-associated epigenetic biomarkers. Moreover, compositions and kits containing probes that specifically detect “molecular age” epigenetic signatures in biological samples may be useful for providing valuable clues to forensic experts involved in criminal investigation regarding gerontological traits of their subjects and/or suspects. In the context of high throughput screening of candidate drugs, there is a need for in vitro platforms that serve as objective beacons (e.g., epigenetic markers) for reliably and accurately assessing, at a molecular level, the effects of various test agents on aging and tissue rejuvenation. Compositions and kits containing probes that specifically detect “molecular age” epigenetic signatures in biological samples may also be useful during the basic research and development phase of novel products regarding the gerontological traits of samples treated with different compounds under development.

### SUMMARY

Provided herein are programs, systems, and methods for detecting gerontological epigenetic markers in tissue specific biological samples and using the information obtained from the detection to diagnose subjects (or samples obtained from the subjects), classify them (e.g., in age cohorts) and also to stratify them based on likelihood of developing age-associated indications such as degenerative diseases and/or immunodeficiency. In some embodiments, the programs, systems and methods of the disclosure allows a user, e.g., a clinician or patient, to overcome the core challenges of existing gerontological classification systems and methods based on skin typing non-quantitative data, as detailed above.

The disclosure relates, in part, to novel epigenetic markers and or their combination, such as methylation markers, which were identified using Machine Learning algorithms based thereon from a dataset of 249 human epidermal and/or dermal samples, each one profiled using genome-wide 450,000+methylation (CpG) probes. The methylation markers are scored based on predictive powers, as assessed by linear regression.

The age calculating tool of the instant disclosure principally comprises the following components: (a) a selected, modified, noise-free composite dataset; (b) a specific algorithm that is trained with the noise-free composite dataset of (a); and (c) a validation or testing dataset that is different from the noise-free composite training dataset.

FIG. 1 illustrates an exemplary experimental design of the age-prediction methodology according to various embodiments. In specific implementations, three datasets were used to build and also test the systems and methods of the disclosure. The specific datasets, GSE51954, E-MTAB-4385, GSE90124, are available in public databanks and each comprise epigenetic data, including additional information such as tissue, gender and age composition. About 508 samples (40 dermis, 146 epidermis, 322 whole skin) were used in the buildup, each sample had more than 450,000 CpG/probes/features. In order to build a machine learning algorithm that is able to predict age accurately, these datasets were merged, preprocessed, normalized, age-balanced and divided in training subset and testing subsets (see e.g., FIG. 2 and FIG. 3). This particular step includes, e.g., (a) homogenous processing of the raw data of each dataset to generate a set of probes with methylation levels comparable among the three datasets, comprising a unique and normalized dataset containing 508 samples; (b) removing cross-reactive probes, the sex-specific probes and probes that are not present in the methylation array such as INFINIUM Methylation EPIC kit; (c) pre-selecting more relevant probes by combining the results of a wrapper to estimate the importance based on three different methodologies: glmnet-lasso, xgboost, and ranger, resulting in an aggregate of about 300 probes; and (d) selecting the samples in the training dataset in order to have a balanced distribution between the ages (cut-off of 5 samples per age window, wherein an age window is about 7 years). The balanced-training dataset included 249 samples and the remaining 259 samples were used for the testing dataset.

Next, the age-calculating or age-predicting algorithm of the present disclosure was developed. Herein, several Machine Learning (ML) algorithms were applied, in each case, a 50 fold resampling cross-validation was used for optimization of the tuning parameters. Model prediction errors were computed using mean absolute error (MAE) and/or root mean squared error (RMSE) and the fitness levels and significance of the applied regression models were evaluated by computing Pearson's correlation coefficient using the training data (e.g., smaller MAE or RMSE scores indicate better predictive algorithm and an R² value of ˜1.0 indicates better fit) (see e.g., FIG. 4). Subsequently, an optimal regression was selected (generated with Ridge regression machine learning algorithm, which penalizes the size of parameter estimates by shrinking them to zero, in order to decrease complexity of the model while including all the variables in the model).

ENGINE was validated using the testing dataset (259 samples—see e.g., FIG. 5A-FIG. 5C), where the R² and RMSE values were evaluated. Using this method, a significance of each of the 300 set of probes to serve as biomarkers related to age was validated. The relevance of each biomarker with respect to the calculated age of the biological sample (e.g., skin sample) was deciphered (FIG. 6 shows the first 100 deciphered biomarkers). Further, the results were additionally validated by predicting the age of an external dataset of skin biopsies, in which accuracy of ENGINE was compared with knowns system, described by Horvath (see e.g., FIG. 7).

Comparative assessment of the methylation markers of the disclosure with that disclosed in Horvath et al., Genome Biol., 14, R115, 2013; US 2016-0222448 and Horvath et al., Aging 10, 1758-1775, 2018 indicate that the methylation markers of the disclosure are new and also superior to Horvath in terms of predictive power. For example, in linear regression analysis, the correlation coefficient between sample age and methylation status at the external dataset of skin biopsies was about 0.96, demonstrating a specific and robust association between the markers of the disclosure and age and high prediction accuracy (see e.g., FIG. 7A). In contrast, the correlation coefficient between Horvath's markers and age, as applied also to the external dataset of skin biopsies, was only about 0.90 for 1^{st} Horvath Molecular Clock and about 0.95 for 2^{nd} Horvath Molecular Clock (FIG. 7B and FIG. 7C). The improved accuracy with the methods of the disclosure was apparent throughout the subject cohort, even in the case of quinquagenarian or older subjects (i.e., >50 years). Furthermore, the difference between the chronological age and the predicted age (Δ), as determined by the systems and methods of the disclosure, was consistently smaller than Horvath's methods. For instance, with the instant methods, mean A was about 1.2 years (range of −8.3 years to 9.2 years; standard deviation of 4.6 years), while for 1^{st} Horvath Molecular Clock, mean A was −14.1 years (range of −26.7 years to −5.6 years; standard deviation of 15.7 years), and for 2^{nd} Horvath Molecular Clock, mean A was 5.7 years (range of −3.7 years to 13 years; standard deviation of 7.6 years). Furthermore, Horvath's method consistently underestimated the sample predicted age (i.e., predicted age <<actual age). See e.g., Table 4. These results showed that the systems and methods of the disclosure are significantly superior to art-existing methods for predicting age of biological samples.

The disclosure relates to the following exemplary, non-limiting embodiments:

In some embodiments, the disclosure relates to systems for calculating age of a biological sample, comprising: a data acquisition unit comprising (a) a receiver for receiving a plurality of methylome datasets from a plurality of heterogeneous samples of different age or age groups, wherein each dataset comprises a plurality of methylation markers; (b) a processor for homogenizing the plurality of methylome datasets and merging the homogenized dataset into a single data frame, thereby generating a processed dataset comprising a string of homogenized and merged methylation markers; (c) a filter for eliminating confounding markers from the processed dataset of (b), wherein filtration step comprises: removing cross-reactive markers in the processed dataset; normalizing the dataset; removing not available markers in the processed dataset; and/or removing sex-specific markers from the processed dataset; (d) an identifier for identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers; and (e) a selector for selecting a training dataset of samples, each already containing the relevant and unique markers of (d), wherein the selection step comprises balancing the age distribution of samples from which the relevant and unique markers are obtained.

In some embodiments, the disclosure relates to systems for calculating age of a biological sample, comprising: a marker identification unit configured to identify a plurality of age-specific methylation markers in a training dataset, wherein the marker identification unit is optionally communicatively connected to a data acquisition unit and comprises: (a) a classification engine configured to statistically classify each relevant marker in the training dataset on the basis of a relevance score which indicates a level of a statistical association between the marker and the age, wherein the methylation markers comprises the markers listed in Table 1, wherein the markers in Table 1 are listed in descending order of relevance score, and wherein the classification engine utilizes a machine learning (ML) model; and optionally (b) a validation unit for validating the trained machine learning algorithm with a validation dataset.

In some embodiments, the disclosure relates to systems for calculating age of a biological sample, comprising an analyzing unit comprising: a detector for detecting the methylation status of age-specific, unique and relevant methylation markers (e.g., identified as above) or a gene linked to said methylation marker or locus thereto in a biological sample; and (b) an age assessor which calculates the age of the biological sample based on the detected methylation status of the sample.

In some embodiments, the disclosure relates to systems for selecting markers for a training dataset to predict age of a biological sample, comprising: (1) a data acquisition unit comprising a) a receiver for receiving a plurality of methylome datasets from a plurality of heterogeneous samples of different age or age groups, wherein each dataset comprises a plurality of methylation markers; b) a processor for homogenizing the plurality of methylome datasets and merging the homogenized dataset into a single data frame, thereby generating a processed dataset comprising a string of homogenized and merged methylation markers; c) a filter for eliminating confounding markers from the processed dataset of (b), wherein filtration step comprises: removing cross-reactive markers in the processed dataset; normalizing the dataset; removing not available markers in the processed dataset; and/or removing sex-specific markers from the processed dataset; d) an identifier for identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers; e) a selector for selecting a training dataset of samples, each already containing the relevant and unique markers of (d), wherein the selection step comprises balancing the age distribution of samples from which the relevant and unique markers are obtained; optionally (2) a marker identification unit configured to identify a plurality of age-specific methylation markers in the training dataset of e), the marker identification unit communicatively connected to the data acquisition unit, comprising: f) a classification engine configured to statistically classify each relevant marker in the training dataset of e) on the basis of a relevance score which indicates a level of a statistical association between the marker and the age, wherein the methylation markers comprises the markers listed in Table 1, wherein the markers in Table 1 are listed in descending order of relevance score, and wherein the classification engine utilizes a machine learning (ML) model; and g) optionally a validation unit for validating the trained machine learning algorithm of (f) with a validation dataset; and further optionally (3) an analyzing unit comprising: h) a detector for detecting the methylation status of age-specific, unique and relevant methylation markers identified in (e) or a gene linked to said methylation marker or locus thereto in a biological sample; and i) an age assessor which calculates the age of the biological sample based on the detected methylation status of the sample. Preferably, the systems of the disclosure for calculating age of a biological sample comprise (1) the data acquisition unit; (2) the marker identification unit; and (3) the analyzing unit, as described above.

In some embodiments, the disclosure relates to systems for calculating age of a biological sample, comprising: (1) a data acquisition unit comprising a) a receiver for receiving a plurality of methylome datasets from a plurality of heterogeneous samples of different age or age groups, wherein each dataset comprises a plurality of methylation markers; b) a processor for homogenizing the plurality of methylome datasets and merging the homogenized dataset into a single data frame, thereby generating a processed dataset comprising a string of homogenized and merged methylation markers; c) a filter for eliminating confounding markers from the processed dataset of (b), wherein filtration step comprises: removing cross-reactive markers in the processed dataset; normalizing the dataset; removing not available markers in the processed dataset; and/or removing sex-specific markers from the processed dataset; d) an identifier for identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers; e) a selector for selecting a training dataset of samples, each already containing the relevant and unique markers of (d), wherein the selection step comprises balancing the age distribution of samples from which the relevant and unique markers are obtained; optionally (2) a marker identification unit configured to identify a plurality of age-specific methylation markers in the training dataset of e), the marker identification unit communicatively connected to the data acquisition unit, comprising: f) a classification engine configured to statistically classify each relevant marker in the training dataset of e) on the basis of a relevance score which indicates a level of a statistical association between the marker and the age, wherein the methylation markers comprises the markers listed in Table 1, wherein the markers in Table 1 are listed in descending order of relevance score, and wherein the classification engine utilizes a machine learning (ML) model; and g) optionally a validation unit for validating the trained machine learning algorithm of (f) with a validation dataset; and further optionally (3) an analyzing unit comprising: h) a detector for detecting the methylation status of age-specific, unique and relevant methylation markers identified in (e) or a gene linked to said methylation marker or locus thereto in a biological sample; and i) an age assessor which calculates the age of the biological sample based on the detected methylation status of the sample. Preferably, the systems of the disclosure for calculating age of a biological sample comprise (1) the data acquisition unit; (2) the marker identification unit; and (3) the analyzing unit, as described above.

In some embodiments, the disclosure relates to computer readable media comprising computer-executable instructions, which, when executed by a processor, cause the processor to carry out a method or a set of steps for diagnosing aging or an age-related disease in a subject, the method or the set of steps comprising, (a) receiving a plurality of methylome datasets from a plurality of heterogeneous samples of different age or age groups, wherein each dataset comprises a plurality of methylation markers; (b) homogenizing the plurality of methylome datasets and merging the homogenized dataset into a single data frame, thereby generating a processed dataset comprising a string of homogenized and merged methylation markers; (c) filtering confounding markers from the processed dataset of (b), wherein filtration step comprises: removing cross-reactive markers in the processed dataset; normalizing the dataset; removing individually not available markers in the processed dataset; and/or removing sex-specific markers from the processed dataset; (d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers; and (e) selecting a training dataset from the pool of relevant and unique markers of (d), wherein the selection step comprises balancing the age distribution of samples from which the relevant and unique markers are obtained.

In some embodiments, the disclosure relates to computer readable media comprising computer-executable instructions, which, when executed by a processor, cause the processor to carry out a method or a set of steps for diagnosing aging or an age-related disease in a subject, the method or the set of steps comprising training a machine-learning algorithm comprising the Ridge regression machine learning algorithm with a training dataset comprising methylation markers (e.g., aforementioned filtered methylation markers), thereby generating a plurality of age-specific, unique and relevant methylation markers, e.g., the methylation markers listed in Table 1, wherein the markers in Table 1 are listed in descending order of relevance score; and optionally validating the trained machine learning algorithm with a validation dataset.

In some embodiments, the disclosure relates to computer readable media comprising computer-executable instructions, which, when executed by a processor, cause the processor to carry out a method or a set of steps for diagnosing aging or an age-related disease in a subject, the method or the set of steps comprising detecting the methylation status of age-specific, unique and relevant methylation markers (e.g., identified as above) or a gene linked to said methylation marker or locus thereto in a biological sample; and calculating the age of the biological sample based on the detected methylation status of the sample.

In some embodiments, the disclosure relates to computer readable media comprising computer-executable instructions, which, when executed by a processor, cause the processor to carry out a method or a set of steps for diagnosing aging or an age-related disease in a subject, the method or the set of steps comprising, (A) a pre-analytical data processing, filtering, selection and balancing steps; optionally (B) a system setup step; and further optionally (C) an analytical step, wherein the pre-analytical step (A) comprises: (a) receiving a plurality of methylome datasets from a plurality of heterogeneous samples of different age or age groups, wherein each dataset comprises a plurality of methylation markers; (b) processing to homogenize the plurality of methylome datasets and merging the homogenized dataset into a single data frame, thereby generating a processed dataset comprising a string of homogenized and merged methylation markers; (c) filtering confounding markers from the processed dataset of (b), wherein filtration step comprises: removing cross-reactive markers in the processed dataset; normalizing the dataset; removing individually not available markers in the processed dataset; and/or removing sex-specific markers from the processed dataset; (d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers; and (e) selecting a training dataset from the pool of relevant and unique markers of (d), wherein the selection step comprises balancing the age distribution of samples from which the relevant and unique markers are obtained; wherein the system setup step (B) comprises (f) training a machine-learning algorithm comprising a Ridge regression machine learning algorithm with the training dataset of (e), thereby generating a plurality of age-specific, unique and relevant methylation markers, e.g., the methylation markers listed in Table 1, wherein the markers in Table 1 are listed in descending order of relevance score; and (g) optionally validating the trained machine learning algorithm of (f) with a validation dataset; and wherein the analytical step (C) comprises (h) detecting the methylation status of age-specific, unique and relevant methylation markers identified in (e) or a gene linked to said methylation marker or locus thereto in the subject's biological sample; and (i) calculating the age of the subject's biological sample based on the detected methylation status of the subject's biological sample, wherein the markers in Table 1 are listed in descending order of relevance to the age of the subject's biological sample, and wherein if the calculated age is greater than the actual age of the subject, then the subject is diagnosed with aging or having an age-related disease. Preferably, the computer readable media of the disclosure comprise computer-executable instructions, which, when executed by a processor, cause the processor to carry out a method or a set of steps for predicting aging or an age-related disease in a subject, the method or the set of steps comprising, (A) the pre-analytical data processing, filtering, selection and balancing steps; (B) the system setup step; and (C) the analytical step, as described above.

In some embodiments, the disclosure relates methods for calculating an age of a biological sample, comprising, detecting the methylation status of age-specific, unique and relevant methylation markers or a gene linked to said methylation marker or locus thereto in the biological sample; and determining the age of the biological sample based on the detected methylation status of the biological sample, wherein age-specific, unique and relevant methylation markers are identified with a trained machine-learning algorithm comprising a Ridge regression machine learning algorithm and the machine learning algorithm is optionally validated with a validation dataset comprising processed markers. Preferably, the training dataset and/or the validation dataset comprises processed, filtered, selected and age-balanced methylation markers, wherein the processing, filtering, selecting and balancing steps include (a) receiving a plurality of methylome datasets from a plurality of heterogeneous samples of different age or age groups, wherein each dataset comprises a plurality of methylation markers; (b) processing to homogenize the plurality of methylome datasets and merging the homogenized dataset into a single data frame, thereby generating a processed dataset comprising a string of homogenized and merged methylation markers; (c) filtering confounding markers from the processed dataset of (b), wherein filtration step comprises: removing cross-reactive markers in the processed dataset; normalizing the dataset; removing individually not available markers in the processed dataset; and/or removing sex-specific markers from the processed dataset; (d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers; and (e) selecting a training dataset from the pool of relevant and unique markers of (d), wherein the selection step comprises balancing the age distribution of samples from which the relevant and unique markers are obtained.

In some embodiments, the disclosure relates methods for calculating an age of a biological sample, comprising, training a machine-learning algorithm comprising a Ridge regression machine learning algorithm with a training dataset comprising methylation markers, thereby generating a plurality of age-specific, unique and relevant methylation markers, e.g., the methylation markers listed in Table 1, wherein the markers in Table 1 are listed in descending order of relevance score; optionally validating the trained machine learning algorithm with a validation dataset; detecting the methylation status of age-specific, unique and relevant methylation markers or a gene linked to said methylation marker or locus thereto in the biological sample; and determining the age of the biological sample based on the detected methylation status of the biological sample. In some embodiments, a first predicted age is determined based on the methylation status and a second predicted age is determined by performing an operation (e.g., addition or subtraction) on the first predicted age. Specifically, the operation comprises an addition or subtraction of a delta age (δ), derived from a validation dataset of samples obtained from the subject, e.g., as provided in a hash table of Table 4.

In some embodiments, the disclosure relates methods for calculating an age of a biological sample, comprising, (A) a pre-analytical data processing, filtering, selection and balancing steps; optionally (B) a system setup step; and further optionally (C) an analytical step, wherein the pre-analytical step (A) comprises: a) receiving a plurality of methylome datasets from a plurality of heterogeneous samples of different age or age groups, wherein each dataset comprises a plurality of methylation markers; b) processing to homogenize the plurality of methylome datasets and merging the homogenized dataset into a single data frame, thereby generating a processed dataset comprising a string of homogenized and merged methylation markers; c) filtering confounding markers from the processed dataset of (b), wherein filtration step comprises: removing cross-reactive markers in the processed dataset; normalizing the dataset; removing unavailable markers in the processed dataset; and/or removing sex-specific markers from the processed dataset; d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers; e) selecting a training dataset from the pool of relevant and unique markers of (d), wherein the selection step comprises balancing the age distribution of samples from which the relevant and unique markers are obtained; wherein the system setup step (B) comprises f) training a machine-learning algorithm comprising a Ridge regression machine learning algorithm with the training dataset of e), thereby generating a plurality of age-specific, unique and relevant methylation markers, e.g., the methylation markers listed in Table 1, wherein the markers in Table 1 are listed in descending order of relevance score; and g) optionally validating the trained machine learning algorithm of (f) with a validation dataset; and wherein the analytical step (C) comprises h) detecting the methylation status of age-specific, unique and relevant methylation markers identified in (e) or a gene linked to said methylation marker or locus thereto in the biological sample; and i) determining the age of the biological sample based on the detected methylation status of the biological sample. Preferably, the methods for calculating an age of a biological sample of the disclosure comprise (A) the pre-analytical data processing, filtering, selection and balancing steps; (B) the system setup step; and (C) the analytical step, as described above.

In some embodiments, provided herein are systems, computer-readable media, and/or methods per the foregoing or the following, wherein the methylation markers comprise levels and/or activity of methylated genomic DNA (gDNA) in the samples.

In some embodiments, provided herein are systems, computer-readable media, and/or methods according to the foregoing or the following, wherein the cross-reactive markers are identified by comparing the dataset of (b) with a standard, non-specific probe dataset.

In some embodiments, provided herein are systems, computer-readable media, and/or methods according to the foregoing or the following, wherein the unavailable markers comprise markers that are not included in the pool of markers which are assayable with the methylation assay instrument.

In some embodiments, provided herein are systems, computer-readable media, and/or methods according to the foregoing or the following, wherein the sex-specific markers comprise markers that are specific to a single sex.

In some embodiments, provided herein are systems, computer-readable media, and/or methods according to the foregoing or the following, wherein the correlation or regression comprises application of a regression analysis comprising glmnet-lasso, xgboost, and ranger.

In some embodiments, provided herein are systems, computer-readable media, and/or methods according to the foregoing or the following, wherein the age balancing step comprises not having more than n samples per age window of y years, beginning with age z years, wherein n, y, and z are integers >0; preferably, wherein n=5 or 6; y=7 years or 8 years; and z=16 years to 20 years; especially, wherein n=5, y=7 years and z=18 years.

In some embodiments, provided herein are systems, computer-readable media, and/or methods according to the foregoing or the following, wherein the machine-learning algorithm is based on Ridge Regression machine learning algorithm, which penalizes the size of parameter estimates by shrinking them to zero, in order to decrease complexity of the model while including all the variables in the model.

In some embodiments, provided herein are systems, computer-readable media, and/or methods according to the foregoing or the following, wherein the detection of methylation status comprises methylome by sequencing or methylation array analysis of the genomic DNA.

In some embodiments, provided herein are systems, computer-readable media, and/or methods according to the foregoing or the following, wherein the methylation status comprises level and/or amount of methylation markers or pattern of methylation markers in the biological sample.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the methylation markers are selected from the methylation markers in Table 1, wherein the structure of each methylation marker is provided by the respective Probe ID Nos., the nucleotide sequences and methylated residues therein, as indicated by nucleotides inside large parenthesis, is provided by the respective SEQ ID Nos.; or a gene linked to said methylation marker or locus thereto. Preferably, the methylation markers are listed in Table 1 in order of their relevance with calculated age of the biological sample. More preferably, the method comprises detecting a signature comprising about 10, 20, 30, 40, 50, 60, 70, 80, 100, 125, 150, 175, 200, 225, 250, 300 or all the markers from Table 1. Especially, the signature used in calculating the age includes markers having the highest relevance to age, wherein the markers are listed in Table 1 in decreasing order of relevance. That is, the markers are listed in Table 1 in order of the relative weights (or modifiers) that are applied to them (from highest to lowest) when they are used to calculate the age of the biological sample.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the plurality of methylation markers comprises markers having the C/G sequences set forth in Table 1. Preferably, the plurality of markers comprises about 10, 20, 30, 40, 50, 60, 70, 80, 100, 125, 150, 175, 200, 225, 250, 300 or all the markers from Table 1.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the plurality of methylation markers comprises markers having the C/G sequences set forth in Table 1. Preferably, the plurality of markers comprises about 1-10 markers, 1-20 markers, 1-30 markers, 1-40 markers, 1-50 markers, 1-60 markers, 1-70 markers, 1-80 markers, 1-90 markers, 1-100 markers, 1-125 markers, 1-150 markers, 1-175 markers, 1-200 markers, 1-225 markers, 1-250 markers, 1-275 markers, or 1-300 markers markers of Table 1.

Preferably, the methylation markers are listed in Table 1 in order of their relevance with the age of the biological sample. More preferably, the method comprises detecting a signature comprising about 10, 20, 30, 40, 50, 60, 70, 80, 100, 125, 150, 175, 200, 225, 250, 300, or all the markers from Table 1. Especially, the signature used in calculating the age includes markers having the highest relevance to age, wherein the markers are listed in Table 1 in decreasing order of relevance. That is, the markers are listed in Table 1 in order of the relative weights (or modifiers) that are applied to them (from highest to lowest) when they are used to calculate the age of the biological sample.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the methylation markers are selected from the methylation markers linked to at least one gene in Table 1 or a locus thereto. Preferably, the sequence identifier numbers (SEQ ID Nos.) of the methylation markers, as recited in Table 1, indicate relevance of the methylation marker with the age of the biological sample, wherein markers with smaller SEQ ID NO. are more relevant than markers with larger SEQ ID NO. That is, the sequence identifiers are listed in Table 1 in order of the relative weights (or modifiers) that are applied to them when they are used to calculate the age of the biological sample.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the methylation markers are selected from cg06279276 and cg00699993, wherein the structure of each methylation marker is provided by the respective Probe ID Nos., the nucleotide sequences and methylated residues therein, as indicated by nucleotides inside large parenthesis, is provided by the respective SEQ ID Nos., which are set forth in:

(a) CCGCCGCTGGTCCTTGGCGCGCAAATAGCGGGCGAAGTCAAAGGGTCCCG TAGGCGTGGG[CG]GCGCCGGTGTGTCCCCTTCGTAGGCCGGCGGGGCTGCACCCGCG TCGGGTAACTGGAACG(cg06279276); and

(b) CGCACGAAGGTAGCTCCGGGCGGGGAGCGAGGCGCTGTCCTCGGTGCTG AAAGGCCGAGG[CG]CGCGGTGGGCGCGACAGCCCCGGAGACCCGAGGTCTCGCGGA GGGACAGCGGCTACGGGC (cg00699993); or a gene linked to said methylation marker or locus thereto. Preferably, the methylation markers, in order of their relevance with calculated age of the biological sample, comprise both cg06279276 and cg00699993.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the methylation markers comprise at least one marker from cg06279276 and cg00699993 (preferably both) and at least one marker (preferably a plurality of markers) from cg17484671; cg11344566; cg24809973; cg03200166; cg06782035; cg02352240; cg25351606; cg07547549; cg03354992; cg00699993; cg02611848; cg07640648; cg18235734; cg06279276; cg00748589; cg23368787; cg02383785; cg02961707; cg15475851; cg07171111; cg05080154; cg03422911; cg14462779; cg16061498; cg04467618; cg02891686; cg12969644; cg25509871; cg09017434; cg17508941; cg12374721; cg11071401; cg06458239; cg05771369; cg25645064; cg14371731; cg19556343; cg22158769; cg10729426; cg16181396; cg00049664; cg13473356; cg05404236; cg16295725; cg21800232; cg23437843; cg24202131; cg15779837; cg04875128; cg06488443; cg24213719; cg25936177; cg17833476; cg12852499; cg18671949; cg16991515; cg06784991; cg00194126; cg00511674; cg08032924; cg18795809; cg18866015; cg10286969; cg21572722; cg23967544; cg11498607; cg14676592; cg10269365; cg01682111; cg10501210; cg27345346; cg08097417; cg19456540; cg04528819; cg10977667; cg19200589; cg23291886; cg10911990; cg06785999; cg24715245; cg18867659; cg10755058; cg07060233; cg18533201; cg03507326; cg06971096; cg26329178; cg24317217; cg24719321; cg14226702; cg03970036; cg21186299; cg15568145; cg06365535; cg01359962; cg07116393; cg13696942; cg09370594; cg25763393; cg24136205; cg06571559; cg13592721; cg23995459; cg23136139; cg11970349; cg06287137; cg21269897; cg18988435; cg14663984; cg18371700; cg12242474; cg26115667; cg23156348; cg13337731; cg09393254; cg02081006; cg06520675; cg00323305; cg10196902; cg21353911; cg21091227; cg19026977; cg08079908; cg02983163; cg21901946; cg17040303; cg09551472; cg13140267; cg11716026; cg25273520; cg06432426; cg24813736; cg17486097; cg26792755; cg26856080; cg06385324; cg04811592; cg03735496; cg14772615; cg24914355; cg13141009; cg14979301; cg09785958; cg26620450; cg21467631; cg20223728; cg24888989; cg06617961; cg25636665; cg11027140; cg24794228; cg05437148; cg18151345; cg06144905; cg10635145; cg21449170; cg01994205; cg15911409; cg03553786; cg24340081; cg13601993; cg18413131; cg07674022; cg08964780; cg23298047; cg08259925; cg24261921; cg13289553; cg26782833; cg18119885; cg04306050; cg11325997; cg00081714; cg24580076; cg24636999; cg25303383; cg01672943; cg07312601; cg12778178; cg16023306; cg05722918; cg22572614; cg10346212; cg14942863; cg03930964; cg05030953; cg27304144; cg12794224; cg17028652; cg24458609; cg26454158; cg15481429; cg08386537; cg19233923; cg01414572; cg06517429; cg06760904; cg00059424; cg11002227; cg25371803; cg20642765; cg08734053; cg11567723; cg16897193; cg23021855; cg08261702; cg18088844; cg11594299; cg16025094; cg15309223; cg05156137; cg03335886; cg01717881; cg03031988; cg04738656; cg23229770; cg07299526; cg20355806; cg02268620; cg26050838; cg05335473; cg13009608; cg04631458; cg26777345; cg22946147; cg22425860; cg00151919; cg19255191; cg22872989; cg10286959; cg21877956; cg17279592; cg02064158; cg25584787; cg09113665; cg13282195; cg03873281; cg00841725; cg16758041; cg12528144; cg19136783; cg00798886; cg11732282; cg12213687; cg16937168; cg14866740; cg18703066; cg19772114; cg07139350; cg13614741; cg04172115; cg01146808; cg06826289; cg23124451; cg05200380; cg00874055; cg00307483; cg09165041; cg05266663; cg13868165; cg21943004; cg15577927; cg13159054; cg04056904; cg12373003; cg11510999; cg02291532; cg26376566; cg14101501; cg18268220; cg11457534; cg25463688; cg09643312; cg12682862; cg20145610; cg07608813; cg19359218; cg11251319; cg07417733; cg10316834; cg25548869; cg04775710; cg01885291; cg00356811; cg05238905; cg12612947; cg15921240; cg04195863; cg09822726; cg10645314; cg03705220; cg05020775; cg07023563; cg27511169; cg03209395; cg23288827; cg08984586; cg03835983; cg04808059; and cg08540010; or a gene linked to said methylation marker or locus thereto. Particularly, the additional methylation marker includes a plurality, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 70, 75, 80, 85, 90, 95, 100, 110, 120, 130, 140, 150, 160, 170, 180, 190, 200, 220, 240, 260, 280, 300, or all of the foregoing markers. Preferably, the methylation markers herein are listed in order of their association with age of the biological sample. That is, the markers are listed herein in order of the relative weights (or modifiers) that are applied to them when they are used to calculate the age of the biological sample.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the methylation markers comprise at least one marker from;

cg17484671; cg11344566; cg24809973; cg03200166; cg06782035; cg02352240; cg25351606; cg07547549; cg03354992; cg00699993; cg02611848; cg07640648; cg18235734; cg06279276; cg00748589; cg23368787; cg02383785; cg02961707; cg15475851; cg07171111; cg05080154; cg03422911; cg14462779; cg16061498; cg04467618; cg02891686; cg12969644; cg25509871; cg09017434; cg17508941; cg12374721; cg11071401; cg06458239; cg05771369; cg25645064; cg14371731; cg19556343; cg22158769; cg10729426; cg16181396; cg00049664; cg13473356; cg05404236; cg16295725; cg21800232; cg23437843; cg24202131; cg15779837; cg04875128; cg06488443; cg24213719; cg25936177; cg17833476; cg12852499; cg18671949; cg16991515; cg06784991; cg00194126; cg00511674; cg08032924; cg18795809; cg18866015; cg10286969; cg21572722; cg23967544; cg11498607; cg14676592; cg10269365; cg01682111; cg10501210; cg27345346; cg08097417; cg19456540; cg04528819; cg10977667; cg19200589; cg23291886; cg10911990; cg06785999; cg24715245; cg18867659; cg10755058; cg07060233; cg18533201; cg03507326; cg06971096; cg26329178; cg24317217; cg24719321; cg14226702; cg03970036; cg21186299; cg15568145; cg06365535; cg01359962; cg07116393; cg13696942; cg09370594; cg25763393; cg24136205; cg06571559; cg13592721; cg23995459; cg23136139; cg11970349; cg06287137; cg21269897; cg18988435; cg14663984; cg18371700; cg12242474; cg26115667; cg23156348; cg13337731; cg09393254; cg02081006; cg06520675; cg00323305; cg10196902; cg21353911; cg21091227; cg19026977; cg08079908; cg02983163; cg21901946; cg17040303; cg09551472; cg13140267; cg11716026; cg25273520; cg06432426; cg24813736; cg17486097; cg26792755; cg26856080; cg06385324; cg04811592; cg03735496; cg14772615; cg24914355; cg13141009; cg14979301; cg09785958; cg26620450; cg21467631; cg20223728; cg24888989; cg06617961; cg25636665; cg11027140; cg24794228; cg05437148; cg18151345; cg06144905; cg10635145; cg21449170; cg01994205; cg15911409; cg03553786; cg24340081; cg13601993; cg18413131; cg07674022; cg08964780; cg23298047; cg08259925; cg24261921; cg13289553; cg26782833; cg18119885; cg04306050; cg11325997; cg00081714; cg24580076; cg24636999; cg25303383; cg01672943; cg07312601; cg12778178; cg16023306; cg05722918; cg22572614; cg10346212; cg14942863; cg03930964; cg05030953; cg27304144; cg12794224; cg17028652; cg24458609; cg26454158; cg15481429; cg08386537; cg19233923; cg01414572; cg06517429; cg06760904; cg00059424; cg11002227; cg25371803; cg20642765; cg08734053; cg11567723; cg16897193; cg23021855; cg08261702; cg18088844; cg11594299; cg16025094; cg15309223; cg05156137; cg03335886; cg01717881; cg03031988; cg04738656; cg23229770; cg07299526; cg20355806; cg02268620; cg26050838; cg05335473; cg13009608; cg04631458; cg26777345; cg22946147; cg22425860; cg00151919; cg19255191; cg22872989; cg10286959; cg21877956; cg17279592; cg02064158; cg25584787; cg09113665; cg13282195; cg03873281; cg00841725; cg16758041; cg12528144; cg19136783; cg00798886; cg11732282; cg12213687; cg16937168; cg14866740; cg18703066; cg19772114; cg07139350; cg13614741; cg04172115; cg01146808; cg06826289; cg23124451; cg05200380; cg00874055; cg00307483; cg09165041; cg05266663; cg13868165; cg21943004; cg15577927; cg13159054; cg04056904; cg12373003; cg11510999; cg02291532; cg26376566; cg14101501; cg18268220; cg11457534; cg25463688; cg09643312; cg12682862; cg20145610; cg07608813; cg19359218; cg11251319; cg07417733; cg10316834; cg25548869; cg04775710; cg01885291; cg00356811; cg05238905; cg12612947; cg15921240; cg04195863; cg09822726; cg10645314; cg03705220; cg05020775; cg07023563; cg27511169; cg03209395; cg23288827; cg08984586; cg03835983; cg04808059; and cg08540010, or a gene linked to said methylation marker or locus thereto. Preferably, the methylation markers herein are listed in order of their association with age of the biological sample. That is, the markers are listed herein in order of the relative weights (or modifiers) that are applied to them when they are used to calculate the age of the biological sample.

In some embodiments, the disclosure relates to a method for calculating an age of a biological sample, comprising, detecting, status of methylation markers in a genomic DNA (gDNA) of the biological sample; and determining the age of the sample based on the status of the detected methylation markers, wherein the methylation markers comprise cg06279276 or cg00699993 (preferably both); or a gene linked to the methylation marker or locus thereto.

In some embodiments, the disclosure relates to a method for calculating an age of a biological sample, comprising, detecting, status of methylation markers in a genomic DNA (gDNA) of the biological sample; and determining the age of the sample based on the status of the detected methylation markers, wherein the methylation markers comprise a plurality of methylation markers that are listed in order of their association with age of the biological sample, the methylation markers are selected from the markers in Table 1; or a gene linked to said methylation marker or locus thereto.

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the plurality of methylation markers comprises methylation markers in gene B3GNT9, or a locus thereto, or GRIA2, or a locus thereto (preferably both).

In some embodiments, the disclosure relates to a method for calculating an age of a tissue specific biological sample, comprising, detecting, status of methylation markers in a genomic DNA (gDNA) of the biological sample; and determining the age of the sample based on the status of the detected methylation markers, wherein the plurality of methylation markers comprises methylation markers in a gene selected from CNTNAP5; SYT7; MARCH11; SLC12A5; GRIA2; C2orf65; DLL3; B3GNT9; ATP4A; EVI5L; INA; SALL3; RYR2; DUPD1; TCF21; SOD3; RASEF; PLD3; C17orf93; PRAC; CACNA1G; ZNF549; B4GALNT1; ZMIZ1; NCAM2; LOC375196; LOC100271715; ZIC1; CMTM2; PEX5L; IRS2; ZNF518B; ANKRD34B; ZNF167; BRUNOL4; GRIN2D; OTUD7A; TBR1; TLX3; LOC728392; HIST1H2BK; ZYG11A; NR4A2; ZNF518B; DCC; PRSS27; ELOVL2; RUNX1; CCDC140; UNKL; C19orf55; SIX6; CLIC6; PAX9; UCHL1; NETO2; ENTPD3; SLC12A5; GDF6; LOC100128788; SRRM2; PTPRN; HPSE2; BSX; PTPRN; VGF; PRDM2; TBX4; C3orf39; MUL1; DBX1; LINGO3; ZNF578; ZIC5; DIP2C; HIST1H4I; ZYG11B; RASGEF1A; GPR78; DNAJC5G; AGRN; CLIC6; SDCBP2; TRAF3; MLXIPL; MCHR2; PRDM6; F1141350; THRB; SIM2; POM121L2; SNRNP200; H19; UNC5D; MRPS33; TRIM59; SNHG9; SNORA78; RPS2; MITF; GREB1L; HOXD13; PEX5L; P2RX2; NRN1; KIF15; KIAA1143; MIR1826; CTNNA2; GPR144; ZNF577; FBRS; SLC15A3; PIPDX; BDNF; KLF14; POU4F1; CXCR7; LOC285375; NKAIN3; NR6A1; NUDT16P; TRPC3; MIR196B; HTR1A; SLC6A20; SUB1; AMMECR1L; ATP5G3; AMH; C7orf20; DNAH8; BCO2; PAX9; MRTO4; UCKL1AS; UCKL1; POP4; SLC5A8; TNFSF10; BCR; HLA-C; HSPG2; AKAP12; ADRB1; LRRC55; ZNF136; MCTP2; LOC440925; OTUB1; CASP7; MYT1L; PES1; GMPS; CCT3; Clorf182; MLF2; NOVA2; APLF; FBXO48; LOC728743; GIPR; RADIL; CPLX2; TMEM59; C1orfi83; RCAN1; GJB6; RPH3AL; BAT1; CCDC87; CCS; DPEP1; MIR24-1; C9orf3; CASP2; TPD52; ZNF804B; MGC26647; SLC25A15; COX5B; CD164L2; ME1; WDR27; RTN4RL1; C5orf36; TMEM188; NAPRT1; PDLIM4; MCF2L; NDUFB6; LDB2; DHX29; SKIV2L2; ARL6IP6; PRPF40A; COL4A1; SNED1; CDC40; WASF1; VPS13D; ZNF783; TNXB; PRDM1; GLT1D1; CBX7; GPR137B; WASF2; LOC728448; EPHB2; FAM19A5; OR4D11; ISM1; ITGB7; THBS1; PSEN1; EHBP1; SLC38A6; IGSF9B; CD302; RARS; MCOLN1; TRIM26; ATP8B3; MCM4; PRKDC; HLA-A; IER3; TNFAIP8L1; PPIL4; TOP2B; ZNF141; SNRPN; SNURF; TANC2; ALLC; LHX3; SNPH; ARHGEF10L; GOLSYN; SPNS2; RNF44; COL9A3; TOX2; TMEM189; and TMEM189-UBE2V1; or a locus linked to the gene.

In some embodiments, the disclosure relates to a method for determining an age of a tissue specific biological sample comprising ovaries, testis, kidney, skin, blood, saliva, sperm, heart, brain, kidney, or liver sample. In some embodiments, the disclosure relates to a method for determining an age of a tissue specific biological sample comprising epidermal or dermal cells or fibroblasts. Particularly under these embodiments, the detection of the status of methylation markers comprises detection of a level or pattern of methylation markers.

In some embodiments, the disclosure relates to a method for determining an age of a tissue specific biological sample comprising methylation sequencing of a DNA (e.g., DNA) obtained from a biological sample, e.g., ovaries, testis, kidney, skin, blood, saliva, sperm, heart, brain, kidney, or liver. Preferably, the sample is obtained from a human, e.g., human patient.

In some embodiments, the disclosure relates to a kit for calculating an age of a biological sample, comprising, probes for detecting, status of methylation markers in a genomic DNA (gDNA) of the biological sample; vessels for holding the biological sample; optionally together with instructions for performing the detection, wherein the methylation markers comprises a plurality of the methylation markers of Table 1; or a gene linked to the methylation marker or a locus thereto. Preferably, the kit comprises probes for detecting a plurality of markers comprising about 5, 10, 20, 30, 40, 50, 60, 70, 80, 90, 100, 125, 150, 175, 200, 225, 250, 275, 300, or all the markers from Table 1.

In some embodiments, the disclosure relates to a kit for calculating an age of a biological sample, comprising, probes for detecting status of methylation markers in a genomic DNA (gDNA) of the biological sample; vessels for holding the biological sample; optionally together with instructions for performing the detection, wherein the methylation markers comprises cg06279276 and cg00699993, preferably both cg06279276 and cg00699993; or the methylation status of a gene linked to the methylation marker or a locus thereto.

In some embodiments, the disclosure relates to a kit for calculating an age of a biological sample, comprising, probes for detecting, status of methylation markers in a genomic DNA (gDNA) of the biological sample; vessels for holding the biological sample; optionally together with instructions for performing the detection, wherein the methylation markers comprise at least 20 methylation markers listed in Table 1, wherein the structure of each methylation marker is provided by the respective ILLUMINA Probe ID Nos., the nucleotide sequences and methylated residues therein, as indicated by nucleotides inside large parentheses, is provided by the respective SEQ ID Nos., and optionally by the recited gene or a locus to the gene.

Preferably, the kits comprise probes for detecting a plurality of methylation markers comprising at least 5, 10, 20, 30, 40, 50, 60, 70, 80, 90, 100, 125, 150, 175, 200, 225, 250, 275, 300, or all the markers from Table 1. Particularly, the kits comprise probes for detecting a plurality of methylation markers comprising markers having the nucleic acid sequences set forth in (1) SEQ ID Nos: 1-20; (2) SEQ ID Nos: 1-40; (3) SEQ ID Nos: 1-60; (4) SEQ ID Nos: 1-80; (5) SEQ ID Nos: 1-100; (6) SEQ ID Nos: 1-120; (7) SEQ ID Nos: 1-140; (8) SEQ ID Nos: 1-160; (9) SEQ ID Nos: 1-180; (10) SEQ ID Nos: 1-200; (11) SEQ ID Nos: 1-220; (12) SEQ ID Nos: 1-240; (13) SEQ ID Nos: 1-260; (14) SEQ ID Nos: 1-280; or (15) SEQ ID Nos: 1-300. Especially, the kits comprise probes for detecting a plurality of methylation markers comprising all the markers of Table 1.

The disclosure relates to kits for calculating an age of a biological sample, comprising probes for detecting status of methylation markers in a genomic DNA (gDNA) of the biological sample; vessels for holding the biological sample; optionally together with instructions for performing the detection, wherein the methylation markers are selected from cg17484671; cg11344566; cg24809973; cg03200166; cg06782035; cg02352240; cg25351606; cg07547549; cg03354992; cg00699993; cg02611848; cg07640648; cg18235734; cg06279276; cg00748589; cg23368787; cg02383785; cg02961707; cg15475851; cg07171111; cg05080154; cg03422911; cg14462779; cg16061498; cg04467618; cg02891686; cg12969644; cg25509871; cg09017434; cg17508941; cg12374721; cg11071401; cg06458239; cg05771369; cg25645064; cg14371731; cg19556343; cg22158769; cg10729426; cg16181396; cg00049664; cg13473356; cg05404236; cg16295725; cg21800232; cg23437843; cg24202131; cg15779837; cg04875128; cg06488443; cg24213719; cg25936177; cg17833476; cg12852499; cg18671949; cg16991515; cg06784991; cg00194126; cg00511674; cg08032924; cg18795809; cg18866015; cg10286969; cg21572722; cg23967544; cg11498607; cg14676592; cg10269365; cg01682111; cg10501210; cg27345346; cg08097417; cg19456540; cg04528819; cg10977667; cg19200589; cg23291886; cg10911990; cg06785999; cg24715245; cg18867659; cg10755058; cg07060233; cg18533201; cg03507326; cg06971096; cg26329178; cg24317217; cg24719321; cg14226702; cg03970036; cg21186299; cg15568145; cg06365535; cg01359962; cg07116393; cg13696942; cg09370594; cg25763393; cg24136205; cg06571559; cg13592721; cg23995459; cg23136139; cg11970349; cg06287137; cg21269897; cg18988435; cg14663984; cg18371700; cg12242474; cg26115667; cg23156348; cg13337731; cg09393254; cg02081006; cg06520675; cg00323305; cg10196902; cg21353911; cg21091227; cg19026977; cg08079908; cg02983163; cg21901946; cg17040303; cg09551472; cg13140267; cg11716026; cg25273520; cg06432426; cg24813736; cg17486097; cg26792755; cg26856080; cg06385324; cg04811592; cg03735496; cg14772615; cg24914355; cg13141009; cg14979301; cg09785958; cg26620450; cg21467631; cg20223728; cg24888989; cg06617961; cg25636665; cg11027140; cg24794228; cg05437148; cg18151345; cg06144905; cg10635145; cg21449170; cg01994205; cg15911409; cg03553786; cg24340081; cg13601993; cg18413131; cg07674022; cg08964780; cg23298047; cg08259925; cg24261921; cg13289553; cg26782833; cg18119885; cg04306050; cg11325997; cg00081714; cg24580076; cg24636999; cg25303383; cg01672943; cg07312601; cg12778178; cg16023306; cg05722918; cg22572614; cg10346212; cg14942863; cg03930964; cg05030953; cg27304144; cg12794224; cg17028652; cg24458609; cg26454158; cg15481429; cg08386537; cg19233923; cg01414572; cg06517429; cg06760904; cg00059424; cg11002227; cg25371803; cg20642765; cg08734053; cg11567723; cg16897193; cg23021855; cg08261702; cg18088844; cg11594299; cg16025094; cg15309223; cg05156137; cg03335886; cg01717881; cg03031988; cg04738656; cg23229770; cg07299526; cg20355806; cg02268620; cg26050838; cg05335473; cg13009608; cg04631458; cg26777345; cg22946147; cg22425860; cg00151919; cg19255191; cg22872989; cg10286959; cg21877956; cg17279592; cg02064158; cg25584787; cg09113665; cg13282195; cg03873281; cg00841725; cg16758041; cg12528144; cg19136783; cg00798886; cg11732282; cg12213687; cg16937168; cg14866740; cg18703066; cg19772114; cg07139350; cg13614741; cg04172115; cg01146808; cg06826289; cg23124451; cg05200380; cg00874055; cg00307483; cg09165041; cg05266663; cg13868165; cg21943004; cg15577927; cg13159054; cg04056904; cg12373003; cg11510999; cg02291532; cg26376566; cg14101501; cg18268220; cg11457534; cg25463688; cg09643312; cg12682862; cg20145610; cg07608813; cg19359218; cg11251319; cg07417733; cg10316834; cg25548869; cg04775710; cg01885291; cg00356811; cg05238905; cg12612947; cg15921240; cg04195863; cg09822726; cg10645314; cg03705220; cg05020775; cg07023563; cg27511169; cg03209395; cg23288827; cg08984586; cg03835983; cg04808059; and cg08540010; wherein the structure of each methylation marker is provided by the respective Probe ID Nos., the nucleotide sequences and methylated residues therein, as indicated by nucleotides inside large parentheses, is provided by the respective SEQ ID Nos., or a gene linked to said methylation marker or locus thereto. Preferably, the kits comprise probes for detecting the methylation markers cg06279276 and/or cg00699993 or a gene linked to said methylation marker or locus thereto; especially probes for detecting both cg06279276 and cg00699993 or a gene linked to said methylation marker or locus thereto. In some embodiments, the kits comprise probes specific for markers listed herein in order of the relative weights (or modifiers) that are applied to the markers when they are used to calculate the age of the biological sample.

In some embodiments, the disclosure relates to a computer readable medium comprising computer-executable instructions, which, when executed by a processor, cause the processor to carry out a method or a set of steps for identifying methylation markers in a genetic dataset received from a subject's sample, wherein the methylation markers comprises a level or pattern of methylation in the genomic DNA (gDNA), the medium comprising machine learning techniques to calculate linear regression coefficients to methylation markers. In some embodiments, the algorithm is trained with a compendium of methylation markers each of which is annotated with age and the algorithm computes the predictive power of each marker using a rigorous mathematical algorithm. Particularly, the algorithm comprises a regression model comprising a machine learning algorithm, e.g., the Ridge Regression machine learning algorithm, which penalizes the size of parameter estimates by shrinking them to zero in order to decrease complexity of the model, while including all the variables in the model.

In certain embodiments, determining the age of the biological sample may comprise applying a linear regression model to predict sample age based on a weighted average of the methylation marker levels plus an offset. In some embodiments, a first predicted age is determined based on the methylation status and a second predicted age is determined by performing an operation (e.g., addition or subtraction) on the first predicted age. Specifically, the operation comprises an addition or subtraction of a delta age (δ), derived from a validation dataset of samples obtained from the subject, e.g., as provided in a hash table of Table 4. In such embodiments, the second predicted age may provide a more accurate estimate of the actual age of the sample. In some embodiments, prediction or calculation of the age is performed using a regression model, e.g., using a regression curve shown in FIG. 5.

In some embodiments, the disclosure relates to a system for identifying an age of a biological sample, comprising: (a) an optional counter configured to count numbers and/or levels of methylation markers in a genomic DNA (gDNA) of the biological sample and output a methylation data of the sample, wherein the methylation markers comprises the markers listed in Table 1, wherein the structure of each methylation marker is provided by the respective ILLUMINA Probe ID Nos., the nucleotide sequences and methylated residues therein, as indicated by nucleotides inside large parenthesis, is provided by the respective SEQ ID Nos.; and (b) a computing device comprising, (1) a methylation analyzer that is configured to detect patterns and/or levels of methylation markers in the sample's methylation data, wherein the analyzer is communicatively connected to the counter when the counter is present; (2) an age identifier engine configured to predict age of the sample based on the patterns and/or levels of methylation markers; and (3) a display communicatively connected to the computing device and configured to display a report containing the biological sample's predicted age. Preferably in the systems of the disclosure, the plurality of methylation markers comprises at least 5, 10, 20, 30, 40, 50, 60, 70, 80, 90, 100, 125, 150, 175, 200, 225, 250, 275, or all the markers (e.g., 300) from Table 1.

In some embodiments, the disclosure relates to a method of screening an anti-aging agent, comprising, contacting the agent with a cell for a period sufficient to induce epigenetic changes in the cell; determining a modulation of a plurality of methylation markers selected from methylation markers of Table 1 in the cell; and selecting the test agent based on the modulation of the methylation markers. Preferably, the screening methods include determining a modulation of a plurality of methylation markers comprising at least 5, 10, 20, 30, 40, 50, 60, 70, 80, 90, 100, 125, 150, 175, 200, 225, 250, 275, or all the markers (e.g., 300) from Table 1 in the cell; and selecting the test agent based on the modulation of the methylation markers. Especially, the screening methods include determining a modulation of all of the methylation markers in Table 1 in the cell; and selecting the test agent based on the modulation of the methylation markers.

In some embodiments, the plurality of methylation markers comprises markers having the C/G sequences set forth in (1) SEQ ID Nos: 1-20; (2) SEQ ID Nos: 1-40; (3) SEQ ID Nos: 1-60; (4) SEQ ID Nos: 1-80; (5) SEQ ID Nos: 1-100; (6) SEQ ID Nos: 1-120; (7) SEQ ID Nos: 1-140; (8) SEQ ID Nos: 1-160; (9) SEQ ID Nos: 1-180; (10) SEQ ID Nos: 1-200; (11) SEQ ID Nos: 1-220; (12) SEQ ID Nos: 1-240; (13) SEQ ID Nos: 1-260; (14) SEQ ID Nos: 1-280; or (15) SEQ ID Nos: 1-300.

In some embodiments, the modulation comprises increase in methylation levels. In some embodiments, the modulation comprises a reduction in methylation levels. In some embodiments, the cell is a skin cell, e.g., a fibroblast cell or keratinocyte cell.

In some embodiments, the disclosure relates to a method for identifying a subject for aging or having an age-related disease comprising: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating the age of the subject's biological sample based on the status of the detected methylation markers, wherein if the calculated age of the sample is greater than the subject's actual age, then the subject is positively identified as aging or having an age-related disease.

In some embodiments, the disclosure relates to a method of prognosticating a subject for developing aging or an age-related disease comprise the following steps: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues there, as indicated by nucleotides inside large parenthesis, is provided by the respective SEQ ID Nos., or a gene linked to the methylation marker or a locus thereto; (b) calculating the age of the subject's biological sample based on the status of the detected methylation markers, wherein if the calculated age of the sample is greater than the subject's actual age, then the subject is prognosticated as being at risk for developing aging or an age-related disease.

In some embodiments, the disclosure relates to a method for determining the efficacy of a drug or a therapy against aging or an age-related disease comprise the following steps: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating a first calculated age of the subject's biological sample based on the status of the detected methylation marker; (c) administering to the subject, an anti-aging drug or therapy if the first calculated age of the subject's sample is greater than the subject's actual age; (d) detecting the status of a plurality of the methylation markers of (a) in the genomic DNA (gDNA) of the biological sample of the subject treated with the anti-aging drug or therapy and calculating a second calculated age of the test compound-contacted biological sample based on the status of the methylation markers detected in (a); and (e) determining the effectiveness of the anti-aging drug or therapy based on the modulation of the second calculated age compared to the first calculated age.

In some embodiments, the modulation comprises increase in methylation levels. In some embodiments, the modulation comprises a reduction in methylation levels. In some embodiments, the cell is a skin cell, e.g., a fibroblast cell or keratinocyte cell.

### BRIEF DESCRIPTION OF THE DRAWINGS

The details of one or more embodiments of the disclosure are set forth in the accompanying drawings/tables and the description below. Other features, objects, and advantages of the disclosure will be apparent from the drawings/tables and detailed description, and from the claims.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are purely representative and do not limit the disclosure.

FIG. 1 illustrates an exemplary experimental design of the age-prediction methodology of the present disclosure.

FIG. 2A and FIG. 2B respectively shows Beta values of the dataset before and after the preprocessing and normalization steps, using the systems and methods of the disclosure.

FIG. 3A and FIG. 3B respectively shows age distribution between the training and testing datasets, using the systems and methods of the disclosure.

FIG. 4 shows performance comparison of the models of the systems and methods of the disclosure. FIG. 4 shows mean absolute error (MAE) and/or root mean squared error (RMSE), along with fitness levels and significance of the indicated regression models, as evaluated by computing Pearson's correlation coefficient using the training data (e.g., smaller MAE or RMSE scores indicate better predictive algorithm and an R² value that ˜1.0 indicates better fit).

FIG. 5A, FIG. 5B, and FIG. 5C show results of age-prediction analysis, as determined by the systems and methods of the disclosure, using the testing dataset of 259 samples, containing 300 predictors. FIG. 5A shows the correlation between predicted and chronological age (R=0.91; p=<2.2E-16, with a RMSE of 5.16 years). FIG. 5B and FIG. 5C show that when evaluating the same testing dataset, better accuracy was obtained with epidermis only samples (R=0.97; p<2.2E-16) (FIG. 5B) as compared to whole skin samples (R=0.82; p<2.2E-16) (FIG. 5C), when the samples were split according to the tissue source.

FIG. 6 shows a bar chart of the relative importance (or relevance) of top 100 probes for calculating age of biological samples, as determined using the systems and methods of the disclosure.

FIG. 7A, FIG. 7B, and FIG. 7C show scatter plots showing correlation between the predicted age, as determined using the methods of the present disclosure (FIG. 7A) and prior methods (FIG. 7B and FIG. 7C), and the chronological age of an independent set of skin samples. A statistically significant association between the predicted age and chronological age was observed with the instant methods and systems (Pearson correlation coefficient (PCC) r=0.96; p=8.2×10⁻⁹). Using the same external dataset of skin biopsies, it was established that the power of the instant methods to accurately predict age was also superior to prior methods such as Horvath Molecular Clocks (1^{st} Horvath Molecular Clock: PCC r=0.9; p=2.5×10⁻⁶ (FIG. 7B); 2^{nd} Horvath Molecular Clock: PCC r=0.95; p=1.4×10⁻⁸ (FIG. 7C)).

FIG. 8A and FIG. 8B show applications of the systems and methods of the disclosure. FIG. 8A shows the ability of the of the systems and methods of the disclosure to predict age differences in fibroblast (FB) monoculture obtained from donors of different age was evaluated (29y means the cell donor was 29 years old, 84y means the cell donor was 84 years old, and p22 means the cell passage number is 22). FIG. 8B shows the ability of the systems and methods of the disclosure to detect the effect of cell passaging on cell culture from the same donor (p11 means the cell passage number is 11 and p19 means the cell passage number is 19).

FIG. 9 shows a diagram of the computer system of the present disclosure.

FIG. 10 shows a schematic chart of the method of the disclosure.

FIG. 11A, FIG. 11B, FIG. 11C and FIG. 11D show schematic representations of the system(s) of the disclosure. FIG. 11A shows a schematic representation of an integrated system.

FIG. 11B shows a schematic representation of a semi-integrated system. FIG. 11C shows a schematic representation of a semi-discrete system. FIG. 11D shows a schematic representation of a discrete system.

FIG. 12 shows an embodiment of the specific workflow of the disclosure.

FIG. 13 shows an exemplary Age Prediction/Calculation tool of the present disclosure.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements A, B, C), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. The terminology used in the description of the disclosure herein is for describing particular embodiments only and is not intended to be limiting of the disclosure. Further, unless otherwise required by context, singular terms shall include pluralities and plural terms shall include the singular. Generally, nomenclatures utilized in connection with, and techniques of molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (3^{rd} ed., Cold Spring Harbor Laboratory Press, Cold Spring Harbor, N.Y. 2000); J. Perbal et al., A Practical Guide to Molecular Cloning, John Wiley and Sons (1984); Brown (Ed), Essential Molecular Biology: A Practical Approach, Volumes 1 and 2, JUL Press (1991); Glover & Hames (Eds.), Current Protocols in Molecular Biology, Greene Pub. Associates (1988); Harlow & Lane (Eds.) Antibodies: A Laboratory Manual, Cold Spring Harbor Laboratory, (1988), and Coligan et al. (Eds.) Current Protocols in Immunology, John Wiley & Sons (1988).

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features. For example, one of skill in the art would be aware of “linkage disequilibrium” which relates to the non-random association of alleles at two or more loci that descend from single, ancestral chromosomes. As outlined below the present disclosure describes a methylation status comprising a series of CpG sites associated with aging or the propensity for aging. The CpG sites of the present disclosure include related sites in linkage disequilibrium. Moreover, determining the methylation status of the CpG sites of the present disclosure includes determining the methylation status of other markers in linkage disequilibrium with the particular CpG sites.

The in vitro methods of the present disclosure can be performed as an assay. As one of skill in the art would appreciate, an assay is an investigative (analytic) procedure or method for qualitatively assessing or quantitatively measuring the presence or amount or the functional activity of a target. For example, an assay can assess methylation of various CpG sites.

In an example, a method or assay according to the present disclosure may be incorporated into a treatment regimen. For example, a method of treating aging in a subject in need thereof may comprise performing an assay that embodies the methods of the present disclosure. In an example, a clinician or similar may wish to perform or request performance of an assay according to the present disclosure before administering or modifying treatment to a patient. For example, a clinician may perform or request performance of an assay according to the present disclosure on a subject before electing to administer or modify therapy such as caloric restriction. In another example, a method or assay according to the present disclosure may be incorporated in an R&D experiment. For example, a method of detecting the effect of a specific molecule over the molecular age of a biological sample may comprise performing an assay that embodies the methods of the present disclosure. In an example, the molecule that promotes the higher age reversal may be chosen from a group of molecules according to the data generated by an assay that embodies the methods of the present disclosure.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be expressly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following descriptions.

The methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software, including, software on cloud. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions, which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Methylation sequencing technology enables research on a large scale. Particularly, the methods and systems of the disclosure can utilize de-identified, clinical information and biological data for medically relevant associations. The methods and systems disclosed can comprise a high-throughput platform for discovering and validating epigenetic factors that cause or influence a range of diseases, e.g., aging. The disclosure provides an objective method for monitoring such diseases, such as progression, deceleration, and even regression of aging.

The various embodiments of the present disclosure are further described in detail in the paragraphs below.

### Definitions

As used in the description of the disclosure and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The word “about” means a range of plus or minus 10% of that value, e.g., “about 5” means 4.5 to 5.5, “about 100” means 90 to 110, etc., unless the context of the disclosure indicates otherwise, or is inconsistent with such an interpretation. For example in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Where a range of values is provided in this disclosure, it is intended that each intervening value between the upper and lower limit of that range and any other stated or intervening value in that stated range is encompassed within the disclosure. For example, if a range of 1 μM to 8 μM is stated, it is intended that 2 μM, 3 μM, 4 μM, 5 μM, 6 μM, and 7 μM are also explicitly disclosed.

As used herein, the term “plurality” can be 2, 3, 4, 5, 6, 7, 8, 9, 10, 15, 20, 30, 40, 50, 75, 100, 125, 150, 175, 200, 225, 250, 275, 300, or more entities (e.g., markers). Preferably, the term “plurality” means at least 10, 20, 50, 100, 125, 150, 175, 200, 225, 250, 275, or 300 (+/−25) entities.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within 10%, or within 5% or less, e.g., with 2%.

As used herein, the term “detecting,” refers to the process of determining a value or set of values associated with a sample by measurement of one or more parameters in a sample, and may further comprise comparing a test sample against reference sample. In accordance with the present disclosure, the detection of tumors includes identification, assaying, measuring and/or quantifying one or more markers.

As used herein, the term “diagnosis” refers to methods by which a determination can be made as to whether a subject is likely to be suffering from a given disease or condition, including but not limited diseases or conditions characterized by genetic variations. The skilled artisan often makes a diagnosis based on one or more diagnostic indicators, e.g., a marker, the presence, absence, amount, or change in amount of which is indicative of the presence, severity, or absence of the disease or condition. Other diagnostic indicators can include patient history; physical symptoms, e.g., weight loss, osteoporosis, vision loss; phenotype; genotype; or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given characteristic.

As used herein, “biological data” can refer to any data derived from measuring biological conditions of human tissues or organs, animals or other biological organisms including plants and microorganisms. The measurements may be made by any tests, assays or observations that are known to physicians, scientists, diagnosticians, or the like. Biological data can include, but is not limited to, clinical tests and observations, physical and chemical measurements, genomic determinations, genomic sequencing data, exome sequencing data, methylome sequencing data, epigenetic data (e.g., EPIGENIE), proteomic determinations, drug levels, hormonal and immunological tests, neurochemical or neurophysical measurements, mineral and vitamin level determinations, genetic and familial histories, and other determinations that may give insight into the state of the individual or individuals that are undergoing testing. As used herein, “phenotypic data” refer to data about phenotypes. Phenotypes are discussed further below.

As used herein, the term “subject” means an individual. In one aspect, a subject is a mammal such as a human. In one aspect, a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., cows, pigs, goats), laboratory animals (e.g., mouse, rabbit, rat, gerbil, guinea pig, etc.) and avian species (e.g., chickens, turkeys, ducks, etc.). Subjects can also include, but are not limited to fish (for example, zebrafish, goldfish, tilapia, salmon, and trout), amphibians and reptiles. Preferably, the subject is a human subject. Especially, the subject is a human patient.

The term “age-associated disorder” in the context of a “subject” is used to describe a disorder observed with the biological progression of events occurring over time in a subject. Preferably, the subject is a human. Non-limiting examples of age-associated disorders include, but are not limited to, hypertension, atherosclerosis, diabetes mellitus, dementia, skin disorders or structural alterations. An age-associated disorder may also be a cell proliferative disorder. Examples of age-associated disorders that are cell proliferative disorders include colon cancer, lung cancer, breast cancer, prostate cancer, and melanoma, amongst others. An age-associated disorder is further intended to mean the biological progression of events that occur during a disease process that affects the body, which mimic or substantially mimic all or part of the aging events which occur in a normal subject, but which occur in the diseased state over a shorter period. Particularly, the age-associated disorder is a “memory disorder” or “learning disorder” which is characterized by a statistically significant decrease in memory or learning assessed over time. In some embodiments, the age-associated disorder is a skin disorder, e.g., wrinkles, lines, dryness, itchiness, age-spots, bedsores, dyspigmentation, infection (e.g., fungal infection), and/or a reduction in a skin property selected from clarity, texture, elasticity, color, tone, pliability, firmness, tightness, smoothness, thickness, radiance, evenness, laxity, and oiliness.

The term “sample” as used herein refers to a composition that is obtained or derived from a subject of interest that contains a cellular and/or other molecular entity that is to be characterized and/or identified, for example based on physical, biochemical, chemical and/or physiological characteristics. Preferably, the sample is a “biological sample,” which means a sample that is derived from a living entity, e.g., cells, tissues, organs, in vitro engineered organs and the like. In some embodiments, the source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or 2D and 3D cultured cells or cell lines, cell supernatants, cell lysates, platelets, serum, plasma, vitreous fluid, ocular fluid, lymph fluid, synovial fluid, follicular fluid, seminal fluid, amniotic fluid, milk, whole blood, urine, cerebrospinal fluid (CSF), saliva, sputum, tears, perspiration, mucus, tumor lysates, skin punch or biopsy, and tissue culture medium, as well as tissue extracts such as homogenized tissue, tumor tissue, and cellular extracts. Samples further include biological samples that have been manipulated in any way after their procurement, such as by treatment with reagents, solubilized, or enriched for certain components, such as proteins or nucleic acids, or embedded in a semi-solid or solid matrix for sectioning purposes, e.g., a thin slice of tissue or cells in a histological sample. Preferably, samples include skin, including skin punch or biopsy, skin cells, and cultured cells and cell lines derived from skin cells. Samples may contain environmental components, such as, e.g., water, soil, mud, air, resins, minerals, etc. In certain embodiments, a sample may comprise biological specimen containing DNA (for example, genomic DNA or gDNA), RNA (including mRNA, tRNA and all other classes), protein, or combinations thereof, obtained from a subject (such as a human or other mammalian subject).

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin (e.g., keratinocytes), liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

The terms “polynucleotide” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., USA; as NEUGENE) polymers, and other synthetic sequence-specific nucleic acid polymers providing that the polymers contain nucleobases in a configuration which allows for base pairing and base stacking, such as is found in DNA and RNA. In addition, there is no intended distinction in length between the two terms.

As used herein, “nucleotide” refers to molecules that, when joined, make up the individual structural units of the nucleic acids (e.g., RNA/DNA). A nucleotide is composed of a nucleobase (nitrogenous base), a five-carbon sugar (either ribose or 2-deoxyribose), and one phosphate group. “Nucleic acids” as used herein are polymeric macromolecules made from nucleotides. In DNA, the purine bases are adenine (A) and guanine (G), while the pyrimidines are thymine (T) and cytosine (C). RNA uses uracil (U) in place of thymine (T). The term includes derivatives of the bases, e.g., methyl-cytosine (mC), N6-methyladenosine (m6A), etc.

As used herein, a “nucleic acid,” “polynucleotide,” or “oligonucleotide” can be a polymeric form of nucleotides of any length, can be DNA or RNA, and can be single- or double-stranded. Nucleic acids can include promoters or other regulatory sequences. Oligonucleotides can be prepared by synthetic means. Nucleic acids include segments of DNA, or their complements spanning or flanking any one of the polymorphic sites. The segments can be between 5 and 100 contiguous bases and can range from a lower limit of 5, 10, 15, 20, or 25 nucleotides to an upper limit of 10, 15, 20, 25, 30, 50, or 100 nucleotides (where the upper limit is greater than the lower limit). Nucleic acids between 5-10, 5-20, 10-20, 12-30, 15-30, 10-50, 20-50, or 20-100 bases are common. A reference to the sequence of one strand of a double-stranded nucleic acid defines the complementary sequence and except where otherwise clear from context, a reference to one strand of a nucleic acid also refers to its complement. Complementation can occur in any manner, e.g., DNA=DNA; DNA=RNA; RNA=DNA; RNA=RNA, wherein in each case, the “=” indicates complementation. Complementation can occur between two strands or a single strand of the same or different molecule.

A nucleic acid may be naturally or non-naturally polymorphic, e.g., having one or more sequence differences (e.g., additions, deletions and/or substitutions) as compared to a reference sequence. A reference sequence may be based on publicly available information (e.g., the U.C. Santa Cruz Human Genome Browser Gateway or the NCBI website or may be determined by a practitioner of the present disclosure using methods well known in the art (e.g., by sequencing a reference nucleic acid).

As used herein, the term “genomic DNA” refers to double stranded deoxyribonucleic acid that constitutes the genome of an organism, and that is passed along in equal proportions to the daughter cells as a result of a cell division of a parental cell. The term “genome” as used herein means the total set of genes and regulatory regions carried by an individual or cell, which define the individual or cell as belonging to a particular genus and species. For example, DNA in a chromosome is regarded genomic DNA under the scope of this definition, because a chromosome is part of the genome of an organism, and is passed along in equal proportions to F1 cells as a result of a cell division of a P1 cell.

As used herein, the term “germline DNA” refers to DNA isolated or extracted from a subject's germline cells, e.g., peripheral mononuclear blood cells, including lymphocytes that are in turn obtained from circulating blood.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “locus” refers to a specific position along a chromosome or DNA sequence. Depending upon context, a locus could be a gene, a marker, a chromosomal band or a specific sequence of one or more nucleotides. Typically, loci are in proximity to the genes/markers they are associated with, e.g., within 5 kilo bases (kb), within 4 kb, within 2 kb, within 1 kb, within 800 base pairs (bp), within 500 bp, within 400 bp, within 300 bp, within 200 bp, within 100 bp, within 50 bp, within 30 bp, within 20 bp, or fewer bp of named gene or CpG.

As used herein, the term “allele” refers to one of a pair or series, of forms of a gene or non-genic region that occur at a given locus in a chromosome. In a normal diploid cell there are two alleles of any one gene (one from each parent), which occupy the same relative position (locus) on homologous chromosomes. Within a population, there may be more than two alleles of a gene. SNPs also have alleles, e.g., the two (or more) nucleotides that characterize the SNP.

As used herein, the terms “probe” or “primer” refer to a nucleic acid or oligonucleotide that forms a hybrid structure with a sequence in a target region of a nucleic acid due to complementarity of the probe or primer sequence to at least one portion of the target region sequence.

The term “label” as used herein refers, for example, to a compound that is detectable, either directly or indirectly. The term includes colorimetric (e.g., luminescent) labels, light scattering labels or radioactive labels. Fluorescent labels include, inter alia, the commercially available fluorescein phosphoramidites such as FLUOREPRIME™ (Pharmacia™) FLUOREDITE™ (Millipore™) and FAM™ (ABI™) (see, e.g., U.S. Pat. Nos. 6,287,778 and 6,582,908).

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer may range from, e.g., 10 to 50 nucleotides; preferably 12 to 30 nucleotides. Typically, primers have sufficient complementary to hybridize with a template. Site/area of the template to which a primer hybridizes is termed “primer site.” Directionality of hybridization is generally denoted in terms of 5′ to 3′ end of the linear polynucleotide, wherein a 5′ upstream primer hybridizes with the 5′ end of the sequence to be amplified and a 3′ downstream primer that hybridizes with the complement of the 3′ end of the sequence to be amplified.

The term “complementary” as used herein refers to the hybridization or base pairing, e.g., via hydrogen bonds, between nucleotides or nucleic acids, such as, for instance, between the two strands of a double stranded DNA molecule or between an oligonucleotide primer and a primer. Complementary polynucleotides may be aligned at least 70%, at least 80%, at least 90%, at least 95%, at least 98%, at least 99% or a greater percentage, e.g., 99.9%.

The term “hybridization,” as used herein, refers to any process by which a strand of nucleic acid bonds with a complementary strand through base pairing. For example, hybridization under high stringency conditions could occur in about 50% formamide at about 37° C. to about 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 μg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature. Variations on the above ranges and conditions are well known in the art.

The term “hybridization complex” as used herein, refers to a complex formed between two nucleic acid sequences by virtue of the formation of hydrogen bonds between complementary bases. A hybridization complex may be formed in solution or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

As used herein, the term “epigenetic profile” refers to epigenetic modifications such as methylation including hypermethylation and hypomethylation, RNA/DNA interactions, expression profiles of non-coding RNA, histone modification, changes in acetylation, ubiquitination, phosphorylation and sumoylation, as well as chromatin altered transcription factor levels and the like leading to activation or deactivation of genetic locus expression. In an embodiment, the extent of methylation is determined as well as any changes therein. In an aspect, the epigenetic modification is an increase or decrease in methylation or an alteration in distribution of methylation sites or other epigenetic sites.

As used herein, the term “methylome” refers to the methylation profile of the genome. It may comprise the totality and the pattern of the positions of methylated cytosine (mC) of DNA. In some embodiments, the term “methylome” represents a collective set of genomic fragments comprising methylated cytosines, or alternatively, a set of genomic fragments that comprise methylated cytosines in the original template DNA.

As used herein, the term “marker” refers to a characteristic that can be objectively measured as an indicator of normal biological processes, pathogenic processes or a pharmacological response to a therapeutic intervention, e.g., treatment with an anti-cancer agent. Representative types of markers include, for example, molecular changes in the structure (e.g., sequence) or number of the marker, comprising, e.g., gene mutations, gene duplications, or a plurality of differences, such as somatic alterations in gDNA, copy number variations, tandem repeats, gene expression level or a combination thereof. The term “marker” includes products of genes, e.g., mRNA transcript and the protein product, including variants thereof, such as, for example, splice variants of primary mRNA and the polypeptide products thereof. Markers include differentially expressed gene products, e.g., over-expression, under-expression, knockout, constitutive expression, mistimed expression, compared to controls. Markers of the disclosure further include cis-regulatory elements and/or trans-regulatory elements. As is known in the art, “cis-regulatory elements” are present on the same molecule of DNA as the gene they regulate whereas “trans-regulatory elements” can regulate genes distant from the gene from which they were transcribed. Representative examples of cis-regulatory elements include, e.g., promoters, enhancers, repressors, etc. Representative examples of trans-regulatory elements include e.g., DNA sequences that encode transcription factors. The trans-regulation or cis-regulation could be at the level of transcription or methylation. In some embodiments, cis-regulatory elements are often binding sites for one or more trans-acting factors.

As used herein, the term “methylation” will be understood to mean the presence of a methyl group added to a nucleotide. The nucleobases of DNA/RNA can be derivatized. DNA methylation refers to the addition of a methyl (CH₃) group to the DNA strand itself, often to the fifth carbon atom of a cytosine ring. This conversion of cytosine bases to 5-methylcytosine is catalyzed by DNA methyltransferases (DNMTs). These modified cytosine residues usually are next to a guanine base (CpG methylation) and the result is two methylated cytosines positioned diagonally to each other on opposite strands of DNA. RNA can also be methylated similarly. N6-methyladenosine is the most common and abundant methylation modification in RNA molecules (mRNA) in eukaryotes followed by 5-methylcytosine (5-mC). Preferably, the term “methylation” denotes a product formed by the action of a DNA methyltransferase enzyme to a cytosine base or bases in a region of nucleic acid, e.g., genomic DNA.

The term “methylation marker” as used herein refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites may encompass the mRNA-encoding regions, the intron regions, or promoter/enhancer regions of the indicated genes. Thus, the regions can begin upstream of a gene promoter and extend downstream into the transcribed region.

The term “methylation status” as used herein refers to the presence or absence of methylation in a specific nucleic acid region e.g., genomic region. In the context of the present disclosure, the term “methylation status” encompasses methylation status or hydroxymethylation status of “—C-phosphate-G-” (CpG) sites or “—C-phosphate-any base (N)-phosphate-G” (CpNpG) sites and genes. The term “methylation status” also encompasses methylation status of non-CpG sites or non-CG methylation. In particular, the present disclosure relates to detection of “methylation status” of cytosine (5-methylcytosine). A nucleic acid sequence may comprise one or more such CpG methylation sites.

In some embodiments, the “methylation status” is indicative of a level of the methylation in a nucleic acid. Herein, the methylation level may be expressed in any numeric form, e.g., total count, arithmetic mean, e.g., average per million base pairs (bp), geometric mean, etc. Counts may be obtained using, e.g., quantitative bisulfite pyrosequencing with the PSQ HS 96A pyrosequencing system (Qiagen, Germantown, Md., USA) following bisulfite modification of genomic DNA using EZ DNA methylation GOLD KITS (Zymo Research, Irvine, Calif., USA).

In some embodiments, the methylation status is indicative of a pattern of the methylation in a nucleic acid. Epigenetic probing to determine methylation pattern can involve imaging stretched single molecules of DNA. The imaging can include simultaneously localizing the position of a DNA origami probe on a single molecule of DNA and reading the origami “barcode”. An exemplary method is described in US Pub. No. 2016/0168632.

In the context of a gene or template DNA, its methylation status can include determining a methylation status of a methylation marker within or flanking about 10 bp to 50 bp, about 50 to 100 bp, about 100 bp to 200 bp, about 200 bp to 300 bp, about 300 to 400 bp, about 400 bp to 500 bp, about 500 bp to 600 bp, about 600 to 700 bp, about 700 bp to 800 bp, about 800 to 900 bp, 900 bp to 1 kb, about 1 kb to 2 kb, about 2 kb to 5 kb, or more of a named gene, or CpG position. The process may include “selective detection” of methylated nucleobase. Herein, the phrase “selectively detecting” refers to methods wherein only a finite number of methylation marker or genes (comprising methylation markers) are measured rather than assaying essentially all potential methylation marker (or genes) in a genome. For example, in some aspects, “selectively detecting” methylation markers or genes comprising such markers can refer to measuring no more than 2400, 2350, 2300, 2250, 2200, 2150, 2100, 2050, 2000, 1950, 1900, 1850, 1800, 1750, 1700, 1650, 1600, 1550, 1500, 1450, 1400, 1350, 1300, 1250, 1200, 1150, 1,000, 950, 900, 850, 800, 750, 700, 650, 600, 550, 500, 450, 400, 350, 300, 275, 250, 225, 200, 175, 150, 125, 100, 50, 25, 20, or 10 different methylation markers or genes comprising methylation markers. Preferably, selective detection of methylation markers comprises detecting a subset of the markers or genes of Table 1.

As used herein, the term “differential methylation” shall be taken to mean a change in the relative amount of methylation of a nucleic acid e.g., genomic DNA, in a biological sample e.g., such as a cell or a cell extract, or a body fluid (such as blood), obtained from a subject. In one example, the term “differential methylation” is an increased level of methylation of a nucleic acid. In another example, the term “differential methylation” is a decreased level of methylation of a nucleic acid. In the present disclosure, “differential methylation” is generally determined with reference to a baseline level of methylation for a given genomic region. For example, the level of differential methylation may be at least 2% greater or less than a baseline level of methylation, for example at least 5%, at least 15%, at least 20%, at least 25%, at least 30%, at least 40%, at least 50%, at least 60%, at least 70%, at least 80%, at least 90%, at least 100%, at least 120%, at least 200%, e.g., about 300%. Thus, the level of differential methylation may be at least 2%, at least 15%, at least 20%, or at least 25% greater than or less than a baseline level of methylation in a reference genome. Evaluation of methylation status may be performed independently of a reference genome, for example, using cross-mapping and motif enrichment analysis for interpreting the identified differentially methylated regions in the absence of a reference genome (Klughammer et al. Cell Rep., 13(11): 2621-2633, 2015).

As used herein, a “reference level of methylation” shall be understood to mean a level of methylation detected in a corresponding nucleic acid from a normal or healthy cell or tissue or body fluid, or a data set produced using information from a normal or healthy cell or tissue or body fluid. Commercial or in-house controls with low and high methylation may be used to verify biases (Langevin et al., Epigenetics 7: 291-299, 2012; Sandoval et al., Epigenetics 6: 692-702, 2011). Biases may be addressed by aligning to a common reference followed by filtering of variable CpG sites, and genotyping using bisulfite-converted DNA (Wulfridge et al., BioRxi, Jan. 31, 2016). In the context of methylation arrays, datasets on genome-wide DNA methylation measured in various reference samples (e.g., cord whole blood) may be employed in parallel to the test sample (e.g., blood, saliva, placenta, saliva, adipose).

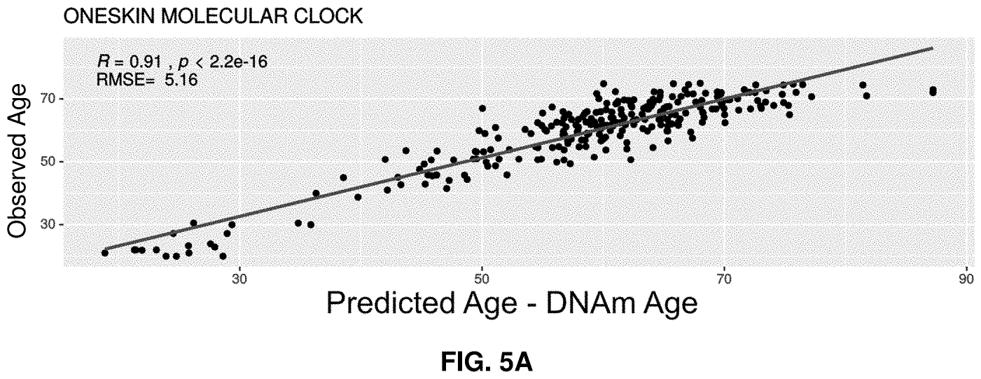

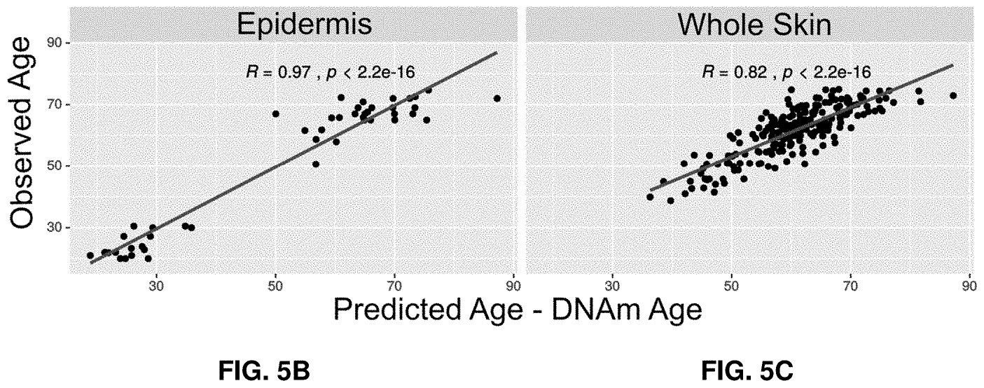

In some embodiments, to determine a “reference level of methylation,” artificial plasmid constructs with pre-defined sequences that represent exactly 0%-(M0) and 100%-methylation (M100) of genes may be used (Yu et al., PLoS One, 10(9):e0137006, 2015). Accordingly, a “reference level of methylation” may be a level of methylation in a corresponding nucleic acid from: (i) a sample comprising a normal cell; (ii) a sample from a reference genome assembly; (iii) a sample from a synthetic sample; (iv) a data set comprising measurements of methylation for a healthy individual or a population of healthy individuals; (vi) a data set comprising measurements of methylation for a normal individual or a population of normal individuals; and (vii) a data set comprising measurements of methylation from the subject being tested wherein the measurements are determined in a baseline sample (e.g., cord blood). In some embodiments, the reference level of methylation may be a level of methylation determined for one or more CpG dinucleotide sequences within a corresponding methylation array like the 450K BEADCHIP dataset, EPIC or other similar dataset (Illumina, Inc., San Diego, Calif., USA) or measured by a sequencing method as Methyl-Seq and others. The reference levels may, optionally, be stored in said tangible computer-readable medium. In certain aspects, determining the age of the biological sample may comprise applying a linear regression model to predict sample age based on a weighted average of the methylation marker levels plus an offset. In some embodiments, prediction or calculation of the age is performed using a regression model, e.g., using a regression curve shown in FIG. 5.

As used herein, the term “sequencing” or “sequence” as a verb refers to a process whereby the nucleotide sequence of DNA, or order of nucleotides, is determined, such as a nucleotide order AGTCC, etc. The term “sequence” as a noun refers to the actual nucleotide sequence obtained from sequencing; for example, DNA having the sequence AGTCC. Wherein the “sequence” is provided and/or received in digital form, e.g., in a disk or remotely via a server, “sequencing” may refer to a collection of DNA that is propagated, manipulated and/or analyzed using the methods and/or systems of the disclosure.

As used herein, the term “threshold value” means a cutoff value. Threshold values in the context of age determinations may be representative of error, which may be determined statistically using standard approaches, e.g., standard error of mean (SEM) or standard deviation (SD). In some embodiments, the threshold value may include 1, 2 or 3 standard deviations (preferably one standard deviation) of the mean difference between the calculated age and the actual age across n samples, wherein the n samples are obtained from the same subject or different subjects (preferably different subjects who are similar to each other with respect to demographic factors such as race, ethnicity, gender, and/or actual age). The threshold value may be subject-specific, in which case, the difference between calculated age and actual age is determined for the same subject for y preceding years. Alternately, the threshold-value may be population-specific, in which case, the difference between calculated age and actual age is determined for a population of n subjects of any given age or age distribution (e.g., between 50 and 55 years). Still further, the threshold value may be representative of a global population.

The term “methylation sequencing” as used herein refers to detection of methylated nucleobase, e.g., mC. The term includes high-throughput sequencing technologies, such as MeDIP, RRBS, HELP, and METHYLC-SEQ. For example, METHYLC-SEQ can be used to directly sequence the sodium bisulfite converted DNA fragment by next generation sequencing (NGS). Especially, the methylation level of single base pairs over the whole genome or fragment thereof can be obtained through an analysis of methylation sequencing results. Methylation sequencing can include DNA sequencing, wherein, the position of the methylated nucleobase is denoted inside large parenthesis ([ ]). In some embodiments, methylation sequencing includes DNA methylation profiling of single cells (or small cell populations), using, e.g., micro whole genome bisulfite sequencing (μWGBS).

As used herein, the term “variant” refers to a methylation sequence in which the structure of the nucleic acid differs from a reference sequence, for example by a difference of at least one methylated nucleobase. A result of the variation may be no change, differentially expressed gene, a change in gene transcription (e.g., rate of mRNA synthesis), a change in translation (e.g., rate of protein synthesis), including, changes in levels or activity of the gene product (e.g., protein).

The term “genetic variant” refers to a nucleotide sequence in which the sequence differs from the sequence most prevalent in a population, for example by one nucleotide, in the case of the SNPs Non-limiting examples of genetic variants include frameshift, stop gained, start lost, splice acceptor, splice donor, stop lost, in frame indel, missense, splice region, synonymous and copy number variants (CNV). Non-limiting types of CNVs include deletions and duplications.

As used herein, “methylation variant data” refer to data obtained by identifying the methylation variants in a subject's nucleic acid, relative to a reference nucleic acid sequence.

As used herein, the term “bin” refers to a group of DNA/RNA sequences grouped together, such as in a “genomic bin” or “transcript bin”. In a particular case, the bin may comprise a group of markers that are binned based on association with a gene of interest or a locus thereto.

As used herein, the term “signature” comprises a collection of markers, e.g., methylation markers comprising C/G nucleic acid sequences, ILLUMINA Probe ID numbers (CG) annotating to the nucleic acid sequences, including genes linking to the nucleic acids, or loci related thereto. A signature may comprise a combination of these markers, e.g., a specific methylation site (as indicated by ILLUMINA probe ID) and a global methylation profile in a gene of interest. Signatures typically comprise about 5, 10, 20, 30, 40, 50, 75, 100, 150, 175, 200, 225, 250, 275, 300 (+/−25) entities or more markers. Preferably, signatures typically comprise about 10, 20, 50, 100, 125, 150, 175, 200, 225, 250, 275, or 300 (+/−25) entities or more markers.

As used herein, the term “screen” refers to a specific biological or biochemical assay which is directed to measurement of a specific condition or phenotype that a molecule induces in a target, e.g., target in silico system (e.g., computational modeling software based on energy considerations), target cell-free systems (e.g., BIACORE systems), target cells, tissues, organs, organ systems, or organisms.

As used herein, the term “selecting” in the context of screening compounds or libraries includes both (a) choosing compounds from a group previously unknown to be modulators of a condition or phenotype (e.g., cancer); and (b) testing compounds that are known to be inhibitors or activators of the condition or phenotype (e.g., cancer). Both types of compounds are generally referred to herein as “test compounds.” The test compounds may include, by way of example, polypeptides (e.g., small peptides, artificial or natural proteins, antibodies), polynucleotides (e.g., DNA or RNA), carbohydrates (small sugars, oligosaccharides, and complex sugars), lipids (e.g., fatty acids, glycerolipids, sphingolipids, etc.), mimetics and analogs thereof, and small organic molecules having a molecular weight of less than about 10 KDa, preferably less than about 5 KDa, especially less than about 1 KDa (e.g., about 300 daltons to about 800 daltons). The test compounds may be provided in library formats known in the art, e.g., in chemically synthesized libraries, recombinantly-expressed libraries (e.g., phage display libraries), and in vitro translation-based libraries (e.g., ribosome display libraries).

As used herein the term “small molecule” may include a small organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the compound may be an inorganic compound. Inorganic compounds are derived from mineral sources and include all compounds without carbon atoms (except carbon dioxide, carbon monoxide and carbonates). Preferably, the small molecule has a molecular weight of less than about 10000 atomic mass units (amu), or less than about 5000 amu such as 1000 amu, 500 amu, and even less than about 250 amu. The size of a small molecule can be determined by methods well-known in the art, e.g., mass spectrometry. In some embodiments, the small molecule has a molecular weight of less than about 10 KDa, preferably less than about 5 KDa, especially less than about 1 KDa (e.g., about 300 daltons to about 800 daltons). Small molecules may be designed, for example, in silico based on the crystal structure of potential drug targets, where sites presumably responsible for the biological activity and involved in the regulation of expression of genes identified herein, can be identified and verified in in vivo assays such as in vivo HTS (high-throughput screening) assays. Small molecules can be part of libraries that are commercially available, for example from CHEMBRIDGE Corp., San Diego, USA. In contrast, a “large molecule” has a molecular weight of greater than about 5 KDa, preferably greater than about 20 KDa, especially greater about 100 KDa.

As used herein, the term “drug” relates to compounds, which have at least one biological and/or pharmacologic activity. Preferably, the drug is a compound used or a candidate compound intended for use in the treatment, cure, prevention or diagnosis of a disease or intended to be used to enhance physical or mental well-being.

As used herein, the term “prodrug” includes compounds that are generally not biologically and/or pharmacologically active. After administration, the prodrug is activated, typically in vivo by enzymatic or hydrolytic cleavage and converted to a biologically and/or pharmacologically active compound, which has the intended medical effect, i.e. is a drug that exhibits a biological and/or pharmacologic effect. Prodrugs are typically formed by chemical modification of biologically and/or pharmacologically active compounds. Conventional procedures for the selection and preparation of suitable prodrug derivatives are described, for example, in Design of Prodrugs, ed. H. Bundgaard, Elsevier, 1985.

As used herein, the term “second messengers” refers to molecules that relay signals from receptors on the cell surface to target molecules inside the cell, in the cytoplasm or nucleus. For example, second messengers are involved in the relay of the signals of hormones or growth factors and are involved in signal transduction cascades. Second messengers may be grouped in three basic groups: hydrophobic molecules (e.g., diacyglycerol, phosphatidylinositols), hydrophilic molecules (e.g., cAMP, cGMP, IP3, Ca2+) and gases (e.g., nitric oxide, carbon monoxide).

The term “metabolites” as used herein corresponds to its generally accepted meaning in the art, i.e. metabolites are intermediates and products of metabolism and may be grouped in primary (e.g., involved in growth, development and reproduction) and secondary metabolites.

As used herein, “aptamers” refer to molecules, e.g., oligonucleic acid or peptide molecules that bind a specific target molecule. Aptamers are usually created by selecting them from a large random sequence pool, but natural aptamers also exist in riboswitches. Further, they can be combined with ribozymes to self-cleave in the presence of their target molecule. More specifically, aptamers can be classified as DNA or RNA aptamers or peptide aptamers. Whereas the former consist of (usually short) strands of oligonucleotides, the latter consist of a short variable peptide domain, attached at both ends to a protein scaffold. Nucleic acid aptamers are nucleic acid species that may be engineered through repeated rounds of in vitro selection or equivalently, systematic evolution of ligands by exponential enrichment (SELEX) to bind to various molecular targets such as small molecules, proteins, nucleic acids, and even cells, tissues and organisms. Peptide aptamers consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein, which has good solubility properties. Peptide aptamer selection can be made using, e.g., yeast two-hybrid system.

As used herein, the term “oligosaccharides” refers to saccharide (e.g., sugar) polymers containing a small number of component sugars such as, e.g., at least (for each value) 3, 4, 5, 6, 7, 8, 9, 10, 11, 12, 13, 14, or at least 15 monosaccharides. They may be, e.g., O- or N-linked to amino acid side chains of polypeptides or to lipid moieties.

As used herein, an “antibody” includes whole antibodies and any antigen-binding fragment or a single chain thereof. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments to a preselected target. Examples of binding fragments encompassed within the term “antigen binding portion” of an antibody include (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment, which consists of a VH domain; and (vi) an isolated complementarity determining region (CDR).

As used herein, the term “monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity that have variable and constant regions derived from human germline immunoglobulin sequences.

An “interaction” as used herein is either a direct physical interaction, also referred to as “binding”, or an indirect interaction mediated by other constituents that may or may not be endogenous components of the system, e.g., cell. As defined in the main embodiment, said reaction, preferably binding, occurs within the cell. In other embodiments, indirect interactions, such as triggering of signaling pathways resulting in genetic or epigenetic changes, which manifest at the cellular, tissue, organ or even organismal level, are also included within this term.

As used herein, the term “determining an interaction” includes determining presence or absence of a given interaction, detecting whether a previously unknown interaction occurs, quantifying interactions, wherein said interactions may include known as well as previously unknown interactions. The methods disclosed herein also extends to observing an interaction, wherein said observing may also include observing or monitoring over time and/or at more than one location, preferably locations within a site of interest, e.g., CpG site, gene located in a particular chromosome, or a specific locus in the gene. Methods of quantifying such interactions include both dry science (e.g., use of computational software) as well as wet science (e.g., determination of methylated sites using methylome sequencing) or semi-wet science (e.g., using INFINIUM chips). The interaction to be determined is preferably a change in the methylation status.

As used herein, the terms “treat,” “treating,” or “treatment of,” refers to reduction of severity of a condition or at least partially improvement or modification thereof, e.g., via complete or partial alleviation, mitigation or decrease in at least one clinical symptom of the condition, e.g., cancer.

As used herein, the term “administering” is used in the broadest sense as giving or providing to a subject in need of the treatment, a composition such as a drug. For instance, in the pharmaceutical sense, “administering” means applying as a remedy, such as by the placement of a drug in a manner in which such molecule would be received, e.g., intravenous, oral, topical, buccal (e.g., sub-lingual), vaginal, parenteral (e.g., subcutaneous; intramuscular including skeletal muscle, cardiac muscle, diaphragm muscle and smooth muscle; intradermal; intravenous; or intraperitoneal), topical (i.e., both skin and mucosal surfaces), intranasal, transdermal, intra articular, intrathecal, inhalation, intraportal delivery, organ injection (e.g., eye or blood, etc.), or ex vivo (e.g., via immunoapheresis).

As used herein, “contacting” means that the composition comprising the active ingredient is introduced into a sample containing a target, e.g., a protein target, a cell target, in an appropriate environment, e.g., within a software application, a BIACORE system, a test tube, flask, tissue culture, chip, array, plate, microplate, capillary, or the like, and incubated at a temperature and time sufficient to permit binding (e.g., target binding to an unknown binding partner) or vice versa (e.g., a binding partner binding to an unknown target). In the in vivo context, “contacting” means that the therapeutic or diagnostic molecule is introduced into a patient or a subject for the treatment of a disease, and the molecule is allowed to come in contact with the patient's target tissue, e.g., skin tissue or blood tissue, in vivo or ex vivo.

As used herein, the term “therapeutically effective amount” refers to an amount that provides some improvement or benefit to the subject. Alternatively stated, a “therapeutically effective” amount is an amount that will provide some alleviation, mitigation, or decrease in at least one clinical symptom in the subject. Methods for determining therapeutically effective amount of the therapeutic molecules, e.g., anticancer agents or antibodies, are known in the art, and may include in vitro assays or in vivo pharmacological assays.

As used herein, the term “modulate,” with reference to an interaction between a target and its partner means to regulate positively or negatively the normal biological function of a target. Thus, the term modulate can be used to refer to an increase, decrease, masking, altering, overriding or restoring the normal functioning of a target. A modulator can be an agonist, a partial agonist, or an antagonist, a cofactor, an allosteric activator or inhibitor or the like.

As used herein, the term “inhibit” refers to reduction in the amount, levels, density, turnover, association, dissociation, activity, signaling, or any other feature associated with a target agent, e.g., a protein or a nucleic acid (e.g., mRNA) or a target feature, e.g., skin wrinkle.

As used herein, the term “pharmaceutically acceptable” means a molecule or a material that is not biologically or otherwise undesirable, i.e., the molecule or the material can be administered to a subject without causing any undesirable biological effects such as toxicity.

As used herein, the term “carrier” denotes buffers, adjuvants, dispersing agents, diluents, and the like. For instance, the peptides or compounds of the disclosure can be formulated for administration in a pharmaceutical carrier in accordance with known techniques. See, e.g., Remington, The Science & Practice of Pharmacy (9^{th} Ed., 1995). In the manufacture of a pharmaceutical formulation according to the disclosure, the peptide or the compound (including the physiologically acceptable salts thereof) is typically admixed with, inter alia, an acceptable carrier. The carrier can be a solid or a liquid, or both, and is preferably formulated with the peptide or the compound as a unit-dose formulation, for example, a tablet, which can contain from about 0.01 or 0.5% to about 95% or 99%, particularly from about 1% to about 50%, and especially from about 2% to about 20% by weight of the peptide or the compound. One or more peptides or compounds can be incorporated in the formulations of the disclosure, which can be prepared by any of the well-known techniques of pharmacy.

### I. Methods

The methods of the present disclosure are used to detect age of a sample or an individual or the propensity to age in a subject based on methylation status. Various methods are available to those of skill in the art to determine methylation status. In some instances, it may be desirable to assess methylation status using a particular method. For example, a suitable method for assessing methylation status is exemplified below.

In some embodiments, the methods of the disclosure are carried out on a sample obtained from subjects. Preferably, the sample comprises skin, blood (including whole blood), blood plasma, blood serum, hemolysate, lymph, synovial fluid, spinal fluid, urine, cerebrospinal fluid, stool, sputum, mucus, amniotic fluid, lacrimal fluid, cyst fluid, sweat gland secretion, bile, milk, tears, saliva, earwax, skin or other tissues cells. The sample may be treated to remove particular cells using various methods such as such centrifugation, affinity chromatography (e.g., immunoabsorbent means), immunoselection and filtration. Thus, in an example, the sample can comprise a specific cell type or mixture of cell types isolated directly from the subject or purified from a sample obtained from the subject (e.g., purifying T-cells from whole blood). In an example, the biological sample is peripheral blood mononuclear cells (pBMC). In other examples, the sample may be selected from the group consisting of B cells, dendritic cells, granulocytes, innate lymphoid cells (ILCs), megakaryocytes, monocytes/macrophages, natural killer (NK) cells, platelets, red blood cells (RBCs), T cells, thymocytes. In some embodiments, the sample may comprise skin cells, hair follicle cells, sperm, etc. Samples (e.g., skin, muscle, cartilage, fat, liver, lung, neural/brain, blood tissue) can be acquired directly from subjects/patients with skin that is naturally aged (i.e., elderly donors) or prematurely aged (e.g., individuals with progeria, etc.) without the need for artificial aging using a skin age inducing agent. In an exemplary embodiment, the samples are obtained from subjects greater than about 35 years of age.

The sample may be purified using conventional methods to obtain sub-populations of cells. For example, Fibroblast and keratinocyte cells can be purified using different enzymes to digest the skin (e.g. Trypsin or dispase), as well different cell culture media. pBMC can be purified from whole blood using various known Ficoll based centrifugation methods (e.g., Ficoll-Hypaque density gradient centrifugation). Other cells such as T-cells can also be purified by selecting for the appropriate phenotype using techniques such as immunomagnetic cell sorting (e.g., DYNABEADS, Invitrogen, Carlsbad, Calif., USA). For example, T-cells can be purified using a two-step selection process that firstly removes CD8+ cells and then selects CD4+ cells. Cell population purity can be confirmed by assessing the appropriate markers such as CD19-FITC, CD3-PE, CD8-PerCP, CD11 c-PE Cy7, CD4-APC and CD14-APC Cy7 using commercially available antibodies (e.g., BD Biosciences).

After sample preparation, DNA is extracted from the sample for methylation analysis. In an example, the DNA is genomic DNA. Various methods of isolating DNA, in particular genomic DNA are known to those of skill in the art. In general, known methods involve disruption and lysis of the starting material followed by the removal of proteins and other contaminants and finally recovery of the DNA. For example, techniques involving alcohol precipitation; organic phenol/chloroform extraction and salting out have been used for many years to extract and isolate DNA. One example of DNA isolation is exemplified below (e.g. Qiagen All-prep kit). However, there are various other commercially available kits for genomic DNA extraction (Thermo-Fisher, Waltham, Mass.; Sigma-Aldrich, St. Louis, Mo.). Purity and concentration of DNA can be assessed by various methods, for example, spectrophotometry.

In some embodiments, the genetic data comprising a compendium of methylation markers, e.g., CpG, is received in an appropriate format (e.g., raw data such as, e.g., idat file, fastq file or processed data, e.g., BED format or WIG format (.bed or .wig) or a variant thereof). See Kent et al., Bioinformatics, 26 (17), 2204-2207, 2010. Wiggle (WIG) format is an older format for display of dense, continuous data such as GC percent, probability scores, and transcriptome data. Wiggle data elements are usually equally sized. In contrast, A BED file (BED) is a tab-delimited text file that defines a feature track. The BED file format is described on the U.C.S.C. Genome Bioinformatics website. Certain repositories such as Illumina provide complete datasets in downloadable BED format. A representative example is Illumina's TRUSIGHT Autism Content Set BED File A (deposited: Feb. 5, 2013), which is available via the web at support(dot)illumina(dot)com/downloads(dot)html. The IDAT file is a proprietary format used to store BEADARRAY data from the myriad of genome-wide profiling platforms on offer from Illumina Inc and is output directly from a scanner/reader and stores summary intensities for each probe-type on an array in a compact manner (Smith et al., F1000Research, 2:264, 2013). FASTQ format is a text-based format for storing both a biological sequence (usually nucleotide sequence) and its corresponding quality scores. Both the sequence letter and quality score are each encoded with a single ASCII character for brevity (Cock et al., Nucleic Acids Research, 38 (6): 1767-1771, 2009).

The disclosure further relates to profiling methylation status of a polynucleotide (e.g., human chromosome) directly after a sample is obtained. Here, the subject's sample containing DNA may be profiled, e.g., using methylation sequencing (MS). Methylation sequencing can be carried out by bisulfite treatment of DNA following by sequencing. The treatment of DNA with bisulfite converts cytosine residues to uracil, but leaves 5-methylcytosine residues unaffected. Therefore, after sequencing, cytosine residues represent methylated cytosines in the genome. One variant of bisulfite sequencing is reduced representation bisulfite sequencing (RRBS), which was developed as a cost-efficient method to profile areas of the genome that have a high CpG content. In RRBS, genomic DNA is digested using the restriction endonuclease MspI, which recognizes the sequence 5′-CCGG-3′. MspI is actually part of an isoschizomer pair with HpaII, which are restriction enzymes that are specific to the same recognition sequence. However, MspI can recognize methylated cytosines, whereby HpaII cannot. This property makes HpaII-MspI pair to a valuable tool for rapid methylation analysis.

The methylation data obtained via bisulfite sequencing or RRBS can be converted to an appropriate format, e.g., GRanges, BED or WIG, using appropriate tools. In some embodiments, genomic ranges as provided in the software package (e.g., Granges) may be used (Lawrence et al., PLoS Comput Biol., 9(8):e1003118, 2013). Granges class represents a collection of genomic ranges that each have a single start and end location on the genome and it can be used to store the location of genomic features such as contiguous binding sites, transcripts, and exons. These objects can be created by using the GRanges constructor function.

Preferably, the methylation status of a sample may be assessed using a methylation array, e.g. an ILLUMINA™ DNA methylation array (or using a PCR protocol involving relevant primers). The array will output methylation status in terms of levels of methylation in a subset of the DNA. The β value of methylation, which equals the fraction of methylated cytosines in a location in a segment of DNA, can be calculated from raw files. The disclosure can also be applied to any other approach for quantifying DNA methylation at locations near the genes as disclosed herein. DNA methylation can also be quantified using many currently available assays which include, but not restricted to: (a) molecular brake light assay; (b) methylation-specific Polymerase Chain Reaction; (c) whole genome bisulfite sequencing (BS-Seq); (d) The Hpall tiny fragment Enrichment by Ligation-mediated PCR (HELP) assay; (e) Methyl Sensitive Southern Blotting (similar to the HELP assay but uses Southern blotting); (f) ChIP-on-chip assay; (g) Restriction landmark genomic scanning; (h) Methylated DNA immunoprecipitation (MeDIP); and (i) pyrosequencing of bisulfite treated DNA, (j) Array based methods, such as comprehensive high-throughput arrays for relative methylation and others. Preferably, the methodology involves whole genome bisulfite sequencing (BS-Seq).

Accordingly, alternatively to using datasets, the disclosure relates to use of native biological samples containing methylation markers in genomic DNA that are processed in line with Illumina's instructions, as provided in Document #11322460 (version 2; Nov. 17, 2016). The DNA samples are then hybridized to the probes in the HUMANMETHYLATION450 BEADCHIP, INFINIUM METHYLATION EPIC KIT, or any equivalent methylation array chip. Methylation markers are detected using reagents and detectors provided by Illumina or other companies. See, Horvath et al., Genome Biology, 14:R115, 2013. These hybridization reactions yield counts, which are indicative of levels or patterns of methylation—the more probes that hybridize the more cells have this exact methylation.

However, it is not necessary to access the methylation levels on the entire genome. For example, methylation sequencing can be performed on a chromosomal DNA within a DNA region or portion thereof (e.g., having at least one cytosine residue) selected from the CpG loci identified in Table 1. In some embodiments, the methylation level of all cytosines within at least 20, 50, 100, 200, 500 or more contiguous base pairs of the CpG loci is also determined. In some embodiments, the methylation level of the cytosine at positions indicated by [C/G] in the sequences of Table 1 is determined, e.g., at least one marker from Table 1 is determined. A plurality of CpG loci identified in Table 1 may also be assessed and their methylation level determined. Once the methylation status of a CpG locus of interest is determined, it may be possible to normalize (e.g., compare) to the methylation status of a control locus. Typically, the control locus will have a known, relatively constant, methylation level. For example, the control can be previously determined to have no, some or a high amount of methylation (or methylation level), thereby providing a relative constant value to control for error in detection methods, etc., unrelated to the presence or absence of cancer. In some embodiments, the control locus is endogenous, e.g., is part of the genome of the individual sampled. For example, in mammalian cells, the testes-specific histone 2B gene (hTH2B in human) gene is known to be methylated in all somatic tissues except testes. Alternatively, the control locus can be an exogenous locus, e.g., a DNA sequence spiked into the sample in a known quantity and having a known methylation level.

The methylation sites in a DNA region can reside in non-coding transcriptional control sequences (e.g., promoters, enhancers, introns, etc.), in other intergenic sequences such as, but no limited to, repetitive sequences, or in coding sequences, including exons of the associated genes. In some embodiments, the methods comprise detecting the methylation level in the promoter regions (e.g., comprising the nucleic acid sequence that is about 1.0 kb, 1.5 kb, 2.0 kb, 2.5 kb, 3.0 kb, 3.5 kb or 4.0 kb 5′ from the transcriptional start site through to the transcriptional start site) of one or more of the associated genes identified in Table 1.

To determine methylation status of only a portion of the genome, random shearing or fragmenting of the genomic DNA may be carried out using routine tools. For example, the DNA may be cut with methylation-dependent or methylation-sensitive restriction enzymes; and the digested or native (uncut) DNA may be analyzed. Selective identification can include, for example, separating cut and uncut DNA (e.g., by size) and quantifying a sequence of interest that was cut or, alternatively, that was not cut. Alternatively, the method can encompass amplifying intact DNA after restriction enzyme digestion, thereby only amplifying DNA that was not cleaved by the restriction enzyme in the area amplified. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using conventional, real-time, quantitative PCR.

The methods may include quantifying the average methylation density in a target sequence within a population of genomic DNA. For example, the genomic DNA may be contacted with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The methylation level of a CpG loci can be determined by providing a sample of genomic DNA comprising the CpG locus, cleaving the DNA with a restriction enzyme that is either methylation-sensitive or methylation-dependent, and then quantifying the amount of intact DNA or quantifying the amount of cut DNA at the locus of interest. The amount of intact or cut DNA will depend on the initial amount of genomic DNA containing the locus, the amount of methylation in the locus, and the number (e.g., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample.

In some embodiments, a “METHYLIGHT” assay is used alone or in combination with other methods to detect methylation level. Briefly, in the METHYLIGHT process, genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA (or alternatively to methylated sequences that are not converted), amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with METHYLIGHT can include sodium bisulfite as well as primers or detectably-labeled probes (including but not limited to TAQMAN or molecular beacon probes) that distinguish between methylated and unmethylated DNA that have been treated with bisulfite. Other kit components can include, e.g., reagents necessary for amplification of DNA including but not limited to, PCR buffers, deoxynucleotides; and a thermostable polymerase.

In some embodiments, a Methylation-sensitive Single Nucleotide Primer Extension (MS-SNUPE) reaction is used alone or in combination with other methods to detect methylation level. The MS-SNUPE technique is a quantitative method for assessing methylation differences at specific CpG sites based on bisulfite treatment of DNA, followed by single-nucleotide primer extension. Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Typical reagents (e.g., as might be found in a typical MS-SNUPE-based kit) for MS-SNUPE analysis can include, but are not limited to: PCR primers for specific gene (or methylation-altered DNA sequence or CpG island); optimized PCR buffers and deoxynucleotides; gel extraction kit; positive control primers; MS-SNUPE primers for a specific gene; reaction buffer (for the MS-SNUPE reaction); and detectably-labeled nucleotides. Additionally, bisulfite conversion reagents may include DNA denaturation buffer; sulfonation buffer; DNA recovery regents or kit (e.g., precipitation, ultrafiltration, affinity column); desulphonation buffer; and DNA recovery components.

In some embodiments, a methylation-specific PCR (“MSP”) reaction is used alone or in combination with other methods to detect DNA methylation. An MSP assay entails initial modification of DNA by sodium bisulfite, converting all unmethylated, but not methylated, cytosines to uracil, and subsequent amplification with primers specific for methylated versus unmethylated DNA.

In another example, methylation status can be determined using assays such as bisulfite MALDI-TOF methylation, methylation sensitive PCR, methylation specific melting curve analysis (MS-MCA), high resolution melting (MS-HRM), MALDI-TOF MS, methylation specific MLPA; combination of methylated-DNA precipitation and methylation-sensitive restriction enzymes (COMPARE-MS), methylation sensitive oligonucleotide microarray, antibody immunoprecipitation, pyrosequencing, NEXT generation sequencing, DEEP sequencing. Such assays are available commercially.

Additional methods for detecting methylation levels can involve genomic sequencing before and after treatment of the DNA with bisulfite. When sodium bisulfite is contacted to DNA, unmethylated cytosine is converted to uracil, while methylated cytosine is not modified. Such additional embodiments include, but are not limited to the use of array-based assays such as the Illumina® HUMAN INFINIUM METHYLATION EPIC BEADCHIP (or equivalent) and multiplex PCR assays. In one embodiment, the multiplex PCR assay is Patch-PCR. Patch-PCR can be used to determine the methylation level of a certain CpG loci. See Varley et al., Genome Research, 20:1279-1287, 2010. In some embodiments, restriction enzyme digestion of PCR products amplified from bisulfite-converted DNA is used to detect DNA methylation levels.

Additional methylation level detection methods include, but are not limited to, methylated CpG island amplification and those described in, e.g., U.S. Pub. No. 2005/0069879; Rein et al., Nucleic Acids Res. 26 (10): 2255-64, 1998; Olek et al., Nat. Genet. 17(3): 275-6, 1997; and WO 00/70090.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602. Amplifications may be monitored in “real time.” Kits for the above methods can include, e.g., one or more of methylation-dependent restriction enzymes, methylation-sensitive restriction enzymes, amplification (e.g., PCR) reagents, probes and/or primers.

When performing the methods of the present disclosure, the methylation status of multiple sites will be assessed. In an example, the methylation status of the CpG sites of the present disclosure can be combined to produce a multivariate methylation pattern or methylation signature indicative of aging or a propensity to develop aging in a subject. Such a pattern or signature can be used as a comparative reference for determining an epigenetic age of the subject. In some embodiments, the methylation status of at least two CpG sites selected from the markers shown in Table 1 are determined. For instance, the methylation status of about 2, 3, 4, 5, 7, 10, 15, 20, 25, 30, 35, 40, 45, 50, 60, 75, 100, 125, 150, 200, 175, 225, 250, 275, or more, e.g., 300 CpG sites from the markers of Table 1 may be determined. Preferably, the methods include detection of the methylation status of a plurality of markers of Table 1.

In some embodiments, the methylation status of the top 2, 3, 4, 5, 7, 10, 15, 20, 22, 25, 30, 35, 40, 45, 50, 55, 65, 70, 75, 100, 125, 150, 175, 200, 225, 250, 275, or a larger number, e.g., top 300, of the highest relevant markers in Table 1 may be determined, wherein the relative importance of the markers provided by the sequence identifier number (SEQ ID NO). More specifically, a smaller SEQ ID NO indicates a more relevant marker. In particular, the methylation status of the top 2, 3, 4, 5, 6, 7, 10, 15, 20, 22, 25, 30, 35, 40, 45, 50, 55, 65, 70, 75, 100, 125, 150, 175, 200, 250, 275, or a larger number, e.g., top 300, of the markers of Table 1 are determined.

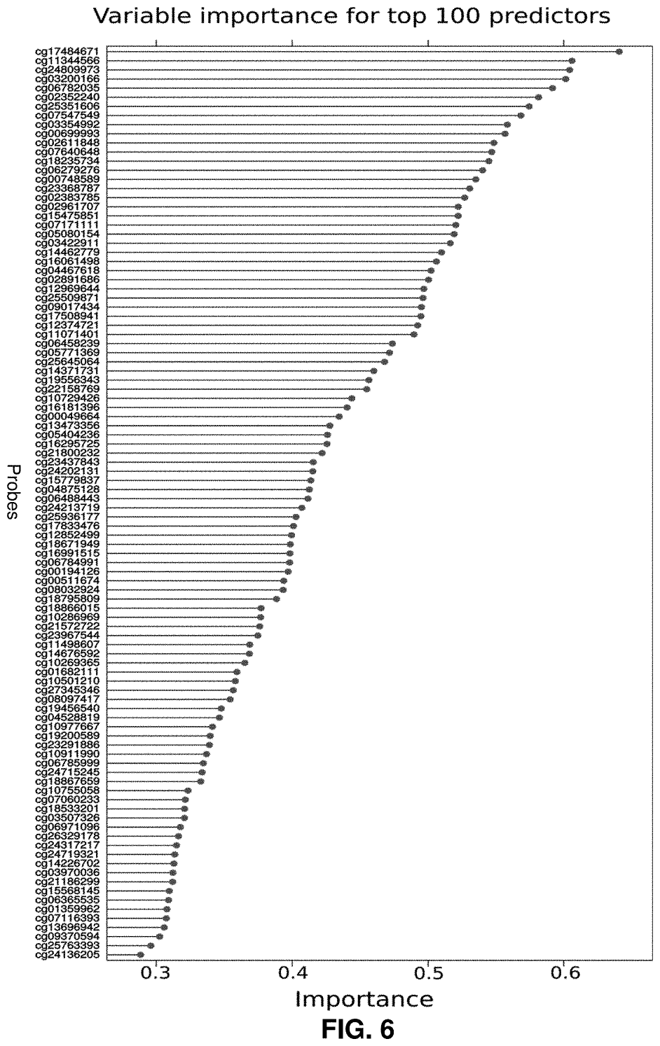

In some embodiments, the methylation status of at least 2, e.g., 2, 3, 4, 5, 7, 10, 15, 20, 25, 30, 35, 40, 45, 50, 60, 70, 75, 80, 90, or more, e.g., 100, markers shown in FIG. 6 may be determined, wherein the recited ILLUMINA Probe ID number (CG) annotates to the sequence of the nucleic acids provided by the respective SEQ ID Nos. in Table 1, including genes or loci related thereto. More specifically, the methylation status of the following markers in FIG. 6, with decreasing relevance to the calculated age of the biological sample, are determined: cg17484671; cg11344566; cg24809973; cg03200166; cg06782035; cg02352240; cg25351606; cg07547549; cg03354992; cg00699993; cg02611848; cg07640648; cg18235734; cg06279276; cg00748589; cg23368787; cg02383785; cg02961707; cg15475851; cg07171111; cg05080154; cg03422911; cg14462779; cg16061498; cg04467618; cg02891686; cg12969644; cg25509871; cg09017434; cg17508941; cg12374721; cg11071401; cg06458239; cg05771369; cg25645064; cg14371731; cg19556343; cg22158769; cg10729426; cg16181396; cg00049664; cg13473356; cg05404236; cg16295725; cg21800232; cg23437843; cg24202131; cg15779837; cg04875128; cg06488443; cg24213719; cg25936177; cg17833476; cg12852499; cg18671949; cg16991515; cg06784991; cg00194126; cg00511674; cg08032924; cg18795809; cg18866015; cg10286969; cg21572722; cg23967544; cg11498607; cg14676592; cg10269365; cg01682111; cg10501210; cg27345346; cg08097417; cg19456540; cg04528819; cg10977667; cg19200589; cg23291886; cg10911990; cg06785999; cg24715245; cg18867659; cg10755058; cg07060233; cg18533201; cg03507326; cg06971096; cg26329178; cg24317217; cg24719321; cg14226702; cg03970036; cg21186299; cg15568145; cg06365535; cg01359962; cg07116393; cg13696942; cg09370594; cg25763393; and/or cg24136205.

In some embodiments, the methylation status of a significant number of the methylation markers shown in Table 1 may be determined. Herein, the term “a significant number” denotes at least 10%, at least 20%, at least 30%, at least 40%, at least 50%, at least 60%, at least 70%, at least 80%, at least 90%, at least 95%, or 100% (e.g., all) of the markers shown in Table 1 and/or Figures (e.g., FIG. 6). In some embodiments, the methods of the disclosure comprise detection of the markers of Table 1.

As is recognized in molecular biology, the markers (e.g., CpG sites) can reside within or overlapping genes or regulatory regions thereof or a locus thereto. For example, CpG sites may reside upstream of genes important for aging. Thus, in an example, the methods of the present disclosure encompass assessing methylation sites in coding and non-coding regions such as introns, in or across intron/exon boundaries, in or across splicing regions of the gene transcripts. Thus, by assessing multiple selected CpG sites, the methods of the present disclosure can encompass assessing methylation status of genes. In some embodiments, the sites may be at locus of a gene. Exemplary genes/loci whose methylation status may be assessed using the methods of the present disclosure are provided in Table 1.

In some embodiments, the methods of the present disclosure encompass assessing the methylation status of one or more genes or gene loci selected from the group shown in Table 1. For example, the methylation status of at least 2, 3, 4, 5, 6, 7, 8, 9, 10, 11, 12, 13, 14, 15, 16, 17, 18, 19, 20, 25, 30, 35, 40, 45, 50, 60, 75, 100, 125, 150, 175, 200, 225, 250, or more, e.g., all the genes or gene loci of Table 1 can be assessed. In some embodiments, the methylation markers in gene or gene loci in Table 1 are ordered in the order of relevance to the biological age, wherein genes/gene loci at the top of Table 1 have greater relevance than genes/gene loci at the bottom of Table 1. In some embodiments, the methods comprise assessing the methylation status of a plurality of the genes in Table 1.

All selected CpG sites of the present disclosure need not be completely methylated to indicate age. For example, predictive CpG methylation status can range from about 10% to about 90%, from about 20% to about 80%, from about 25% to about 75%, from about 30% to about 70% methylated CpG sites in a particular gene or regulatory region thereof. In some embodiments, predictive CpG methylation status is at least about 10%, 15%, 20%, 25%, 30%, 35%, 40%, 45%, 55%, 60%, 65%, 70%, 75%, 80%, 85%, 90%, 95%, or greater %, e.g., about 99% or even 100% methylation of CpG sites in a particular gene or regulatory region thereof.

The methylation status of the CpG sites of the present disclosure can be represented in various ways. In one example, determining the methylation status comprises calculating the ratio between methylated and unmethylated alleles for each CpG site and/or gene assessed. In an example, the ratio based on the methylated and unmethylated status can be represented as:

(methylated allele status)÷((un-methylated allele status+methylated allele status)×100)=methylation ratio.

In some embodiments, the methylation status for each allele is determined using a methylation array such as an INFINIUM HUMANMETHYLATION450 BEADCHIP exemplified below. The ratio based on the methylated and unmethylated intensity can be represented as:

(methylated allele intensity)÷((un-methylated allele intensity+methylated allele intensity)×100)=methylation ratio.

In some embodiments, the process of determining the methylation ratio can be performed for each CpG assessed and the resulting ratios can be added together to provide a score.

Because the predictive power of the identified CpG sites is sometimes additive or even synergistic (e.g., greater than additive), one of skill will appreciate that a methylation score indicative of aging or propensity for aging will largely depend on the number of CpG sites assessed. For example, when the methylation status of the 300 CpG sites shown in Table 1 are assessed, a methylation level of at least about 2, 3, 4, 5, 6, 7, 8, 9, 10, 11, 12, 13, 14, 15, 16, 17, 18, 19, 20, 20, 25, 30, 35, 40, 45, 50, 60, 75, 100, 125, 150, 200, 250, 275, or more, e.g., 300 of the CpG sites is indicative of aging or a propensity for aging.

A methylation status indicative of aging or a propensity for aging can be identified by assessing the CpG sites of the present disclosure relative to a control. Representative types of controls that may be used in the methods of the disclosure have been outlined above. In some embodiments, both positive and negative controls may be used in the methods of the present disclosure. For example, the positive control may comprise a sample obtained from a geriatric subject and the negative control may comprise a sample obtained from a neonate. To limit genetic variability, the positive and negative controls may be matched with respect to lineage (e.g., ancestry), race, gender, and the like, to the test sample. A plurality of controls may be used.

Various methods can be used to determine a change in the methylation status in the test sample relative to the control. For example, a change may be evident from a side by side comparison of methylation status between a test sample and a control(s). In another example, methylation status of test samples and controls can be compared statistically to identify a statistically significant difference in methylation status. There are a number of statistical tests for identifying a statistically significant difference in methylation status that vary significantly, including the conventional t-test. However, it may be generally more convenient appropriate and/or accurate to use other common tests to assess for such statistical significance such as ANOVA, Kruskal-Wallis, Wilcoxon, Mann-Whitney and odds ratio (OR). In certain embodiments, determining the age of the biological sample may comprise applying a linear regression model to predict sample age based on a weighted average of the methylation marker levels plus an offset.

The next step includes determination of age based on the methylation status. Generally, this step includes using a regression model, e.g., using a regression curve shown in FIG. 5, to calculate or predict an age of the biological sample. In some embodiments, a first predicted age is determined based on the methylation status and a second predicted age is determined by performing an operation (e.g., addition or subtraction) on the first predicted age. Specifically, the operation comprises an addition or subtraction of a delta age (δ), derived from a validation dataset of samples obtained from the subject, e.g., as provided in a hash table of Table 4. In such embodiments, the second predicted age may provide a more accurate estimate of the actual age of the sample. Performing the operative step may depend on which age group the first predicted age falls on. For e.g., if the predicted age is greater than 55 years, the operative step may be performed to calculate a second predictive age that is closer to, or more accurately reflective of, actual age.

### II. Workflow

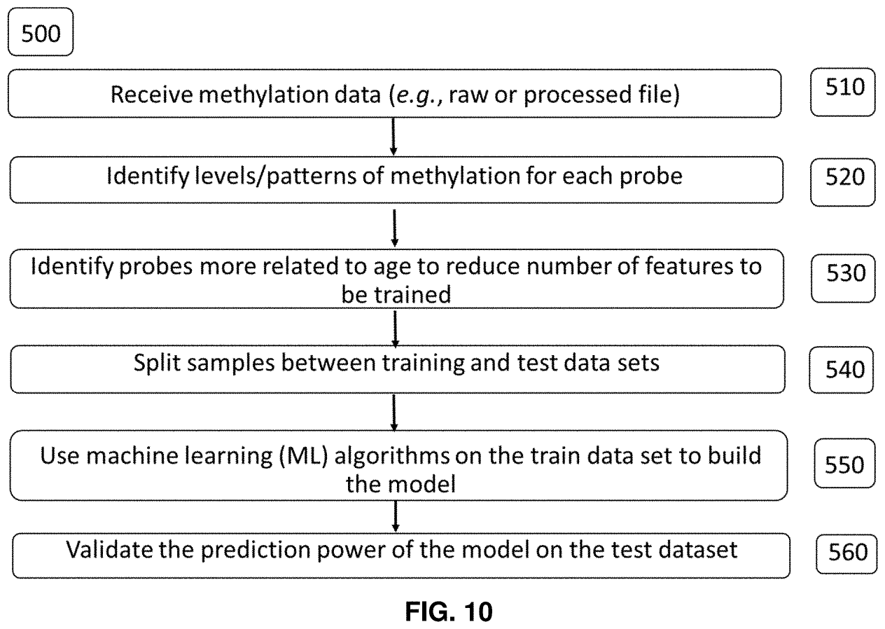

FIG. 10 is a flow chart illustrating a method 500 for diagnosing aging or a disease related thereto, e.g., neurodegeneration. Method 500 is illustrative only and embodiments can use variations of method 500. Method 500 can include steps for receiving methylation sequence data (e.g., in FASTQ/WIG/BED format); methylation array data (e.g., idat, BED, Matrix format); counting the number/levels of methylation markers; methylation analyzer (which optionally maps to genes); a regression model that is configured to systematically filter noise in the methylation data; and/or displaying the results.

In step 510 of method 500 of FIG. 10, a compendium of methylation markers is received from a subject. Any form of genetic data, e.g., raw data or process data, may be received. In some embodiments, the compendium of genetic markers is received in a methylation call format (idat or fastq) file.

In step 520 of method 500 of FIG. 10, the level or pattern of methylation of each marker is identified. Identification may include, e.g., bisulfite sequencing, which can be performed with most methylation sequencers. Sequencing may involve counting, which establishes a baseline level of methylation in reference and test samples from which a global estimate can be made. Methylation patterns may be analyzed using art-known methods, e.g., tilting microarray (Lippman et al., Nat. Methods 2, 219-224, 2005) or base-specific cleavage mass spectrometry (Ehrich et al., PNAS USA, 102, 15785, 2005).

In step 530 of method 500 of FIG. 10, the methylation markers that are related to age are identified. For example, markers that are differentially present in aged samples compared to non-aged samples may be identified using routine techniques, e.g., logistic regression, non-logistic regression, or the like. This step reduces the number of features that are utilized in training the machine learning (ML) algorithm. It should be noted that this step is optional in the case of human skin samples as markers that are differentially present in aged samples have already been identified using the instant systems/methods and are disclosed in Table 1 and/or Figures (e.g., FIG. 6). However, in the case of unknown samples, e.g., non-human samples, this step may be performed to crosscheck and/or validate markers that correlate with age.

In step 540 of method 500 of FIG. 10, the samples may be optionally split between training or test data sets. If the algorithm has already been trained with a representative data set, e.g., a dataset obtained from an in silico genetic data repository, then the samples need not be split. However, if the data set is archetypical or original, then it may be split to train the machine-learning algorithm and perform the desired analysis, e.g., determination of ROC values.

In step 550 of method 500 of FIG. 10, a machine learning approach may be incorporated to systematically eliminate or reduce noise. The approach may be applied at any step of the method, although it may be advantageous to implement the machine learning algorithm after the methylation markers have been identified in step 520 and/or parsed in step 530. In this regard, in the purely illustrative method of FIG. 10, a machine learning (ML) algorithm is optionally applied at step 550 to build the model. The ML algorithm may comprise employing a machine learning algorithm such as, e.g., using a Ridge regression machine learning algorithm to analyze actual patient samples to identify signatures that discriminate between true aging methylation markers and noise.

In some embodiments, the ML is trained with a dataset. For example, the dataset may include epidermal and/or dermal and/or whole skin samples from subjects, both male and female, who are about 18 years to about 90 years of age. The association between specific methylation markers and aging is identified using a robust mathematical regression. The markers that are highly specific and tightly associated with aging, as identified using the robust mathematical regression, are then studied for the features, including, association with any aging-related genes or signatures. A representative method is described in the Examples. It should be noted that the training step is optional in the case of human skin samples as markers that are differentially present in aged samples have already been identified using the instant systems/methods and are disclosed in Table 1 and/or Figures (e.g., FIG. 6). However, in the case of unknown samples, e.g., non-human samples, this step may be performed to train the algorithm to identify which of the markers of Table 1 are more tightly (or loosely) associated with aging.

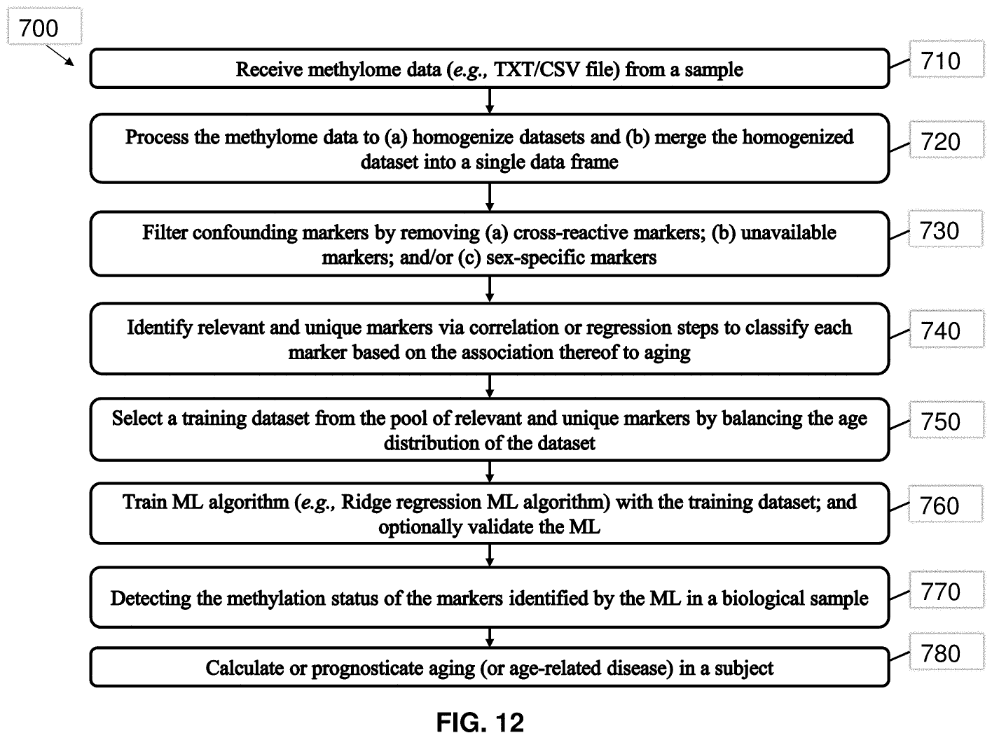

FIG. 12 shows a workflow illustrating an embodiment method 700 for developing a model for calculating or predicting the age of biological samples (e.g., skin, sperm, eggs, etc.). Method 700 is illustrative only and embodiments can use variations of method 700. Method 700 can include steps for pre-analytical data processing; removing confounding markers; and performing the analysis, e.g., calculating the age or predicting the age of biological samples.

In step 710 of method 700 of FIG. 12, a plurality of methylome datasets from a plurality of heterogeneous samples of different age or age groups, wherein each dataset comprises a plurality of methylation markers, is received in a file. Additionally, a feature annotation such as tissue, gender, ethnicity and age composition may be included.

In step 720 of method 700 of FIG. 12, the methylome datasets are processed. This step may include homogenization of the methylome datasets and merging the homogenized dataset into a single data frame to generate a string of homogenized and merged methylation markers.

In step 730 of method 700 of FIG. 12, confounding markers are filtered. For instance, cross-reactive markers, unavailable markers, and/or sex-specific markers may be filtered from the processed dataset.

In step 740 of method 700 of FIG. 12, relevant markers are identified from the filtered markers. The identification method may include carrying out a plurality of correlation or regression steps to classify each marker based on the association thereof to aging, combining the results of each regression or correlation step to identify relevant markers, and eliminating redundant markers. Implementation of these steps, either in series or together with a single step, results in a pool of relevant markers.

In step 750 of method 700 of FIG. 12, a training dataset is selected from the pool of relevant markers. The selection step may include balancing the age distribution of samples from which the relevant markers are obtained. This may be achieved by ensuring that not more than n samples per age window of y years, beginning with age z years, wherein n, y, and z are integers >0. In one specific embodiment, the selection step is implemented to ensure that not more than 5 samples per age window of 7 years, beginning with age 18 years is included in the dataset. This minimizes or eliminates potential age bias, which may be introduced as a result of over-representation of certain age/age groups in the dataset.

The aforementioned steps are implemented to systematically eliminate or reduce confounding markers and identify markers that are relevant to age. Additionally, by implementing the balancing step, a training dataset is selected which is representative of various age groups in a population.

In some embodiments, the workflow may be terminated after the training dataset is obtained. In some embodiments, the workflow is carried out to include downstream steps including machine learning, optionally together with the validation step; and the analysis steps for determining age of a biological sample (e.g., skin tissue of a human subject).

In some embodiments, the filtered and balanced training dataset is processed by an algorithm to identify markers that are associated with aging. For instance, in step 760 of method 700 of FIG. 12, the machine-learning algorithm is trained with the training dataset of step 750. In some embodiments, this may include employing a Ridge regression machine-learning algorithm, which generates a plurality of age-specific and relevant methylation markers with respect to age. In this step, a validation step may be further used to validate and/or fine-tune the trained machine-learning algorithm.

It should be noted that the workflow may be carried out with a trained machine learning module or algorithm. That is, in some embodiments, the age determination workflow 700 may be initiated using a trained machine learning module without the need to implement upstream steps 710 to 750.

In a subsequent step of the age determination workflow 700, methylation data of a biological sample (e.g., skin tissue) is analyzed. For instance, in step 770 of method 700 of FIG. 12, methylation status of age-specific and relevant methylation markers are detected in a biological sample. The detection step may be preceded by a sample processing step. In some embodiments, the sample may be processed at site, for example, by coupling a methylation sequencer (e.g., bisulfite sequencer). In other embodiments, sample processing is not needed as the methylation data of the sample (or subject) are received separately (e.g., in a file) and the methylation status of the age-specific and relevant methylation markers in the dataset are analyzed directly. As mentioned previously, analysis of methylation status may include determination of the levels and/or patterns of methylation markers, e.g., one or more of the markers of Table 1 and/or FIG. 6, in the sample.

In step 770 of method 700 of FIG. 12, the age of the biological sample is calculated based on the detected methylation status of the biological sample. In some embodiments, prediction or calculation of the age is performed using a regression model, e.g., using a regression curve shown in FIG. 5.

With routine tweaks, the aforementioned workflow may be used in other applications, e.g., identifying subjects (e.g., who are abnormally aging), identifying subjects at risk for developing age-related diseases; identifying subjects who can undergo conception (e.g., via in vitro fertilization) or serve as sperm donors; or determining the efficacy of age-reversing drugs or therapy in vitro, ex vivo or in vivo.

The architecture of the machine learning approach will be discussed in greater detail below.

Machine Learning (ML)

Not being bound to a single embodiment and purely for the purpose of illustration, a machine learning algorithm was built in two parts (A) and (B). The first part (A) includes selecting three public datasets, e.g., (1) Dataset GSE51954 (accessioned Mar. 23, 2015; see, Vandiver et al., Genome Biol 2015 Apr. 16; 16:80); (2) Dataset GSE90124 (accessioned Jan. 4, 2017; see, Roos et al., J Invest Dermatol 2017 April; 137(4):910-920); and (3) Dataset E-MTAB-4385 (released on Mar. 24, 2016 in ARRAYEXPRESS database; see, Bormann et al., Aging Cell, 15(3):563-71, 2016). All the information in the datasets were available on the public domain, and criteria such as tissue, gender and age composition were used in the selection. This strategy allowed use of 508 samples (40 dermis, 146 epidermis, whole skin 322), wherein each sample comprised more than 450,000 CpG/probes/features. In order to build a regression model based on a machine learning algorithm able to predict age in an accurate way these datasets were merged, preprocessed, divided into training subset and testing subsets, and age-balanced as described next. First, a merging script was written to obtain the raw data of each dataset, extract the methylation matrices and turn them into data frames. The merge script also extracted the meta-data and labeled the data. All data were then joined into a single data frame generating a list of methylation levels with 508 samples. Second, a second script was written for preprocessing the data to remove the cross-reactive probes (Chen et al., Epigenetics, 8(2):203-9, 2013). This helps to reduce the number of probes to the ones that are specific in their hybridization pattern, which reduces computational cost of the downstream steps and delivers, to the algorithm, probes that represent meaningful differential data points. Then this same script was used to remove unavailable probe holders, if any were any present. Finally, the script removed the sex-specific chromosome-related probes and the probes that are not present in a methylation array such as the INFINIUM METHYLATION EPIC Kit. The sex-specific probes were removed so the dataset represented the differences of methylation related to the age of the samples and not to their gender, as the sexual probes could create a bias and mistakenly train the algorithm to select probes that are also important for age but are gender specific. The probes that were not present in the methylation array such as INFINIUM METHYLATION EPIC Kit were removed as a practical decision. It should be noted that the removal of unavailable probes is due to limitation of the INFINIUM commercial kit as old datasets used kits that are not represented in the kit have limited use in quantifying age of unknown samples. Should a kit cover the entire methylome, then it is possible to carry out the method or devise the workflow without removing the unavailable probes. Third, a third script was utilized to perform feature selection. The third script combined the results of three different methodologies; glmnet-lasso, xgboost, and ranger.

Each the aforementioned methodologies, run by the script, provided a list of the most relevant features/probes with respect to its mathematical model for predicting a parameter of interest, in this case, age. The script took the results of each one, combined them and maintained a unique probe on the cases that one probe was present in more than one of the results. The net result is a set of 300 relevant probes from each sample. Finally, samples were selected for the training dataset in order to have a balanced distribution between the ages, with the criteria of not having more than 5 samples per age window of 7 years, beginning with age 18. The balanced-training dataset had 249 samples and the 259 rest of samples were used for the testing dataset. To balance the age distribution of the training dataset allows the algorithm to be able to predict ages without bias to certain ages that could be overrepresented in the training dataset and perform equally along younger or older samples in terms of age quantification.

For developing and testing the algorithm, Several Machine Learning algorithms implemented by the caret package for R environment were tested. In each case, a 50 fold resampling cross-validation was used for optimization of the tuning parameters. Model prediction errors were computed using mean absolute error (MAE) and/or root mean squared error (RMSE) and the fitness levels and significance of the applied regression models were evaluated by computing Pearson's correlation coefficient using the training data (e.g., smaller MAE or RMSE scores indicate better predictive algorithm and an R2 value that ˜1.0 indicates better fit). The best performance was obtained with the Ridge Regression machine learning algorithm, which penalizes the size of parameter estimates by shrinking them to zero in order to decrease complexity of the model, while including all the variables in the model. In step 560 of method 500 of FIG. 10, the prediction power of the model on the test dataset is validated, e.g., using a probability model such as logistic regression. Optionally, a resampling may be performed to obtain an unbiased appraisal of the model's likely future performance.

### III. Applications

Method of Screening Compounds Useful in Reversing Aging or Treating Age-Related Diseases

It should be appreciated that, with some modifications, the compound discovery workflows disclosed herein, can also be broadly used for screening and discovery of compounds that may be useful in preventing or curing (i.e., reversing) a number of well-known age-related diseases and conditions. An exemplary list of age-related diseases for which compounds can be screened is provided below.

Macular Degeneration

Age Macular Degeneration (AMD) constitutes a leading cause of blindness in industrialized countries, affecting approximately 8% of the population within ages 45-85 years. It is estimated that 196 million affected people in 2020. AMD's primary cause is the loss of retinal pigmented cells, which leads to photoreceptor death.

It is well documented in medical literature that, with age, both photoreceptors and the retinal pigment epithelium show slow degenerative changes, followed by their demise and often accompanied by the development of a neovascular membrane. Moreover, chronic and repetitive non-lethal retinal pigment epithelium (RPE) injuries (together with an oxidative environment) appear to be important factors for development of AMD.

Cellular senescence (i.e., aging) has also been associated with the disease, which may corroborate the role of aging in this pathology. In vitro evidence supports this hypothesis, being that, the exposure of RPE cells to senescence-inducing stimuli, such as H₂O₂, promotes senescence-associated secretory phenotype (SASP) expression that is characterized by the production and release of specific soluble molecules, such as pro-inflammatory cytokines, which are linked to AMD pathogenesis.

Despite this evidence, no evaluation of the age-related biomarkers (e.g., epigenetic, genetic, etc.) of the RPE cells has been performed. In addition, by collecting tissue of AMD and non-AMD donors, it will be possible to confirm the hypothesis that precocious senescence may cause AMD and that anti-aging strategies may successfully prevent AMD.

Although much progress has been made recently in the management of the later stages of AMD, no agents have yet been developed for the early stages or for prophylactic use. This might be finally achieved through prevention of cellular senescence.

Dementia

Considering age-related cognitive decline, age is the primary risk factor for many neurodegenerative diseases including Alzheimer's disease (AD), Parkinson's disease and dementia, which is an umbrella term used to describe diseases that cause dysfunction or death of neurons. Neural cells in AD patients show strong immunoreactivity for p16Ink4a a biomarker of aging, which is not presented in non-senescent, terminally differentiated neurons. In addition, telomeres tend to be shorter in patients with dementia compared to healthy ones and senescent astrocytes contribute to AD. Age-related biomarkers (e.g., epigenetic, genetic, etc.) of the brain is currently a target of research, being that such molecular evidence of aging is highly associated with cognitive decline. Therefore, there is increasing evidence that cellular senescence (i.e., aging) may be related to neuron dysfunction associated with dementia.

Despite such evidence, current studies are mainly observational and do not propose interventional strategies. By measuring age-related biomarkers (e.g., epigenetic, genetic, etc.) of brain tissue prior to and after molecule testing, it may be possible to screen novel molecules with anti-aging potential for the brain, and, possibly, preventive effect over such pathology.

Atherosclerosis

Atherosclerosis is frequently the underlying cause of cardiovascular diseases, which are the primary cause of mortality in the Western world. This disease is highly influenced by age, in addition to environmental factors. Corroborating such observation, it has been well documented in medical literature that, during atherosclerotic plaque formation and expansion, senescent (i.e., aged) vascular smooth muscle and endothelial cells can be found. Two mechanisms of senescence induction in this context are cellular proliferation, as well as oxidative stress. Because of the complex signaling between endothelial and smooth muscle cells, and immune cells recruited to plaques, these findings raise the possibility of a multistep role of senescent cells in atherogenesis and the possibility that anti-aging therapeutic compounds may be discovered to prevent or reverse atherosclerosis.

Cancer

Cancer constitutes a pathology associated with cellular proliferation, independently from external stimuli. Most cancers are associated with aging. Confirming such an observation, DNA aging (as quantified by age-related biomarkers) has been linked with cancer risk factors (e.g., breast cancer risk) which raises the possibility that anti-aging therapeutic compounds may be discovered to prevent or cure cancer.

In some embodiments, the aforementioned methods for screening compounds that modulate aging or a disease-related thereto comprises the following steps: (a) detecting the status of a plurality of methylation markers from Table 1 in a genomic DNA (gDNA) of a biological sample and calculating a first age of the subject's biological sample based on the status of the detected methylation markers, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) contacting the biological sample with a test compound; and (c) detecting the status of a plurality of the methylation markers of (a) in the genomic DNA (gDNA) of the biological sample contacted with the test compound and calculating a second age of the test compound-contacted biological sample based on the status of the methylation markers detected in (a); wherein if the second calculated age of the biological sample is modulated compared to the first calculated age of the biological sample, then the test compound is identified as modulating aging or a disease-related thereto. Herein, a difference between the subject's first calculated age and second calculated age (δ) can be used in the identification of modulating test compounds. For instance, a threshold δ may be first computed using known samples to determine a standard error rate, and this threshold value may be used to reliably ascertain whether the modulating effect of a specific compound is due to pure chance or due to its biological property.

In some embodiments, an absolute delta (δ) greater than 1 month, 6 months, 1 year, 2 years, 3 years, 4 years, 5 years, 6 years, 7 years, 8 years, 9 years, 10 years, or 11 years, or more, e.g., 12 years (preferably about 5 years) can be used as a threshold for making such determinations. More specifically, in some aspects, a positive delta (+δ), e.g., a δ of +5 years, may be used as threshold for identifying whether a test compound is a promoter of aging or an age-related disease. Conversely, a negative delta (−δ), e.g., a δ of −5 years, may be used as threshold for identifying whether a test compound is a reverser of aging or an age-related disease.

Preferably, the screening methods of the disclosure are carried out in high throughput screening (HTS) format. Herein, a small-molecule drug discovery project usually begins with screening a large collection of compounds against a biological target that is believed to be associated with a certain disease, e.g., aging. The goal of such screening is generally to identify interesting, tractable starting points for medicinal chemistry. Despite the fact that screening of huge libraries containing as many as one million compounds can now be accomplished in a matter of days in pharmaceutical companies, the number of compounds that eventually enter the medicinal chemistry phase of lead optimization is still largely limited to a couple of hundred compounds at best. In that regard, it is generally well understood that one significant challenge to the early hit-to-lead process of drug discovery is selecting the most promising compounds from primary HTS results. In current HTS data analysis, an activity cutoff value is usually set to allow selection of a certain number of compounds whose tested activities are greater than (or less than, depending upon the application) this threshold. The selected compounds are called “primary hits” and are subject to retesting for confirmation. Following such retesting and confirmation, confirmed or validated primary hit compounds are grouped into families. Based upon further evaluation or additional chemical exploration, the families that exhibit certain desired or promising characteristics (such as, for example, a certain degree of structure-activity relationship (SAR) among the compounds in the family, advantageous patent status, amenability to chemical modification, favorable physicochemical and pharmacokinetic properties, and so forth) are selected as lead series for subsequent analysis and optimization.

In accordance with some embodiments, for example, a high-throughput screening hit identification method may generally comprise: selecting a family of compounds to be analyzed; evaluating the family of compounds in accordance with a relationship characteristic; and prioritizing ones of the compounds in accordance with evaluation methodology of the disclosure (e.g., analyzing changes in expression, levels, or activities of the biomarkers of the disclosure). Some such methods may further comprise selectively repeating the selecting and the evaluating until a predetermined number of families of compounds has been selected and evaluated.

In the evaluation step, a probability score is assigned to the family of compounds and such assigning may comprise, e.g., computing a non-parametric probability score, calculating the probability score based upon an hypergeometric probability distribution, or both. The evaluating may be executed in accordance with a structure-activity relationship analysis, for instance, or in accordance with a mechanism-activity relationship. Some exemplary methods for evaluation of screened compounds comprise ranking the compounds in accordance with an activity criterion; in methods employing such ranking, the prioritizing may further comprise analyzing selected ones of the compounds in accordance with the ranking and the evaluating.

In some embodiments, a computer-readable medium encoded with data and instructions for high-throughput screening hit selection may be used. The data and instructions may cause an apparatus executing the instructions to: identify a family of compounds to be analyzed; rank each respective compound to be analyzed with respect to an activity criterion (e.g., changes in levels or activity of one of the markers of Table 1 or gene linked to the marker or a locus thereto); evaluate the family of compounds in accordance with a relationship characteristic; and prioritize ones of the compounds in accordance with results of the evaluation and in accordance with rank.

The computer-readable medium may be further encoded with data and instructions causing an apparatus executing the instructions selectively to repeat identifying a family of compounds and evaluating the family of compounds. In some embodiments, the data and instructions may further cause an apparatus executing the instructions to assign a probability score to the family of compounds; as set forth below, this may involve computing a non-parametric probability score, calculating the probability score based upon an hypergeometric probability distribution, or both. For example, the algorithms and scoring methods of the present disclosure may be implemented in this step. For some applications, the computer-readable medium may be further encoded with data and instructions causing an apparatus executing the instructions to evaluate the family of compounds in accordance with a structure-activity relationship analysis or in accordance with a mechanism-activity relationship analysis.

In some implementations, an exemplary high-throughput screening system may generally comprise: a processor operative to execute data processing operations; a memory encoded with data and instructions accessible by the processor; and a hit selector operative, in cooperation with the processor, to: identify a family of compounds to be analyzed; evaluate the family of compounds in accordance with a relationship characteristic; and prioritize ones of the compounds in accordance with results of the evaluation and in accordance with a rank for each respective compound, the rank being associated with an activity criterion.

Embodiments are disclosed wherein the hit selector is further operative selectively to repeat identifying a family of compounds and evaluating the family of compounds. The hit selector may be further operative to assign a probability score to the family of compounds.

In some systems, the hit selector is further operative to evaluate the family of compounds in accordance with a structure-activity relationship analysis; additionally or alternatively, the hit selector may be further operative to evaluate the family of compounds in accordance with a mechanism-activity relationship analysis.

Patient Identification, Disease Prognosis and/or Theranostic Applications

In some embodiments, the methods of the present disclosure can be used to identify subjects of interest. The methods can be used in a pre-screening or prognostic manner to assess whether a subject has or is likely to develop an age-related disorder, and if warranted, a further definitive diagnosis can be conducted. For example, the methods described herein can be used to screen or prognosticate whether a subject has or is likely to develop hypertension, atherosclerosis, diabetes mellitus, dementia, skin disorders, and other age-related diseases.

In some embodiments, the methods of the present disclosure can be used to determine the therapeutic effectiveness of a drug or therapy (e.g., in theranostic applications). For example, the methods of the present disclosure can be used to determine a subject's response to anti-hypertensive drugs (e.g., a diuretic). In this example, a reduction in methylation of the CpG sites of the present disclosure is indicative of a positive response to the therapy. For example, a patient may provide a sample before therapy is initiated and provide additional samples over time as treatment progresses. The initial sample can be used as a baseline and a decrease in methylation indicates that the patient is responding to the therapy. In another example, a sample can be obtained from patients subject to the therapy and compared with a control sample. Such assessments can be repeated at various time points as treatment progresses and/or escalates to detect whether the subject is responding to therapy.

In some embodiments, the methods of identifying a subject for aging or having an age-related disease comprise the following steps: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating the age of the subject's biological sample based on the status of the detected methylation markers, wherein if the calculated age of the sample is greater than the subject's actual age, then the subject is positively identified as aging or an age-related disease. Herein, the difference between the subject's actual age and calculated age (Δ) can be used in the positive identification of subjects. In some embodiments, an absolute delta (Δ) greater than 1 month, 6 months, 1 year, 2 years, 3 years, 4 years, 5 years, 6 years, 7 years, 8 years, 9 years, 10 years, or 11 years, or more, e.g., 12 years, can be used as a threshold for the positive identification of subjects. For instance, if the subject's calculated age exceeds the subject's actual age by a number that is greater than the threshold, then the subject is identified as aging abnormally. Preferably, a threshold Δ of about 5 years can be used in identifying subjects that are aging abnormally.

As is evident from the foregoing, the instant systems and methods can be used to identify subjects who are experiencing premature aging (or with age-related disease) as well as subjects with delayed onset of aging (or with no age-related disease). For instance, if the calculated age >actual age by at least the threshold level (e.g., about 5 years), then the subject may be identified as having premature aging; and if the calculated age <actual age by at least the threshold level (e.g., about 5 years), then the subject may be identified as having delayed onset of aging.

Preferably, the subjects who are identified for premature aging or delayed onset aging comprise subjects who are older than 40 years; preferably older than 50 years; more preferably older than 60 years; and especially older than 70 years, e.g., between 50-90 years.

Once the subject is positively screened for aging or age-related diseases in accordance with the foregoing, further tests may be carried out. Such further tests include, e.g., genetic tests, physiological tests (e.g., monitoring blood pressure), psychological evaluations, evaluation of family history, or a combination thereof. Specific tests for monitoring hypertension, atherosclerosis, diabetes mellitus, dementia, skin disorders, and other age-related diseases, may also be carried out. In some embodiments, the methods of prognosticating a subject for developing aging or an age-related disease comprise the following steps: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating the age of the subject's biological sample based on the status of the detected methylation markers, wherein if the calculated age of the sample is greater than the subject's actual age, then the subject is prognosticated as being at risk for developing aging or an age-related disease. Here too, a difference between the subject's actual age and calculated age (Δ) can be used in the prognostication of aging or age-related diseases, wherein, a greater Δ is associated with greater risk of developing aging or age-related disease. In some embodiments, a threshold delta (Δ) of 1 month, 6 months, 1 year, 2 years, 3 years, 4 years, 5 years, 6 years, 7 years, 8 years, 9 years, 10 years, or 11 years, or more, e.g., 12 years, can be used in making a high-confidence prediction, the delta value differing from one subject class to another (e.g., teenage vs. geriatric subjects). In some embodiments, the threshold Δ of about 5 years is used in the prognostication.

In some embodiments, the methods of determining the efficacy of a drug or a therapy against aging or an age-related disease comprise the following steps: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating a first calculated age of the subject's biological sample based on the status of the detected methylation marker; (c) administering to the subject, an anti-aging drug or therapy if the first calculated age of the subject's sample is greater than the subject's actual age; (d) detecting the status of a plurality of the methylation markers of (a) in the genomic DNA (gDNA) of the biological sample of the subject treated with the anti-aging drug or therapy and calculating a second calculated age of the test compound-contacted biological sample based on the status of the methylation markers detected in (a); and (e) determining the effectiveness of the anti-aging drug or therapy based on the modulation of the second calculated age compared to the first calculated age. Herein, if the second calculated age is less than the first calculated age (preferably the difference between the first and second calculated age is greater than a threshold level, e.g., 5 years), then the anti-aging drug or therapy is deemed effective. Conversely, if the difference between the first and second calculated age is negative (i.e., second calculated age >first calculated age) or the difference is less than a threshold level (e.g., 5 years), then the anti-aging drug or therapy is deemed ineffective.

In some embodiments, the methods of determining efficacy of a drug or therapy against aging or an age-related disease includes carrying out the aforementioned steps in a patient who is suffering from aging or the age-related disease. In such instances, the methods may comprise (a) administering to the patient, an anti-aging drug or therapy; (b) detecting the status of a plurality of the methylation markers of (a) in the genomic DNA (gDNA) of the biological sample of the subject treated with the anti-aging drug or therapy and calculating a second calculated age of the test compound-contacted biological sample based on the status of the methylation markers detected in (a); and (e) determining the effectiveness of the anti-aging drug or therapy based on the modulation of the second calculated age compared to the first calculated age.

Method of Treatment

The methods of the present disclosure can be incorporated into methods of treating aging or age-related disorders. If aging or a propensity to develop aging is detected in a subject using the methods of the present disclosure, the subject can be directed or prescribed an appropriate treatment for the condition. For example, aging detected using the methods of the present disclosure may be treated with a pharmacological agent. Suitable exemplary therapies include, but are not limited to, nutritional therapy, e.g., caloric restriction, use of bioactive compounds such as resveratrol, epigenetic modifiers (e.g., sulforaphane, epigallocatechin-3-gallate (EGCG), quercetin, and genistein); exercise therapy or a combination thereof. See, Kim et al., Prey Nutr Food Sci. 22(2): 81-89, 2017.

In some embodiments, the methods of treating aging or an age-related disease comprise the following steps: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating a first calculated age of the subject's biological sample based on the status of the detected methylation marker; (c) administering to the subject, an anti-aging drug or therapy if the first calculated age of the subject's sample is greater than the subject's actual age; (d) detecting the status of a plurality of the methylation markers of (a) in the genomic DNA (gDNA) of the biological sample of the subject treated with the anti-aging drug or therapy and calculating a second calculated age of the biological sample of the treated subject based on the status of the methylation markers detected in (a); and (e) continuing anti-aging drug treatment or therapy until the second calculated age is within a threshold level of the subject's actual age. Herein, a predetermined threshold level (e.g., 5 years) may be used to determine the duration of drug treatment or therapy. Methods of determining threshold levels are outlined in the Examples section. For instance, the respective age of various samples of the subject (e.g., dermis, epidermis, basement membranes, etc. of skin tissues) may be subject to analysis of methylation markers in accordance with the present disclosure and the calculated age of these samples are compared with the subject's actual age to arrive at a threshold value. For e.g., the threshold value may include 1, 2 or 3 standard deviations (preferably one standard deviation) of the mean difference between the calculated age and the actual age across n samples, wherein the n samples are obtained from the same subject or different subjects (preferably different subjects who are similar to each other with respect to demographic factors such as race, ethnicity, gender, and/or actual age).

Other Applications

The data presented herein may serve as a foundation for the sperm diagnostic tests to assess the risk of transmission of epigenetic alterations through the male germ line that may cause disease, or increase the risk of disease development, in offspring. Potential methodologies to screen for important methylation alterations in sperm include without limitation, region specific bisulfate pyrosequencing, array based methylation analysis (e.g., Illumina HUMAN METHYLATION450 array), or methyl sequencing (whole genome, region specific, or methyl capture sequencing, or MeDIP sequencing). Two broad applications include the analysis of risk to patients attempting to conceive, as well as the possible use of selecting sperm using sperm selection procedures that may transmit a lower risk.

In some embodiments, provided herein are methods of assessing risk of developing conception-related complications in subjects attempting to conceive, comprising: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating the age of the subject's biological sample based on the status of the detected methylation markers, wherein if the calculated age of the sample is greater than the subject's actual age, then the subject is identified as being at risk for developing conception-related complications. Herein, the difference between the subject's actual age and calculated age (Δ) can be used in the positive identification of subjects. In some embodiments, a delta (Δ) greater than 1 month, 6 months, 1 year, 2 years, 3 years, 4 years, 5 years, 6 years, 7 years, 8 years, 9 years, 10 years, or 11 years, or more, e.g., 12 years, can be used as a threshold for the assessment of risk. For instance, if the subject's calculated age exceeds the subject's actual age by a number that is greater than the threshold, then the subject is identified as being at risk of developing complications during conception and/or pregnancy. Preferably, a threshold Δ of about 5 years is used in identification of the subjects that are at risk for developing complications during conception and/or pregnancy.

In some embodiments, provided herein are methods of assessing health of sperm samples from donors, comprising: (a) detecting the status of a plurality of methylation markers from Table 1 in a genomic DNA (gDNA) of the subject's biological sample (e.g., sperm sample), wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating the age of the subject's biological sample (e.g., sperm sample) based on the status of the detected methylation markers, wherein if the calculated age of the biological sample (e.g., sperm sample) is greater than the subject's actual age, then the subject is identified as being an unhealthy donor and/or if the calculated age of the biological sample (e.g., sperm sample) is lesser than the subject's actual age, then the subject is identified as being a healthy donor. Herein, a level of difference between the subject's actual age and calculated age (Δ) is used in characterizing healthy versus unhealthy donors. In some embodiments, a delta (Δ) greater than 1 month, 6 months, 1 year, 2 years, 3 years, 4 years, 5 years, 6 years, 7 years, 8 years, 9 years, 10 years, or 11 years, or more, e.g., 12 years, can be used as a threshold for the assessment of healthy or unhealthy donors. For instance, if the subject's calculated age exceeds the subject's actual age by a number that is greater than the threshold, then the subject is identified as being an unhealthy donor. Conversely, if the subject's calculated age is below the subject's actual age by a number that is greater than the threshold, then the subject is identified as being a healthy donor. Preferably, a threshold Δ of about 5 years is used in identification of the subjects that are healthy/unhealthy sperm donors.

### III. Compositions and Kits

This disclosure also provides kits for the detection and/or quantification of the diagnostic biomarkers of the disclosure, or expression or methylation level thereof using the methods described herein.

The kits for detection of methylation level can comprise at least one polynucleotide that hybridizes to one of the CpG loci identified in Table 1 (or a nucleic acid sequence at least 90%, 92%, 95% and 97% identical to the CpG loci of Table 1), or that hybridizes to a region of DNA flanking one of the CpG identified in Table 1, and at least one reagent for detection of gene methylation. Reagents for detection of methylation include, e.g., sodium bisulfite, polynucleotides designed to hybridize to sequence that is the product of a biomarker sequence of the disclosure if the biomarker sequence is not methylated, and/or a methylation-sensitive or methylation-dependent restriction enzyme. The kits can provide solid supports in the form of an assay apparatus that is adapted to use in the assay. The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

In some embodiments, the kits of the disclosure comprise one or more (e.g., 1, 2, 3, 4, or more) different polynucleotides (e.g., primers and/or probes) capable of specifically amplifying at least a portion of a DNA region where the DNA region includes one of the CpG Loci identified in Table 1. Optionally, one or more detectably-labeled polypeptides capable of hybridizing to the amplified portion can also be included in the kit. In some embodiments, the kits comprise sufficient primers to amplify 2, 3, 4, 5, 6, 7, 8, 9, 10, or more different DNA regions or portions thereof, and optionally include detectably-labeled polynucleotides capable of hybridizing to each amplified DNA region or portion thereof. The kits further can comprise a methylation-dependent or methylation sensitive restriction enzyme and/or sodium bisulfite.

### IV. Computer Implemented Methods and Systems

The methods of the present disclosure may be implemented by a system. In an example, the system is a computer system comprising one or a plurality of processors which may operate together (referred to for convenience as “processor”) connected to a memory. The memory may be a non-transitory computer readable medium, such as a hard drive, a solid state disk or CD-ROM. Software, that is executable instructions or program code, such as program code grouped into code modules, may be stored on the memory, and may, when executed by the processor, cause the computer system to perform functions such as determining that a task is to be performed to assist a user to determine the methylation status of CpG sites in DNA obtained from the subject, the CpG sites being selected from the present disclosure (e.g., Table 1); receiving data indicating the methylation status of CpG sites in DNA obtained from the subject; processing the data to detect aging or the propensity to develop aging based on a methylation status of the CpG sites; outputting the existence of aging or a propensity for aging in a subject.

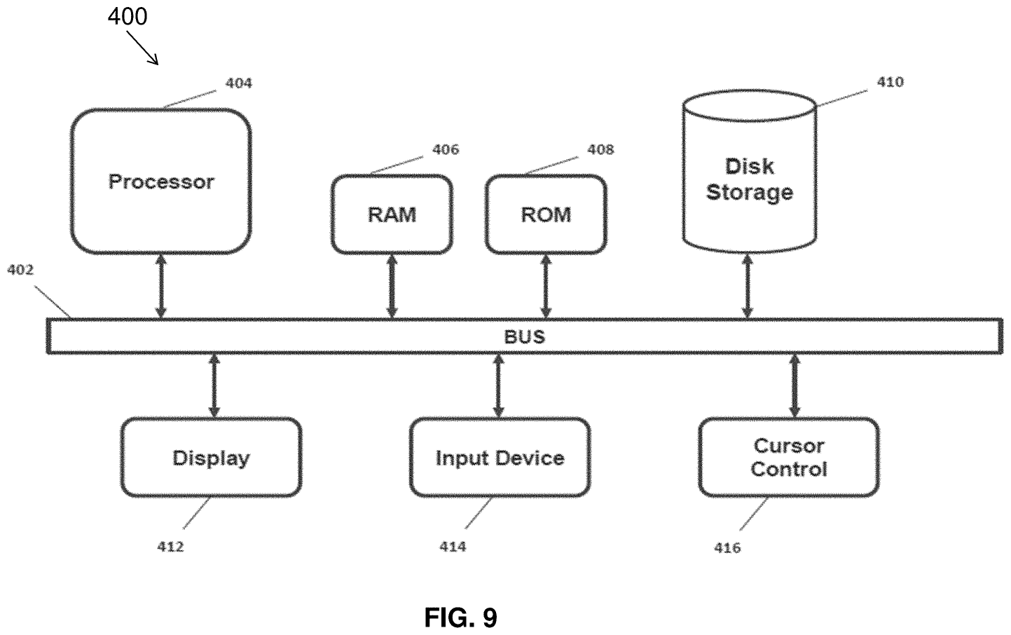

In some embodiments, the diagnostic methods of the disclosure are implemented on a computer system. Purely as a representative example, the schematic representation of such computer systems is provided in FIG. 9. FIG. 9 shows a block diagram that illustrates a computer system 400, upon which, embodiments or portions of the embodiments, of the present disclosure may be implemented. In various embodiments of the present disclosure, computer system 400 can include a bus 402 or other communication mechanism for communicating information, and a processor 404 coupled with bus 402 for processing information. In various embodiments, computer system 400 can also include a memory, which can be a random access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions. In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (x, y and z) cursor movement are also contemplated herein.

Consistent with certain implementations of the present disclosure, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, e.g., telephone modem connections, wide area networks (WAN), local area networks (LAN), infrared data connections, NFC connections, etc.

It should be appreciated that the methodologies described herein, including flow charts, diagrams and accompanying disclosure can be implemented using computer system 400 as a standalone device or on a distributed network of shared computer processing resources such as a cloud-computing network.

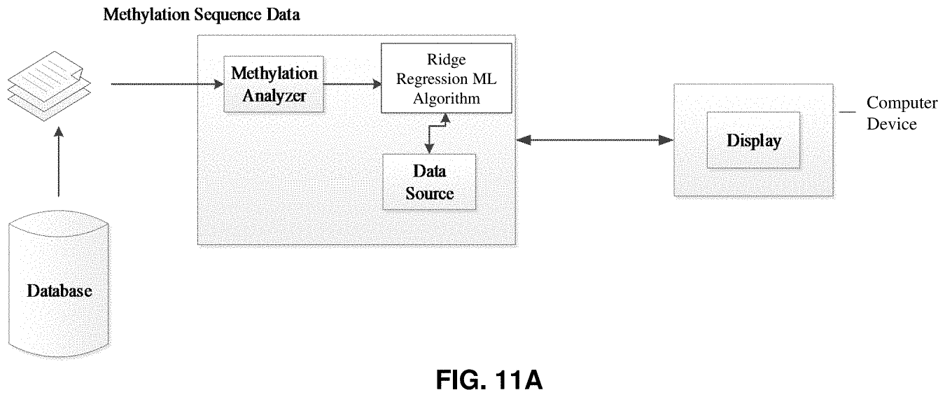

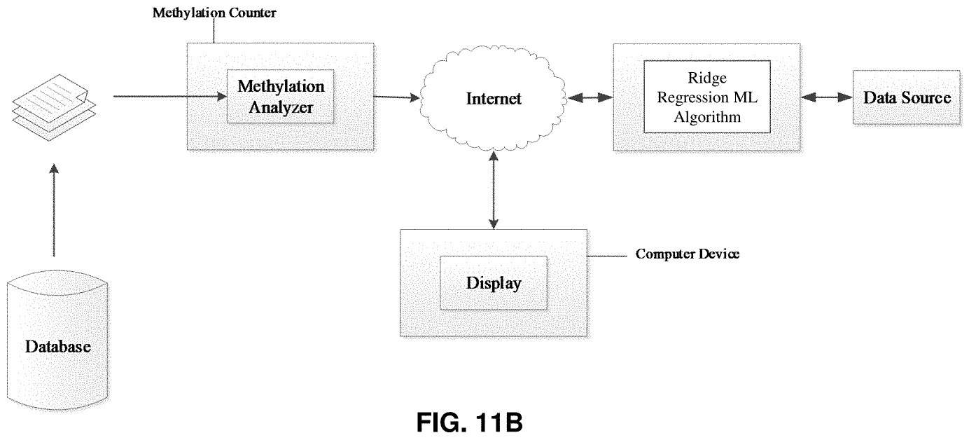

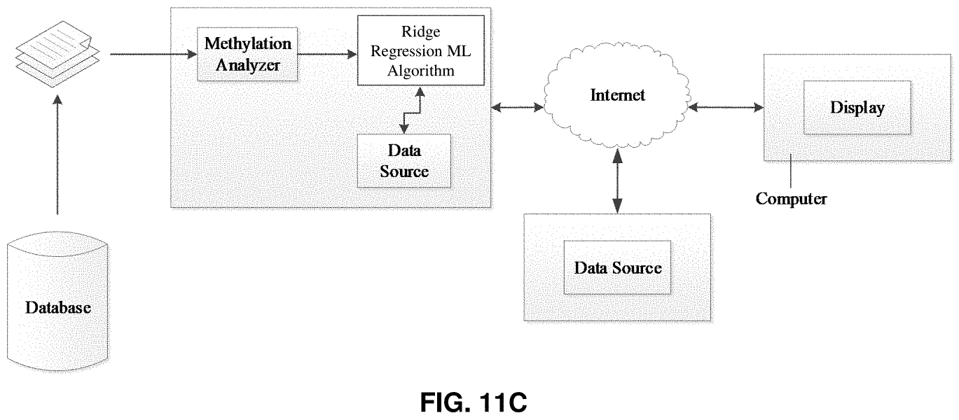

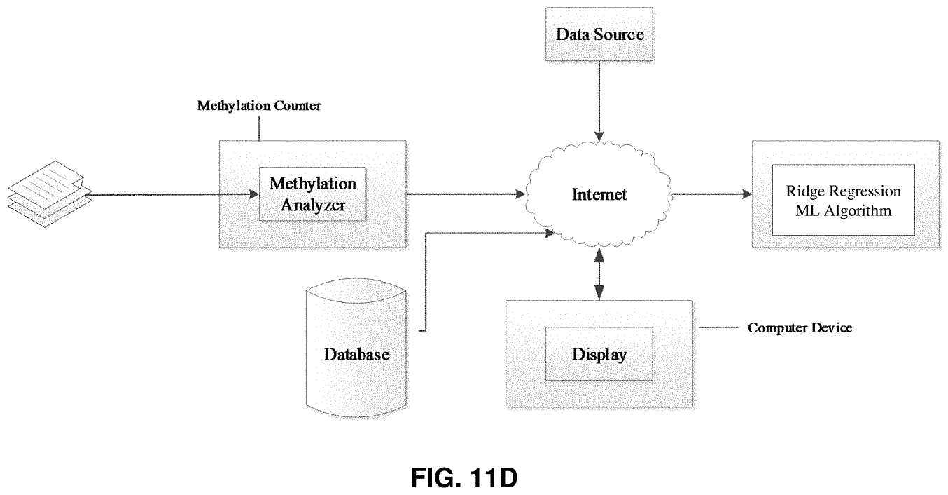

FIG. 11 provides schematic representations of various system architectures that can be employed to practice the methods of the disclosure.

FIG. 11A provides a schematic representation of an integrated system. Methylation sequence data, which can be made available on point (e.g., via a standalone sequence) or via a database (e.g., as FASTQ, IDAT, WIG or BED file), is received by the methylation sequence analyzer. The methylation sequence analyzer is capable of determining a level (e.g., via counting methylation annotation representative of bisulfite sequencing data) or pattern of methylation data in the received dataset. The methylation analyzer filter noise contained in the data and/or to improve search for markers that are associated with the disease (e.g., aging). The machine learning model may be trained with a training dataset comprising actual biological samples (e.g., dermal or epidermal or whole skin samples) of patients, whose age are known. Listings of markers that have the highest predictive significance are provided in Table 1 and/or FIG. 6 (horizontal bars are representative of predictive significance of the marker). Accordingly, in some embodiments, the output of the methylation analyzer may be matched with the markers that are recited in Table 1 and/or FIG. 6; and a result of process be displayed in the display monitor. Optionally, the display monitor is a part of a computer device that receives the outputs of the methylation analyzer and/or the machine learning algorithm and performs mathematical analyses (e.g., regression analysis) to indicate whether results of the methylation analyses permit reliable and/or accurate inferences about the sample/subject's trait to be made. Such a computer system may also allow a user (e.g., a scientist or a clinician) to evaluate the results and input recommendations and other notes based on such evaluations.

FIG. 11B provides a schematic representation of a semi-integrated system. A difference between the semi-integrated system and the integrated system of FIG. 11A is that the output of the methylation analyzer (which has been filtered and optionally weighed based on a machine learning-mediated filtering/weighing process or a static matching process with the top 20%, top 50% or top 80% of markers listed in Table 1) is analyzed in real time over an internet (or cloud) and assessments are made in real time by comparing to existing datasets. The results of the analyses are outputted via a computer display that may be located distally from the marker analyzer module.

FIG. 11C provides a schematic representation of a semi-discrete system. A difference between the semi-discrete system and the semi-integrated system of FIG. 11B is that the machine learning model (or even a static listing of prominent methylation markers) need not be housed within or in close proximity to the methylation analyzer. In fact, the methylation data processed by the methylation analyzer may be continuously processed, in real time, to dynamically provide information about associations between the markers and the traits of interest.

FIG. 11D provides a schematic representation of a completely discrete system. A difference between the fully discrete system and the semi-discrete system of FIG. 11D is the central location of the cloud/internet, which contains methylation data from not only the subject in question, but also an entire database of other subjects (who may be optionally matched to the subject in question based on race, gender, age, and other phenotypic traits). The patient's methylation status, as determined by the methylation analyzer, including other subjects (as inputted by the database) is analyzed by a machine learning algorithm, which has been trained by a data source. The output of the algorithm, as applied on the patient's dataset, is then compared to the output of the network on the in silico dataset, and the predictive accuracy of both the system and also the subject's genetic dataset, is outputted onto a display monitor via a computer. A non-limiting representative methodology is provided in the Examples section, wherein, “molecular clock” markers of Horvath, as applied to the actual patient datasets accessioned in GEO or ARRAYEXPRESS are comparatively assessed for fitness and error compared to the markers of Table 1 and/or FIG. 6, which were uncovered using the methodology of the disclosure.

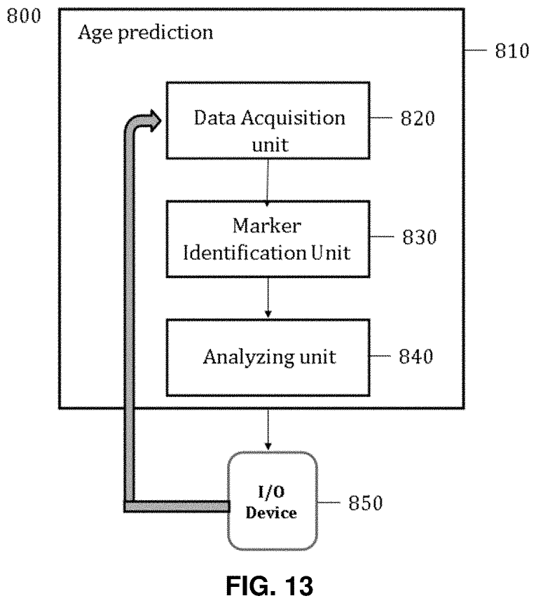

FIG. 13 shows a schematic diagram of a representative system 800 of the disclosure. Specifically, a representative Age prediction/calculating unit 810 is shown, which is useful for calculating or predicting the age of a biological sample (e.g., skin tissue, sperm, eggs, etc.).

Age prediction/calculating Unit 810 generally comprises three modules and can be communicatively connected to an input/output device (I/O device). It should be noted that the various modules may be provided separately or in an integrated unit (as shown).

A first module, Data Acquisition module 820 contains components and/or software for a) receiving a plurality of methylome datasets; b) homogenizing the methylome datasets and merging the homogenized dataset into a single data frame; c) filtering confounding markers from the processed dataset (e.g., by removing cross-reactive markers; not available markers; and/or sex-specific markers); d) identifier for identifying relevant markers from the filtered markers; and e) selecting a training dataset from the pool of relevant markers, e.g., by balancing the age distribution of samples. The Data Acquisition module 820 may be equipped to receive epigenetic data (raw or pre-processed data) containing information about levels and/or patterns of methylated genomic DNA and/or position thereof (e.g., at specific chromosomal segments, in specific genes or locus thereto).

In some embodiments, the disclosure relates to a standalone Data Acquisition module 820, which provides filtered markers that are age-balanced, which may be processed by the downstream modules, e.g., Marker Identification module. The components and/or software in the standalone Data Acquisition module 820 are as described above.

Preferably, the Data Acquisition module 820 is communicatively connected to a second module, the Marker Identification module 830. The connection may be wired connection or wireless connection. Marker Identification module 830 contains components and/or software for identifying a plurality of age-specific methylation markers in the dataset using an output of the Data Acquisition module 820. Marker Identification module 830 may classify each relevant and unique marker in the dataset based on a relevance score which indicates a level of a statistical association between the marker and the age. Marker Identification module 830 preferably includes a classification engine utilizes a machine learning (ML) regression model. Marker Identification module 830 may optionally contain a control validation module for validating the results trained machine learning algorithm.

In some embodiments, the disclosure relates to a standalone Marker Identification module 830, which identifies a plurality of age-specific methylation markers in a dataset. The standalone Marker Identification module 830 may be integrated to the upstream Data Acquisition module 820 and/or to the downstream to the Analyzing module 840 using standard methods, e.g., using wiring cables and/or connectors or wirelessly. The components and/or software in the standalone Marker Identification module 830 are as described above.

Preferably, Marker Identification module 830 is further communicatively connected to a third module, the Analyzing module 840. Analyzing module 840 contains components and/or software for detecting the methylation status of age-specific methylation markers identified by the ML or a gene linked to the methylation marker or locus thereto in a biological sample and assessing the age of the biological sample based on the detected methylation status of the biological sample.

In some embodiments, the disclosure relates to a standalone Analyzing module 840, which detects the methylation status of age-specific methylation markers identified by the ML (or a gene linked to the methylation marker or locus thereto) in a biological sample. The standalone Analyzing module 840 may be integrated to the upstream Identification module 830 using standard methods, e.g., using wiring cables and/or connectors or wirelessly. The components and/or software in the standalone Analyzing module 840 are as described above.

In some embodiments, Analyzing module 840 may be connected downstream to one or more components and/or systems. For instance, as shown in FIG. 13, Analyzing module 840 may be communicatively connected to an input/output (I/O) device, e.g., a server or a computer or a smartphone, which in turn may be connected to the Age prediction/calculation unit 810. Ideally, the I/O device has a display, wherein the output, i.e., whether the sample is an aged sample (e.g., >70 years), is displayed.

Machine Learning (ML) Algorithm

By way of illustration only, the disclosure relates to algorithms and software involved in running the diagnostic engine of the disclosure (Engine). In some embodiments, Engine utilizes a classifier that classifies methylation markers based on one or more parameters that give rise to epigenetic variants that may lead to one or more functional effects, e.g., altered transcription, altered gene expression, altered levels of gene product (e.g., mRNA or protein) and/or altered activity of the gene product. Automated classifiers are an integral part of the fields of data mining and machine learning. There has been widespread use of automated classifying engines to make classifying decisions. Preferably, the classifiers of the disclosure are capable of formalizing methylation data into categorized outcomes, e.g., grouped based on prognostic or diagnostic significance. The classifiers of the disclosure can be programmed into computers, robots and artificial intelligence agents for the same types of applications as neural networks, random forests, support vector machines and other such machine learning methods.

Accordingly, in some embodiments, the systems and methods of the disclosure include a classifier based on a Ridge Regression machine learning algorithm, which penalizes the size of parameter estimates by shrinking them to zero in order to decrease complexity of the model, while including all the variables in the model.

The disclosure further relates to computer-readable storage medium containing a program for detecting methylation markers comprising methylated cytosine (e.g., [C/G]) in a sequencing read (e.g., methylome sequencing using bisulfate sequencing) or hybridization data or other, the program comprising a Ridge regression machine learning algorithm.

In another embodiment, a benchmark dataset from published reports may be used. For example, as described in detail in the Examples, (A) a gene expression omnibus (GEO) dataset GSE51954 (submitted: Oct. 31, 2013; updated: Dec. 27, 2017; Vandiver et al., Genome Biol., 2015). The GSE51954 dataset comprises 429.944 probes, from DNA methylation profiling of epidermal and dermal samples obtained from sun-exposed and sun-protected body sites from younger (<35 years old) and older (>60 years old) individuals, and includes about 78 samples of skin tissue. Analysis of the dataset was performed using the Engine of the disclosure; (B) GEO Dataset GSE90124 (accessioned Jan. 4, 2017; see, Roos et al., J Invest Dermatol 2017); and (C) Dataset E-MTAB-4385 (released on Mar. 24, 2016 in ARRAYEXPRESS database; see, Bormann et al., Aging Cell, 2016). The GSE90124 dataset comprises genome-wide genomic DNA profiling of human skin samples using BEADCHIP. The skin tissue DNA was derived from a peri-umbilical punch biopsy (adipose tissue was removed from the biopsy before freezing) from 322 healthy female twins of the TWINS UK cohort. Family structure is present in this data. The E-MTAB-4385 dataset includes human epidermis methylomes (N=108) that were obtained using BEADCHIP array-based profiling of 450,000 methylation marks in various age groups. The combination of the three dataset resulted in 508 samples (40 dermis, 146 epidermis, whole skin 322), each sample had more than 450,000 CpG/probes/features Analysis of the dataset was performed using the Engine of the disclosure. The methylation markers identified by Engine was more tightly associated with age in comparison to the markers disclosed by Horvath et al. (Genome Biol., 2013).

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1: Computational Methodology to Identify Markers

Training dataset: Genome wide DNA methylation profiling of epidermal, dermal and whole skin samples obtained from human subjects, which have been deposited in various databases, were used as benchmark. Dataset GSE51954; Dataset GSE90124; and (C) Dataset E-MTAB-4385, allowing to use 508 samples (40 dermis, 146 epidermis, whole skin 322), each sample had more than 450,000 CpG/probes/features. The entire contents of these datasets are incorporated herein by reference. The beta values of three studies were combined in the following manner: GSE51954 dataset comprising 429,944 probes, 78 samples+GSE90124 dataset comprising 450,531 probes, 322 samples+E-MATB-4385 dataset comprising 411,873 probes, 108 samples. The combination results in a matrix of 344,422 probes and 508 samples.

From the aforementioned datasets (GSE51954, GSE90124 and E-MTAB-4385), 508 samples were compiled. The datasets comprise methylation markers that are represented by Illumina CpG identifier number (Illumina Inc., San Diego, Calif., USA). The sequences related to the markers and the genes associated therewith are provided in the INFINIUM HUMAN METHYLATION 450K v1.2 Product Files or INFINIUM METHYLATION EPIC v1.0 B4 Product Files. More specifically, the comma separated variable (CSV) file entitled “Manifest File,” which was deposited May 23, 2013 (for 450K) and on Sep. 19, 2017 (for EPIC) and made available for download via FTP (at ftp(dot)illumina(dot)com/downloads/ProductFiles/HumanMethylation450/HumanMethylation450 15017482 v1-2(dot)csv or ftp(dot)illumina(dot)com/downloads/productfiles/methylationEPIC/infinium-methylationepic-v-1-0-b4-manifest-file-csv.zip), provides detailed guidance on the site of the methylation (as indicated by large brackets [C/G]), the nucleotide sequence(s) of the methylated molecule as well as the gene or locus containing the methylation marker.

A representative table containing marker/probe names (as indicated by their ILLUMINA ID Nos. and/or GENBANK gene names) is provided in Table 1.

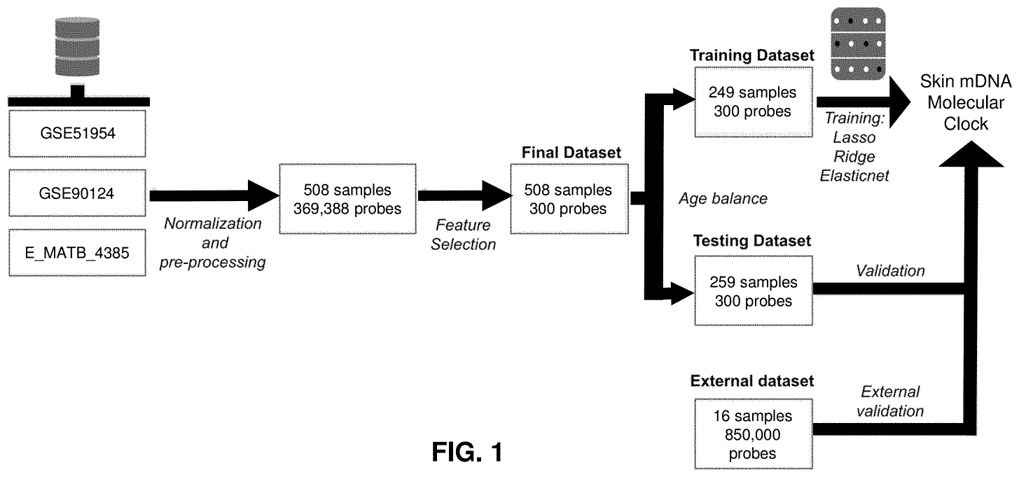

An exemplary experimental design of the age-prediction methodology according to the various embodiments is illustrated in FIG. 1. Three public datasets were selected (GSE51954, E-MTAB-4385, GSE90124), as described above. The datasets were selected based on their tissue, gender and age composition. The datasets include 508 samples (40 dermis, 146 epidermis, and 322 whole skin), wherein each sample included more than 450,000 CpG/probes/features. The main characteristics of the cohort is described in Table 2.

TABLE 2

| | Number | Number | | | | | | Number |
|---|---|---|---|---|---|---|---|---|
| | of | of | Type of | | | Donor | | of |
| Dataset ID | probes | samples | sample | Sex | Ethnicity | Age | Platform | probes |
| GSE51954 | 429,944 | 78 | 40 dermis | 43 f | caucasian | 20-95 | Human | 485,512 |
| | | | 38 epidermis | 35 m | | | Methylation | |
| | | | | | | | 450 | |
| GSE90124 | 450,531 | 322 | 322 whole | 322 f | caucasian | 39-83 | Human | 450,531 |
| | | | skin | | | | Methylation | |
| | | | | | | | 450 | |
| E_MATB_ | 411,873 | 108 | 108 | 108 f | caucasian | 18-78 | Human | 410,942 |
| 4385 | | | epidermis | | | | Methylation | |
| | | | | | | | 450 | |

To build a machine-learning (ML) algorithm able to predict age accurately, these datasets were merged, preprocessed, and divided into an age-balanced training subset and testing sub sets.

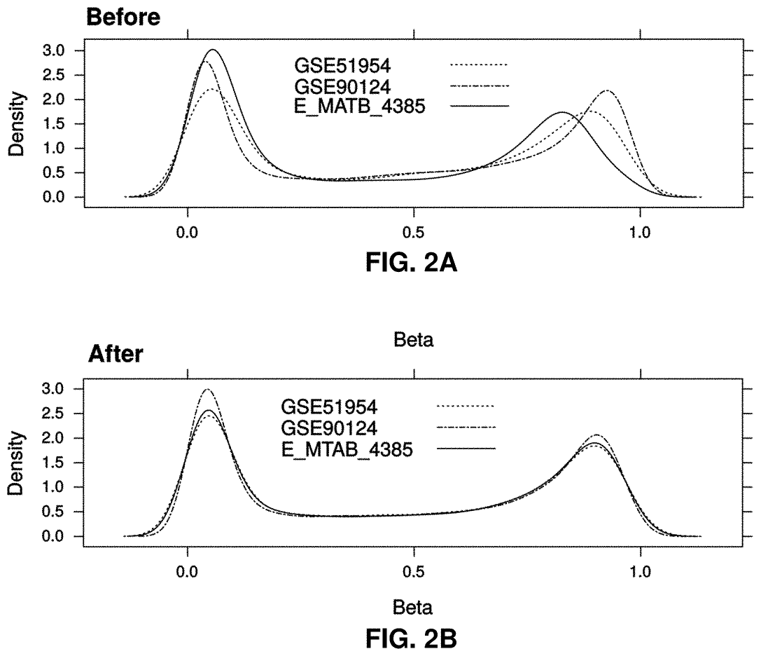

First, an in house script was employed, which obtained the raw data of each dataset, extracted the methylation matrices and turned the extracted datasets into data frames. The script also extracted the meta-data and labeled all the data. The composite data was then joined into a single data frame generating a list of methylation levels with 508 samples. FIG. 2 shows Beta values of the dataset before (FIG. 2A) and after (FIG. 2B) the preprocessing and normalization steps using the systems and methods of the disclosure.

Second, a second in house script was implemented for preprocessing the data that removed the cross-reactive probes by comparing them with the file for the non-specific probes. Typically, the non-specific probes are provided in comma-separated variable (CSV) format for a particular manufacturer (e.g., ILLUMINA). By implementing this step, the number of probes that are used in the analysis is greatly reduced, which permits reduction of cost of the downstream computational steps ahead and delivers probes that represent meaningful differential data points, which probes are then implemented in the ML step. The same script was used to remove the unavailable probe holders (if present), and remove sex-specific probes and the probes that are not present in the assay system. The sex-specific probes were removed so the dataset represented the differences of methylation related to the age of the samples and not to their gender. This step minimizes gender bias, and eliminates the possibility that ML algorithm may be driven to select probes that are also important for age but gender specific. The removal of probes not included in the assay system allowed alignment and better integration of the system/methods of the disclosure with the current technology.

Third, a feature selection step was implemented with a script, which combined the results of a wrapper to estimate the importance based on three different methodologies: glmnet-lasso, xgboost, and ranger. Each one of these methodologies, run by the script, provided a list of the most relevant features/probes regard its own mathematical model for predicting a feature of interest (e.g., age or risk of developing age-related disease). The script integrated the results of the regression/correlation methods and maintained unique probe set by eliminating redundancies. The pre-analytical steps generated a pool of 300 probes from each sample.

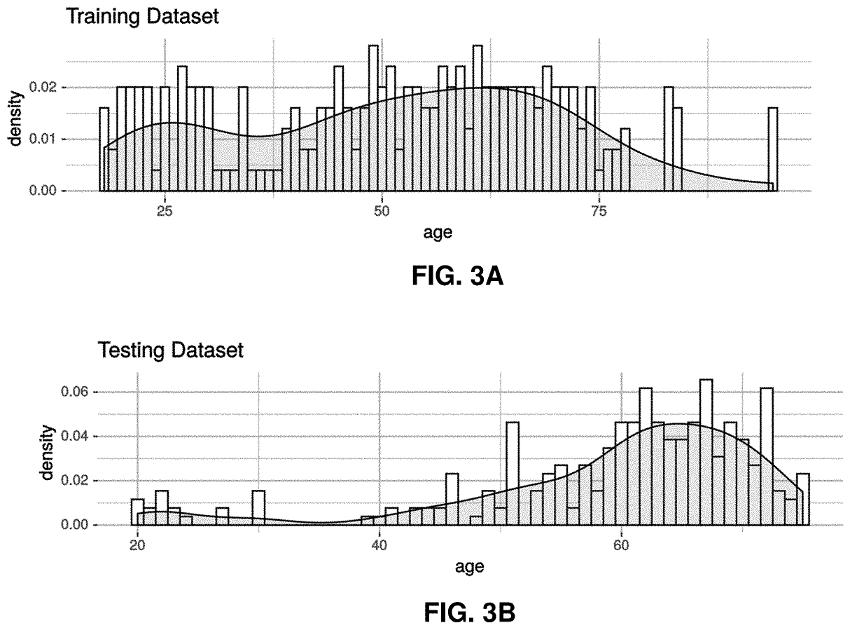

Fourth, samples were selected for the training dataset by ensuring the resulting pool included a balanced distribution between the ages. Several criteria were implemented to balance age distribution, including, having, at most, 5 samples per age window of 7 years, beginning with age 18. The balanced-training dataset had 249 samples. The remaining 259 samples were used for the testing dataset. This step greatly minimizes bias towards certain ages that could be overrepresented in the training dataset, thereby allowing the predicting algorithm to perform equally well among diverse age groups. Age distribution between training and testing datasets are shown in FIG. 3A and FIG. 3B, respectively, and in Table 3 below.

TABLE 3

| | Number of | | | | | | |
|---|---|---|---|---|---|---|---|
| Dataset | samples | Type of sample | | Sex | Ethnicity | Age | |
| Training | 249 | 40 | dermis | 214 f | caucasian | Min. | 18.00 |
| | | 99 | epidermis | 35 m | | 1st Qu. | 35.70 |
| | | 110 | whole skin | | | Median | 53.37 |
| | | | | | | Mean | 51.56 |
| | | | | | | 3rd Qu. | 66.21 |
| | | | | | | Max. | 95.00 |
| Testing | 259 | 0 | dermis | 259 f | caucasian | Min. | 20.00 |
| | | 47 | epidermis | 0 m | | 1st Qu. | 54.59 |
| | | 212 | whole skin | | | Median | 62.46 |
| | | | | | | Mean | 59.38 |
| | | | | | | 3rd Qu. | 67.67 |
| | | | | | | Max. | 74.97 |

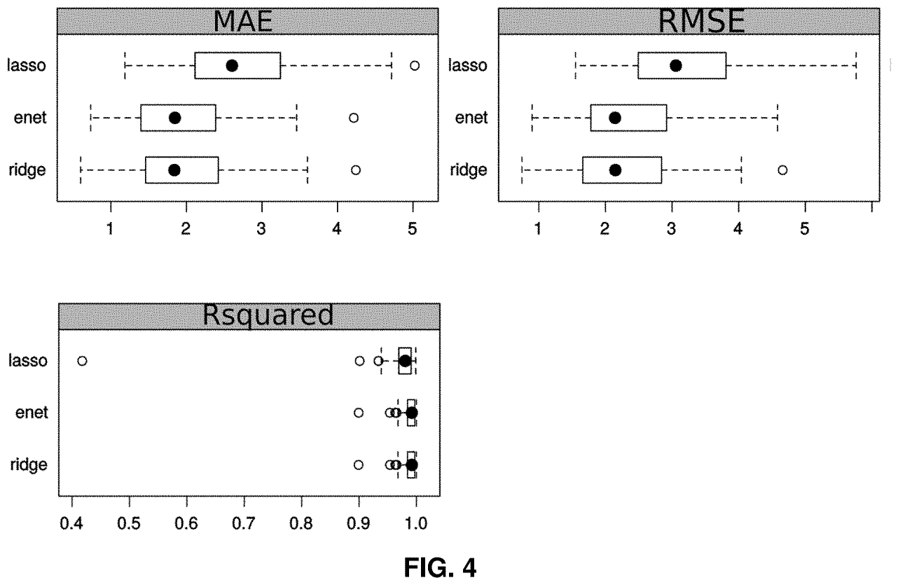

Next, the training dataset was applied to build a ML-based regression model. Several ML algorithms were tested, in each one a 50 fold resampling cross-validation was used for optimization of the tuning parameters. Model prediction errors were computed using mean absolute error (MAE) and/or root mean squared error (RMSE) and the fitness levels and significance of the applied regression models were evaluated by computing Pearson's correlation coefficient using the training data (e.g., smaller MAE or RMSE scores indicate better predictive algorithm and an R² value of about or nearing 1.0 indicates a better fit). (FIG. 4) Ridge Regression ML algorithm, which penalizes the size of parameter estimates by shrinking them to zero, in order to decrease complexity of the model while including all the variables in the model, delivered the best performance.

Results: After the 50 fold resampling cross-validation, the best model was obtained with fraction=1 and lambda=0.04037017, corresponding to a regression model with R² of 0.99, RMSE of 2.48 years, and MAE of 2.06 years.

### Example 2: Validation and Accuracy of the Skin-Specific Molecular Clock to Predict Age

The ML-based regression model of the disclosure was validated using the testing dataset (259 samples), where the R2 were evaluated (FIG. 5). The relationship of the 300 individual probes as biomarkers of age of samples, was validated, each displaying a degree of relevance to the age (FIG. 6 and Table 1). The Ridge Regression model of the disclosure was able to predict age of the testing dataset with high accuracy. The correlation between predicted and chronological age was 0.91 (p<2.2E-16) with a RMSE of 5.16 years (FIG. 5A). When evaluating the same testing dataset, a slightly better accuracy was obtained with epidermis samples only (R=0.97; p<2.2E-16) (FIG. 5B) as compared to whole skin samples (R=0.82; p<2.2E-16) (FIG. 5C).

### Example 3: Applying the Skin-Specific Molecular Clock to Predict Age of External Data and Comparing Accuracy of Skin-Specific Molecular Clock to Other Molecular Clocks

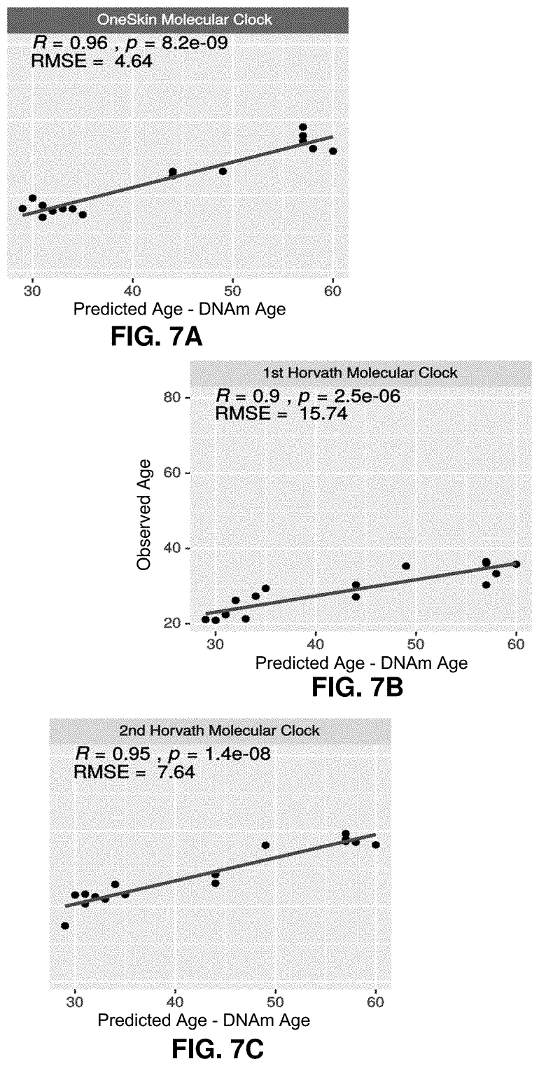

Next, the accuracy of the algorithms and systems (ENGINE) was validated using an external dataset of 16 whole skin biopsies. The methylation profiles of the 16 samples were assessed using the EPIC array. The fitness levels and significance of the applied regression models were evaluated by computing Pearson's correlation coefficient. A high accuracy of prediction was obtained in evaluating the external dataset. The correlation between predicted and chronological age was 0.96 (p<8.2E-9) with a RMSE of 4.64 years (FIG. 7A).

A comparison between the engine and state of art methods (Horvath's 1^{st} and 2^{nd} Molecular Clocks) was also performed using the external biopsies dataset. The fitness levels and significance of the applied regression models were evaluated by computing Pearson's correlation coefficient. Accuracy of age-calculating algorithm compared with Horvath's methods are shown in FIG. 7B (1^{st} Horvath Molecular Clock) and FIG. 7C (2^{nd} Horvath Molecular Clock).

Beta values from test data set (16 samples) were also used to obtain the methylation DNA age according to Horvath's Molecular Clocks, following manual instructions. The fitness levels and significance of the applied regression models were evaluated by computing Pearson's correlation coefficient. Accuracy of age-calculating algorithm was compared with Horvath's methods. The comparative assessment for all the individual samples is shown in Table 4, below. As can be seen, the differential between calculated age and actual (chronological age), as indicated by delta (Δ), is smaller with the instant methods and there is also lesser variability in the calculations.

TABLE 4
A listing of the various samples in the validation dataset and prediction of their
epigenetic age using 1^{st} Horvath Molecular Clock (HW1) and 2^{nd} Horvath Molecular Clock
(HW2) and the ML-based regression model (ENGINE) of the present disclosure.

| | Chronol. | ENGINE | | HW1 | | HW2 Predicted | |
|---|---|---|---|---|---|---|---|
| Sample ID | Age | Predicted age | delta | Predicted age | delta | age | delta |
| 18-0053 | 30 | 39.2 | 9.2 | 20.9 | −9.1 | 43 | 13 |
| 18-0079b | 35 | 34.8 | −0.2 | 29.4 | −5.6 | 43.1 | 8.1 |
| 18-0080b | 57 | 54.4 | −2.6 | 36.1 | −20.9 | 59.3 | 2.3 |
| 18-0081b | 31 | 34.1 | 3.1 | 22.5 | −8.5 | 40.6 | 9.6 |
| 18-0098b | 34 | 36.4 | 2.4 | 27.3 | −6.7 | 45.8 | 11.8 |
| 18-0117b | 57 | 58.1 | 1.1 | 36.5 | −20.5 | 57.8 | 0.8 |
| 18-0140 | 58 | 52.4 | −5.6 | 33.3 | −24.7 | 57 | −1 |
| 18-0147 | 44 | 46.3 | 2.3 | 27.1 | −16.9 | 46.1 | 2.1 |
| 18-0148 | 49 | 46.3 | −2.7 | 35.3 | −13.7 | 56.2 | 7.2 |
| 18-0149b | 32 | 35.8 | 3.8 | 26.2 | −5.8 | 42.5 | 10.5 |
| 18-0158 | 33 | 36.4 | 3.4 | 21.3 | −11.7 | 41.9 | 8.9 |
| 18-0159 | 44 | 45.1 | 1.1 | 30.3 | −13.7 | 48.4 | 4.4 |
| 18-0171b | 57 | 55.8 | −1.2 | 30.3 | −26.7 | 57.2 | 0.2 |
| 18-0172 | 31 | 37.3 | 6.3 | 22.4 | −8.6 | 43.2 | 12.2 |
| 18-0173 | 29 | 36.4 | 7.4 | 21.1 | −7.9 | 34.8 | 5.8 |
| 18-0193 | 60 | 51.7 | −8.3 | 35.8 | −24.2 | 56.3 | −3.7 |

The data, which are shown in FIG. 7 and Table 4, show that the ENGINE not only accurately calculated age of unknown biological samples, but its calculations were superior to Horvath's Molecular Clocks. For example, Pearson correlation in the present training data (observed age versus methylation predicted age) showed stronger statistical association between the markers of the disclosure and age (r=0.96, p 8.2E-09), which compares very favorably to 1^{st} Horvath's Molecular Clock (r=0.90, p 2.5E-06) and 2^{nd} Horvath's Molecular Clock (r=0.95, p 1.4E-08). Moreover, the RMSE was significantly smaller for the ENGINE of the present disclosure (4.64 years) versus 1^{st} and 2^{nd} Horvath's Molecular Clocks (15.74 and 7.64 years, respectively). The improved predictive accuracy with ENGINE was observed across all samples, from young adults (e.g., <35 years old) to older subjects (e.g., >55 years old). These observations of ENGINE's superior predictive potential were both surprising and unexpected.

### Example 4: Applications of Skin-Specific Molecular Clock

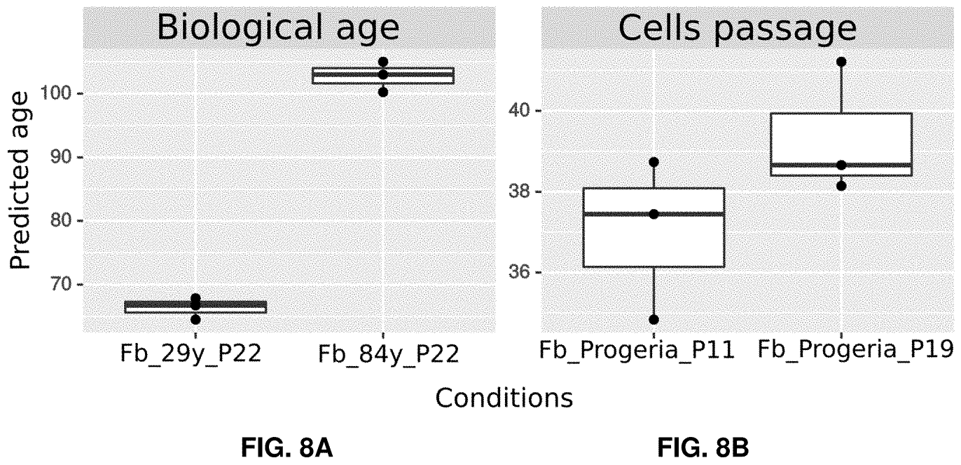

The ability of the ENGINE of the present disclosure to predict age differences in fibroblast (FB) monoculture obtained from donors of different age was evaluated. The predicted age of fibroblasts derived from a 29-year old donor was determined to be 66.37 years (mean age), while the predicted age of fibroblasts derived from a 89-year old donor was determined to be 102.7 years (mean age), both at passage 22, p value=0.001, T-Test (FIG. 8A).

The ability of the ENGINE of the present disclosure to detect the effect of cell culture passages was also evaluated. The age predicted for progeria cells at passage 11 was 37.00 years (mean age), while that of progeria cells at passage 19 was predicted to be 39.34 years (mean age) (FIG. 8B). Thus, besides being able to significantly capture the effect of natural aging on fibroblasts from donors of different ages, the ENGINE of the present disclosure was also able to detect the effect of cell passaging on cell cultures and cell culture age.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications, accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

TABLE 1

| SEQ | | | | | UCSC_ | UCSC_ | |
|---|---|---|---|---|---|---|---|
| ID | PROBE ID | | | | RefGene_ | RefGene_ | |
| NO | NO | chr | pos | strand | Name | Group | Forward_Sequence |
| 1 | cg17484671 | chr1 | 31158158 | - | | | GAGGCTCCTCCGGGAAAGCTC |
| | | | | | | | CTTCTGCTCCAGGTGACAGCG |
| | | | | | | | GAGAGAGATGCCACCGCG[CG] |
| | | | | | | | GCGACCGGCAGGGCCGCGTC |
| | | | | | | | CCCTCTGCGTCCTAGCACAGCG |
| | | | | | | | ACGCCCCGCCCGCCACCC |
| 2 | cg11344566 | chr2 | 124782885 | + | CNTNAP5; | 5′UTR; | CCCGCTCGCCTATAAGGAGCT |
| | | | | | CNTNAP5 | 1stExon | GTCCGCCACCCGGGTGCTGAT |
| | | | | | | | TCCAGCTCTCGCGCCCGA[CG] |
| | | | | | | | AGGTGGATTTGGCTGTCCACC |
| | | | | | | | GAGCTCCGGCGCCTGTCGTTCT |
| | | | | | | | AATTGGGTTTGGATTTG |
| 3 | cg24809973 | chr8 | 72468820 | + | | | TCGGTCTTCTCCCGCCCCTCCC |
| | | | | | | | TCCCTTCCCCGCCTCTCCCCCA |
| | | | | | | | AGCTCCTCAGTGGCCG[CG]GC |
| | | | | | | | CCGTCAACACTGTCGCGCAGT |
| | | | | | | | CACTGGCGCAGGTTCCCAGCT |
| | | | | | | | CTCAGCTGGGGGTTTC |
| 4 | cg03200166 | chr11 | 61335254 | + | SYT7 | Body | CTGCACCCCGGCGGGCGCACA |
| | | | | | | | GACGGTCCCCAGCGGCGGCCT |
| | | | | | | | GGGCCAGCGGCGAAGCAG[CG] |
| | | | | | | | GCAGACGGTTCTCCGGCCCCC |
| | | | | | | | GCCGCCCCCTCACCGCTCCCGG |
| | | | | | | | GGCAATCTGGCGCTCAG |
| 5 | cg06782035 | chr5 | 16179135 | + | MARCH11 | Body | CCGTGGTGCTGAAAGCTTGAC |
| | | | | | | | CGGCGCGAGCTGGAGCCGCCA |
| | | | | | | | CCGGCTGCCTCGGGGTCT[CG] |
| | | | | | | | CCGGGCCTTACCTGCTCCGCGC |
| | | | | | | | CCTGGAAGCAGATCTTGCAGA |
| | | | | | | | TGGGCTGGTGGTGCTGG |
| 6 | cg02352240 | chr16 | 51188372 | + | | | TTGTCTCGGTCCCAAGTTCCGT |
| | | | | | | | GGTTCGCTGGTGCGGGCGCTG |
| | | | | | | | CAGTGTCAGGGCGCTGG[CG]A |
| | | | | | | | GGCTCCGCGTGCCGCGATGCA |
| | | | | | | | AAGAAATACATCAATAAAAAC |
| | | | | | | | AGAAGCAGAGTGGGGGT |
| 7 | cg25351606 | chr6 | 100917427 | + | | | ACAGTCGCAGCTTAACCCCGTT |
| | | | | | | | GGGGGCGCCGCCCCGCTGAG |
| | | | | | | | GTGGTTGCGTCTCCAAGT[CG] |
| | | | | | | | TGAGCCTCCAATAGCTGCTCCC |
| | | | | | | | GCTTTCGCGTCGCAACCCCAG |
| | | | | | | | GACCCCGGGAAATTACC |
| 8 | cg07547549 | chr20 | 44658225 | - | SLC12A5; | Body; Body | TTGCAGCCTGGAGCTCAGCTC |
| | | | | | SLC12A5 | | CATTGGAATGCTCCGGGCGCT |
| | | | | | | | GTCCAAGGTGCTGGAATG[CG] |
| | | | | | | | CCGCGCCCGGGGGCAGAGCT |
| | | | | | | | GCGGGCCGGGGGATTATCGCT |
| | | | | | | | GCCCACGGCTTCGGGCTGA |
| 9 | cg03354992 | chr10 | 88149475 | - | | | TCCTGTGCTCCCAGGTCTGGGC |
| | | | | | | | GTTAGGATTCTCTCAGTCCCGG |
| | | | | | | | AGCCACGCCGGCTGAC[CG]CA |
| | | | | | | | GGGCTCGGGGAGCGCGGCTG |
| | | | | | | | GGCCCCTTTTCCCGGGTCCGG |
| | | | | | | | GAAGCGCCGGGCCACGC |
| 10 | cg00699993 | chr4 | 158141570 | - | GRIA2; | TSS200; | CGCACGAAGGTAGCTCCGGGC |
| | | | | | GRIA2; | TSS1500; | GGGGAGCGAGGCGCTGTCCTC |
| | | | | | GRIA2 | TSS200 | GGTGCTGAAAGGCCGAGG[CG] |
| | | | | | | | CGCGGTGGGCGCGACAGCCC |
| | | | | | | | CGGAGACCCGAGGTCTCGCGG |
| | | | | | | | AGGGACAGCGGCTACGGGC |
| 11 | cg02611848 | chr2 | 74875387 | + | C2orf65 | TSS1500 | AGCCTGCGAAGTGGTGCCGGC |
| | | | | | | | TGCTCTCGGGCTGCCCTCCCTC |
| | | | | | | | CCCGAGGCGTGGAGAAC[CG]T |
| | | | | | | | ACCTGTCTTCGGAAGACGGAG |
| | | | | | | | GCCCCCTCACCTGGTCCTCCCG |
| | | | | | | | GCTCTCAGCGTGCGCC |
| 12 | cg07640648 | chr19 | 39993697 | + | DLL3; | Body; Body | TCGCGGTGCGGTCCGGGACTG |
| | | | | | DLL3 | | CGCCCCTGCGCACCGCTCGAG |
| | | | | | | | GACGAATGTGAGGCGCCG[CG] |
| | | | | | | | TGAGTCCTGCGTTCGACCCCA |
| | | | | | | | CCCCGTCCCAGCCGGGGACCC |
| | | | | | | | CGGCCCCTCCTGAGCGTC |
| 13 | cg18235734 | chr1 | 91301731 | + | | | GGCCGCAGGGAGAACTCGCCT |
| | | | | | | | CCCCGCCCCGGCACGGGCACT |
| | | | | | | | GTCTGCGGCCACGTGCCC[CG] |
| | | | | | | | GAGGTCGCGGCCCAACCAGCC |
| | | | | | | | CCGCCGACTTGTTCCGCTTTCG |
| | | | | | | | CCCCAGCCCCCGGCGGG |
| 14 | cg06279276 | chr16 | 67184164 | - | B3GNT9 | Body | CCGCCGCTGGTCCTTGGCGCG |
| | | | | | | | CAAATAGCGGGCGAAGTCAAA |
| | | | | | | | GGGTCCCGTAGGCGTGGG[CG] |
| | | | | | | | GCGCCGGTGTGTCCCCTTCGT |
| | | | | | | | AGGCCGGCGGGGCTGCACCC |
| | | | | | | | GCGTCGGGTAACTGGAACG |
| 15 | cg00748589 | chr12 | 11653486 | - | | | CCGGTGCGCCGGGCTCTACCT |
| | | | | | | | CAAGGAGCTCAGGGCCATCGT |
| | | | | | | | GCTGAACCAACAGAGGCT[CG] |
| | | | | | | | TCCGCACCCAGCGCCAGAGCA |
| | | | | | | | TCGACGAGCTGGAGCGGCGG |
| | | | | | | | CTGAACGAGCTGAGCGCCT |
| 16 | cg23368787 | chr19 | 36049342 | + | ATP4A | Body | GTTGAAGGGTATCTCGCAGAC |
| | | | | | | | TTTTGGGAAGCGGTCCCGGTA |
| | | | | | | | GCCCATGGCGTTGCCCAG[CG] |
| | | | | | | | TCAGCTCCGAGAACTTGAGCA |
| | | | | | | | GCGCCGTCTCCGATGCGTCTCC |
| | | | | | | | AATCACGATGCGCTGGG |
| 17 | cg02383785 | chr7 | 127808848 | + | | | TCACCTAGGGCGGAGGCGCAA |
| | | | | | | | GCTCTGCTGGGTGCTCTCCGCC |
| | | | | | | | CCCTTGATCGCCGCTCT[CG]GT |
| | | | | | | | TTTCAGCACCAGGATCCGGAC |
| | | | | | | | AGCTCCCCACCTGGCCCTGAG |
| | | | | | | | GGGCCTCTTTCCTTGC |
| 18 | cg02961707 | chr19 | 7927974 | - | EVI5L; | Body; Body | GGCCGAGATGCGGCAGCGCAT |
| | | | | | EVI5L | | TGCCGAGCTGGAGATCCAGGT |
| | | | | | | | GATCGGCGGGGCCGGGGT[CG] |
| | | | | | | | GGGGGCGGGGGCGGGGGCA |
| | | | | | | | GGGCCCGGGGCAGGAGCGGG |
| | | | | | | | GCCGGACCCCAGGCCCAGCAT |
| 19 | cg15475851 | chr10 | 105037349 | - | INA | 1stExon | GTTCATCGAGAAGGTGCATCA |
| | | | | | | | GCTGGAGACGCAGAACCGCGC |
| | | | | | | | GTTGGAGGCCGAGCTGGC[CG] |
| | | | | | | | CGCTGCGACAGCGCCACGCT |
| | | | | | | | GAGCCGTCGCGCGTCGGCGAG |
| | | | | | | | CTCTTCCAGCGCGAGCTGC |
| 20 | cg07171111 | chr4 | 10462903 | + | | | GCCAGGCGCTGGAGCGTGGCT |
| | | | | | | | AAGGCAGGGACCACGTCCCAG |
| | | | | | | | CCGCCCTTTCCCGCCCTG[CG]G |
| | | | | | | | CGCAGGCCCACTCTCTTGGCTC |
| | | | | | | | TCCTGGCCCGCACACTCAGCTC |
| | | | | | | | GGCCGCCGCGGCTGC |
| 21 | cg05080154 | chr18 | 76739409 | + | SALL3 | TSS1500 | AGTGGAAGGGAGGGGGAACG |
| | | | | | | | CAGGGGAGGGAGAGGAGGG |
| | | | | | | | GAGGAGCCGCGCGGCCCGCG |
| | | | | | | | C[CG]CTTCCGAACCGGAAAGT |
| | | | | | | | TGGTCTTGCCGAAGTCCTGCCA |
| | | | | | | | CCCCGGCGTGCGCACTCCGCT |
| 22 | cg03422911 | chr1 | 237205295 | - | RYR2 | TSS1500 | CTCGGAAGGGGCAGGGGAAT |
| | | | | | | | GAGCCCAGGGACCCCAGCGG |
| | | | | | | | GGCGCAGGTAGGAGGCTGTG |
| | | | | | | | [CG]CTCGCCGGGTGCGCTCCG |
| | | | | | | | GCCCCGATTCCCAGCGCAGCC |
| | | | | | | | AGTAAGTGGCGCTGGGCCTCG |
| 23 | cg14462779 | chr10 | 76803669 | - | DUPD1 | Body | CACTGAGGTCGAAGGTGGGCA |
| | | | | | | | GGTCGTCGGCCTCCACGCCGT |
| | | | | | | | GGTACTGGATGTCCATGT[CG] |
| | | | | | | | CGGTAGTAGTCGGGCCCAGTG |
| | | | | | | | TCCACGTTCCAGCGGCCGTGG |
| | | | | | | | GCCGCGTTCAGCACGTGC |
| 24 | cg16061498 | chr18 | 55095886 | + | | | CTCGGGAGGCGCTTTGCCTTT |
| | | | | | | | GAGGAAGATGGAGAGGAGTC |
| | | | | | | | GGGAGAAGCGCCTAGAAAC[CG] |
| | | | | | | | CATTGATTTAGACATCAATC |
| | | | | | | | CTGGCCGGCTCCCTCCGCCTGC |
| | | | | | | | CGAGCTGCGGGGCCGCGC |
| 25 | cg04467618 | chr6 | 134210946 | + | TCF21; | 1stExon; | GCTGGACACGCTCAGGCTGGC |
| | | | | | TCF21 | 1stExon | GTCCAGCTACATCGCCCACTTG |
| | | | | | | | AGGCAGATCCTGGCTAA[CG]A |
| | | | | | | | CAAATACGAGAACGGGTACAT |
| | | | | | | | TCACCCGGTCAACCTGGTGAG |
| | | | | | | | TGCTCCCGGGGCTGCAG |
| 26 | cg02891686 | chr4 | 24801425 | + | SOD3 | Body | GCAGCCCCGGGTGACCGGCGT |
| | | | | | | | CGTCCTCTTCCGGCAGCTTGCG |
| | | | | | | | CCCCGCGCCAAGCTCGA[CG]C |
| | | | | | | | CTTCTTCGCCCTGGAGGGCTTC |
| | | | | | | | CCGACCGAGCCGAACAGCTCC |
| | | | | | | | AGCCGCGCCATCCACG |
| 27 | cg12969644 | chr9 | 85678242 | - | RASEF | TSS200 | CCGCGCAGGTGGGGGAGACC |
| | | | | | | | TGGCTGGCCGGAACTGGGATT |
| | | | | | | | CGGGGGGAGCATTGCCCTT[CG] |
| | | | | | | | GCGTAAGCGCTGCTCAGGT |
| | | | | | | | AGAGCCCAGCGCTCCGCTTCTC |
| | | | | | | | CACAGAACGTGCTGGCGCG |
| 28 | cg25509871 | chr19 | 40871557 | + | PLD3; | 5′UTR; 5′UTR | GTAAATGAGAAAAGACGTGA |
| | | | | | PLD3 | | GGTTCCTTTTGTTCTTTACCTGT |
| | | | | | | | GGCCTCCCTGCCCTACA[CG]G |
| | | | | | | | GGACTCTAGGGTGGAATGTAG |
| | | | | | | | CAAAGCCCATCCACCAGCCAT |
| | | | | | | | GTACTACCCCCCAACCC |
| 29 | cg09017434 | chr5 | 16179660 | + | MARCH11; | 1stExon | GCGGGGGAGGTTGCGGGGGA |
| | | | | | | | GGCTCGGCGTCCCCGCTCTCC |
| | | | | | | | GCCCCGCGACACCGACTGC[CG] |
| | | | | | | | CCGTGGCCGCCCTCAAAGCTC |
| | | | | | | | ATGGTTGTGCCGCCGCCGCCC |
| | | | | | | | TCCTGCCGGCCCGGCTGG |
| 30 | cg17508941 | chr7 | 19183280 | + | | | TGGTACTAGCACGTCACCTAG |
| | | | | | | | AAGGAAGAATCCTGGAATGGC |
| | | | | | | | ACGGGTCCAAACTAGAGG[CG] |
| | | | | | | | GCCTCTCAGCATGGACCCGCTT |
| | | | | | | | CAACCTCATCTGCATGGCAGG |
| | | | | | | | CGTTTTGCAAGGCGTCA |
| 31 | cg12374721 | chr17 | 46799640 | + | C17orf93; | TSS1500; | GGCTCCCAAATTCCTGGGAGA |
| | | | | | PRAC | Body | CCCTCTCCCAGGGCCTCCTGAT |
| | | | | | | | GCAGCTACCATACTGAG[CG]A |
| | | | | | | | TCCGTCGATAACGCCCTTGGCC |
| | | | | | | | CACCGATCAGTTTACCTTATTA |
| | | | | | | | GAGAGAAAAGCACTC |
| 32 | cg11071401 | chr17 | 48637194 | + | CACNA1G; | TSS1500; | AGGTTCCTTCTTAGGGGTCCTC |
| | | | | | CACNA1G; | TSS1500; | GCTCTGCTCCGCAGCCCCTCCT |
| | | | | | CACNA1G; | TSS1500; | GGGGATCCGGGCTCTG[CG]GT |
| | | | | | CACNA1G; | TSS1500; | CCAGCGCGACCTGCCTGGGGC |
| | | | | | CACNA1G; | TSS1500; | CACGTGTTCAAGCACGAAGCC |
| | | | | | CACNA1G; | TSS1500; | CCTGCGTGGAGTCCAC |
| | | | | | CACNA1G; | TSS1500; | |
| | | | | | CACNA1G; | TSS1500; | |
| | | | | | CACNA1G; | TSS1500; | |
| | | | | | CACNA1G; | TSS1500; | |
| | | | | | CACNA1G; | TSS1500; | |
| | | | | | CACNA1G; | TSS1500; | |
| | | | | | CACNA1G; | TSS1500; | |
| | | | | | CACNA1G; | TSS1500; | |
| | | | | | CACNA1G | TSS1500 | |
| 33 | cg06458239 | chr19 | 58038573 | - | ZNF549 | TSS200 | TGACCCTAGTTTGATGGGTTTT |
| | | | | | | | TTCCTTTGTCCTCTCTTTCTTGG |
| | | | | | | | ATTGAGTCCTCACAG[CG]CGG |
| | | | | | | | CGGACTGCGGCGTGGTAGGA |
| | | | | | | | ACTACACCACCCAGAATACTGT |
| | | | | | | | GCGCCGAGCGTGCCG |
| 34 | cg05771369 | chr12 | 58021713 | - | B4GALNT1 | Body | GGGAGGTTGCCTCCAGGCGG |
| | | | | | | | GCCTGGGATAGGGGACCCGA |
| | | | | | | | AGGGGTCAAGGTCTGCGCTC |
| | | | | | | | [CG]GTGCCTTCGGGGGTACCCC |
| | | | | | | | TGCCCCATCCTCTTCCGCTTCA |
| | | | | | | | CCCCTGCAGGACCCAGACA |
| 35 | cg25645064 | chr3 | 147096130 | + | | | CTGGACGACTGTGGCTGGGAT |
| | | | | | | | GGCCTCCCGGCAGTAATCTTG |
| | | | | | | | CGCAAACACCCTGCCACG[CG] |
| | | | | | | | CAAGGACGCCAGCTCAGACAC |
| | | | | | | | GCAGCGCCCCGCGCATACAAA |
| | | | | | | | GGAATGTTCCCTCTTTAA |
| 36 | cg14371731 | chr10 | 81003175 | - | ZMIZ1 | Body | GGCGGCGGCCCCATTAGCGGA |
| | | | | | | | GCCTCCGCCTATGATTGGCTTC |
| | | | | | | | GCCCGGGAAGCTGGAGA[CG] |
| | | | | | | | GGCGATGAATAATTGATGTGT |
| | | | | | | | GCGGTGCGGTAGCCGGACGG |
| | | | | | | | CGGCGGCGGTGGCGGGCAG |
| 37 | cg19556343 | chr21 | 22370046 | - | NCAM2 | TSS1500 | AGCGCCTGAGGAGACAGACA |
| | | | | | | | GTGTAGACTTTAGGGTACAAT |
| | | | | | | | TGCTTCCCCTCTGTCGCGG[CG] |
| | | | | | | | GGGTGGGGAGCGTGGGAAGG |
| | | | | | | | GGACAGCCGCGCAAGGGGCC |
| | | | | | | | AGCCTGCTCCAGGTTTGAGC |
| 38 | cg22158769 | chr2 | 39187539 | + | LOC375196; | TSS200; Body | AGAGCGCTACGTCGCCGGCGG |
| | | | | | LOC100271715 | | GCAGCAGCAGCGCCTACAAAC |
| | | | | | | | TGGAGGCGGCGGCGCAGG[CG] |
| | | | | | | | CACGGCAAGGCCAAGCCGCT |
| | | | | | | | GAGCCGCTCTCTCAAAGAGTT |
| | | | | | | | CCCGCGTGCGCCGCCAGCC |
| 39 | cg10729426 | chr19 | 58038585 | - | ZNF549 | TSS200 | GATGGGTTTTTTCCTTTGTCCT |
| | | | | | | | CTCTTTCTTGGATTGAGTCCTC |
| | | | | | | | ACAGCGCGGCGGACTG[CG]G |
| | | | | | | | CGTGGTAGGAACTACACCACC |
| | | | | | | | CAGAATACTGTGCGCCGAGCG |
| | | | | | | | TGCCGGGGCCTTAGACC |
| 40 | cg16181396 | chr3 | 147126206 | + | ZIC1 | TSS1500 | GAATGAAAGGGGCCCAAGTA |
| | | | | | | | GGGAACAGGAGTGAGGAGAG |
| | | | | | | | ACAGGGTTAGCGGGGGCAGT |
| | | | | | | | [CG]AAGGAGACAACGGAAAGG |
| | | | | | | | CAGAAAACAGAAAAATAACGC |
| | | | | | | | AAGAGAGAGAAAAAGTAAAG |
| | | | | | | | G |
| 41 | cg00049664 | chr16 | 66613334 | - | CMTM2 | TSS200 | GGCGCGTGGAGGGTGGGAGG |
| | | | | | | | ATCCGGCCGCTGCCGGGCGGA |
| | | | | | | | TGGGAGCTGCGCGAGGAGA[CG] |
| | | | | | | | GGCGCGCGTGGAGAGGGC |
| | | | | | | | GCGGGAGTTGGCATTCGGTGG |
| | | | | | | | TCCTGGCAGTTAGCTGAGCAC |
| 42 | cg13473356 | chr3 | 179754613 | - | PEX5L | TSS200 | GCGCTGCGGGCTGCCGGGAAC |
| | | | | | | | TGTTCTCCGCTCGGGGTGCTG |
| | | | | | | | AAAGCGGACGCGGGAGAG[CG] |
| | | | | | | | CGCAGAGAAGGCGAGGAG |
| | | | | | | | CCGGGTCGGCCAGGCTCTCCT |
| | | | | | | | GCAGGCGCGGGTCCTGCTCGC |
| 43 | cg05404236 | chr13 | 110437093 | - | IRS2 | 1stExon | CGAGCCGTGGCCGCTGCTGGA |
| | | | | | | | CGACAGGGAGCCGGGGCTGG |
| | | | | | | | TGGCGGCGGGCGGCGAGTG[CG] |
| | | | | | | | CCACGGGCATGGACATGGA |
| | | | | | | | GCGGCTGTGTTGCAGCGCGCC |
| | | | | | | | CCCTGCCGGCAGCAGCGCCA |
| 44 | cg16295725 | chr4 | 10459219 | + | ZNF518B | TSS200 | AGAGCGGGGAGCCTCAGACCC |
| | | | | | | | AGCCGAGCCCCACTTCTGGGC |
| | | | | | | | TTAGAGCTTGACCCAACA[CG] |
| | | | | | | | TTCGCACCGTAGCGAGCGAGG |
| | | | | | | | TCCACATTTAGCCATGCCGCAG |
| | | | | | | | GCAAAAGAAGGATTCGG |
| 45 | cg21800232 | chr5 | 79866368 | + | ANKRD34B | TSS200 | GCTGGAAGCTCCGCCTTCTGTC |
| | | | | | | | CCCGTAAGTCCCACCCCCGTCC |
| | | | | | | | CCCGCTTCGGCCACCG[CG]CTT |
| | | | | | | | CGGCCACGGCGACTTGGCCAA |
| | | | | | | | CAACAGCGGCAGCAGGGTCTC |
| | | | | | | | CCCATTGAGGGAAGC |
| 46 | cg23437843 | chr3 | 44596360 | - | ZNF167; | TSS1500;TS | TATAACTGACTGCTCAGGATAT |
| | | | | | ZNF167 | S1500 | GCCAGGCCTTTTGCTATGTAGT |
| | | | | | | | GTCTGTTAACCTCATG[CG]GT |
| | | | | | | | GCTCCCAGCCCTGTGAGGTAC |
| | | | | | | | GCATTATGCTCTGCATTTTTTTC |
| | | | | | | | AGATGAGAAAACAG |
| 47 | cg24202131 | chr18 | 34855482 | - | BRUNOL4; | Body; Body; | CACAGTCGCGGGACAGGTGCG |
| | | | | | BRUNOL4; | Body; Body | GAGAGAGCTGTGGCAGGCAG |
| | | | | | BRUNOL4; | | GAGCTGGATCGCAGCGACT[CG] |
| | | | | | BRUNOL4 | | GCCTCCTCCCGCCTGCAGGG |
| | | | | | | | CAGGCTGCACCCTGAGGAGCA |
| | | | | | | | GAGACCCTGGGCTGACCCC |
| 48 | cg15779837 | chr19 | 48918116 | + | GRIN2D | Body | CTCTCTTCATGAGAGAGTCTAA |
| | | | | | | | GGAGGGGGTCCCCAAACTCCC |
| | | | | | | | CAAGCCTGGTCACTGCC[CG]C |
| | | | | | | | AGCCCTCCACCGGATGCCCCCC |
| | | | | | | | GCCCGGAAAAGCGCTGCTGCA |
| | | | | | | | AGGGTTTCTGCATCGA |
| 49 | cg04875128 | chr15 | 31775895 | - | OTUD7A | Body | CGGCGCGCGCCGGGCTGTAGC |
| | | | | | | | TCTGCGACGACAGCGAGCGGT |
| | | | | | | | TCTGCTGCGGGTACGTGG[CG] |
| | | | | | | | CACGGCCGCAGCGCCCCCACG |
| | | | | | | | GCCGGCGCGCACGCCTCGTCC |
| | | | | | | | CGCGCGCCCGACGCCTGC |
| 50 | cg06488443 | chr2 | 162280341 | + | TBR1 | Body | GCACTGGCCGCCCGCTCGGCT |
| | | | | | | | ACTACGCCGACCCGTCGGGCT |
| | | | | | | | GGGGCGCCCGCAGTCCCC[CG] |
| | | | | | | | CAGTACTGCGGCACCAAGTCG |
| | | | | | | | GGCTCGGTGCTGCCCTGCTGG |
| | | | | | | | CCCAACAGCGCCGCGGCC |
| 51 | cg24213719 | chr18 | 60263646 | + | | | ACCGGGTGGGCTCTGCTTCCC |
| | | | | | | | CGGGACCCCACTCTGACCCCAT |
| | | | | | | | CCCCTAAGCCGCTCCCG[CG]A |
| | | | | | | | GCACCTCAGCTCCGCTCCCGCG |
| | | | | | | | CGGGTCAGCAATTCGAAGTCC |
| | | | | | | | GCCCCAGACCCCTGGG |
| 52 | cg25936177 | chr15 | 89313056 | - | | | AATCATTTTTTTTTAGCTTGAA |
| | | | | | | | ACCAAAGCAAACAAGCGCGCA |
| | | | | | | | CAGAGAAGCCCATTCTC[CG]C |
| | | | | | | | GGCCGGCGCGGCAGCCTGGCC |
| | | | | | | | GCTGTGGGTAGCTCAGGGACG |
| | | | | | | | CACAGAGGCCCGGCTGT |
| 53 | cg17833476 | chr5 | 170736201 | + | TLX3 | TSS200 | ATGAGAGGAGAGAGGCTTGTT |
| | | | | | | | GATCGCAGCCAATGGCTGCGG |
| | | | | | | | CAGGAGAGGAATTAGCAG[CG] |
| | | | | | | | GAAACTCCAGGTTCGGTTCAA |
| | | | | | | | GAAAGATGACACAGAGCCTGT |
| | | | | | | | CGGGCCCGCGCACTCTTG |
| 54 | cg12852499 | chr13 | 79170959 | - | | | ATTCATTTTATTTCCAGAACTCT |
| | | | | | | | CCGACCATAAATTATTCAAAGA |
| | | | | | | | GTAAGCCAACCCGAG[CG]GG |
| | | | | | | | GCGGCCGCGCGCCTTCCCCAC |
| | | | | | | | GCGCGCCGGGCTGGCTCTGGC |
| | | | | | | | CGCTCAGCTCACCCGA |
| 55 | cg18671949 | chr17 | 5404581 | + | LOC728392 | TSS200 | TCTGCGCAGCAAGGTTTGTCTC |
| | | | | | | | CATGGCAACCAGACTGGCGGC |
| | | | | | | | GCAAGGGGGAGGAAACG[CG] |
| | | | | | | | AGCCGCTGGCTGGGACCCCGG |
| | | | | | | | GGCACTAGTAGGCTTGGCACC |
| | | | | | | | TAAGAAGCCGAAATGCAA |
| 56 | cg16991515 | chr6 | 27107019 | - | HIST1H2BK; | 3′UTR; | GTCCCCTCCCCCAATGCAGAG |
| | | | | | HIST1H4I | TSS200 | GGACTTCCCGCCAAAGCTCTTC |
| | | | | | | | CGGTTTTCAGTCTGGTC[CG]CA |
| | | | | | | | GAGGTTACCCATAAAAGAAAG |
| | | | | | | | CTGCCATCACAGGCAGCAGAC |
| | | | | | | | CTTTGTTCTCTGACCA |
| 57 | cg06784991 | chr1 | 53308768 | - | ZYG11A | Body | GGCGAGTCTCCTGGGACGCTG |
| | | | | | | | CCGAGGCACTTGCTGGGGAGT |
| | | | | | | | GTGGCCCGCGCGGGGCTG[CG] |
| | | | | | | | GTCTAGATGCCGAGCCCCTTC |
| | | | | | | | CAGGCGCAGGCGTCGCTGCGG |
| | | | | | | | AGGTGCGTTGTCGGGGGA |
| 58 | cg00194126 | chr2 | 157186312 | - | NR4A2 | Body | GAGAGATCCCGGGTCGTCCCA |
| | | | | | | | CATGGGGCTGTGCTGCACCTG |
| | | | | | | | GAAGCCCGGGGTGGTGGG[CG] |
| | | | | | | | TCGGGGGCGAGGAGGGCTTG |
| | | | | | | | TAGTAAACCGACCCGGAGTGC |
| | | | | | | | GGCATCATCTCCTCAGACT |
| 59 | cg00511674 | chr16 | 78080068 | - | | | CCTCCAGGCCTGCAGCCACGC |
| | | | | | | | TTGGCGCTGTCCGCTAGGGCC |
| | | | | | | | AGGTGCTGAAGTGTTGGC[CG] |
| | | | | | | | CGAGCGGAGCTGCTGCAGCGC |
| | | | | | | | TGGCTTCCCCGGGCCGCTGCG |
| | | | | | | | GGTGGACTTGGACAACAT |
| 60 | cg08032924 | chr16 | 66613096 | - | CMTM2 | TSS1500 | GAACACCTGCTTCCTCTCGTTG |
| | | | | | | | CCTTGTGTGAAAGTCGCGTTGT |
| | | | | | | | ATTTTCCTGCGCTTGG[CG]CTG |
| | | | | | | | CGCCCGCGGAGCTCAGGGCCG |
| | | | | | | | TGACCCGGTGCTCGCAGCCCC |
| | | | | | | | CCGACCCCGCAGCGG |
| 61 | cg18795809 | chr4 | 10458531 | - | ZNF518B | 5′UTR | GCCCTCGGAGGAGGCATCCTT |
| | | | | | | | CATAACGCTGGGGGCGGGGA |
| | | | | | | | GCGCAGGCCGGGCCAGCGG[CG] |
| | | | | | | | CCACACGAACGGCCCCGCG |
| | | | | | | | GGACGCTGCCACCCCCGCCTC |
| | | | | | | | GGTCGCCCCGGCGCGTCGGC |
| 62 | cg18866015 | chr18 | 49868552 | + | DCC | Body | CGAGGGATTCAGACAGTCAAG |
| | | | | | | | CGCCAAGGCAGCCCGAGGCTC |
| | | | | | | | CCCAAAGCCTCGCTCGGC[CG] |
| | | | | | | | CACGCGGGCAGGAATCTGCGC |
| | | | | | | | TTGCACTCGGGCTCAGCTCCTC |
| | | | | | | | ATCTTCCTTTGGCCAGA |
| 63 | cg10286969 | chr16 | 2765843 | + | PRSS27 | Body | GGCTTCCGTTGCGCTGGATGC |
| | | | | | | | TGACTTGCCAGGGCCACTCGC |
| | | | | | | | CCTCCTGCGTGTCCTGCC[CG]C |
| | | | | | | | CCACCATTCGGTTCAGCATCCT |
| | | | | | | | GGGGCGACCACAGGCTGGGG |
| | | | | | | | GAGCATGGGGAGCGGGT |
| 64 | cg21572722 | chr6 | 11044894 | + | ELOVL2 | TSS1500 | GGCCGGGCGGCGATTTGCAG |
| | | | | | | | GTCCAGCCGGCGCCGGTTTCG |
| | | | | | | | CGCGGCGGCTCAACGTCCA[CG] |
| | | | | | | | GAGCCCCAGGAATACCCAC |
| | | | | | | | CCGCTGCCCAGATCGGCAGCC |
| | | | | | | | GCTGCTGCGGGGAGAAGCAG |
| 65 | cg23967544 | chr5 | 172672684 | + | | | TTTCCTCCAGGAAAGATAAAG |
| | | | | | | | TAATCGATAGGGTCTTTTAAAT |
| | | | | | | | AGCTCCGCGTTTCCTGT[CG]G |
| | | | | | | | GAGAGGAGTATCAGCGCGCG |
| | | | | | | | CACCAAATCTGCTCTGGTATGT |
| | | | | | | | CACCTTATCTCTCGTCC |
| 66 | cg11498607 | chr21 | 36399226 | + | RUNX1 | Body | TGCAAAAGCTGCCTGCCCGCG |
| | | | | | | | CGTTATCAGCGGCGCGCAGGC |
| | | | | | | | CTGTGGTTTTCTCGCTCT[CG]C |
| | | | | | | | AACCCTGCTTTAACTGCCGGTT |
| | | | | | | | TATTTTTCGACAAACAGGATGC |
| | | | | | | | CTCCATCTGAGGCTG |
| 67 | cg14676592 | chr16 | 49910862 | + | | | GCCGGGATCCGAGAACCCAAA |
| | | | | | | | GCCCCGCAAACTGCGCAGGCC |
| | | | | | | | CAGTAGGGGCTCGCAAAC[CG] |
| | | | | | | | GGGGCCCCAGGGTTCTCACTG |
| | | | | | | | GCCAGCATACTTGTGTAGAAC |
| | | | | | | | TTTGTTTTTTCTTTTTGG |
| 68 | cg10269365 | chr2 | 223166989 | + | CCDC140 | 5′UTR | AGTTCTCCCTCGCAGCCCGTTT |
| | | | | | | | GGATGCGTGCGTCTACAGCCC |
| | | | | | | | AGTCGCACTTTGGTGAC[CG]G |
| | | | | | | | CCTGGGCTGTGAAGCACCCTTT |
| | | | | | | | AGCGAACAGCCTCCGCACTTG |
| | | | | | | | GGGACACTGGCACAAG |
| 69 | cg01682111 | chr16 | 1430087 | + | UNKL | TSS1500 | GCCTGCCCTGCAGGACCCTCCT |
| | | | | | | | CCCTCCCAAGTCCGCGTGCCTG |
| | | | | | | | CCCAGCCCCATCTAAA[CG]CG |
| | | | | | | | GGGTACGGAGCTCGCAGGTCT |
| | | | | | | | CTCTTAATCTGAAACCTGTTCC |
| | | | | | | | TATGAAGTGTAAGAT |
| 70 | cg10501210 | chr1 | 207997020 | + | | | ACGTGGGGGAAGAAGGGGGT |
| | | | | | | | TACGCCATCAAGTCCTGAAGC |
| | | | | | | | CCGTCGGACCACCCATCGC[CG] |
| | | | | | | | CCTGCGCAGACCCAAATCTTG |
| | | | | | | | GTCCCGCCGTAAGGTGCCGCA |
| | | | | | | | GTCCCGAATGTTCCAGAA |
| 71 | cg27345346 | chr19 | 36259144 | + | C19orf55 | 3′UTR | ATCCCGTGCTGCAGGTGCTAA |
| | | | | | | | GAGCCCATAGGGCAGAGCTGA |
| | | | | | | | GTCGGCAGAAAAGGTGAC[CG] |
| | | | | | | | ACCCTCCATCCCCAGAGTCTA |
| | | | | | | | TGACACTGGGCCCCGGAGACC |
| | | | | | | | TCTGAGACCCGGTTAGGC |
| 72 | cg08097417 | chr7 | 130419133 | - | KLF14 | TSS1500 | CCGGCTAAGTCATGTTTAACA |
| | | | | | | | GCCTCAGAAATTATCTTGTCTC |
| | | | | | | | CGCGTTCTTTCTTCTGC[CG]GC |
| | | | | | | | GAGCCAGGTAATGGTAACAGA |
| | | | | | | | GCGAAACTCCCCAGTCGGAAC |
| | | | | | | | TTCTGGGTTGCAGCAG |
| 73 | cg19456540 | chr14 | 60976285 | + | SIX6 | 1stExon | CTGCCCGTGGCCCCTGCGGCC |
| | | | | | | | TGCGAGGCCCTCAACAAGAAT |
| | | | | | | | GAGTCGGTGCTACGCGCA[CG] |
| | | | | | | | AGCCATCGTGGCCTTTCACGGT |
| | | | | | | | GGCAACTACCGCGAGCTCTAT |
| | | | | | | | CATATCCTGGAAAACCA |
| 74 | cg04528819 | chr7 | 130418315 | - | KLF14 | 1stExon | GCAGCCCGGGAAGGGGCATT |
| | | | | | | | GGTGGCGCTTGGCAGCAGGTG |
| | | | | | | | TGACAGACCTCCTCCGGGG[CG] |
| | | | | | | | CCTGATCCGCGGCGGGGGCG |
| | | | | | | | GGGCCTGCCCCTAGGGCCCCT |
| | | | | | | | CCAGAGAACCCACCAGAGG |
| 75 | cg10977667 | chr16 | 31053799 | + | | | CAACTGGGCGAGCTGTGCATG |
| | | | | | | | GGGCGTGGCTAAGGCCGTGGT |
| | | | | | | | TTGGTTACGATTGGCCAG[CG] |
| | | | | | | | GGACTTAAGTGTTGTCTCTGAA |
| | | | | | | | GAGCATGGACATTAGTCTGGA |
| | | | | | | | GGGTCCTGGAAGAGTGA |
| 76 | cg19200589 | chr21 | 36041605 | + | CLIC6 | TSS200 | CGGCTAAACCTTTGCCGCAGG |
| | | | | | | | ATCCCGGAGCCGGCGTCCTTC |
| | | | | | | | AAGGAGCACAGAGGGCCC[CG] |
| | | | | | | | TAGCACGCCCCTTGCCCAGCG |
| | | | | | | | CCACCGACCCTTAAGCAGCGT |
| | | | | | | | CAAGGAAGGAGTCCCGAT |
| 77 | cg23291886 | chr4 | 174440681 | + | | | TGGATTCCACCCCAGCCCGCCC |
| | | | | | | | CCTCCCCACGCACACAGCCAC |
| | | | | | | | GGCCCCTCGCGTCTTCG[CG]G |
| | | | | | | | CACGTTAATTAAATGCGGAAA |
| | | | | | | | ACAGACAGAGGCTGATGTCAT |
| | | | | | | | TGCTCTCACAAGATCAT |
| 78 | cg10911990 | chr14 | 37129141 | + | PAX9 | 5′UTR | AACTGCTAAAGCTCTCGCAGA |
| | | | | | | | GTCCCCAGACCCCCCGCGGGA |
| | | | | | | | CATGAGGTCTTGCCTGTT[CG]T |
| | | | | | | | ATGCGAACATCCTTGTACCCGC |
| | | | | | | | CTAGCAGCCCTGCAGACTGCA |
| | | | | | | | AATTTTCCCTGGGTGC |
| 79 | cg06785999 | chr14 | 60975964 | + | SIX6; SIX6 | 1stExon; | GCCGAGCCCGAACCCCAAGCC |
| | | | | | | 5′UTR | GCGGAGCCAGCACCTCCTCCA |
| | | | | | | | GTCGGGGTCGTCCGCTCC[CG] |
| | | | | | | | GCCGTTGAGCCACCGCCGCCA |
| | | | | | | | CCCGGTAGTGTGTCCCGCTGC |
| | | | | | | | CCCAATCCGCCTCATCAA |
| 80 | cg24715245 | chr4 | 41258794 | - | UCHL1 | TSS200 | TCTCCACAACCACCAGATTATC |
| | | | | | | | TCACCGGCGAGTGAGACTGCA |
| | | | | | | | AGGTTTGGGGGCCCGGC[CG]T |
| | | | | | | | ACCACTCCGCGCTGCGCACGG |
| | | | | | | | GGGGTTCGTACCCATCTGGCC |
| | | | | | | | GCGACCGTCCGTTTCCC |
| 81 | cg18867659 | chr16 | 47178357 | - | NETO2 | TSS1500 | ACCTCCATTCAAGGTCAAAACT |
| | | | | | | | TTGCCCAGCTCAGCCTTGCTCG |
| | | | | | | | ACCCTGGGCAGGGAAG[CG]C |
| | | | | | | | GGACATCGGCAGAGGGAGCC |
| | | | | | | | CGAGGCTCTCCGTGCCCTTCGC |
| | | | | | | | GCCGGTGAGTTCCCGAC |
| 82 | cg10755058 | chr3 | 40428713 | + | ENTPD3; | 1stExon; | GGCGCCGCCTCCCGGCGTCTG |
| | | | | | ENTPD3 | 5′UTR | AGCTGACACCTCCTTAGCGCTG |
| | | | | | | | GCCGCGGGCCGCCTCTG[CG]G |
| | | | | | | | CAGCGCTAGTCGCCTTCTCCGA |
| | | | | | | | ATCGGCTCCGCACAGGTAAGA |
| | | | | | | | TCAGGGGACCCGGCGC |
| 83 | cg07060233 | chr20 | 44687092 | - | SLC12A5; | 3′UTR; | CAGTCCTTTTCCGAGATGAGGT |
| | | | | | SLC12A5 | 3′UTR | GAGACAAGGGTCCAACTTTTC |
| | | | | | | | CTGGATTCGCCTCCCAG[CG]G |
| | | | | | | | ACGTGAGCTTCCACTGCGGCT |
| | | | | | | | GCAGAGACGCGAGCAACCTCT |
| | | | | | | | TCTCATCGGCTCTTATG |
| 84 | cg18533201 | chr8 | 97157453 | + | GDF6 | Body | GCGGTTGCTGGGGTCCCCGCG |
| | | | | | | | CGCGCGCCTCGGCCTCCCCGG |
| | | | | | | | CGTCCAGCTCGCCCCATG[CG] |
| | | | | | | | GCCCGCAGCTCCAAGCACAGC |
| | | | | | | | TGCTTCCAGGGCTGGTGGCGC |
| | | | | | | | AGGCCCTGCCACACGTCG |
| 85 | cg03507326 | chr16 | 2801952 | - | LOC100128788; | Body; | CCTGCCTTGTTCCTGTATGTGC |
| | | | | | SRRM2 | TSS1500 | CGCTTCACCGGTATCACGTCCT |
| | | | | | | | GGGTCTGGTGGGACCC[CG]GC |
| | | | | | | | CTGGCTGCCCTACCGGAAGCT |
| | | | | | | | AAGAAAACTCCTCCCCCAGGG |
| | | | | | | | GTGGCCGTCGGGCCTC |
| 86 | cg06971096 | chr2 | 220173591 | + | PTPRN | Body | CACTGCCCAGAGATCACCGTTC |
| | | | | | | | CCTCATTCTCCCCGCCACCTCC |
| | | | | | | | CCTTCCCATTCCTCAG[CG]CCT |
| | | | | | | | GTCACCACCTCCCAGGCGCCTC |
| | | | | | | | GGAGCAAGTGGCTTCTCCTGT |
| | | | | | | | GGTCTCGCAGCCGG |
| 87 | cg26329178 | chr10 | 100227782 | + | HPSE2; | Body; Body; | ACTCGGCGCTGGGCTCTCCCG |
| | | | | | HPSE2; | Body; Body | GGCTCCGGGTCCCCGGCTGCC |
| | | | | | HPSE2; | | CCCGGCCGCCAGTCGGGT[CG] |
| | | | | | HPSE2 | | GCCCCGCACCTGTTTGTGCTTT |
| | | | | | | | GCAGGCTCCCGGCCCCCTCGC |
| | | | | | | | TGAGCGAGGAAGCTGGT |
| 88 | cg24317217 | chr3 | 70231495 | + | | | AACGTCTGGCAGAGCTCACAG |
| | | | | | | | ACGTCGTTTTCCACTCGGCACC |
| | | | | | | | AAATGTTTTACAGTCTT[CG]TG |
| | | | | | | | AGCCCATATAGATTCTGGCTTC |
| | | | | | | | TGCCCAGTCGTTTGTTTGAAAC |
| | | | | | | | TGTAGGCTCTGAGA |
| 89 | cg24719321 | chr11 | 122850490 | - | BSX | Body | AAAAGAAAATCGGAAAATAGA |
| | | | | | | | TCCGGAGGCTGTTTAAAAATG |
| | | | | | | | TCTTCTTGGAGAGACTTC[CG]T |
| | | | | | | | AGGGTCGGCCAGCGCGGAGT |
| | | | | | | | CTTCAGTTGCGCCTGGCCAAGT |
| | | | | | | | TTTTTGCAAACGTCAAA |
| 90 | cg14226702 | chr9 | 1047220 | + | | | CACGGCCTGACCCCTTTTAAGA |
| | | | | | | | GAGGGACCTCAAGAGGGGAG |
| | | | | | | | CTGAATTCCTTGAGCCCT[CG]C |
| | | | | | | | CTTTCAATCAAGTTTTCAAGGC |
| | | | | | | | ACGCTTTGGCCGGGCCCTCCC |
| | | | | | | | GGACTGGCTGTGCTGC |
| 91 | cg03970036 | chr2 | 220174232 | + | PTPRN | TSS200 | CATGCCCCTCTCGCTGCAACGC |
| | | | | | | | GGCCAACCGCAGGCGGGTGCT |
| | | | | | | | GACGACACCTCCACCCC[CG]G |
| | | | | | | | CTCGTAAGCTAATTTGCGTCAC |
| | | | | | | | ATATGGCGTAAGAGCCCTGTC |
| | | | | | | | GGAGCGGGGGACCTAC |
| 92 | cg21186299 | chr7 | 100808810 | - | VGF; VGF | 1stExon; | GCCGGGGTAGGAGCGACGGT |
| | | | | | | 5′UTR | CGAGGTCTGGCGTCCCGTGGG |
| | | | | | | | CTGGGCTCAGCTGGGTCGG[CG] |
| | | | | | | | CGGCTCCGGGCGGCTAGCT |
| | | | | | | | CGCTCCGGCTTCAGCACGCTG |
| | | | | | | | GACAGCGCCCGCGCCTCCAC |
| 93 | cg15568145 | chr1 | 14113203 | - | PRDM2; | Body; 3′UTR; | CTCAAAAATCCTAACATTCAGC |
| | | | | | PRDM2; | Body; 3′UTR | TGATTGCCGGCAGGCTTAGAG |
| | | | | | PRDM2; | | TCAGGCATCTGCTGCTT[CG]GT |
| | | | | | PRDM2 | | GGGGGCCCAACGCGCATGCTG |
| | | | | | | | GGCGCCCGGGTGATTGAGATC |
| | | | | | | | CAAAGAGAAGGGCACT |
| 94 | cg06365535 | chr17 | 59534102 | + | TBX4 | Body | GGCTGCGCCAGCCGTCGGGTA |
| | | | | | | | GAAGTCGGGCGTCGGTCTGTC |
| | | | | | | | TGCGGGGCCGCCTGTGTC[CG] |
| | | | | | | | TCTTTCCGTCCGATTGTCGGCA |
| | | | | | | | GGACTCGCTTTCAGGAGGACC |
| | | | | | | | TGGCTGCATTCAGGACG |
| 95 | cg01359962 | chr3 | 43148002 | - | C3orf39 | TSS1500 | TGTCCAGTCCTCAAGGGCAGC |
| | | | | | | | TACTTATGGCTGTGGCATCTGG |
| | | | | | | | CATTCCCGCGGATTCTC[CG]AA |
| | | | | | | | TATACATATGCCCCTATTTCTT |
| | | | | | | | GAGTTATGAATTTTAGATCTTT |
| | | | | | | | TGACTTCTTTTTTA |
| 96 | cg07116393 | chr1 | 20834843 | + | MUL1 | TSS200 | GAGCGATTGGGGAGCTGAGC |
| | | | | | | | GACCACCCACCGCTCCATGGC |
| | | | | | | | CGTCCCCTTCGAAACACGG[CG] |
| | | | | | | | CACTGGCCATGACTGACTCGC |
| | | | | | | | CCATCGCCCTGGTTTCCGTCCC |
| | | | | | | | TCTGGTTTCCTGGGGTT |
| 97 | cg13696942 | chr11 | 20180666 | - | DBX1 | Body | ACGCCTCGCAACCTCTGAACCA |
| | | | | | | | GAGCATAACCCCGAGGGGTG |
| | | | | | | | GACGGAGAAATACGGCTT[CG] |
| | | | | | | | GAGCAGGGAGCGATGGGCCG |
| | | | | | | | GGGCTGGGGCGCCGCCCTGCC |
| | | | | | | | TCGCGCAAAGAAGGGGGAC |
| 98 | cg09370594 | chr19 | 2291872 | + | LINGO3 | 5′UTR | TCCTGCGCACCTGCGGGCGGG |
| | | | | | | | CGGGGAGCGGGCAGCGTTAG |
| | | | | | | | CACCGTTAGCACCCCTCCG[CG] |
| | | | | | | | GCGCCTCTGCCGCCAGCCCGC |
| | | | | | | | CCCTAACCCGTCCCAGCACGG |
| | | | | | | | CGGCTCGCTCCTGTAAAC |
| 99 | cg25763393 | chr19 | 52956832 | - | ZNF578; | 1stExon; | GGAAGTGAATCATGGGGCGT |
| | | | | | ZNF578 | 5′UTR | GAACTCGCAAGCGCAGTTTCC |
| | | | | | | | TGAAGACCCGGAAGCCGAT[CG] |
| | | | | | | | CGTGGGGAGCCGGTCTTGG |
| | | | | | | | AGCAGCGGGTGAGTTTCCCTT |
| | | | | | | | TGTCTAGATTAGATCCGCTT |
| 100 | cg24136205 | chr13 | 100624293 | - | ZIC5 | TSS200 | CCGGGGATGCCCAAGTTGCAC |
| | | | | | | | TTGCAGAAAGTTTGAGCCTGG |
| | | | | | | | CCTGCGCGCGCAGCGCCC[CG] |
| | | | | | | | CTCTTCCTTGACGCACCTCGCG |
| | | | | | | | GAGCGCGCGCCGGCACGCGG |
| | | | | | | | GCAGAGGGCGCGGGGTGG |
| 101 | cg06571559 | chr10 | 670787 | - | DIP2C | Body | TGAACCCTCCCCAGGAGCTCA |
| | | | | | | | CCTGGGGCACCCACGAGAAAA |
| | | | | | | | CTACGGAAGCTGTGAAGA[CG] |
| | | | | | | | GAGGTGTGCATGTGGCCGGG |
| | | | | | | | AGAACCCGGGGGGGGAGCCG |
| | | | | | | | CACTGGGGACAGAGGGGTGG |
| 102 | cg13592721 | chr6 | 27107393 | + | HIST1H2BK; | 3′UTR; | CACCGCCATGGACGTGGTCTA |
| | | | | | HIST1H4I | 1stExon | CGCGCTCAAGCGCCAGGGCCG |
| | | | | | | | CACCCTCTATGGCTTCGG[CG] |
| | | | | | | | GCTAAATGGCATTTTGAAGCC |
| | | | | | | | CAGTCATTCTCTAAAAAGGCCC |
| | | | | | | | TTTTTAGGGCCCCTAAG |
| 103 | cg23995459 | chr1 | 53191787 | + | ZYG11B | TSS1500 | CTGAGCCAAGAATGATCCCTA |
| | | | | | | | GAGAAGAATCTGAGAGGCCA |
| | | | | | | | GAGGATTGGAAGAATTAAG[CG] |
| | | | | | | | AATTTTGAAATAACCAAGAG |
| | | | | | | | TTATGACAATAGTAGTAATGA |
| | | | | | | | ATGACAGTGAACCAGAAGC |
| 104 | cg23136139 | chr10 | 43697918 | - | RASGEF1A | Body | CCAGCACAGGGCCTAGGGCAT |
| | | | | | | | GGGGACTGGCCCTCTTGGCTG |
| | | | | | | | AAACGACTCCGACCCTCT[CG] |
| | | | | | | | GAAGATGCCCGCGCGGCCTCT |
| | | | | | | | GCCCCCGGGGAGAGGGGACT |
| | | | | | | | GTGCCCGATGCTCAGGCGC |
| 105 | cg11970349 | chr4 | 8582287 | - | GPR78 | TSS200 | CGCGAACCAGGGCTGGGAGG |
| | | | | | | | CTCGGCTGGAGGTGTGACCAG |
| | | | | | | | GGCAGGGACTGACCTGGCC[CG] |
| | | | | | | | GAACAGAAGCGCGCAGAGT |
| | | | | | | | CCCATCCTGCCACGCCACGAG |
| | | | | | | | GAGAGAAGAAGGAAAGATAC |
| 106 | cg06287137 | chr2 | 27497831 | + | DNAJC5G | TSS1500 | TAGTGACTTTTGGAAAAGGCT |
| | | | | | | | CAATACATCATTTTAATGAGAC |
| | | | | | | | GTGCAAACTCATCATTA[CG]AT |
| | | | | | | | ATACTAGGAGAAATGCTTTGA |
| | | | | | | | CAGACGAAGTGGGAACAACTG |
| | | | | | | | GGAGAGTGAATGATGG |
| 107 | cg21269897 | chr6 | 27107002 | + | HIST1H2BK; | 3′UTR; | GCCTGTTTCCCTTTTAGGTCCC |
| | | | | | HIST1H4I | TSS200 | CTCCCCCAATGCAGAGGGACT |
| | | | | | | | TCCCGCCAAAGCTCTTC[CG]GT |
| | | | | | | | TTTCAGTCTGGTCCGCAGAGG |
| | | | | | | | TTACCCATAAAAGAAAGCTGC |
| | | | | | | | CATCACAGGCAGCAGA |
| 108 | cg18988435 | chr18 | 12287275 | - | | | CTGCTCAGGGCTTCCTCAAGGT |
| | | | | | | | GAGCTCAAGACCCGCAGGGCT |
| | | | | | | | TCCCTATGGCAAGCCGT[CG]A |
| | | | | | | | GGCTTTCTTTGGATGCAGGTG |
| | | | | | | | GCCGCAGAGCGCTCATGCGGC |
| | | | | | | | GTCGGTGCTGGCAGCCA |
| 109 | cg14663984 | chr1 | 969042 | + | AGRN | Body | TGAACGCCCGCAGCCTCAGTC |
| | | | | | | | CCACCCCCGGCCCAGCCCCAG |
| | | | | | | | CGCCCCCAGTCCCACCCC[CG] |
| | | | | | | | GCCCCAGCTTCAGCCTCAGCG |
| | | | | | | | CCCCCAGGCCCAGCCCCAGTC |
| | | | | | | | CCACCCCCAGTCCCAACA |
| 110 | cg18371700 | chr21 | 36041579 | + | CLIC6 | TSS200 | GGGTCCTGCGCAAGGCCCCAG |
| | | | | | | | TGCCCCGGCTAAACCTTTGCCG |
| | | | | | | | CAGGATCCCGGAGCCGG[CG]T |
| | | | | | | | CCTTCAAGGAGCACAGAGGGC |
| | | | | | | | CCCGTAGCACGCCCCTTGCCCA |
| | | | | | | | GCGCCACCGACCCTTA |
| 111 | cg12242474 | chr20 | 1293682 | - | SDCBP2; | Body; Body | CCTGGGGCTGCACTCCGAAAC |
| | | | | | SDCBP2 | | ACTCCACTGTACCATTCACAAA |
| | | | | | | | GGCATGGGCTTCCCTGG[CG]T |
| | | | | | | | CGGCTGTCTACACCGTCGCCTG |
| | | | | | | | GAAGCTAGATGCCCTGGGCAG |
| | | | | | | | CGAAGGGCAGGTGGGG |
| 112 | cg26115667 | chr14 | 103294656 | - | TRAF3; TRAF3; | 5′UTR; | AGCTTTCAGAAAGACTGCAAT |
| | | | | | TRAF3 | 5′UTR; | GCAGCGGTTACCAAAGTCCTT |
| | | | | | | 5′UTR | GTTAATATGGAAACAACT[CG] |
| | | | | | | | TGGTGAAGCCTTTTGCTCCCCT |
| | | | | | | | TCACAACTGCTGACTGTTGCCT |
| | | | | | | | GCAGTCGGAAGGAGGA |
| 113 | cg23156348 | chr11 | 124981869 | + | | | TGGGCCATTGGTCAGTCTAGC |
| | | | | | | | CTGAGGGCGGGTTGTTGGGCG |
| | | | | | | | GAAGAGAGAGACTTCTTC[CG] |
| | | | | | | | GCCTCACTCGCTGTCACCATAG |
| | | | | | | | AGATTGCCCATCCAGGCAGCG |
| | | | | | | | AAGCAGCAGGGCCAGGC |
| 114 | cg13337731 | chr7 | 73011308 | - | MLXIPL; | Body; Body; | CTTGCTCCGGCTTAGCTGTGCA |
| | | | | | MLXIPL; | Body; Body | CGGGCAGAACCGTGAGGCTAC |
| | | | | | MLXIPL; | | TGGGGCTGGCCCACCCC[CG]G |
| | | | | | MLXIPL | | CATCTATCAAGACCCCATCCTG |
| | | | | | | | CCCCTCCCAAGAGTCCACACCC |
| | | | | | | | CTTTTAGGTACAGGC |
| 115 | cg09393254 | chr6 | 100442118 | - | MCHR2; | TSS200; | ACTTCATCCAATCCGAGCATCG |
| | | | | | MCHR2 | TSS200 | GGTGCGTCGTGCTCTTTTCTAG |
| | | | | | | | GAGCGTGGGGTGCCTT[CG]CG |
| | | | | | | | AATAAAATCTGAAGGCATCTCT |
| | | | | | | | GCTCTCGCGGAGCTTGTTCTTT |
| | | | | | | | CTTATTTTCAAGTG |
| 116 | cg02081006 | chr5 | 122430434 | + | PRDM6 | Body | ATTGCCCTATAGTTTTGTAGGA |
| | | | | | | | GAGAGTGGAGCCAGCCCAGA |
| | | | | | | | CCCGCTTCGATCTCCTCT[CG]C |
| | | | | | | | GGCTCCTATTCATCATCTCCGC |
| | | | | | | | ATTGTATATGGCAGCCTCGCA |
| | | | | | | | GGGGCAGGGGCCGGCG |
| 117 | cg06520675 | chr10 | 102996310 | + | FLJ41350 | Body | CGCGCGGCGCCCAATTCCCCG |
| | | | | | | | CGGAGGGGAGTAGCCAATTAA |
| | | | | | | | GGCACTTGAAAAGGGAGT[CG] |
| | | | | | | | GGTGGAAGATCCCCCGCCCAC |
| | | | | | | | CAGTATCCTGGATTTACCCAGG |
| | | | | | | | TCGAGTTCAGAGAGCCT |
| 118 | cg00323305 | chr3 | 24537182 | - | THRB; THRB; | TSS1500; | GGAAAGAATGGGGAACGAGT |
| | | | | | THRB | TSS1500; | GACACCGGGACCGGAGGGCG |
| | | | | | | TSS1500 | AGTCTTCCAGGAGCACGTCT[CG] |
| | | | | | | | GCCTTCTTTGCCCGGCCCGA |
| | | | | | | | CCGGCCCGACCCGTGCCGCAG |
| | | | | | | | CGCTCCTCCCTCCGCTCCT |
| 119 | cg10196902 | chr5 | 172823642 | - | | | TTTGGATGTTGGCACAAGGCT |
| | | | | | | | GCCTGCTTGCATTAGAACTCAG |
| | | | | | | | CCGGCAAGGAAAGCAGG[CG] |
| | | | | | | | GCTCAAAGACTGGGTCAGCCT |
| | | | | | | | CAGGGACTGGATGGGGATGG |
| | | | | | | | AGCTTTCAGAGGAGTGGCC |
| 120 | cg21353911 | chr2 | 186603398 | - | | | GATGGTTTCAGAGAAAGATGA |
| | | | | | | | AGTTTCAACTGTGGTCCTCTCA |
| | | | | | | | GATCAGGCCTCTCGGAC[CG]A |
| | | | | | | | TTTTCCCAGCTCTGCGGGCGCT |
| | | | | | | | CTACGCGCTGGCGCGAGCCGC |
| | | | | | | | CCCTCAGGAGGCCACC |
| 121 | cg21091227 | chr18 | 4454304 | - | | | TCGCCCAGCCCAGAGGAGAGG |
| | | | | | | | TCCCTGTTTGGCCTTGGTTCCA |
| | | | | | | | GCCCGGCTCATTCAATT[CG]CT |
| | | | | | | | GAATGTCGGGTCTCCCGGCCC |
| | | | | | | | GCCCCGCGATTCTCCGGGAAT |
| | | | | | | | TGGCCTTGGCCGCGGG |
| 122 | cg19026977 | chr5 | 172999989 | - | | | CCATGGGCTGCCCATTGCCACC |
| | | | | | | | TCTGGGCAGCCCTCCTTGATG |
| | | | | | | | GTGTGGAGTCCGCGGTC[CG]C |
| | | | | | | | ATTGGTTAACTTAACTGTGCTT |
| | | | | | | | CCTCAGATCCAGTCTGGAATTA |
| | | | | | | | ATTATTGAATTGTAT |
| 123 | cg08079908 | chr2 | 176997277 | + | | | ATTGCCTTTGTTCTGTTCGCCG |
| | | | | | | | CTGGTTTTAAACCAGCTTGCTG |
| | | | | | | | TGTGCATCTCAGACGT[CG]GT |
| | | | | | | | TGGTACGTCCTCCGCTGTTCTT |
| | | | | | | | CAGGAAAGCGATAGCCTCACC |
| | | | | | | | TATTTGAAACAAGCC |
| 124 | cg02983163 | chr21 | 47010461 | + | | | CCGTGCCCGCCCCGGGAGTTC |
| | | | | | | | GAAGGGTGCTGGGGCCGAGG |
| | | | | | | | GGAAGGCTCTGGTCGGCGG[CG] |
| | | | | | | | TCAGCGGCAGCTCCCAGAC |
| | | | | | | | GACCTAGGACTGCAAAGGGCC |
| | | | | | | | CAGGACGGGGGGCGGGGCGG |
| 125 | cg21901946 | chr7 | 127744210 | + | | | CTCGGCAACGCGCCCTCGGCC |
| | | | | | | | CGCAGCCTCCTGCCCCCTGTGC |
| | | | | | | | CCCGCTTCGGCCCCCAG[CG]C |
| | | | | | | | AGCTGCAGAGGGGCCCCCCTC |
| | | | | | | | GACGCATACACTCAAGAGCCC |
| | | | | | | | GACCGCGCGGCTGAAAT |
| 126 | cg17040303 | chr21 | 38070535 | - | SIM2; SIM2 | TSS1500; | TCTTTAGGTCCAAAATGACCCT |
| | | | | | | TSS1500 | GAAGGAGAGTCCAGAATGCCC |
| | | | | | | | AGTGGCCGCGTCTGCAA[CG]G |
| | | | | | | | AGTCTTCTTTCTCCAATTGCCTT |
| | | | | | | | CTGCCCCATCACCATGGGCCCC |
| | | | | | | | ACCTGCGCCACCTG |
| 127 | cg09551472 | chr6 | 27280195 | - | POM121L2 | TSS200 | GACACGCGGGACTTCGGCAGT |
| | | | | | | | CCCAGTAACTTGCTTTGCTGTT |
| | | | | | | | CTGAGACCTCAGCGGGG[CG] |
| | | | | | | | GTCAGACCTCTGCTGTCTCCGC |
| | | | | | | | AGCGAGTTGCAGTACTTGGCG |
| | | | | | | | CGGGGAGAGGAACTCGA |
| 128 | cg13140267 | chr2 | 96971704 | - | SNRNP200 | TSS1500 | GGGCCGAAAACCCCATTTCCG |
| | | | | | | | TTTGAGGTAACTAAAGTACCC |
| | | | | | | | AGCGAGCAAGGTGACTTG[CG] |
| | | | | | | | CGTGTGTCTGTGTTTGTGTGTT |
| | | | | | | | TTAATGATTGGCGCCTTGCTTT |
| | | | | | | | GGGTTTCTCTTCTGTG |
| 129 | cg11716026 | chr11 | 2016937 | - | H19 | Body | GGATGATGTGGTGGCTGGTGG |
| | | | | | | | TCAACCGTCCGCCGCAGGGGG |
| | | | | | | | TGGCCATGAAGATGGAGT[CG] |
| | | | | | | | CCGGTGCGGGGTGGGTGCTGC |
| | | | | | | | GGGCGCTGCTGTTCCGATGGT |
| | | | | | | | GTCTTTGATGTTGGGCTG |
| 130 | cg25273520 | chr15 | 59713427 | - | | | TGAACTCTGCATTCCTAACAGT |
| | | | | | | | AGAGGGGCTCGTGTTCTTGTG |
| | | | | | | | CATAGATCACACTTCGA[CG]G |
| | | | | | | | GCAATGTTCTAGGTAGAATTG |
| | | | | | | | GAGCTCAGTGGAAAGGCAGAT |
| | | | | | | | CCCTGACAGCTTGAACA |
| 131 | cg06432426 | chr2 | 484825 | - | | | ATAGAAGAGGTATTTGCAAGT |
| | | | | | | | TCAATCGAGCCACACGTAGGA |
| | | | | | | | CCATACACGGAAGTGAAC[CG] |
| | | | | | | | TGTGAGGAATGTGTGTGGGAG |
| | | | | | | | AGTTCGCGTGAAGTCTGCGTG |
| | | | | | | | CACAAGGCAGCGGCGGCC |
| 132 | cg24813736 | chr5 | 63255045 | - | | | TCGTAAGGATAAAATTGCTCTT |
| | | | | | | | TCAGGTTTTACTGGGGGAGCC |
| | | | | | | | AGCTGGAGCCTTGGGCA[CG]C |
| | | | | | | | GCGCCCTGGGGAACCTTTCCTC |
| | | | | | | | TTTGCCGCCCCTGCGTGTCGCC |
| | | | | | | | CCTTTAAAGCCTTCT |
| 133 | cg17486097 | chr8 | 35093411 | - | UNC5D | Body | TGGCTCCCGTGGCTGGGGCTG |
| | | | | | | | TGCTTCTGGGCGGCAGGGACC |
| | | | | | | | GCGGCTGCCCGAGGTAAG[CG] |
| | | | | | | | CTGGGCGGAGCGGGCAGCTG |
| | | | | | | | GGGGCGAGGGCGCAGGGGCG |
| | | | | | | | CCAGCCTGACGGAGCGGGAC |
| 134 | cg26792755 | chr7 | 140714919 | - | MRPS33; | TSS200; | TTACTGGCTCCCCCTCCTGAGG |
| | | | | | MRPS33 | TSS1500 | CCTCCGAGGTGTACCTGGCGC |
| | | | | | | | CTGCGCAGTAAGGCTAG[CG]C |
| | | | | | | | CGCCGCCTGTGCGGAGGACCC |
| | | | | | | | GGGGAGGTGGTGGGCTGGGG |
| | | | | | | | AGAGTTAGAAAGGTCTGG |
| 135 | cg26856080 | chr3 | 160167746 | - | TRIM59 | TSS200 | AACTGCAAGGCATCGGCCAAT |
| | | | | | | | GGGAACTATTGCTGGGCTCGT |
| | | | | | | | TCGAAAGTAAACGGTGGA[CG] |
| | | | | | | | GCGCGGCCCGAGGCAGGTGG |
| | | | | | | | CGGGAGTCAGTTTAAGGCTGG |
| | | | | | | | CGCCCAGCTTTCCGCGCCT |
| 136 | cg06385324 | chr16 | 2014621 | + | SNHG9; | TSS1500; | GCGGTTCCCCATCCCAGGGCC |
| | | | | | SNORA78; | TSS1500; | ACCAGGGCCCCCGGGCCCCCC |
| | | | | | RPS2 | Body | CGCTGCACCGGCGTCATC[CG] |
| | | | | | | | CCATTTGCTGGGAAAAGCGAC |
| | | | | | | | AAGAAGGAACTAGTCAGTGTG |
| | | | | | | | GCCTACGCATCTGGCAGC |
| 137 | cg04811592 | chr3 | 69834386 | + | MITF; MITF | Body; Body | GGGCACTTGAACATTCTTCATG |
| | | | | | | | AGGGCTGAGGCAGGCAAGCT |
| | | | | | | | GAGTGGAGCAGTGAGTCA[CG] |
| | | | | | | | GCGTGCTGCGGCAGTGGTGT |
| | | | | | | | CCTGAAATAACAGCAAGCAGC |
| | | | | | | | AGCAGCAGCAGCAGCAGTA |
| 138 | cg03735496 | chr18 | 18822637 | + | GREB1L | 5′UTR | GCCGTGCCTGCCTTCCCTGCCG |
| | | | | | | | CCTCGCGTCGCCCACCGAAGG |
| | | | | | | | GACCCGGCCGTGCTGTC[CG]C |
| | | | | | | | GCCCAGAGGCCGAAGGCCTGT |
| | | | | | | | CACCGGGCTCTACTCGCTGCCT |
| | | | | | | | TTGTGGCGGGAGCGAG |
| 139 | cg14772615 | chr6 | 33116235 | + | | | ACCAAATACATAGGTTTTGGC |
| | | | | | | | AGCACATAGATTTCTGTGGTTT |
| | | | | | | | TGCTATGCTTTTAGCAG[CG]G |
| | | | | | | | CTGTAAAAAGCATTGCACACT |
| | | | | | | | AAGCATTGCTAGATTGCCAAA |
| | | | | | | | CAAACCTAATTACATTT |
| 140 | cg24914355 | chr2 | 176959229 | + | HOXD13 | Body | ATCCCAGCCTAATTTTTCTTGT |
| | | | | | | | GCTTTTGTTTGTATCAGGGGAT |
| | | | | | | | GTGGCTCTAAATCAGC[CG]GA |
| | | | | | | | CATGTGCGTCTACCGAAGAGG |
| | | | | | | | GAGGAAGAAGAGAGTGCCTT |
| | | | | | | | ACACCAAACTGCAGCTT |
| 141 | cg13141009 | chr3 | 179660224 | - | PEX5L | Body | GGGATGTGTCCGCAGTTGCCA |
| | | | | | | | GAGCAATGACAACACTGCGGG |
| | | | | | | | ACCGCGGAGGCGGCTGGG[CG] |
| | | | | | | | GGGCTGGAGCCTGTGACCGC |
| | | | | | | | GCCCGCTGCGCGCATGCCCAA |
| | | | | | | | GGCCCCAGCGCTTCTGCAG |
| 142 | cg14979301 | chr5 | 42994123 | - | | | TTTTAAACTCCCATGGAAGTCA |
| | | | | | | | GGAAATGCCGGCAAAAGCGAT |
| | | | | | | | TTCTGGTTTACGAAGCT[CG]GT |
| | | | | | | | TTGACGATAGCAATTTCCGCCG |
| | | | | | | | AACGCGACTTTTTCCTCTTGTG |
| | | | | | | | GACCAAGTCGGGAT |
| 143 | cg09785958 | chr13 | 113274490 | + | | | TCGACGTGCCAAGAACCTGGA |
| | | | | | | | CAGCTCTCAGCCGAGACCCTTC |
| | | | | | | | ATCTGGTGACGAATGGA[CG]T |
| | | | | | | | TGAGTGAGTGCTCAAGCTCAG |
| | | | | | | | ACAGCTGCCTAACAAGGTTCTC |
| | | | | | | | GAAGTCCCCGCCACAC |
| 144 | cg26620450 | chr12 | 133195061 | + | P2RX2 | TSS1500; | CGGCCTGGACGGGGTGGGGG |
| | | | | | P2RX2; | TSS1500; | GCGCCGCGGAGGCCGGCGGG |
| | | | | | P2RX2; | TSS1500; | ACTTCCCATGTCTTTCTCCT[CG] |
| | | | | | P2RX2; | TSS1500; | AGCTCGGAAAAAGTTCCCACC |
| | | | | | P2RX2; | TSS1500; | CGGGGAATCCCGACCCTCCAA |
| | | | | | P2RX2 | TSS1500 | CTTCGAGACCGCCGGTTC |
| 145 | cg21467631 | chr2 | 602296 | + | | | GGAAGCCCCGACCCTGCAGTG |
| | | | | | | | CTGAGGGAGCGGCCCCGTTCC |
| | | | | | | | TGCCTCCGCCAAAACTGT[CG] |
| | | | | | | | AGTGTTCTGTTACTGACAACCG |
| | | | | | | | AACATTCCCAGCTAAAACAAA |
| | | | | | | | GCTTGTCCTATGCCGCC |
| 146 | cg20223728 | chr6 | 6006398 | - | NRN1 | Body | TGTTAAAATATGTGGTCTGAA |
| | | | | | | | GTTCCCTATCACTCTCGATTTG |
| | | | | | | | CCCACCAGCCGGGTCTG[CG]G |
| | | | | | | | TGCCCGTGCAAACGCTGCAGC |
| | | | | | | | TAGGATATAGGGGGGAGGAG |
| | | | | | | | GGGCGGGAGAATGACAAA |
| 147 | cg24888989 | chr3 | 44803291 | - | KIF15; | 1stExon; | CGTCCGATCCAAGCGCCAAAT |
| | | | | | KIF15; | 5′UTR; | TCAAATTTGCGGCCATCTTGAG |
| | | | | | KIAA1143 | TSS200 | CGGGCGGAATTCAGTCG[CG]C |
| | | | | | | | GCGGTGCAGTCGGGAGGTGG |
| | | | | | | | AGGCACCGGCTGCATTGTTTTC |
| | | | | | | | GGGATCGAGGGGTGAGG |
| 148 | cg06617961 | chr16 | 33965255 | + | MIR1826 | TSS1500 | ACCGTGCTGTGGGGGCGGGA |
| | | | | | | | ATCCCCGGGCGCCCGTGGGGT |
| | | | | | | | GCTGTCAGTGTTCGCCCTC[CG] |
| | | | | | | | CCCCCGTGGTCGACACCGCCTC |
| | | | | | | | CCTGTGTTGTGAAACCTTCCTA |
| | | | | | | | CCCCTCTCTGGAGTCT |
| 149 | cg25636665 | chr2 | 80549579 | - | CTNNA2; | Body; Body | CGGAGCCACTTCCCTGAAAGC |
| | | | | | CTNNA2 | | CAGTGAACCTATTTACCATTGT |
| | | | | | | | CATAGTAACACACAATT[CG]G |
| | | | | | | | GCCCACGTAGACTTAATCCCG |
| | | | | | | | AGAGGCAATTGTTCCCTTGCTT |
| | | | | | | | GGGCGGCTACGCTCCC |
| 150 | cg11027140 | chr9 | 127212625 | - | GPR144 | TSS1500 | CTCCCACCCACCTGGAGGCAG |
| | | | | | | | GTCTCTGTCTGGCTGGGCCGG |
| | | | | | | | GTGGGGGGCCCAAGAGGG[CG] |
| | | | | | | | GGGTGGGGAGCGGAAAGG |
| | | | | | | | GGCGTGGCCGAGGGGCGGGG |
| | | | | | | | TCTCCCGGGCCGAGGGGCGG |
| | | | | | | | GA |
| 151 | cg24794228 | chr19 | 52391166 | + | ZNF577; | Body; 5′UTR; | CTGCTGGAGGCGAGTCAGGG |
| | | | | | ZNF577; | 5′UTR; | ACCCGAAGTCTCTAAACACTCG |
| | | | | | ZNF577; | 1stExon; | CCTCTACCCGCCGCCCCG[CG] |
| | | | | | ZNF577; | 1stExon | AACCCCACACACTGCAGACGC |
| | | | | | ZNF577 | | GACACTCGCAAGTTTCGGGGA |
| | | | | | | | TGGCGGCCGGCGAGGGCC |
| 152 | cg05437148 | chr16 | 30675880 | + | FBRS | 5′UTR | CCGCTAACGCCCTTTCTGGTGA |
| | | | | | | | GTTTGGGGTCCTGGCCGGGGG |
| | | | | | | | GTGGGGGGCCATCACCC[CG]G |
| | | | | | | | GCTCGGGCCCAGTTGGCTTTG |
| | | | | | | | GGGCACCTGAGCCTCAGCAGA |
| | | | | | | | CAGCAGGGCTTGAGGAG |
| 153 | cg18151345 | chr11 | 60720229 | - | SLC15A3; | TSS1500; | ACTTTCAACAAGCCTGCGGGC |
| | | | | | SLC15A3 | TSS1500 | CATAGAGGACCACAAGTGAGT |
| | | | | | | | CGGGATTGAGAGGGACAC[CG] |
| | | | | | | | ACCTCAGACTAAATCAGAGTC |
| | | | | | | | AGCCTCAGAACTCCTAAGCAC |
| | | | | | | | CAGCCCCACCCTGACCTA |
| 154 | cg06144905 | chr17 | 27369780 | + | PIPDX | TSS200 | CTGACCTCACCACCCACCAGG |
| | | | | | | | GAGGTGGGTCTTATTCTGGGC |
| | | | | | | | ATCGTGCCAAGTTCTTAG[CG] |
| | | | | | | | GGGCCCTCTAGAATCTCTAAA |
| | | | | | | | GCAAATCAGGCTGAAGAGGG |
| | | | | | | | GAAAACCAGCAGGGGGAGG |
| 155 | cg10635145 | chr11 | 27742435 | - | BDNF; BDNF; | Body; | GCTTTGCCAAAGCCATCCTGTT |
| | | | | | BDNF; BDNF; | TSS1500; | AATAGTTGATCACATGTTGATG |
| | | | | | BDNF | TSS200; | AGAACCTTTTCTTCTA[CG]AGA |
| | | | | | | TSS200; | GGATTACCCATTACCGGTGAT |
| | | | | | | TSS200 | ATGCACTTCTGACTTATTTCTCT |
| | | | | | | | CCCCCCAACCCCA |
| 156 | cg21449170 | chr7 | 130419062 | + | KLF14 | TSS200 | GCACCGGAGCCCGCGGGGGC |
| | | | | | | | GGCAGAGACCCGCCCCGGCCC |
| | | | | | | | GCAGGACACCCCCTCGGAA[CG] |
| | | | | | | | CGCGGCCCCCCGGCTAAGTC |
| | | | | | | | ATGTTTAACAGCCTCAGAAATT |
| | | | | | | | ATCTTGTCTCCGCGTTCT |
| 157 | cg01994205 | chr13 | 79177467 | - | POU4F1; | 5′UTR; | CAGGGAGGGTGGGATGCATG |
| | | | | | POU4F1 | 1stExon | GCAAAGTGAGGCTGCTTGCTG |
| | | | | | | | TTCATGGACATCATCGTGG[CG] |
| | | | | | | | GCTTGGCATGTATATCCACAA |
| | | | | | | | ACACTCCGAAAGTCCGCGGGA |
| | | | | | | | AAGTGCGTACGCCGGCTC |
| 158 | cg15911409 | chr2 | 237481080 | - | CXCR7 | 5′UTR | CCTTGAACCACTGTTGGCAAA |
| | | | | | | | GGGACAGATAACGAGCCCAG |
| | | | | | | | GGCAGTGTGGGGGACTTTG[CG] |
| | | | | | | | TTTTGAAGTCTGGGTCAGCC |
| | | | | | | | AGATAGTAAGCATCTTTTGCTT |
| | | | | | | | TTCCTGCTATAACAGATA |
| 159 | cg03553786 | chr3 | 13692202 | - | LOC285375 | TSS200 | GGTGGCATGCGGAACTGCGG |
| | | | | | | | ACGGCTGCGCAGGAGCGGAC |
| | | | | | | | AGCGGAGAGGCGGTACTGAC |
| | | | | | | | [CG]GTGCGAGGCGGTGCTGAC |
| | | | | | | | CGGTGCGGGCCGGTGCGGGC |
| | | | | | | | CAGTGCAGGCCAGGCCCGGCC |
| | | | | | | | G |
| 160 | cg24340081 | chr8 | 63614431 | - | NKAIN3 | Body | TTATTTGAAGCCTGTCTTGCAT |
| | | | | | | | GGCCATTTGGAACTGACATTTC |
| | | | | | | | TGCTGCAATTCCAAAG[CG]CG |
| | | | | | | | AACTCCGGGGGCTGAAGTCCA |
| | | | | | | | CCTACGCTCCACTTAACCCCAT |
| | | | | | | | ATACTCAGAATGCGC |
| 161 | cg13601993 | chr9 | 127534760 | + | NR6A1; | TSS1500; | ACCAATCCCTTAGCCCTTTTATT |
| | | | | | NR6A1 | TSS1500 | TTTTTTTTGCCTAATTTTAAGTC |
| | | | | | | | CTCGTCCTGGCATT[CG]CATCC |
| | | | | | | | CTGCTTGGCCTGACCCTTGCCC |
| | | | | | | | ACATTTCGCACCATACCCCGTC |
| | | | | | | | CCTCACCTGCT |
| 162 | cg18413131 | chr3 | 131080697 | + | NUDT16P; | TSS200; Body | TAAGGCGCCCAGGTTCCTCCCC |
| | | | | | NUDT16P | | CTTATCCCTGCAGGGCTGGTG |
| | | | | | | | CCTTGCGGCACCGCCCA[CG]C |
| | | | | | | | TCGGATTGGTCCGAGGTGAGA |
| | | | | | | | TTCGCCCTTGTGCCCTCGTAGG |
| | | | | | | | CCTTCGGAACAGCGGA |
| 163 | cg07674022 | chr4 | 122854330 | - | TRPC3;T | Body; | TTCTGGAATACACACTACCCAC |
| | | | | | RPC3 | TSS200 | TGCAAACCTCTGGCTGCAGGG |
| | | | | | | | GTCGGCTCAGTTGCTAG[CG]A |
| | | | | | | | TACCGTTGCTAACTACTCGCCT |
| | | | | | | | GAAAGTGACACCTGTGATCTA |
| | | | | | | | ACCCTGGCTGCTAGAT |
| 164 | cg08964780 | chr7 | 27209463 | + | MIR196B | TSS1500 | GGAGGAAAAGAGAGGGAGGA |
| | | | | | | | AAGGCAGGGAGAGAGGAATA |
| | | | | | | | AAGGCGGGGAGCAGGCGAGA |
| | | | | | | | [CG]AGAGCAGCTCCGAGAAGC |
| | | | | | | | AGTGTGCGCGCCGCTTTCCCA |
| | | | | | | | AATCTTGCAGCCCAGCGAGCC |
| 165 | cg23298047 | chr15 | 30261418 | + | | | CCAGGCCCTGCGCCCGCGTGC |
| | | | | | | | CGCGGTGTTTTCAGCGGCTGG |
| | | | | | | | CAGGAGCTCCTTCTCAAC[CG]T |
| | | | | | | | TAGCACCCAAAGAGAATCCCA |
| | | | | | | | ACAGCACACTTCCAGCGCGGA |
| | | | | | | | TTAAAACAAACAAACAA |
| 166 | cg08259925 | chr5 | 63257813 | - | HTR1A | TSS1500 | CGCGTTCAGAAGCTCCAGCTG |
| | | | | | | | GGAAACTGGAGTTGGCCTGAA |
| | | | | | | | AGCAGCTCCAGGATCTCC[CG] |
| | | | | | | | GCGGCGGAGAGGTGGCTGGA |
| | | | | | | | ACGTCTGTCTGTCGCTGTCCAT |
| | | | | | | | TTTACTTTGCCGCTCCCG |
| 167 | cg24261921 | chr3 | 45821484 | + | SLC6A20; | Body; Body | TTCCCCGAGCGGGTGGCCCTG |
| | | | | | SLC6A20 | | TTTTTCTCTCCCTTTCTCGCTCC |
| | | | | | | | TACTCCTGTTCTGGCA[CG]GG |
| | | | | | | | CCCCCCGGCTCACCTGGAAGG |
| | | | | | | | AGTGGAAGAGGTACCAGAAG |
| | | | | | | | GCCCAGGCGTTGATGAC |
| 168 | cg13289553 | chr5 | 32585524 | - | SUB1 | TSS200 | AAGGATATTAGCTCTTTCATTC |
| | | | | | | | TCTCAAGGGTCAGATGTAATCT |
| | | | | | | | TCCAACATCTGACTTT[CG]CGT |
| | | | | | | | CACCCATTTAGGAAGAGACGC |
| | | | | | | | GGTCCCTTTAAGGCCCTGGAA |
| | | | | | | | AGGGTCTAAGTGTTG |
| 169 | cg26782833 | chr2 | 128642103 | + | AMMECR1L | 5′UTR | TGCAAACTCTAAATCTGAGGC |
| | | | | | | | AGCCGTGAAGTCCCATGCCCT |
| | | | | | | | GAATCATCTCATCCTTAG[CG]T |
| | | | | | | | CATCAGCAAGAAGGGAGGAC |
| | | | | | | | ACTGAGAATCAAAGGTTTTATT |
| | | | | | | | TATTGAACTCGAGCATG |
| 170 | cg18119885 | chr2 | 2617271 | + | | | TGAGGACACCGCCCCAAACCC |
| | | | | | | | CATGACTCTACCCAGAATGCA |
| | | | | | | | AGCAAGATGGTGCCAGGG[CG] |
| | | | | | | | CACTAAATCCCCAGCATGCAC |
| | | | | | | | TGCGACCGCCCTTAGTAGCAA |
| | | | | | | | GCGTAAACTACAATCCCC |
| 171 | cg04306050 | chr2 | 176046468 | - | ATP5G3; | 1stExon; | GGGCTGCGGCAGAGGTCGAA |
| | | | | | ATP5G3; | 5′UTR; | GGAGTGGGACTCAATGCGCAA |
| | | | | | ATP5G3 | TSS200 | GCGCGGTCCGGCTCTTATT[CG] |
| | | | | | | | CGCCGCAGCACCCGGATGAA |
| | | | | | | | GAAGGCGGGGTTTCGGGTGC |
| | | | | | | | ACCAAGGAAGACACTCAAGG |
| 172 | cg11325997 | chr19 | 2251764 | - | AMH | Body | ACTCATCCCCGAGACCTACCAG |
| | | | | | | | GCCAACAATTGCCAGGGCGTG |
| | | | | | | | TGCGGCTGGCCTCAGTC[CG]A |
| | | | | | | | CCGCAACCCGCGCTACGGCAA |
| | | | | | | | CCACGTGGTGCTGCTGCTGAA |
| | | | | | | | GATGCAGGTCCGTGGGG |
| 173 | cg00081714 | chr5 | 116306180 | - | | | TTTGGATTCCTTCCAACTTTTGC |
| | | | | | | | CACTGCCATCTGCTAGAAACTG |
| | | | | | | | GTTAAAACTGGCAAC[CG]GCC |
| | | | | | | | AAGAGAGATACATCCACTCTT |
| | | | | | | | AAAACCCATGCCCGGAAGTGA |
| | | | | | | | TGCACATTATTTACA |
| 174 | cg24580076 | chr7 | 915073 | + | C7orf20 | TSS1500 | TCTTCTTTTTTATTATAAACAAT |
| | | | | | | | GCTAACCTGTGAGAGTGGGCT |
| | | | | | | | GACCCTGTAAATCCAA[CG]GA |
| | | | | | | | GGAGTCTTCGGACCGAACGGC |
| | | | | | | | GAACCGCCTTCAAACCCCAATT |
| | | | | | | | CTTACAGCCAAGCCG |
| 175 | cg24636999 | chr6 | 38751903 | + | DNAH8 | Body | ATACCTGCATCCTAGAGGACA |
| | | | | | | | GTGCCCCAACCCCCGCAGGGT |
| | | | | | | | GTCGTCCCTAACAGGAAC[CG] |
| | | | | | | | TAGGTAAGCCTTTAATAAGCC |
| | | | | | | | ACTTTTATCAGGCCAGCTGTTT |
| | | | | | | | CTGGGTGCTGTGCTATA |
| 176 | cg25303383 | chr11 | 112046403 | - | BCO2; BCO2 | 1stExon; | CTCCATTTTATCAGGAGTCATT |
| | | | | | | TSS1500 | CTGCCACTGCAGTGGATTTCCT |
| | | | | | | | TCCTGTGATGGTGCAC[CG]GC |
| | | | | | | | TCCCAGGTAGAGGGTTTGCCC |
| | | | | | | | CTTTCTCTTCCTCATCCTCCTCT |
| | | | | | | | TCTTGCCAGTCTGC |
| 177 | cg01672943 | chr14 | 37125292 | + | PAX9 | TSS1500 | TGGCTCCTATAGGTGGCGCTG |
| | | | | | | | TGACAAGGTGCGGTGGCCGG |
| | | | | | | | GAGAGGCGGCTGGGGGACT[CG] |
| | | | | | | | AAGACTGCGGGAAATTTTCT |
| | | | | | | | GCGACTCCGACGCTAACCCGC |
| | | | | | | | TGCTCCCAGCCTCCGCTTC |
| 178 | cg07312601 | chr1 | 19583887 | - | MRTO4 | Body | TCCTGCTATGACAACCAAAAAC |
| | | | | | | | GTCTTTAAATGTTGCCAAATGT |
| | | | | | | | ACCCGGTGAGCAAAAA[CG]TG |
| | | | | | | | CCTAGTAGAGAACCACTGCTCT |
| | | | | | | | AATGTGACCAAGCTGTCCTCAC |
| | | | | | | | TCCTGATTTGTAGG |
| 179 | cg12778178 | chr20 | 62583555 | - | UCKL1AS; | TSS1500; | TTGGGAAGTGGGCAGGAGAC |
| | | | | | UCKL1 | Body | AGCCCAGGGTCGGGGAGGCG |
| | | | | | | | GAGGCTGTCCTGAGCAGGGG |
| | | | | | | | [CG]CAGAGTCCGGGCTCCTGG |
| | | | | | | | GGGCCATGCCACTGGCTGGGC |
| | | | | | | | TGTCTGAACAGCAGAGTGGAC |
| 180 | cg16023306 | chr19 | 30106588 | - | POP4; POP4 | Body; 3′UTR | AGGAACAGACTGGCAGGAAG |
| | | | | | | | CACACCGGGGTTAACACTGGT |
| | | | | | | | TGACTTGAATAGGATTATT[CG] |
| | | | | | | | ATTTTTAAAAATACTTTTCCAT |
| | | | | | | | GTTTTCTGAGTGCTCTATGATA |
| | | | | | | | AATCAGTTGCATCTGT |
| 181 | cg05722918 | chr12 | 101603929 | + | SLC5A8; | 1stExon; | TCGACCCGCTGCCCTGAGTGCT |
| | | | | | SLC5A8 | 5′UTR | CACCACGTGAGGAACTGGAGT |
| | | | | | | | GGCCGAGTTCGCCAAGG[CG]C |
| | | | | | | | CGGGGACACCTGAGCAGATGA |
| | | | | | | | GAACTGGAGCCTCCAGCTGCT |
| | | | | | | | TCCAGCGAATCTACACA |
| 182 | cg22572614 | chr3 | 172241975 | - | TNFSF10 | TSS1500 | AAAGGCAAAGGAAAAAAACAT |
| | | | | | | | GTGGATGTTTTCCAAAATATTA |
| | | | | | | | ACCCCATCACAATGTCT[CG]CT |
| | | | | | | | GTCACTATCCTTTTACAGATTA |
| | | | | | | | GGAAAAGAAGTTACAGGGAG |
| | | | | | | | TTAATTACCCTCAGAT |
| 183 | cg10346212 | chr19 | 384389 | - | | | TGGGTGGGAACAGAACAGCCT |
| | | | | | | | TGGTCGTGGCTGAGGAGAAAT |
| | | | | | | | CCCACAGATGTCACTGGA[CG] |
| | | | | | | | AGGGTGACGGGTGGGGCCGG |
| | | | | | | | GCTTTCCCCTGGGTACAGGCA |
| | | | | | | | CAACCGTGCTCTTCCCTCG |
| 184 | cg14942863 | chr19 | 37894762 | - | | | TGTCTCGTGTTGCTATGAGGTT |
| | | | | | | | TGCATCTGTGTGGCTGGAATA |
| | | | | | | | GCTTGTTTGTGGGGGCC[CG]C |
| | | | | | | | GCGTGACCTGTGTGTGCGTTA |
| | | | | | | | CTGTGTGTGTCTCAGGCAGGA |
| | | | | | | | TAGTGACGGGCCGTGTG |
| 185 | cg03930964 | chr22 | 23522374 | - | BCR; BCR | TSS200; | TGAGGTAGGTGGTGGGGCTTG |
| | | | | | | TSS200 | GGGACACGCGGCTGGACTGG |
| | | | | | | | CCGGAGAAGTCCTCCTGGC[CG] |
| | | | | | | | GAGGGGAGCCAAGTGTTCCT |
| | | | | | | | GTTCCAGGACTGCAGAACTGG |
| | | | | | | | CCCAGACCTCTGTATTGGA |
| 186 | cg05030953 | chr6 | 31241000 | - | HLA-C | TSS1500 | AAAAAAAAATCATAAGGAGCC |
| | | | | | | | CATTAGTTTTAAGGCAGTCACA |
| | | | | | | | CAAAATGTATTAAATAC[CG]A |
| | | | | | | | ATGCAAAGAACCCCCTGCCAG |
| | | | | | | | GCTCTTCTACTGCTTTAGAATT |
| | | | | | | | CTTTCCTCTGCTCCTT |
| 187 | cg27304144 | chr1 | 22211074 | - | HSPG2 | Body | AACGCACCCTTGAAGTCATCG |
| | | | | | | | GGTTGGTCAAAGCGCAGCCTG |
| | | | | | | | ATCTGGTCCCGGAAGCGG[CG] |
| | | | | | | | GGTGCTCTGGCACACGCTGGT |
| | | | | | | | GATGCCAAAGCAGAAGCAGG |
| | | | | | | | GCAGGCAGGCGGCGCTGTG |
| 188 | cg12794224 | chr6 | 151646761 | - | AKAP12; | 5′UTR; | TCCTGGAGCTCAGCAAGGGAG |
| | | | | | AKAP12; | 1stExon; | GGGCCAGCGCCAGCCCGCGTG |
| | | | | | AKAP12 | Body | TGGGTGGCTGGGTGGGGG[CG] |
| | | | | | | | TGGGTGGGGGTCCGCCTATA |
| | | | | | | | ATTATCTGGGGAAATGCATCC |
| | | | | | | | GCGCTCTGCTTTTCGCTGC |
| 189 | cg17028652 | chr10 | 115805442 | + | ADRB1; | 3′UTR; | GTGTTTACTTAAGACCGATAGC |
| | | | | | ADRB1 | 1stExon | AGGTGAACTCGAAGCCCACAA |
| | | | | | | | TCCTCGTCTGAATCATC[CG]AG |
| | | | | | | | GCAAAGAGAAAAGCCACGGA |
| | | | | | | | CCGTTGCACAAAAAGGAAAGT |
| | | | | | | | TTGGGAAGGGATGGGAG |
| 190 | cg24458609 | chr11 | 56948015 | - | LRRC55 | TSS1500 | CGCGGGGCGCGAGGGCTGAG |
| | | | | | | | GCTCTGGGCGTGGCATCACTC |
| | | | | | | | TCGGTCCCTCTGCTGGGGG[CG] |
| | | | | | | | GCGAGGAGAGTGCAGTGTGT |
| | | | | | | | GGAAAGGGATGCTGGGATGA |
| | | | | | | | AGGGTGTGCGCTGAGAGGGG |
| 191 | cg26454158 | chr19 | 12273814 | - | ZNF136 | TSS200 | TGCAGGGGGCAGAGCCCGAA |
| | | | | | | | GCTGTACCCAATCAGGGGCAC |
| | | | | | | | CGGGGAGGAGCTCTGCGAT[CG] |
| | | | | | | | GTCCAATCAGGCGCGCCGTC |
| | | | | | | | GGGGACGCAGCTGCAGACGTT |
| | | | | | | | CAACCTTCTCGCGGGATTT |
| 192 | cg15481429 | chr15 | 94945799 | - | MCTP2; | Body; 3′UTR; | TCTATGAAATGTACCCTTTTCT |
| | | | | | MCTP2; | Body | CTGGTGACATTGGCCCATCCTT |
| | | | | | MCTP2 | | ATGAGCATAATAAAAT[CG]CA |
| | | | | | | | GAATCAAAGCGCTGCAAGAGA |
| | | | | | | | TCTTAAAACCACCTAAGTCTAC |
| | | | | | | | CACTGAGAGCCCAAG |
| 193 | cg08386537 | chr2 | 171569381 | + | LOC440925 | Body | CCAAGGTCACCAACTAGAAAG |
| | | | | | | | TGGCAAGGCGGGAAAAATGTC |
| | | | | | | | TTCAGAGAGTTCGGACTC[CG] |
| | | | | | | | AGCTTTCAACCACCAAGCCACT |
| | | | | | | | AACTTTGACCCTGTTGGCCCAC |
| | | | | | | | TGATGGTTTAACTGGC |
| 194 | cg19233923 | chr11 | 63753598 | - | OTUB1; | 5′UTR; Body; | GGAATGCTGCCTTCGGTGATTT |
| | | | | | OTUB1; | 1stExon | TAATTTCACTTTTCTACTTCTCT |
| | | | | | OTUB1 | | CAATAACAAAATCCG[CG]TTTC |
| | | | | | | | AAACTCCAGGGAAAAGAAAAC |
| | | | | | | | GGAATTGGCTCCAGGAGGATC |
| | | | | | | | TGCAATCACCACCG |
| 195 | cg01414572 | chr12 | 5248588 | + | | | AGTATGTACTTGCTGACCCAAT |
| | | | | | | | TCCTGAATTTTTGCAGGATAAT |
| | | | | | | | TAAGTAGCATTTTCAC[CG]GG |
| | | | | | | | AGTGTAGTCAAATATGATTTGT |
| | | | | | | | ACTGGAGGTCCTTATTCTGCCA |
| | | | | | | | GGTGCGTGCAGAGA |
| 196 | cg06517429 | chr10 | 115439635 | + | CASP7; CASP7; | 5′UTR; | GCCAGGGGCGGTGCAAGCCCC |
| | | | | | CASP7; | 1stExon; | GCCCGGCCCTACCCAGGGCGG |
| | | | | | CASP7; | 1stExon; | CTCCTCCCTCCGCAGCGC[CG]A |
| | | | | | CASP7; | 5′UTR; | GACTTTTAGTTTCGCTTTCGCT |
| | | | | | CASP7; | 1stExon; | AAAGGGGCCCCAGACCCTTGC |
| | | | | | CASP7 | 5′UTR; 5′UTR | TGCGGAGCGACGGAGA |
| 197 | cg06760904 | chr2 | 1827764 | - | MYT1L | Body | TTACGTGGCACAGTGTTGGCC |
| | | | | | | | TGGGCCTCGCCGTCCCTGGCA |
| | | | | | | | CGACCCATGGGATGAGGC[CG] |
| | | | | | | | CGCCTCCCCCCCCAGCGGGGC |
| | | | | | | | CGCCGGGCAGAGGTGATGTG |
| | | | | | | | GGATGCTCAGTGACTTTTT |
| 198 | cg00059424 | chr22 | 30988148 | - | PES1 | TSS1500 | AACGTGGATATACAGGCTTTTC |
| | | | | | | | TGTAATCACCCTGATGACGATT |
| | | | | | | | CATTGACTGTGAGCCT[CG]TT |
| | | | | | | | GCATGTTGGGACGGAGAGGG |
| | | | | | | | GCGGAAGGCTTAGGGACAGC |
| | | | | | | | GCGGTGCCTTCTGGGATG |
| 199 | cg11002227 | chr3 | 155588016 | + | GMPS | TSS1500 | ACTTTCCAAAGCAGCCTTGGCC |
| | | | | | | | TCCTTCATGTCCAGCAACCTGA |
| | | | | | | | GATAAGGCCACGCCAC[CG]GC |
| | | | | | | | TAAGAGTTCCGCCAGGGGCCC |
| | | | | | | | AGCTCTCAGGAGGCCTCTTCG |
| | | | | | | | GTGCCGCCAGCCTCCC |
| 200 | cg25371803 | chr1 | 156308296 | + | CCT3; CCT3; | TSS200; | GGGCACAGGCGCTTGCGCAGT |
| | | | | | C1orf182; | TSS200; | AGGGTGGCCGCTCCCGGCCGC |
| | | | | | CCT3 | 5′UTR; | GTGCAGCGCGAACGTCGG[CG] |
| | | | | | | TSS200 | CAGGCGCCAAGGCTCTGGCA |
| | | | | | | | GTTGGCCAGCACACCACTACG |
| | | | | | | | CATGTGTGTCAACTCTAGG |
| 201 | cg20642765 | chr12 | 6861825 | + | MLF2; MLF2 | Body; 5′UTR | CACTCAGAGCCATCCTCTTCCC |
| | | | | | | | AAAGCTCTGGCCGGTAGCATA |
| | | | | | | | CTCTCCCCTCCTCCCGC[CG]AC |
| | | | | | | | GACACCGTTCTAGATGAGAAT |
| | | | | | | | GCCAAGTGCAGGTCCTCCGCC |
| | | | | | | | CCATTAATGACCCCAG |
| 202 | cg08734053 | chr1 | 35442250 | - | | | GGCAGCTGTTGAGGCTCAGCA |
| | | | | | | | GCGCCAGGCTGAGGGTGTGCA |
| | | | | | | | GGATGTCGAGCGTGGAGG[CG] |
| | | | | | | | GCGCGACACCGGTCTCCGTTG |
| | | | | | | | TCTTCCCCCCCAGCCACCTAGG |
| | | | | | | | GCGCCAGCAGCAGGTGG |
| 203 | cg11567723 | chr7 | 152163944 | - | | | GATGGGGTTTCACCATGTTGG |
| | | | | | | | CCAGGCGGACTCAAACTACTG |
| | | | | | | | ACCTCGTTATTCACCCGG[CG]C |
| | | | | | | | GGCCTCCCAAAGTGCTGGGAT |
| | | | | | | | TATAGTCATGAGCCCGGCCCTC |
| | | | | | | | TTTTTTTTTTTCGTTT |
| 204 | cg16897193 | chr19 | 46443801 | - | NOVA2 | Body | CCAGCGTGTTAAGCGCCGTGC |
| | | | | | | | TGATGGCCAGCAGGTCGGTGC |
| | | | | | | | CTGAGAAGGCGGGCAGCG[CG] |
| | | | | | | | GCGGGAAAGGCCCCCACGCC |
| | | | | | | | AGCCAGCCCGGCGGGGCCCA |
| | | | | | | | GCAGGCCGGAGGCGGCGGCG |
| 205 | cg23021855 | chr2 | 68695071 | + | APLF; | Body; | CGGCTCCTGAAGACCGGCCCT |
| | | | | | FBXO48 | TSS1500 | AGTCCTGGCCGGTTTCCCCACC |
| | | | | | | | GCACTGGTCCGCCGGTC[CG]G |
| | | | | | | | ATTTTAGAAGTTTGGGGCCGC |
| | | | | | | | ACGTTTTTCAGTTACCTTTAAG |
| | | | | | | | CCAATTCACAAACATT |
| 206 | cg08261702 | chr7 | 150103112 | + | LOC728743 | Body | GGCGGGGCCTCAGTCAGGGG |
| | | | | | | | TATAGCTGGGGAGAGTGAGG |
| | | | | | | | AGGCTGCCCAGTCACAGGGC |
| | | | | | | | [CG]GGCTGAGATTGGCCAAGG |
| | | | | | | | GGACTTTGATGATCTGTCTTTG |
| | | | | | | | CAGATGTCAGTGCAGCTGCC |
| 207 | cg18088844 | chr19 | 46171324 | - | GIPR | TSS200 | GGTACCTGTGGGTGGGACAGC |
| | | | | | | | ATGAGAGATTGTACACACTTG |
| | | | | | | | GTGCAGGGGTCCTCAGGA[CG] |
| | | | | | | | ATAAGGACAATTCAGTAACTG |
| | | | | | | | CCCTCCCTCATGACCTTGATGA |
| | | | | | | | CTGCCCCCTGCTCGGCT |
| 208 | cg11594299 | chr7 | 4924002 | - | RADIL | TSS1500 | GGTCAGCTCTGGGGCTCTGGC |
| | | | | | | | CCCAACTGCTCTCCCTGGGGAC |
| | | | | | | | TTGTTTAAAAAGCAGCT[CG]T |
| | | | | | | | GACCTCGGCACTTTGGCTGGG |
| | | | | | | | GTTTTCCCTTTGAGGAATGTGG |
| | | | | | | | GCTAGACCTGGGAGAT |
| 209 | cg16025094 | chr5 | 175298655 | - | CPLX2; | 1stExon; | CAGCTCGCCTGGCGGAATTGC |
| | | | | | CPLX2; | 5′UTR; | ACGCGGCGGCGGGAGCTGGA |
| | | | | | CPLX2 | 5′UTR | ATAGCAGAAGGAACCACCT[CG] |
| | | | | | | | TGGAGTCGGGCCGGAGCCC |
| | | | | | | | TGCAGTGGCTCAGACGGTTGC |
| | | | | | | | AGGGACCGCCAGGTCGGTGC |
| 210 | cg15309223 | chr1 | 54519091 | - | TMEM59; | 1stExon; | CTGGGACTACGAACTTCTTCTC |
| | | | | | C1orf83; | TSS200; | CTAGGCTGGCGTGAGGAGGG |
| | | | | | TMEM59 | 5′UTR | GAATTCAACCATCGCAAG[CG] |
| | | | | | | | TTAGCGCGAAGCGGGGCCTCC |
| | | | | | | | TGACTTCTTCCCTTCGCGGGGC |
| | | | | | | | AGGCTGGGGCATGTAGT |
| 211 | cg05156137 | chr21 | 35898975 | - | RCAN1; RCAN1; | 5′UTR; Body; | AATGCTTTGAAAACTAAAGAA |
| | | | | | RCAN1 | 1stExon | AATCACGTTATATTAGAAGCCT |
| | | | | | | | TACCCTGGTTTCACTTT[CG]CT |
| | | | | | | | GAAGATATCACTGTTTGCCACA |
| | | | | | | | CAGGCAATCAGGGAGCTAAAA |
| | | | | | | | CTGTAGTTAAAGTTT |
| 212 | cg03335886 | chr13 | 20797410 | + | GJB6; GJB6; | Body; Body; | CAGCAGCGCTGGGGTGGAGA |
| | | | | | GJB6; GJB6 | Body; Body | CGAAGATCAGCTGGAGGGCCC |
| | | | | | | | ACAGCCGGATGTGGGACAC[CG] |
| | | | | | | | GGAAAAAGTGGTCATAGCA |
| | | | | | | | CACATTTTTGCATCCCGGTTGC |
| | | | | | | | AGTGTGTTGCAGACGAAGT |
| 213 | cg01717881 | chr17 | 122697 | + | RPH3AL | Body | ACAAGCAGGAGAGAGGGGCC |
| | | | | | | | AGAAGGAAGAAATAAAGACCC |
| | | | | | | | AGCCTCAGTGGGCCAGTGG[CG] |
| | | | | | | | ACGTGAGATCCCAGCAAGG |
| | | | | | | | GCGACATCAGGGAGAGACCCC |
| | | | | | | | AGCAAGGGCTACGTCAGGGT |
| 214 | cg03031988 | chr6 | 31510729 | + | BAT1; BAT1 | TSS1500; | ACCTCAGGTGATCCACCCACTT |
| | | | | | | TSS1500 | CGGCCTCCCAGAGTGCTGGGA |
| | | | | | | | TTACAGGCGTGAGCCAC[CG]C |
| | | | | | | | GCCCGGCCCATTAATACTGTTA |
| | | | | | | | ATTCGAGCAGAATGTTCTTGG |
| | | | | | | | CCCCGCCCCAACAGCC |
| 215 | cg04738656 | chr11 | 66360492 | - | CCDC87; | 1stExon; | GCAGCCGGTGGTAAAACCGCT |
| | | | | | CCDC87; | 5′UTR; | GGAGCTCAGGCTCGGGCTTCG |
| | | | | | CCS | TSS200 | GGGGCTCCATCATAGAGC[CG] |
| | | | | | | | GCGGCCGCCACCGTCCAGGAA |
| | | | | | | | CAGAAAGCCGAGGGGTTACTA |
| | | | | | | | AGGCAACCAGGAGCCCGA |
| 216 | cg23229770 | chr2 | 129491004 | - | | | CAGTTTTGTGCTGAGTAAAGA |
| | | | | | | | ACACGGCTGTTACTGACAGAT |
| | | | | | | | GGACTTGGGTCAGAATCC[CG] |
| | | | | | | | ATTTCACCCTTCCTTTGCTGTAT |
| | | | | | | | TACCTTGCTTGACAGGAGGGC |
| | | | | | | | TGCTGGTCACATACAG |
| 217 | cg07299526 | chr16 | 89702762 | + | DPEP1; DPEP1 | Body; Body | CAGAACAAAGACGCCGTGCGG |
| | | | | | | | AGGACGCTGGAGCAGATGGA |
| | | | | | | | CGTGGTCCACCGCATGTGC[CG] |
| | | | | | | | GATGTACCCGGAGACCTTCCT |
| | | | | | | | GTATGTCACCAGCAGTGCAGG |
| | | | | | | | TGGGGTCCTGACCTGGGT |
| 218 | cg20355806 | chr13 | 114930281 | - | | | GTCTTATTCGCCTCTTGTGACA |
| | | | | | | | CAGCTATGATGTGACGTCCTG |
| | | | | | | | CATTTTACTGATGTGGA[CG]CT |
| | | | | | | | GAGGTCCAAAGACAAGCAGCC |
| | | | | | | | TCCCAGGGACACACGGAGCTG |
| | | | | | | | GAGTCCCCCGAGTCTC |
| 219 | cg02268620 | chr9 | 97847913 | + | MIR24-1; | TSS1500; | GGGCAGAGGCCGTTGCTGACG |
| | | | | | C9orf3 | 3′UTR | GGCCGGCCGCTGCTGCACAGT |
| | | | | | | | CAGCTTGGGTGCGGAGCG[CG] |
| | | | | | | | ATCCTGGAGGATGAGAGACC |
| | | | | | | | ACTTGACCCCAAGGATGCACT |
| | | | | | | | GTCTCCTGCTGGGAATGCT |
| 220 | cg26050838 | chr7 | 142985210 | + | CASP2; | TSS200; | TCCGTGAAGTTATCGCCATAG |
| | | | | | CASP2 | TSS200 | GCCGGCCAGGGGGCGCGAGA |
| | | | | | | | GGCACCGGGGTGATTTCCG[CG] |
| | | | | | | | GGAATCGATAACCAATCGG |
| | | | | | | | ATTCCCAGGCCGAACGGAGCA |
| | | | | | | | CACCCGCCCGCCCTCGCTCT |
| 221 | cg05335473 | chr1 | 84040080 | - | | | CTAGGGCCTAAGGCACAACTG |
| | | | | | | | CCTTGCCCTGGGCTGAATTCTA |
| | | | | | | | CCCTAGGGCAGAGTTTT[CG]G |
| | | | | | | | TGGCCTCGGTGTACTCTTAGTA |
| | | | | | | | GTATTTCTACTAAAAAGCCAAC |
| | | | | | | | ATAGAGGGCATAGAC |
| 222 | cg13009608 | chr8 | 81034420 | - | TPD52; | Body; Body | GTTCTCTCAAGAGAACAAGGA |
| | | | | | TPD52 | | ATCAGGTCTTACTACATAAGG |
| | | | | | | | GCTTTCTCTATGGTGACA[CG]T |
| | | | | | | | CACATCTCAAAACAAAACAGA |
| | | | | | | | AAGTAAGACAAACCAAGCTGT |
| | | | | | | | GATGCAGGAAAACAGAG |
| 223 | cg04631458 | chr7 | 1329462 | - | | | GGCGGGGACGGGGGGAACCC |
| | | | | | | | ATTTGAAATAAATACTTGTGAG |
| | | | | | | | TCTCTGACAGACTCCAGA[CG] |
| | | | | | | | GGCCGTCGACGCCGCCTGGCA |
| | | | | | | | ATGTCTGGGACCTGTCACACTC |
| | | | | | | | TGTGATCGGTCTTTTTA |
| 224 | cg26777345 | chr4 | 99877093 | - | | | TGATGTGTTCCCATAAAACGCC |
| | | | | | | | ACTTAAAAGATTTAAACTTTAG |
| | | | | | | | ATGGTCCAAAAGGAAC[CG]TT |
| | | | | | | | GATGTCAGGACAACCATAAAC |
| | | | | | | | CAAATTTTATCTCATGGGGAAA |
| | | | | | | | TATGAGATTGGATGA |
| 225 | cg22946147 | chr7 | 88425148 | + | ZNF804B; | Body; | GAGTCAGAATGTCAGCACCAT |
| | | | | | MGC26647 | TSS200 | TAAAGGACCAGAGCGCCAAGT |
| | | | | | | | TTCTTAATACGGGTATCT[CG]A |
| | | | | | | | CAAACACTTCAAAGTCACTGCA |
| | | | | | | | GAGGAAGTGTGAATGGCTTAT |
| | | | | | | | TCCTGAATGGTTTATT |
| 226 | cg22425860 | chr4 | 190474719 | + | | | GACAGGGGACTGGAGAGCAG |
| | | | | | | | GAAGACAGGAGAACAAGGAG |
| | | | | | | | ATTTCTCCTCCTTCAGCAGC[CG] |
| | | | | | | | CAGCAGCAACGGCGTGTCCTC |
| | | | | | | | CACAGTTAACTGGAAGAAAAA |
| | | | | | | | GCCTGAGTCCTGGTCTCC |
| 227 | cg00151919 | chr13 | 41363245 | - | SLC25A15 | TSS1500 | TGCCCGGCTAATTCCTGTATTT |
| | | | | | | | TCATACTTAGTTGTATTTCCTAT |
| | | | | | | | TAGGGCCTTGGATCC[CG]AGT |
| | | | | | | | ATAATTTTGTACTCAAATATAA |
| | | | | | | | TTTATAAATAAGGCCTTAGCCT |
| | | | | | | | CCCAACAAGGTCA |
| 228 | cg19255191 | chr2 | 98262923 | + | COX5B | Body | AACGGAGGTGCCGGGTGACCT |
| | | | | | | | TGGGAGGGACCGGGGCTGCC |
| | | | | | | | ACCGGGATGGGGAGGGGTC[CG] |
| | | | | | | | GCCTCCCTTCAAACCTGCGC |
| | | | | | | | CCACCTCAAGCAGAGTGGGTT |
| | | | | | | | CTACATGCTTTTAGACAAA |
| 229 | cg22872989 | chr1 | 27709900 | - | CD164L2 | TSS200 | GCAACCGGGGCGTGGCCAGG |
| | | | | | | | TGGGGGCGTGGCCAGTGGGA |
| | | | | | | | GCGGCAGGTGGGGCGGGGCT |
| | | | | | | | [CG]TCGGTCGGGGCGGAGCC |
| | | | | | | | AGGTGAAGGCGGGGCCAGTT |
| | | | | | | | AGGGGCGTGGCTAGTGTGCGC |
| | | | | | | | GG |
| 230 | cg10286959 | chr8 | 1291957 | + | | | ATGTGCACGACAGTGGAACGG |
| | | | | | | | AGGCCTCTCCAAGAGGCGGGG |
| | | | | | | | GCAGTGCTGTGGGCTTCA[CG] |
| | | | | | | | CCTGCTGTGGCACGAGATCCT |
| | | | | | | | CCCTGCACGTCCACCCGTGACA |
| | | | | | | | GAGCAGATGATGCTCCA |
| 231 | cg21877956 | chr6 | 83926357 | + | ME1 | Body | ACACTTGCTGAGCTATAACCTT |
| | | | | | | | ATGAAAAAAAGAAAGAAAAA |
| | | | | | | | AAGTGTTTATACTTCACA[CG]A |
| | | | | | | | TACAATGTGGTGGGTACGCCA |
| | | | | | | | ATAACTAAGTGAACGGTTACA |
| | | | | | | | TATAATGGTCTATACAA |
| 232 | cg17279592 | chr6 | 170038733 | + | WDR27 | Body | TTCGCAGGGTCCCGTCCCGGG |
| | | | | | | | CCGCAGAGAGCAGCCACCTCC |
| | | | | | | | GGTCCTGGCTCCAGCACA[CG] |
| | | | | | | | GCATTCACTGCCCCGTCGTGAC |
| | | | | | | | CTAACAGGAATGACCACAGAA |
| | | | | | | | GGTTACTATTTCTACTA |
| 233 | cg02064158 | chr17 | 1929356 | - | RTN4RL1 | TSS1500 | TCTCCGCCTGGGTGGGGTGGC |
| | | | | | | | GGCGGGGGGTCTCTGATCTCC |
| | | | | | | | CTTGGTCCACACAGACCC[CG] |
| | | | | | | | CCGGGGGGTTCGCGGAAAAT |
| | | | | | | | GGAGGAGGCGCCGCTTGGAA |
| | | | | | | | AGCGGGTCCCGCAGGGGCCT |
| 234 | cg25584787 | chr5 | 93693854 | - | C5orf36 | Body | TTTATTATCTATAAATGTTTAAT |
| | | | | | | | CAAACTGTGGCATTTTAAAGTC |
| | | | | | | | TTGTTTCAAATTCCT[CG]CCTT |
| | | | | | | | CAGTTGGCCGGTATTCTTACAG |
| | | | | | | | CTTTTTCTTGAGTGCAAGGCAG |
| | | | | | | | CACTGCAACTGC |
| 235 | cg09113665 | chr16 | 50059684 | - | TMEM188 | Body | CTGCTCGGTGTTTTAAAGTTTA |
| | | | | | | | AAGCACACCACTGCGGAAAGG |
| | | | | | | | ATACCCCACCACTCACT[CG]GA |
| | | | | | | | GCAGCTTAGACGCCCCTGTCTT |
| | | | | | | | CTAGAACTAGGCGCTGCCTGG |
| | | | | | | | GTGCCACGAAGATCA |
| 236 | cg13282195 | chr8 | 144660772 | - | NAPRT1 | TSS1500 | CCAGGCCCAACGGCCTCTTTG |
| | | | | | | | GAGCGCAGCCCGGTCTTGGTC |
| | | | | | | | ACCAGAGGTGCCCCCAGT[CG] |
| | | | | | | | CTCGTGTCTCTGCCCTTTGGCC |
| | | | | | | | GGGCAATGAGGTGCAGCTCAG |
| | | | | | | | GACTTGCCAGGCGGCGG |
| 237 | cg03873281 | chr5 | 131608955 | + | PDLIM4; | 3′UTR; 3′UTR | ACCCTCTAGTTTACTTGCTCGG |
| | | | | | PDLIM4 | | GAGAAGAAACTGACTCGTTTT |
| | | | | | | | ATTTAGTGCCTATTTAG[CG]AG |
| | | | | | | | CCCAGAGTAACGTACATTTGT |
| | | | | | | | GCTGTTTTCAATTTTGTGCTAT |
| | | | | | | | CGCAAATCACAAAAA |
| 238 | cg00841725 | chr13 | 113655538 | + | MCF2L; | Body; Body | TATCCCCCTCCCGGTCCTGGAA |
| | | | | | MCF2L | | AAGTAGAGAGGCAGCCGGGA |
| | | | | | | | GCCTGCCTTCTGTGTTCT[CG]G |
| | | | | | | | TGCAGGGGTATTCTGAGAACG |
| | | | | | | | GCCCCTGCTCACACGGGTTTAA |
| | | | | | | | AAGGAACTCAGTGACC |
| 239 | cg16758041 | chr9 | 32573371 | + | NDUFB6; | TSS200; | GACCGGGTGGGGACAAGGAG |
| | | | | | NDUFB6 | TSS200 | TACTCGTAGTTGTGGGGCCTG |
| | | | | | | | AGGAAAGTGACAGATTAGA[CG] |
| | | | | | | | AAAGTATGCTAAATTAGAG |
| | | | | | | | GACTGGAGGTTTTGCTAAGGA |
| | | | | | | | AGAACTTGTATGCTGGGAGG |
| 240 | cg12528144 | chr10 | 102973538 | + | | | GGCAGGAGGGTAGCTGAGAT |
| | | | | | | | GACCGCGAGCCAGTTAGAGGA |
| | | | | | | | ATTTCGCTGCCTCCAGCCC[CG] |
| | | | | | | | CAGCCCGCCGCAGTGCCAAAT |
| | | | | | | | AACAGACGGCAGAGGGCGCT |
| | | | | | | | CCTACCTAACCTTTCCCAT |
| 241 | cg19136783 | chr4 | 16598466 | - | LDB2; LDB2 | Body; Body | TAGCTGGGCCTTTCTGATACAG |
| | | | | | | | GATGCTTAGAAATCTGTAACA |
| | | | | | | | AGCCCTTTTTTCAGCAG[CG]AT |
| | | | | | | | TTGAAATCCTCTTACACTGGAA |
| | | | | | | | ATCCCAACTCATAATATCAGGA |
| | | | | | | | ATTTTGCCTATGTG |
| 242 | cg00798886 | chr5 | 54603441 | + | DHX29; | 5′UTR; | TTTCTTGTTCTTGCCGCCCATG |
| | | | | | SKIV2L2; | TSS200; | TTGCAGCTGTGGCAGAAGATC |
| | | | | | DHX29 | 1stExon | CTTCGCGGCCCAGGCCC[CG]A |
| | | | | | | | CGGTACCACTGCACAGCCGAG |
| | | | | | | | AGCTCTTCACATTCCCCGGCTC |
| | | | | | | | CGGGGCTGCCACCCTG |
| 243 | cg11732282 | chr2 | 153573982 | - | ARL6IP6; | TSS1500; | CTGCTCCGCCGGCGGCCACTG |
| | | | | | PRPF40A; | TSS200; | CCGCTACACATACCAACAAGA |
| | | | | | ARL6IP6 | TSS1500 | AGCGATCTGAGTGGCTGG[CG] |
| | | | | | | | CCCACTGGGGCTAAAGGTTAA |
| | | | | | | | AGGCTGCCCTGCGCTACGGGG |
| | | | | | | | CGGGATCAGCGGGGCCAA |
| 244 | cg12213687 | chr13 | 110802749 | - | COL4A1 | Body | CATTAGCTGAGTCAGGCTTCAT |
| | | | | | | | TATGTTCTTCTCATACAGACTT |
| | | | | | | | GGCAGCGGCTGACGTG[CG]T |
| | | | | | | | GCGCAGCTCCCCTGCCTTCAAG |
| | | | | | | | GTGGACGGCGTAGGCTTCCTA |
| | | | | | | | AAACACGACACAGAGA |
| 245 | cg16937168 | chr2 | 241936844 | + | SNED1 | TSS1500 | AGGGGCAAGCTTTCAGGAGGT |
| | | | | | | | GCCAGTGCAGGGTCAGCTCCT |
| | | | | | | | CCTTAACAATTCTGCACC[CG]G |
| | | | | | | | CCCTGACACCAAGTCTAAAGG |
| | | | | | | | GTCATGAACCTCTGAGTGAAA |
| | | | | | | | ACACCAAGTGCAGGATC |
| 246 | cg14866740 | chr6 | 110501627 | - | CDC40; WASF1; | 5′UTR; | GTTCCATTGCAATCTGTCAGGA |
| | | | | | WASF1; CDC40; | TSS1500; | CCTGGGAGCCTCTTCTTCTTCC |
| | | | | | WASF1; | TSS1500; | GCCCTGGCAGGGTCTC[CG]CA |
| | | | | | WASF1 | 1stExon; | GAAGATTTGTTGCCGTCATGTC |
| | | | | | | TSS1500; | GGCTGCGATTGCAGCTCTGGC |
| | | | | | | TSS1500 | CGCTTCCTATGGTTC |
| 247 | cg18703066 | chr2 | 105363536 | - | | | GTTCTTTTCACGTTGGCGCAAA |
| | | | | | | | TGAGCAATGCGCACGAAGCTG |
| | | | | | | | CTCCATCTCCTCTGCTG[CG]AT |
| | | | | | | | TTCGCTGCCGAAGAGCCGAGG |
| | | | | | | | AAGGTTAGGATGCAATTAACA |
| | | | | | | | GAGCGGAGTGACCTGC |
| 248 | cg19772114 | chr6 | 28829321 | - | | | CACGTGGTTCAACCAGAAGAT |
| | | | | | | | CCGCAGAATCAAGGCCCGGCA |
| | | | | | | | AGCCAAAGGGCGCTGCAT[CG] |
| | | | | | | | CCCCGCGCCCGGAGAGTCGGG |
| | | | | | | | ACCCATCTGGCCCATTGTGCTG |
| | | | | | | | TGCCCTGCTGTGCGTTA |
| 249 | cg07139350 | chr1 | 12416368 | - | VPS13D; | Body; Body | AACTGTCTTTTTAGGCAAGAAA |
| | | | | | VPS13D | | CTGAGCCCACTAAATAGATTCA |
| | | | | | | | GTTTTCACTCTTTTCC[CG]CTTG |
| | | | | | | | ATGGTTTTATTCATTCACCATTT |
| | | | | | | | GCATCTCTTTCAGATAGACTGG |
| | | | | | | | GTGGTATTGAT |
| 250 | cg13614741 | chr7 | 148991738 | - | ZNF783 | Body | CCACCTTGCGCCCAGTGTGGC |
| | | | | | | | CAGAGCTTCGGCCAGAAGGAG |
| | | | | | | | CTCAGTGCGCCGCACCAG[CG] |
| | | | | | | | CGTGCATCGTGGCCCCCGGCC |
| | | | | | | | TTTCGCTGGTGCTCAGTGTCCC |
| | | | | | | | AAGAGCTTCACGCAGCG |
| 251 | cg04172115 | chr6 | 32053728 | + | TNXB | Body | CCCCCGGCCCCTCGGGCACCC |
| | | | | | | | GCATGCGCAGTTGGAAGTAGG |
| | | | | | | | CAAAGGTGTCAGGCTGGG[CG] |
| | | | | | | | GTCCAGACCACACGGAGGCG |
| | | | | | | | CCCTGTCTCATCTCTGCCCAGC |
| | | | | | | | ACCCTCAACTCTCCCAGC |
| 252 | cg01146808 | chr6 | 106551368 | + | PRDM1; | Body; Body | TCCCCCAAACCTGCTGCCTCTG |
| | | | | | PRDM1 | | AAGGCATCTCCACACATTGAC |
| | | | | | | | AGCCAATGCCTTCAGTG[CG]T |
| | | | | | | | TCCTAGGGCAGGTGTCCTGGC |
| | | | | | | | TTGAGTGACTGTCCTCCAATAA |
| | | | | | | | TCAGAGCTCAAACTAA |
| 253 | cg06826289 | chr12 | 129468180 | + | GLT1D1 | 3′UTR | ACAGGCACGTGGGTGACCCGA |
| | | | | | | | GGCTTCTCTGAACACTAGAAA |
| | | | | | | | GCGCTGTGAGTGAGCTCA[CG] |
| | | | | | | | CCCGGCACAGCTCACTTTTCAA |
| | | | | | | | TGGTGGAATTGAAAGTTGTGC |
| | | | | | | | TTTTTAGAAAAGTGGCC |
| 254 | cg23124451 | chr22 | 39548131 | + | CBX7 | Body | TCAGTCTCCCCATATTTACAAT |
| | | | | | | | AAAAGGGGAGCGAGGTGGGA |
| | | | | | | | TGGCGCTGAGGATCCCTA[CG] |
| | | | | | | | TCCGATCCTAATCTCCAGCTCA |
| | | | | | | | GGCAGGCTCGGCCGCCACTAG |
| | | | | | | | CATCCTGGAGCGACAAC |
| 255 | cg05200380 | chr17 | 21179497 | - | | | GGGGACACGTGGGCCTTTCCA |
| | | | | | | | GTTCCCTGCAGCCACCTTTGGT |
| | | | | | | | CTGTAGGAAGGCAGTGG[CG] |
| | | | | | | | CAGGGAGCGGTGGGAGCCCG |
| | | | | | | | GGTCTGCAGGGCTCAAGGTGG |
| | | | | | | | CGACGGCGAAGCGGTCTGC |
| 256 | cg00874055 | chr1 | 236306673 | + | GPR137B | Body | ATTCGGGGCGCTTCTCCGTGC |
| | | | | | | | GCAGCGCGAAGCAGCAGCGC |
| | | | | | | | CTGCACACGCCAGTTAGTA[CG] |
| | | | | | | | GATGGAAGGTGTGCCCCCAA |
| | | | | | | | GGGAGGCCTGAACTCTAGAAT |
| | | | | | | | TTGCCCTGCCTCCCCAGGC |
| 257 | cg00307483 | chr1 | 27817084 | - | WASF2 | TSS1500 | CAAGCCCGTAAACTTTCTGTGG |
| | | | | | | | ACACCCCTCAAGTTGCGCATA |
| | | | | | | | GTGTTGTCCCTTCACTC[CG]GT |
| | | | | | | | CTCAGCCAGGGCAGAAAGTAG |
| | | | | | | | GGTGGGGAGAGTGAGTCACA |
| | | | | | | | AGCTCTATCCCGTCCTG |
| 258 | cg09165041 | chr1 | 40025882 | + | LOC728448 | TSS1500 | GATGGGGCACTAAGGAAGCA |
| | | | | | | | CCAAGCAAGCTCCAGGAGGGA |
| | | | | | | | AAGCAGGCAAGGCTGGAGC[CG] |
| | | | | | | | CAGGGAAAGTAGGCTGCAA |
| | | | | | | | AGGGATGTGATCTTGGCCTTT |
| | | | | | | | AGGATGTCATTTTACTGTCA |
| 259 | cg05266663 | chr1 | 23061564 | - | EPHB2; PEHB2 | Body; Body | AGGCTCAAGGGAGGGTGACA |
| | | | | | | | CTGACTAAGGCTGCACAGCAG |
| | | | | | | | GGCTATGAACCTGCTCTAC[CG] |
| | | | | | | | ACTCCTGTGGCCTGTGGGGCA |
| | | | | | | | TGGTGTGGGAGCATCTTCCTG |
| | | | | | | | AGGCTGCTGTTAAGAACA |
| 260 | cg13868165 | chr22 | 48888380 | + | FAM19A5 | Body | CCTTCTTTCTTTCTCGTGTGCTG |
| | | | | | | | GGATCCATATAGAAGGAGATG |
| | | | | | | | GGCTCCACCGTCTGGC[CG]GA |
| | | | | | | | GAAAGACCTGCAGTCCACCAA |
| | | | | | | | TTAGGCTAGTTGCTATAGTGAC |
| | | | | | | | ACAGCCTTGTCATTT |
| 261 | cg21943004 | chr11 | 59270264 | + | OR4D11 | TSS1500 | CTGCACTCCAGCCTGGGCGAC |
| | | | | | | | AGAGTAAGACTCTGTCTCAAA |
| | | | | | | | AAAAAAAAAAAACATTAT[CG] |
| | | | | | | | AAGTGTGAATTCAAATATGTG |
| | | | | | | | CAGTCTATGGTATGTCAATGAT |
| | | | | | | | AGCTCAACAAAAATTAT |
| 262 | cg15577927 | chr20 | 13201328 | + | ISM1 | TSS1500 | GAACGCCTAGAGAGTCGGACT |
| | | | | | | | CCCCTCCCTTCCCAGGCTCTAC |
| | | | | | | | GGGGCGCCGCGGATCCG[CG] |
| | | | | | | | AACAGCCGTGCCCGGCTAGCG |
| | | | | | | | GGCGGCCCAGCAAGTGTCAAG |
| | | | | | | | ACCCTTCGGAACGACACT |
| 263 | cg13159054 | chr15 | 47721715 | + | | | AAATCTGGAGTAAATTGCTAA |
| | | | | | | | GAGGGATTTTATCTGACTTAG |
| | | | | | | | GTTTGCAATATCTTTGAG[CG]T |
| | | | | | | | ATTGTGTTATCACCCTATTGCA |
| | | | | | | | TATTTGGTGGTAAGGCAACAG |
| | | | | | | | AACACCAACAAAATTA |
| 264 | cg04056904 | chr3 | 182399388 | - | | | ATAATACAAGACACCAGGTAC |
| | | | | | | | ATGGTGATGAGCAAAAACTGG |
| | | | | | | | CCCTTCTCTGTAATTATT[CG]C |
| | | | | | | | AATATAATATTAAACCCAACTT |
| | | | | | | | ACAATAAAAGAAATTCAAAAT |
| | | | | | | | AAAATGGTGCCAGGGA |
| 265 | cg12373003 | chr13 | 31943943 | + | | | TTATGAAATAAAGTCTACATTA |
| | | | | | | | AGAGTATGTGGGGAGCAGGA |
| | | | | | | | GAGGAGGGAACAAAATGC[CG] |
| | | | | | | | AAGACAGAGACAAGAGAGCA |
| | | | | | | | AACGGAATTAAGTGCTTTTCG |
| | | | | | | | ATATAGTTGGAAAGCAGAG |
| 266 | cg11510999 | chr12 | 53591490 | - | ITGB7 | Body | GGAGCTGCTGGGGCTCCCCTA |
| | | | | | | | GGGGGTGGGCGGCGGGCGGG |
| | | | | | | | TCAGCAGAGCGCATTGGAA[CG] |
| | | | | | | | CCAGCCTAGACCTCTGGCCT |
| | | | | | | | GGCCCCGCCTCCCCTAACTCAC |
| | | | | | | | CAGGCCGCAGCGTGACCC |
| 267 | cg02291532 | chr15 | 39874776 | - | THBS1 | Body | CAGCCTGACCGTCCAAGGAAA |
| | | | | | | | GCAGCACGTGGTGTCTGTGGA |
| | | | | | | | AGAAGCTCTCCTGGCAAC[CG] |
| | | | | | | | GCCAGTGGAAGAGCATCACCC |
| | | | | | | | TGTTTGTGCAGGAAGACAGGG |
| | | | | | | | CCCAGCTGTACATCGACT |
| 268 | cg26376566 | chr14 | 73603660 | - | PSEN1; | 5′UTR; | TGGAGTAGGAGAAAGAGGAA |
| | | | | | PSEN1 | 5′UTR | GCGTCTTGGGCTGGGTCTGCT |
| | | | | | | | TGAGCAACTGGTGAAACTC[CG] |
| | | | | | | | CGCCTCACGCCCCGGGTGTGT |
| | | | | | | | CCTTGTCCAGGGGCGACGAGC |
| | | | | | | | ATTCTGGGCGAAGTCCGC |
| 269 | cg14101501 | chr2 | 62932430 | + | EHBP1; | TSS1500; | CCTGGCGGAGATGAGAACAG |
| | | | | | EHBP1; | 5′UTR; | GAGAGAAACCCACAGGCAGCT |
| | | | | | EHBP1 | TSS1500 | GCACTGCCCACAGCTGCAG[CG] |
| | | | | | | | AAGCCAATCTCTAGGTCTGCA |
| | | | | | | | ATCACCCTTAGGGGCCAGAAA |
| | | | | | | | CCCAGCCCCGCACCAGCG |
| 270 | cg18268220 | chr14 | 61492123 | + | SLC38A6 | Body | AGTACTAAGAGTGTTTCAGAT |
| | | | | | | | ATACTAGTTTGTATTGTCTCTT |
| | | | | | | | GGGAAACTAGGATTGGG[CG] |
| | | | | | | | CGCAGATACATCGCCATCTGCT |
| | | | | | | | GGTCAGTTTATCTGTGGTGAA |
| | | | | | | | ACTGCAGCTTTCTTGAG |
| 271 | cg11457534 | chr11 | 133816062 | - | IGSF9B | Body | GAAGATAGGGATGGGGACCC |
| | | | | | | | CGAACTTGAACCACTCTACGAC |
| | | | | | | | ATAGGGTGGGGGCTGTCC[CG] |
| | | | | | | | TCACTGGGTGGATCACGTCGC |
| | | | | | | | ATCGCAGGACCACGCTCTCCCC |
| | | | | | | | AGCTCTTGCCGTCACAA |
| 272 | cg25463688 | chr1 | 235254025 | + | | | AAGCTTGTGGGAGACACAGAG |
| | | | | | | | AGGCAAAAGCTGAGCTGGGA |
| | | | | | | | AAATGGCAAGGCAGGGAGG[CG] |
| | | | | | | | CCAGAGGGAGCACTGCTTA |
| | | | | | | | ACACGTCCGTGGGGCTCCAAG |
| | | | | | | | GCTTTTAATAAAGGGATCCT |
| 273 | cg09643312 | chr2 | 160655081 | - | CD302 | TSS1500 | TGACATTGTATATAACGCCAGT |
| | | | | | | | GCAGTGATCAAACACAGGGCA |
| | | | | | | | CTCGCACTGGGATAATG[CG]A |
| | | | | | | | TTAGCTAATCTACAGCACTTAC |
| | | | | | | | CACATTTCATTAATTGCCCCTCT |
| | | | | | | | AAGGGTCCTTTTCT |
| 274 | cg12682862 | chr5 | 167913491 | - | RARS; | 5′UTR; | GGGGTTTCCGCTTCCGGGAGA |
| | | | | | RARS | 1stExon | GGCTGACCGTTTCCGCTTCCGT |
| | | | | | | | CCACTTGGCGAGTGAGA[CG]C |
| | | | | | | | TGATGGGAGGATGGACGTACT |
| | | | | | | | GGTGTCTGAGTGCTCCGCGCG |
| | | | | | | | GCTGCTGCAGCAGGTTT |
| 275 | cg20145610 | chr6 | 27205816 | + | | | CCATTCACGAGAGGGGCTTCC |
| | | | | | | | TTCCTTTTGACCTTGGGAGGG |
| | | | | | | | GTCCAGAGACCCGGGGGA[CG] |
| | | | | | | | ATCTGGGAGCAGAAGCTGGT |
| | | | | | | | CGTTCTGAGTTTTCCATCCAAA |
| | | | | | | | TGGTTTGCTTATGAAATT |
| 276 | cg07608813 | chr19 | 7587308 | - | MCOLN1 | TSS200 | ACATGGAAGTCACAAGCCTGG |
| | | | | | | | CACCGGATTCGGGGCATGGCC |
| | | | | | | | GGGAGCCAGGGCAGAGCT[CG] |
| | | | | | | | TCGTTGCCAAACTCAGAGTCA |
| | | | | | | | GCCCATCCCCCGCCACCCAGA |
| | | | | | | | GCGCGTCGGCGCTAGGAC |
| 277 | cg19359218 | chr6 | 30181936 | - | TRIM26 | TSS1500 | GCGGGCCGAGACTTGGGTTCC |
| | | | | | | | CCAGGTCCTTGGTGGGGAGGT |
| | | | | | | | TTCCAGGAGGCTCGGGCG[CG] |
| | | | | | | | CCCCCGTCCACGGCCCCGGAA |
| | | | | | | | GCTGACGTCGCCGAAGCGTAC |
| | | | | | | | GCCGCTGCCCAGCCTGCG |
| 278 | cg11251319 | chr19 | 1812732 | - | ATP8B3 | TSS1500 | GGGGTTGAGCATGGCCTTGCG |
| | | | | | | | GAGCAGTGTTATGGTAGGGGC |
| | | | | | | | GGGGCTGGGATCCGGAGC[CG] |
| | | | | | | | TTACAAAGGAGGAAGGCGGG |
| | | | | | | | GCCGCGCAGAGCAGGGTCAG |
| | | | | | | | GGTAGGAGGGCGCTCAGGGT |
| 279 | cg07417733 | chr8 | 48873326 | - | MCM4; PRKDC; | TSS200; | CCAGTTTTCCCGCGAAAACGCT |
| | | | | | PRKDC; MCM4 | TSS1500; | GCCGCGCAGGGGGTCAGACC |
| | | | | | | TSS1500; | ATCTGGACCAAGGGGGGC[CG] |
| | | | | | | TSS200 | AGCGAGGCCTACTTCTGGTTT |
| | | | | | | | ACGCACGGGCGCTGAAAGAA |
| | | | | | | | GCGGCACTGTCCCCCCCTG |
| 280 | cg10316834 | chr1 | 150534265 | - | | | TGAACTCAGTGGCTGCTGTTTT |
| | | | | | | | CTGAGCACCTGAACCCTGTGG |
| | | | | | | | GGGACGACAGAGTTGCC[CG] |
| | | | | | | | AGGCGGCAGGATGTCCCCACA |
| | | | | | | | CTCGCGGTCCCCCGCACATCTT |
| | | | | | | | CCTGTTGCTTTGGGACT |
| 281 | cg25548869 | chr6 | 29910776 | - | HLA-A | Body | CAGGAGACACGGAATGTGAA |
| | | | | | | | GGCCCAGTCACAGACTGACCG |
| | | | | | | | AGTGGACCTGGGGACCCTG[CG] |
| | | | | | | | CGGCTACTACAACCAGAGC |
| | | | | | | | GAGGCCGGTGAGTGACCCCG |
| | | | | | | | GCCGGGGGCGCAGGTCAGGA |
| | | | | | | | C |
| 282 | cg04775710 | chr6 | 30712022 | + | IER3 | Body | CTGGCGCCGGACCTAAGGGGA |
| | | | | | | | GACAAAACAGGAGACAGGTC |
| | | | | | | | AGGTCGAGGCCTCTGGAGT[CG] |
| | | | | | | | GGTCGTTCCCCAGTGACTCC |
| | | | | | | | AGGGCAGCGCACCCCGCGAAT |
| | | | | | | | GCCCACTTCGGCGATACTC |
| 283 | cg01885291 | chr6 | 28984832 | + | | | GAGAACAGCGATTAGGGCCTT |
| | | | | | | | AAACCTCACACCCGAACAAATT |
| | | | | | | | CGGCCGGAGTTACTGAG[CG]G |
| | | | | | | | CAGGCTCTCTGATGGAGATGG |
| | | | | | | | GTGCTTTCAGACTTAAGACGT |
| | | | | | | | GAAAACAAAGATCAGCC |
| 284 | cg00356811 | chr19 | 4639239 | + | TNFAIP8L1; | TSS1500; | CTGTCTGTCTCGTACTCTTATCT |
| | | | | | TNFAIP8L1 | TSS1500 | CTTCCCTTTTCTGTGGCCGGCA |
| | | | | | | | CCCCCACGACGGCCT[CG]CCC |
| | | | | | | | CCGCATCCGGGCCCCTTCGCG |
| | | | | | | | ATTCCGGAGGAATCCCCCAGA |
| | | | | | | | GCCGCCTGACCCCGC |
| 285 | cg05238905 | chr6 | 149867353 | + | PPIL4 | TSS200 | TCGGCGTGCGGGCGCCGGGCT |
| | | | | | | | GCCCAGCTGACTTACGGATCG |
| | | | | | | | GGTTGGTCCCGCCCCCGG[CG] |
| | | | | | | | CGGCCGTTTTGAAAATCCTGGT |
| | | | | | | | CCGCCCTTGGCGATTTTGGTG |
| | | | | | | | GAAGCCTGTCCCTCAGA |
| 286 | cg12612947 | chr3 | 25706262 | + | TOP2B | TSS1500 | TTCTCACACTCCGCGAAGGCCA |
| | | | | | | | GCCACTCGAGTCGCCAGAGTA |
| | | | | | | | GTCGTCCCGGTCGCCGC[CG]C |
| | | | | | | | TGCTTCAAAGGCAGCCTTAGC |
| | | | | | | | CTCGCTGCAGCCCCGATTTCCT |
| | | | | | | | CACACACACACACCGA |
| 287 | cg15921240 | chr4 | 331448 | + | ZNF141 | TSS200 | GCCAAGCACGAAGAGAAAGC |
| | | | | | | | CCCGCCTGAAACTGCCTGGAG |
| | | | | | | | GCCCCCCGGCTGTCACTCT[CG] |
| | | | | | | | CCACATTCCGTGGAGTATGTG |
| | | | | | | | GTTGCAACTTCTGTCACTCAAG |
| | | | | | | | GTCTGATGGCGGGGAGA |
| 288 | cg04195863 | chr15 | 25223574 | - | SNRPN; | Body; 3′UTR; | GTGTATCCTCTTTTTCTCAATGT |
| | | | | | SNURF; | Body; Body; | TTCTATTTCCTTTCCAGGTCCAC |
| | | | | | SNRPN; | Body; Body | CTCCCCCAGGAATG[CG]TCCA |
| | | | | | SNRPN; | | CCAAGACCTTAGCATACTGTTG |
| | | | | | SNRPN; | | ATCCATCTCAGTCACTTTTTCCC |
| | | | | | SNRPN | | CTGCAATGCGT |
| 289 | cg09822726 | chr17 | 61443331 | - | TANC2 | Body | ATTTATTATTAATTGTAGGTGA |
| | | | | | | | ATACTCGTTTTTGTCCACTTTTC |
| | | | | | | | TGTCTAAAATGAGCT[CG]ATG |
| | | | | | | | AGGACAAGAACCTTCTCTGTAT |
| | | | | | | | TGCTCACTGTGTCTTCCTAATG |
| | | | | | | | ATTAGTAGAGTGC |
| 290 | cg10645314 | chr2 | 3704589 | - | ALLC | TSS1500 | CCGCACCGTGAGCTTTGTGACT |
| | | | | | | | GATCCGAGGCGGCGAGCGGG |
| | | | | | | | GGCACTGCACTGCTGTGG[CG] |
| | | | | | | | GGGAAGTCACGGCTGACAAG |
| | | | | | | | AACTGCCAGGGACGAAGCCAC |
| | | | | | | | GTGCATTAATTCATTAAAA |
| 291 | cg03705220 | chr9 | 139089954 | + | LHX3; LHX3 | Body; Body | CCCACATTTTGCAGACAAGGA |
| | | | | | | | TATTTAGTTCCAGAGTGGCTGA |
| | | | | | | | GTGAGTAGCCCGGGTCA[CG]A |
| | | | | | | | GGCAGCCCAAAAGAGAGTGTC |
| | | | | | | | TTGTCCACATTCTGAGGATGG |
| | | | | | | | GCATCAACAGATGGGGA |
| 292 | cg05020775 | chr20 | 1246934 | + | SNPH | TSS200 | CGGCGAGCCGCCGACTGGCTG |
| | | | | | | | GTCCCCTCCATCCACCTCACCC |
| | | | | | | | TCCCCGCCCCTCCCTCC[CG]GC |
| | | | | | | | AGCCCCAGCCCCGGCGAGCAC |
| | | | | | | | CCAGCTAGCCGCCTCCTGCAG |
| | | | | | | | GGGCTCGGGAGAGCAA |
| 293 | cg07023563 | chr1 | 17989633 | - | ARHGEF10L; | Body; Body | TGTGTGGCATCAGGTGTGACT |
| | | | | | ARHGEF10L | | TCTGAGAAGAAACAATCTTGG |
| | | | | | | | CGCGCGCCGCTTGGATGC[CG] |
| | | | | | | | GAGAAAATGGTTCTTGGGTGC |
| | | | | | | | GCTGATCATCCCAGGGGAGGG |
| | | | | | | | GAGGACCTTGCTTGGGCC |
| 294 | cg27511169 | chr8 | 110704116 | - | GOLSYN; | TSS200; | TCCTGCCAGATGAGGGAGCCC |
| | | | | | GOLSYN | TSS200 | CGGCGGAGGCCAGGAGGGCT |
| | | | | | | | TGCGTTGCACAATCTGGAG[CG] |
| | | | | | | | GATCCCCGGGGGCGGCTGAG |
| | | | | | | | GGCCTGGGACCCCAGTCTCCC |
| | | | | | | | TCGAGGTCTTCACTCACCC |
| 295 | cg03209395 | chr7 | 1295653 | - | | | TGGCAGATCAGAGGCAGGCG |
| | | | | | | | GGCCAGGGGCTCTGGTTTACA |
| | | | | | | | CACCAAACCTCCAGGGCTT[CG] |
| | | | | | | | GCTCCAGGGGCCAGCAGCTG |
| | | | | | | | GGTCCACCCTGAGGGAGAGTC |
| | | | | | | | CCCAGGTGAGCGAGAAGCT |
| 296 | cg23288827 | chr17 | 4402117 | - | SPNS2 | TSS200 | CCCACCCCCAGGGCAGCACGT |
| | | | | | | | GCGGGGCGGGGCTGTGGCCC |
| | | | | | | | GAGCCCGGAGCTGATTGGG[CG] |
| | | | | | | | CGGGCCTGGTGGGCGGGGC |
| | | | | | | | CGGGCCGCAGCTGTCAGAGCC |
| | | | | | | | GCGGCGGCGAACGAGGCGCA |
| 297 | cg08984586 | chr5 | 175963618 | + | RNF44 | 5′UTR | CGCTCTCGGAGGGACACCGGG |
| | | | | | | | GGCGGGAGGCGAGACTGCAG |
| | | | | | | | CGCAGGGGCCAGAACGCTG[CG] |
| | | | | | | | ACTTTAAGAGCCGAGGATCC |
| | | | | | | | CGGACCATGTGCTCGGCGTGA |
| | | | | | | | GACAAAAGCAACAACAAAG |
| 298 | cg03835983 | ch20 | 61448085 | + | COL9A3 | TSS1500 | GGAAACTCGCGGGTCTCCCCT |
| | | | | | | | GCCCCTCCCTGAAGGCGGCCC |
| | | | | | | | TTCAGCGCCGCGCGCTTC[CG] |
| | | | | | | | CCCCCACACTCGGGTTGAGGA |
| | | | | | | | GCAAGGAGAGAAAAGAGCGT |
| | | | | | | | CTTTCTCTCTTGCTCAAAG |
| 299 | cg04808059 | chr20 | 42543442 | + | TOX2; TOX2; | TSS1500; | GGGCGGGGCGGGGGCGGGG |
| | | | | | TOX2 | TSS200; | GCGGGGCGCTCCTCTGGGCAC |
| | | | | | | TSS1500 | CGCCCCCGGCCCGCCCCCCG[CG] |
| | | | | | | | CTCGCAGTCCCGCTCGCACA |
| | | | | | | | CTGGCTCCCACCCGCCGCCCGC |
| | | | | | | | CCAGGCACTGCCCGCGGG |
| 300 | cg08540010 | chr20 | 48770450 | + | TMEM189; | TSS200; | CGAGCCGGAGGCTGGGACGC |
| | | | | | TMEM189; | TSS200; | AGCTGGACGCAGCTGGGCGC |
| | | | | | TMEM189; | TSS200; | GGAAGCTTGGGGCGGAGGCG |
| | | | | | TMEM189- | TSS200 | [CG]TGCCCGCCTTCCCAGCTCA |
| | | | | | UBE2V1 | | GCCCCGGCAGGGCTCCCGGCT |
| | | | | | | | CCAGCCCACTGGGAGCTCGC |

### RECITATION OF SELECTED EMBODIMENTS

### Embodiment 1

A system for calculating age of a biological sample, comprising:
(A) a data acquisition unit comprising
   a) a receiver for receiving a plurality of methylome datasets from a plurality of heterogeneous samples of different age or age groups, wherein each dataset comprises a plurality of methylation markers;
   b) a processor for homogenizing the plurality of methylome datasets and merging the homogenized dataset into a single data frame, thereby generating a processed dataset comprising a string of homogenized and merged methylation markers;
   c) a filter for filtering confounding markers from the processed dataset of (b), wherein filtration step comprises:
      1) removing cross-reactive markers in the processed dataset;
      2) removing unavailable markers in the processed dataset; and/or
      3) removing sex-specific markers from the processed dataset;
   d) an identifier for identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
   e) a selector for selecting a training dataset from the pool of relevant and unique markers of (d), wherein the selection step comprises balancing the age distribution of samples from which the relevant and unique markers are obtained.

### Embodiment 2

The system of Embodiment 1, which further comprises
(B) a marker identification unit configured to identify a plurality of age-specific methylation markers in the training dataset of e), the marker identification unit communicatively connected to the data acquisition unit, comprising:
   f) a classification engine configured to statistically classify each relevant and unique marker in the training dataset of e) on the basis of a relevance score which indicates a level of a statistical association between the marker and the age, wherein the methylation markers comprises the markers listed in Table 1, wherein the markers in Table 1 are listed in descending order of relevance score, and wherein the classification engine utilizes a machine learning (ML) model; and
   g) optionally a validation unit for validating the trained machine learning algorithm of (f) with a validation dataset; and

### Embodiment 3

The system of Embodiment 1, which further comprises
(C) an analyzing unit comprising:
   h) a detector for detecting the methylation status of age-specific, unique and relevant methylation markers identified in (e) or a gene linked to said methylation marker or locus thereto in a biological sample; and
   i) an age assessor which calculates the age of the biological sample based on the detected methylation status of the biological sample.

### Embodiment 4

The system of Embodiment 1, which comprises the data acquisition unit (A), the marker identification unit (B) and the analyzing unit (C).

### Embodiment 5

A computer readable medium comprising computer-executable instructions, which, when executed by a processor, cause the processor to carry out a method or a set of steps for diagnosing aging or an age-related disease in a subject, the method or the set of steps comprising, (A) a pre-analytical data processing, filtering, selection and balancing steps; optionally (B) a system setup step; and further optionally (C) an analytical step, wherein the pre-analytical step (A) comprises:
a) receiving a plurality of methylome datasets from a plurality of heterogeneous samples of different age or age groups, wherein each dataset comprises a plurality of methylation markers;
b) processing to homogenize the plurality of methylome datasets and merging the homogenized dataset into a single data frame, thereby generating a processed dataset comprising a string of homogenized and merged methylation markers;
c) filtering confounding markers from the processed dataset of (b), wherein filtration step comprises:
   1) removing cross-reactive markers in the processed dataset;
   2) removing unavailable markers in the processed dataset; and/or
   3) removing sex-specific markers from the processed dataset;
d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
e) selecting a training dataset from the pool of relevant and unique markers of (d), wherein the selection step comprises balancing the age distribution of samples from which the relevant and unique markers are obtained; wherein the optional system setup step (B) comprises
f) training a machine-learning algorithm comprising a Ridge regularized machine learning algorithm with the training dataset of e), thereby generating a plurality of age-specific, unique and relevant methylation markers, wherein the methylation markers comprises the markers listed in Table 1; and
g) optionally validating the trained machine learning algorithm of (f) with a validation dataset; and wherein the further optional analytical step (C) comprises
h) detecting the methylation status of age-specific, unique and relevant methylation markers identified in (e) or a gene linked to said methylation marker or locus thereto in the subject's biological sample; and
i) calculating the age of the subject's biological sample based on the detected methylation status of the subject's biological sample, wherein the markers in Table 1 are listed in descending order of relevance to the age of the subject's biological sample, and wherein if the calculated age is greater than the actual age of the subject, then the subject is diagnosed with aging or having an age-related disease.

### Embodiment 6

The computer readable medium of Embodiment 5, wherein the further optional analytical step further comprises j) comparing the calculated age with a chronological age of the subject to infer a rate at which the subject is aging and evaluating interventions to slow down aging or age-related disease in the subject.

### Embodiment 7

The computer readable medium of Embodiment 6, wherein computer-executable instructions, when executed by a processor, cause the processor to carry out a method or a set of steps for diagnosing aging or an age-related disease in a subject, the method or the set of steps comprising, (A) the pre-analytical data processing, filtering, selection and balancing steps; (B) the system setup step; and (C) the analytical step.

### Embodiment 8

A method for calculating an age of a biological sample, comprising, (A) a pre-analytical data processing, filtering, selection and balancing steps; (B) a system setup step; and (C) an analytical step, wherein the pre-analytical step (A) comprises:
a) receiving a plurality of methylome datasets from a plurality of heterogeneous samples of different age or age groups, wherein each dataset comprises a plurality of methylation markers;
b) processing to homogenize the plurality of methylome datasets and merging the homogenized dataset into a single data frame, thereby generating a processed dataset comprising a string of homogenized and merged methylation markers;
c) filtering confounding markers from the processed dataset of (b), wherein filtration step comprises:
1) removing cross-reactive markers in the processed dataset;
2) removing unavailable markers in the processed dataset; and/or
3) removing sex-specific markers from the processed dataset;
d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
e) selecting a training dataset from the pool of relevant and unique markers of (d), wherein the selection step comprises balancing the age distribution of samples from which the relevant and unique markers are obtained; wherein the system setup step (B) comprises
f) training a machine-learning algorithm comprising a Ridge regression machine learning algorithm with the training dataset of e), thereby generating a plurality of age-specific, unique and relevant methylation markers, wherein the methylation markers comprises the markers listed in Table 1; and
g) optionally validating the trained machine learning algorithm of (f) with a validation dataset; and wherein the analytical step (C) comprises
h) detecting the methylation status of age-specific, unique and relevant methylation markers identified in (e) or a gene linked to said methylation marker or locus thereto in the biological sample; and
i) determining the age of the biological sample based on the detected methylation status of the biological sample.

### Embodiment 9

A method for calculating an age of a biological sample, comprising detecting the methylation status of age-specific, unique and relevant methylation markers in the biological sample and determining the age of the biological sample based on the detected methylation status of the biological sample, wherein the age-specific, unique and relevant methylation markers are identified in a methylome dataset by employing (A) pre-analytical data processing, filtering, selection and balancing steps; and (B) setting-up step, wherein, the pre-analytical data processing, filtering, selection and balancing step (A) comprises:
a) receiving a plurality of methylome datasets from a plurality of heterogeneous samples of different age or age groups, wherein each dataset comprises a plurality of methylation markers;
b) processing to homogenize the plurality of methylome datasets and merging the homogenized dataset into a single data frame, thereby generating a processed dataset comprising a string of homogenized and merged methylation markers;
c) filtering confounding markers from the processed dataset of (b), wherein filtration step comprises:
   1) removing cross-reactive markers in the processed dataset;
   2) removing unavailable markers in the processed dataset; and/or
   3) removing sex-specific markers from the processed dataset;
d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
e) selecting a training dataset from the pool of relevant and unique markers of (d), wherein the selection step comprises balancing the age distribution of samples from which the relevant and unique markers are obtained; and the setting up step (B) comprises
f) training a machine-learning algorithm comprising a Ridge regression machine learning algorithm with the training dataset of e), thereby generating a plurality of age-specific, unique and relevant methylation markers, wherein the methylation markers comprises the markers listed in Table 1; and
g) optionally validating the trained machine learning algorithm of (f) with a validation dataset.

### Embodiment 10

The method of Embodiment 8 or Embodiment 9, wherein the methylation markers comprise levels and/or activity of methylated genomic DNA (gDNA) in the samples.

### Embodiment 11

The method of Embodiment 8 or Embodiment 9, wherein in step c), the cross-reactive markers are identified by comparing the dataset of (b) with a standard, non-specific probe dataset.

### Embodiment 12

The method of Embodiment 8 or Embodiment 9, wherein in step c), the unavailable markers comprise markers that are not included in the pool of markers which are assayable with the methylation assay instrument.

### Embodiment 13

The method of Embodiment 8 or Embodiment 9, wherein in step c), the sex-specific markers comprise markers that are specific to a single sex.

### Embodiment 14

The method of Embodiment 8 or Embodiment 9, wherein in step d), the correlation or regression comprises application of a regression analysis comprising glmnet-lasso, xgboost, and ranger.

### Embodiment 15

The method of Embodiment 8 or Embodiment 9, wherein in step e), the age balancing step comprises not having more than n samples per age window of y years, beginning with age z years, wherein n, y, and z are integers >0.

### Embodiment 16

The method of Embodiment 15, wherein n=5 or 6; y=7 years or 8 years; and z=16 years to 20 years.

### Embodiment 17

The method of Embodiment 15, wherein n=5, y=7 years and z=18 years.

### Embodiment 18

The method of Embodiment 8 or Embodiment 9, wherein in step f), the machine-learning algorithm is based on Ridge regression, which penalizes the size of parameter estimates by shrinking them to zero, in order to decrease complexity of the model while including all the variables in the model.

### Embodiment 19

The method of Embodiment 8 or Embodiment 9, wherein the age of the biological sample is determined using a regression model that predicts sample age based on a weighted average of the methylation marker levels plus an offset, preferably, the offset comprises an addition or subtraction of a delta age (6), derived from a validation dataset of samples obtained from the subject, e.g., as provided in a hash table of Table 4.

### Embodiment 20

The method of Embodiment 8 or Embodiment 9, wherein the methylation status comprises level and/or amount of methylation markers or pattern of methylation markers in the biological sample.

### Embodiment 21

A method for calculating an age of a biological sample, comprising, detecting, status of methylation markers in a genomic DNA (gDNA) of the biological sample; and determining the age of the sample based on the status of the detected methylation markers, wherein the methylation markers, in order of their relevance with calculated age of the biological sample, are selected from cg06279276 and cg00699993, wherein the structure of each methylation marker is provided by the respective Probe ID Nos., the nucleotide sequences and methylated residues therein, as indicated by nucleotides inside large parenthesis, which are set forth in
(a) CCGCCGCTGGTCCTTGGCGCGCAAATAGCGGGCGAAGTCAAAGGGTCCCGTAGGC GTGGG[CG]GCGCCGGTGTGTCCCCTTCGTAGGCCGGCGGGGCTGCACCCGCGTCGGGTAACT GGAACG (cg06279276); and
(b) CGCACGAAGGTAGCTCCGGGCGGGGAGCGAGGCGCTGTCCTCGGTGCTGAAAGGC CGAGG[CG]CGCGGTGGGCGCGACAGCCCCGGAGACCCGAGGTCTCGCGGAGGGACAGCGGC TACGGGC (cg00699993); or a gene linked to said methylation marker or locus thereto.

### Embodiment 22

The method of Embodiment 21, comprising detecting both cg06279276 and cg00699993, wherein the methylation markers are listed in order of their association with age of the biological sample.

### Embodiment 23

The method of Embodiment 21, wherein the gene linked to the methylation marker or locus thereto is selected from B3GNT9 and GRIA2.

### Embodiment 24

A method for calculating an age of a biological sample, comprising, detecting, status of methylation markers in a genomic DNA (gDNA) of the biological sample; and determining the age of the sample based on the status of the detected methylation markers, wherein the methylation markers are selected from methylation markers in a gene selected from CNTNAP5; SYT7; MARCH11; SLC12A5; GRIA2; C2orf65; DLL3; B3GNT9; ATP4A; EVI5L; INA; SALL3; RYR2; DUPD1; TCF21; SOD3; RASEF; PLD3; C17orf93; PRAC; CACNA1G; ZNF549; B4GALNT1; ZMIZ1; NCAM2; LOC375196; LOC100271715; ZIC1; CMTM2; PEX5L; IRS2; ZNF518B; ANKRD34B; ZNF167; BRUNOL4; GRIN2D; OTUD7A; TBR1; TLX3; LOC728392; HIST1H2BK; ZYG11A; NR4A2; ZNF518B; DCC; PRSS27; ELOVL2; RUNX1; CCDC140; UNKL; C19orf55; SIX6; CLIC6; PAX9; UCHL1; NETO2; ENTPD3; SLC12A5; GDF6; LOC100128788; SRRM2; PTPRN; HPSE2; BSX; PTPRN; VGF; PRDM2; TBX4; C3orf39; MUL1; DBX1; LINGO3; ZNF578; ZIC5; DIP2C; HIST1H4I; ZYG11B; RASGEF1A; GPR78; DNAJC5G; AGRN; CLIC6; SDCBP2; TRAF3; MLXIPL; MCHR2; PRDM6; F1141350; THRB; SIM2; POM121L2; SNRNP200; H19; UNC5D; MRPS33; TRIM59; SNHG9; SNORA78; RPS2; MITF; GREB1L; HOXD13; PEX5L; P2RX2; NRN1; KIF15; KIAA1143; MIR1826; CTNNA2; GPR144; ZNF577; FBRS; SLC15A3; PIPDX; BDNF; KLF14; POU4F1; CXCR7; LOC285375; NKAIN3; NR6A1; NUDT16P; TRPC3; MIR196B; HTR1A; SLC6A20; SUB1; AMMECR1L; ATP5G3; AMH; C7orf20; DNAH8; BCO2; PAX9; MRTO4; UCKL1AS; UCKL1; POP4; SLC5A8; TNFSF10; BCR; HLA-C; HSPG2; AKAP12; ADRB1; LRRC55; ZNF136; MCTP2; LOC440925; OTUB1; CASP7; MYT1L; PES1; GMPS; CCT3; Clorf182; MLF2; NOVA2; APLF; FBXO48; LOC728743; GIPR; RADIL; CPLX2; TMEM59; C1orf83; RCAN1; GJB6; RPH3AL; BAT1; CCDC87; CCS; DPEP1; MIR24-1; C9orf3; CASP2; TPD52; ZNF804B; MGC26647; SLC25A15; COX5B; CD164L2; ME1; WDR27; RTN4RL1; C5orf36; TMEM188; NAPRT1; PDLIM4; MCF2L; NDUFB6; LDB2; DHX29; SKIV2L2; ARL6IP6; PRPF40A; COL4A1; SNED1; CDC40; WASF1; VPS13D; ZNF783; TNXB; PRDM1; GLT1D1; CBX7; GPR137B; WASF2; LOC728448; EPHB2; FAM19A5; OR4D11; ISM1; ITGB7; THBS1; PSEN1; EHBP1; SLC38A6; IGSF9B; CD302; RARS; MCOLN1; TRIM26; ATP8B3; MCM4; PRKDC; HLA-A; IER3; TNFAIP8L1; PPIL4; TOP2B; ZNF141; SNRPN; SNURF; TANC2; ALLC; LHX3; SNPH; ARHGEF10L; GOLSYN; SPNS2; RNF44; COL9A3; TOX2; TMEM189; and TMEM189-UBE2V1; or a locus linked to the gene.

### Embodiment 25

The method of Embodiment 24 or Embodiment 36, wherein the methylation marker or locus thereto is provided in Table 1.

### Embodiment 26

A method for calculating an age of a biological sample, comprising, detecting, status of methylation markers in a genomic DNA (gDNA) of the biological sample; and determining the age of the sample based on the status of the detected methylation markers, wherein the methylation markers comprise a plurality of methylation markers that are listed in order of their association with age of the biological sample, the methylation markers are selected from cg17484671; cg11344566; cg24809973; cg03200166; cg06782035; cg02352240; cg25351606; cg07547549; cg03354992; cg00699993; cg02611848; cg07640648; cg18235734; cg06279276; cg00748589; cg23368787; cg02383785; cg02961707; cg15475851; cg07171111; cg05080154; cg03422911; cg14462779; cg16061498; cg04467618; cg02891686; cg12969644; cg25509871; cg09017434; cg17508941; cg12374721; cg11071401; cg06458239; cg05771369; cg25645064; cg14371731; cg19556343; cg22158769; cg10729426; cg16181396; cg00049664; cg13473356; cg05404236; cg16295725; cg21800232; cg23437843; cg24202131; cg15779837; cg04875128; cg06488443; cg24213719; cg25936177; cg17833476; cg12852499; cg18671949; cg16991515; cg06784991; cg00194126; cg00511674; cg08032924; cg18795809; cg18866015; cg10286969; cg21572722; cg23967544; cg11498607; cg14676592; cg10269365; cg01682111; cg10501210; cg27345346; cg08097417; cg19456540; cg04528819; cg10977667; cg19200589; cg23291886; cg10911990; cg06785999; cg24715245; cg18867659; cg10755058; cg07060233; cg18533201; cg03507326; cg06971096; cg26329178; cg24317217; cg24719321; cg14226702; cg03970036; cg21186299; cg15568145; cg06365535; cg01359962; cg07116393; cg13696942; cg09370594; cg25763393; cg24136205; cg06571559; cg13592721; cg23995459; cg23136139; cg11970349; cg06287137; cg21269897; cg18988435; cg14663984; cg18371700; cg12242474; cg26115667; cg23156348; cg13337731; cg09393254; cg02081006; cg06520675; cg00323305; cg10196902; cg21353911; cg21091227; cg19026977; cg08079908; cg02983163; cg21901946; cg17040303; cg09551472; cg13140267; cg11716026; cg25273520; cg06432426; cg24813736; cg17486097; cg26792755; cg26856080; cg06385324; cg04811592; cg03735496; cg14772615; cg24914355; cg13141009; cg14979301; cg09785958; cg26620450; cg21467631; cg20223728; cg24888989; cg06617961; cg25636665; cg11027140; cg24794228; cg05437148; cg18151345; cg06144905; cg10635145; cg21449170; cg01994205; cg15911409; cg03553786; cg24340081; cg13601993; cg18413131; cg07674022; cg08964780; cg23298047; cg08259925; cg24261921; cg13289553; cg26782833; cg18119885; cg04306050; cg11325997; cg00081714; cg24580076; cg24636999; cg25303383; cg01672943; cg07312601; cg12778178; cg16023306; cg05722918; cg22572614; cg10346212; cg14942863; cg03930964; cg05030953; cg27304144; cg12794224; cg17028652; cg24458609; cg26454158; cg15481429; cg08386537; cg19233923; cg01414572; cg06517429; cg06760904; cg00059424; cg11002227; cg25371803; cg20642765; cg08734053; cg11567723; cg16897193; cg23021855; cg08261702; cg18088844; cg11594299; cg16025094; cg15309223; cg05156137; cg03335886; cg01717881; cg03031988; cg04738656; cg23229770; cg07299526; cg20355806; cg02268620; cg26050838; cg05335473; cg13009608; cg04631458; cg26777345; cg22946147; cg22425860; cg00151919; cg19255191; cg22872989; cg10286959; cg21877956; cg17279592; cg02064158; cg25584787; cg09113665; cg13282195; cg03873281; cg00841725; cg16758041; cg12528144; cg19136783; cg00798886; cg11732282; cg12213687; cg16937168; cg14866740; cg18703066; cg19772114; cg07139350; cg13614741; cg04172115; cg01146808; cg06826289; cg23124451; cg05200380; cg00874055; cg00307483; cg09165041; cg05266663; cg13868165; cg21943004; cg15577927; cg13159054; cg04056904; cg12373003; cg11510999; cg02291532; cg26376566; cg14101501; cg18268220; cg11457534; cg25463688; cg09643312; cg12682862; cg20145610; cg07608813; cg19359218; cg11251319; cg07417733; cg10316834; cg25548869; cg04775710; cg01885291; cg00356811; cg05238905; cg12612947; cg15921240; cg04195863; cg09822726; cg10645314; cg03705220; cg05020775; cg07023563; cg27511169; cg03209395; cg23288827; cg08984586; cg03835983; cg04808059; and cg08540010; or a gene linked to said methylation marker or locus thereto; wherein the structure of each methylation marker is provided by the respective Probe ID Nos.

### Embodiment 27

The method of any one of Embodiments 3-26, wherein the biological sample comprises skin, blood, saliva, sperm, heart, brain, kidney, or liver sample.

### Embodiment 28

The method of any one of Embodiments 3-26, wherein the biological sample comprises epidermal or dermal cells or fibroblasts or keratinocytes.

### Embodiment 29

The method of any one of Embodiments 8-28, wherein the detection of the status of methylation markers comprises detection of a level or pattern of methylation markers.

### Embodiment 30

The method of Embodiment 29, wherein the detection of the level of methylation markers comprises treatment of genomic DNA from the sample with a reagent to convert unmethylated cytosines of CpG dinucleotides to uracil and wherein the detection of the pattern of methylation markers comprises identification of methylation levels at age-associated CpG sites.

### Embodiment 31

A kit for calculating an age of a biological sample, comprising, probes for detecting, status of methylation markers in a genomic DNA (gDNA) of the biological sample; vessels for holding the biological sample; optionally together with instructions for performing the detection, wherein the methylation markers are selected from selected from cg17484671; cg11344566; cg24809973; cg03200166; cg06782035; cg02352240; cg25351606; cg07547549; cg03354992; cg00699993; cg02611848; cg07640648; cg18235734; cg06279276; cg00748589; cg23368787; cg02383785; cg02961707; cg15475851; cg07171111; cg05080154; cg03422911; cg14462779; cg16061498; cg04467618; cg02891686; cg12969644; cg25509871; cg09017434; cg17508941; cg12374721; cg11071401; cg06458239; cg05771369; cg25645064; cg14371731; cg19556343; cg22158769; cg10729426; cg16181396; cg00049664; cg13473356; cg05404236; cg16295725; cg21800232; cg23437843; cg24202131; cg15779837; cg04875128; cg06488443; cg24213719; cg25936177; cg17833476; cg12852499; cg18671949; cg16991515; cg06784991; cg00194126; cg00511674; cg08032924; cg18795809; cg18866015; cg10286969; cg21572722; cg23967544; cg11498607; cg14676592; cg10269365; cg01682111; cg10501210; cg27345346; cg08097417; cg19456540; cg04528819; cg10977667; cg19200589; cg23291886; cg10911990; cg06785999; cg24715245; cg18867659; cg10755058; cg07060233; cg18533201; cg03507326; cg06971096; cg26329178; cg24317217; cg24719321; cg14226702; cg03970036; cg21186299; cg15568145; cg06365535; cg01359962; cg07116393; cg13696942; cg09370594; cg25763393; cg24136205; cg06571559; cg13592721; cg23995459; cg23136139; cg11970349; cg06287137; cg21269897; cg18988435; cg14663984; cg18371700; cg12242474; cg26115667; cg23156348; cg13337731; cg09393254; cg02081006; cg06520675; cg00323305; cg10196902; cg21353911; cg21091227; cg19026977; cg08079908; cg02983163; cg21901946; cg17040303; cg09551472; cg13140267; cg11716026; cg25273520; cg06432426; cg24813736; cg17486097; cg26792755; cg26856080; cg06385324; cg04811592; cg03735496; cg14772615; cg24914355; cg13141009; cg14979301; cg09785958; cg26620450; cg21467631; cg20223728; cg24888989; cg06617961; cg25636665; cg11027140; cg24794228; cg05437148; cg18151345; cg06144905; cg10635145; cg21449170; cg01994205; cg15911409; cg03553786; cg24340081; cg13601993; cg18413131; cg07674022; cg08964780; cg23298047; cg08259925; cg24261921; cg13289553; cg26782833; cg18119885; cg04306050; cg11325997; cg00081714; cg24580076; cg24636999; cg25303383; cg01672943; cg07312601; cg12778178; cg16023306; cg05722918; cg22572614; cg10346212; cg14942863; cg03930964; cg05030953; cg27304144; cg12794224; cg17028652; cg24458609; cg26454158; cg15481429; cg08386537; cg19233923; cg01414572; cg06517429; cg06760904; cg00059424; cg11002227; cg25371803; cg20642765; cg08734053; cg11567723; cg16897193; cg23021855; cg08261702; cg18088844; cg11594299; cg16025094; cg15309223; cg05156137; cg03335886; cg01717881; cg03031988; cg04738656; cg23229770; cg07299526; cg20355806; cg02268620; cg26050838; cg05335473; cg13009608; cg04631458; cg26777345; cg22946147; cg22425860; cg00151919; cg19255191; cg22872989; cg10286959; cg21877956; cg17279592; cg02064158; cg25584787; cg09113665; cg13282195; cg03873281; cg00841725; cg16758041; cg12528144; cg19136783; cg00798886; cg11732282; cg12213687; cg16937168; cg14866740; cg18703066; cg19772114; cg07139350; cg13614741; cg04172115; cg01146808; cg06826289; cg23124451; cg05200380; cg00874055; cg00307483; cg09165041; cg05266663; cg13868165; cg21943004; cg15577927; cg13159054; cg04056904; cg12373003; cg11510999; cg02291532; cg26376566; cg14101501; cg18268220; cg11457534; cg25463688; cg09643312; cg12682862; cg20145610; cg07608813; cg19359218; cg11251319; cg07417733; cg10316834; cg25548869; cg04775710; cg01885291; cg00356811; cg05238905; cg12612947; cg15921240; cg04195863; cg09822726; cg10645314; cg03705220; cg05020775; cg07023563; cg27511169; cg03209395; cg23288827; cg08984586; cg03835983; cg04808059; and cg08540010; wherein the structure of each methylation marker is provided by the respective Probe ID Nos., or a gene linked to said methylation marker or locus thereto.

### Embodiment 32

The kit of Embodiment 31, comprising a plurality of probes for detecting, status of one or more methylation markers selected from cg06279276 and cg00699993, preferably both cg06279276 and cg00699993, wherein the structure of each methylation marker is provided by the respective Probe ID Nos., the nucleotide sequences and methylated residues therein, as indicated by nucleotides inside large parenthesis, which are set forth in

(a) CCGCCGCTGGTCCTTGGCGCGCAAATAGCGGGCGAAGTCAAAGGGTCCCGTAGGC GTGGG[CG]GCGCCGGTGTGTCCCCTTCGTAGGCCGGCGGGGCTGCACCCGCGTCGGG TAACTGGAACG (cg06279276); and
(b) CGCACGAAGGTAGCTCCGGGCGGGGAGCGAGGCGCTGTCCTCGGTGCTGAAAGGCCGAGG [CG]CGCGGTGGGCGCGACAGCCCCGGAGACCCGAGGTCTCGCGGAGGGACAGCGGCTACGG GC (cg00699993); or a gene linked to said methylation marker or locus thereto.

### Embodiment 33

The kit of Embodiment 31, comprising a plurality of probes for detecting, status of the methylation markers selected from cg06279276 and cg00699993.

### Embodiment 34

A computer readable medium according to Embodiment 5 or Embodiment 6, comprising computer-executable instructions, which, when executed by a processor, cause the processor to carry out a method or a set of steps for identifying methylation markers in a genetic dataset received from a subject's sample, wherein the methylation markers comprises a level or pattern of methylation in the genomic DNA (gDNA), the medium comprising a Machine learning algorithm.

### Embodiment 35

The computer readable medium of Embodiment 34, comprising computer-executable instructions, wherein the ML is trained with a compendium of methylation markers each of which are annotated with age and the ML computes the predictive power of each marker using a rigorous mathematical algorithm comprising or least absolute shrinkage and selection operator (LASSO), BOOSTING or RANDOM FOREST.

### Embodiment 36

The computer readable medium of Embodiment 34, comprising computer-executable instructions, wherein the ML comprises a Machine learning algorithm comprising linear model (LM); Generalized Linear Model with Stepwise Feature Selection (GLMSTEPAIC); supervised principal components (SUPERPC); k-nearest neighbor (KNN); Penalized Linear Regression (PEN); Boosted Generalized Linear Model (GLMBOOST); Generalized Linear Model (GLM); Ridge Regression (RIDGE); Deep Learning; or least absolute shrinkage and selection operator (LASSO) or a combination thereof.

### Embodiment 37

The computer readable medium of Embodiment 34, comprising computer-executable instructions, wherein ML algorithm comprising Ridge regression.

### Embodiment 38

A system for calculating an age of a biological sample, comprising:
(a) an optional counter configured to count numbers and/or levels of methylation markers in a genomic DNA (gDNA) of the biological sample and output a methylation data of the sample, wherein the methylation markers comprises the markers listed in Table 1, wherein the structure of each methylation marker is provided by the respective ILLUMINA Probe ID Nos., the nucleotide sequences and methylated residues therein, as indicated by nucleotides inside large parenthesis, is provided by the respective SEQ ID Nos.; and
(b) a computing device comprising,
   (1) a methylation analyzer that is configured to detect patterns and/or levels of methylation markers in the sample's methylation data, wherein the analyzer is communicatively connected to the counter when the counter is present;
   (2) an age identifier engine configured to predict age of the sample based on the patterns and/or levels of methylation markers; and
   (3) a display communicatively connected to the computing device and configured to display a report containing the biological sample's calculated age.

### Embodiment 39

The system of Embodiment 1 or Embodiment 38, wherein the methylation markers are selected from cg06279276 and cg00699993, preferably both cg06279276 and cg00699993; or a gene linked to said methylation marker or locus thereto.

### Embodiment 40

A method of screening an anti-aging agent, comprising, contacting the agent with a cell/tissue/organism for a period sufficient to induce epigenetic changes in the cell; determining a modulation of a plurality of methylation markers selected from methylation markers of Table 1 in the cell; and selecting the test agent based on the modulation of the methylation markers.

### Embodiment 41

The method of Embodiment 40, wherein the modulation comprises increase in methylation levels.

### Embodiment 42

The method of Embodiment 40, wherein the modulation comprises a reduction in methylation levels.

### Embodiment 43

The method of Embodiment 40, wherein the cell is a skin cell, e.g., a fibroblast cell and/or keratinocyte cell.

### Embodiment 44

The method of Embodiment 40, wherein plurality of methylation markers comprises at least 5, 10, 20, 30, 40, 50, 100, 150, 200, 250, 300 or all the markers from Table 1.

### Embodiment 45

The method of Embodiment 40, wherein plurality of methylation markers comprises markers having the C/G sequences set forth in (1) SEQ ID Nos: 1-20; (2) SEQ ID Nos: 1-40; (3) SEQ ID Nos: 1-60; (4) SEQ ID Nos: 1-80; (5) SEQ ID Nos: 1-100; (6) SEQ ID Nos: 1-120; (7) SEQ ID Nos: 1-140; (8) SEQ ID Nos: 1-160; (9) SEQ ID Nos: 1-180; (10) SEQ ID Nos: 1-200; (11) SEQ ID Nos: 1-220; (12) SEQ ID Nos: 1-240; (13) SEQ ID Nos: 1-260; (14) SEQ ID Nos: 1-280; or (15) SEQ ID Nos: 1-300.

### Embodiment 46

The method of Embodiment 40, wherein the method comprises (a) detecting the status of a plurality of methylation markers from Table 1 in a genomic DNA (gDNA) of a biological sample and calculating a first age of the subject's biological sample based on the status of the detected methylation markers, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) contacting the biological sample with a test compound; and (c) detecting the status of a plurality of the methylation markers of (a) in the genomic DNA (gDNA) of the biological sample contacted with the test compound and calculating a second age of the test compound-contacted biological sample based on the status of the methylation markers detected in (a); wherein if the second calculated age of the biological sample is modulated compared to the first calculated age of the biological sample, then the test compound is identified as modulating aging or a disease-related thereto.

### Embodiment 47

The method of Embodiment 46, wherein a difference between the subject's first calculated age and second calculated age (δ) is used in the identification of modulating test compounds.

### Embodiment 48

The method of Embodiment 47, wherein a threshold δ is first computed using known samples to determine a standard error rate, and the threshold δ value is used to determine whether the modulating effect of the test compound is due to a biological property thereof.

### Embodiment 49

The method of Embodiment 48, wherein an absolute delta (δ) greater than 1 month, 6 months, 1 year, 2 years, 3 years, 4 years, 5 years, 6 years, 7 years, 8 years, 9 years, 10 years, or 11 years, or more, e.g., 12 years (preferably about 5 years) is used as a threshold δ.

### Embodiment 50

The method of Embodiment 49, wherein a positive delta (+δ), e.g., a δ of +5 years, is used as a threshold for determining whether a test compound is a promoter of aging or an age-related disease or wherein a negative delta (−δ), e.g., a δ of −5 years, is as threshold for determining whether a test compound is a reverser of aging or an age-related disease.

### Embodiment 51

The methods according to any one of Embodiments 46 to 50, wherein the screening methods are carried out in high throughput screening (HTS) format.

### Embodiment 52

A method for identifying a subject for aging or having an age-related disease comprising: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating the age of the subject's biological sample based on the status of the detected methylation markers, wherein if the calculated age of the sample is greater than the subject's actual age, then the subject is positively identified as aging or having an age-related disease.

### Embodiment 53

The method of Embodiment 52, wherein the difference between the subject's actual age and calculated age (Δ) is indicative of whether the subject is aging or has an age-related disease.

### Embodiment 54

The method of Embodiment 53, wherein an absolute delta (Δ) of about 1 month, 6 months, 1 year, 2 years, 3 years, 4 years, 5 years, 6 years, 7 years, 8 years, 9 years, 10 years, or 11 years, or more, e.g., 12 years, is used as a threshold for the positive identification of subjects as aging or having an age-related diseases.

### Embodiment 55

The method of Embodiment 54, wherein a threshold Δ of about 5 years is used in identification of the subjects who are aging or having an age-related disease.

### Embodiment 56

The method of Embodiment 55, wherein a positive Δ (e.g., >5 years) indicates that the subject is aging abnormally.

### Embodiment 57

A method for prognosticating a subject for developing aging or an age-related disease comprising: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating the age of the subject's biological sample based on the status of the detected methylation markers, wherein if the calculated age of the sample is greater than the subject's actual age, then the subject is prognosticated as being at risk for developing aging or an age-related disease and/or if the calculated age of the sample is less than the subject's actual age, then the subject is prognosticated as not being at risk for developing aging or an age-related disease.

### Embodiment 58

The method of Embodiment 57, wherein the difference between the subject's actual age and calculated age (Δ) is indicative of whether the subject is prognosticated as being at risk for aging or having an age-related disease.

### Embodiment 59

The method of Embodiment 58, wherein a delta (Δ) of about 1 month, 6 months, 1 year, 2 years, 3 years, 4 years, 5 years, 6 years, 7 years, 8 years, 9 years, 10 years, or 11 years, or more, e.g., 12 years, is used as a threshold for a reliable prognostication of at-risk subject.

### Embodiment 60

A method for determining the efficacy of a drug or a therapy against aging or an age-related disease comprise the following steps: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating a first calculated age of the subject's biological sample based on the status of the detected methylation marker; (c) administering to the subject, an anti-aging drug or therapy if the first calculated age of the subject's sample is greater than the subject's actual age; (d) detecting the status of a plurality of the methylation markers of (a) in the genomic DNA (gDNA) of the biological sample of the subject treated with the anti-aging drug or therapy and calculating a second calculated age of the test compound-contacted biological sample based on the status of the methylation markers detected in (a); and (e) determining the effectiveness of the anti-aging drug or therapy based on the modulation of the second calculated age compared to the first calculated age.

### Embodiment 61

The method of Embodiment 60, wherein, if the second calculated age is less than the first calculated age, then the anti-aging drug or therapy is deemed effective.

### Embodiment 62

The method of Embodiment 60, wherein, if the second calculated age is greater than the first calculated age, then the anti-aging drug or therapy is deemed ineffective.

### Embodiment 63

The method of Embodiment 60, wherein if the difference between the first and second calculated age is positive (i.e., second calculated age<first calculated age) or the difference is greater than a threshold level (e.g., 5 years), then the anti-aging drug or therapy is deemed effective and if the difference between the first and second calculated age is negative (i.e., second calculated age >first calculated age) or the difference is less than a threshold level (e.g., 5 years), then the anti-aging drug or therapy is deemed ineffective.

### Embodiment 64

A method for treating aging or an age-related disease comprising: (a) detecting the status of a plurality of methylation markers from Table 1 in a genomic DNA (gDNA) of the subject's biological sample, wherein the structure of each methylation marker is provided by the respective Probe ID Nos., the nucleotide sequences and methylated residues therein, as indicated by nucleotides inside large parenthesis, is provided by the respective SEQ ID Nos., or a gene linked to the methylation marker or a locus thereto; (b) calculating a first calculated age of the subject's biological sample based on the status of the detected methylation marker; (c) administering to the subject, an anti-aging drug or therapy if the first calculated age of the subject's sample is greater than the subject's actual age; (d) detecting the status of a plurality of the methylation markers of (a) in the genomic DNA (gDNA) of the biological sample of the subject treated with the anti-aging drug or therapy and calculating a second calculated age of the treated biological sample based on the status of the methylation markers detected in (a); and (e) continuing anti-aging drug treatment or therapy until the second calculated age is within a threshold level of the subject's actual age.

### Embodiment 65

The method of Embodiment 64, wherein the threshold level is about 5 years or less, e.g., about 4 years, about 3 years, about 2 years, about 1 year, about 6 months, or about 1 month.

What is claimed:

1. A system for selecting markers for a training dataset to predict age of a biological sample, comprising:
(A) a data acquisition unit comprising
a) a receiver for receiving a plurality of methylome datasets from a plurality of heterogeneous samples of different age or age groups, wherein each dataset comprises a plurality of methylation markers;
b) a processor for homogenizing the plurality of methylome datasets and merging the homogenized dataset into a single data frame, thereby generating a processed dataset comprising a string of homogenized and merged methylation markers;
c) a filter for filtering confounding markers from the processed dataset of (b), wherein filtration step comprises:
1) removing cross-reactive markers in the processed dataset;
2) removing unavailable markers in the processed dataset; and/or
3) removing sex-specific markers from the processed dataset;
d) an identifier for identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
e) a selector for selecting a training dataset from the pool of relevant and unique markers of (d), wherein the selection step comprises balancing the age distribution of samples from which the relevant and unique markers are obtained.

2. The system of claim 1, which further comprises:
(B) a marker identification unit configured to identify a plurality of age-specific methylation markers in the training dataset of e), the marker identification unit communicatively connected to the data acquisition unit, comprising:
f) a classification engine configured to statistically classify each relevant and unique marker in the training dataset of e) on the basis of a relevance score which indicates a level of a statistical association between the marker and the age, wherein the methylation markers comprises the markers listed in Table 1, wherein the markers in Table 1 are listed in descending order of relevance score, and wherein the classification engine utilizes a machine learning (ML) model; and
g) optionally a validation unit for validating the trained machine learning algorithm of (f) with a validation dataset; and

3. The system of claim 1, which further comprises
(C) an analyzing unit comprising:
h) a detector for detecting the methylation status of age-specific, unique and relevant methylation markers identified in (e) or a gene linked to said methylation marker or locus thereto in a biological sample; and
i) an age assessor which calculates the age of the biological sample based on the detected methylation status of the biological sample.

4. The system of claim 1, which comprises the data acquisition unit (A), the marker identification unit (B) and the analyzing unit (C).

5. A computer readable medium comprising computer-executable instructions, which, when executed by a processor, cause the processor to carry out a method or a set of steps for diagnosing aging or an age-related disease in a subject, the method or the set of steps comprising, (A) a pre-analytical data processing, filtering, selection and balancing steps; optionally (B) a system setup step; and further optionally (C) an analytical step, wherein the pre-analytical step (A) comprises:
a) receiving a plurality of methylome datasets from a plurality of heterogeneous samples of different age or age groups, wherein each dataset comprises a plurality of methylation markers;
b) processing to homogenize the plurality of methylome datasets and merging the homogenized dataset into a single data frame, thereby generating a processed dataset comprising a string of homogenized and merged methylation markers;
c) filtering confounding markers from the processed dataset of (b), wherein filtration step comprises:
1) removing cross-reactive markers in the processed dataset;
2) removing unavailable markers in the processed dataset; and/or
3) removing sex-specific markers from the processed dataset;
d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
e) selecting a training dataset from the pool of relevant and unique markers of (d), wherein the selection step comprises balancing the age distribution of samples from which the relevant and unique markers are obtained; wherein the optional system setup step (B) comprises
f) training a machine-learning algorithm comprising a Ridge regression machine learning algorithm with the training dataset of e), thereby generating a plurality of age-specific, unique and relevant methylation markers, wherein the methylation markers comprises the markers listed in Table 1; and
g) optionally validating the trained machine learning algorithm of (f) with a validation dataset; and wherein the further optional analytical step (C) comprises
h) detecting the methylation status of age-specific, unique and relevant methylation markers identified in (e) or a gene linked to said methylation marker or locus thereto in the subject's biological sample; and
i) calculating the age of the subject's biological sample based on the detected methylation status of the subject's biological sample, wherein the markers in Table 1 are listed in descending order of relevance to the age of the subject's biological sample, and wherein if the calculated age is greater than the actual age of the subject, then the subject is diagnosed with aging or having an age-related disease.

6. The computer readable medium of claim 5, wherein the further optional analytical step further comprises j) comparing the calculated age with a chronological age of the subject to infer a rate at which the subject is aging and evaluating interventions to slow down aging or age-related disease in the subject.

7. The computer readable medium of claim 5, wherein computer-executable instructions, when executed by a processor, cause the processor to carry out a method or a set of steps for diagnosing aging or an age-related disease in a subject, the method or the set of steps comprising, (A) the pre-analytical data processing, filtering, selection and balancing steps; (B) the system setup step; and (C) the analytical step.

8. A method for calculating an age of a biological sample, comprising, (A) a pre-analytical data processing, filtering, selection and balancing steps; (B) a system setup step; and (C) an analytical step, wherein the pre-analytical step (A) comprises:
a) receiving a plurality of methylome datasets from a plurality of heterogeneous samples of different age or age groups, wherein each dataset comprises a plurality of methylation markers;
b) processing to homogenize the plurality of methylome datasets and merging the homogenized dataset into a single data frame, thereby generating a processed dataset comprising a string of homogenized and merged methylation markers;
c) filtering confounding markers from the processed dataset of (b), wherein filtration step comprises:
1) removing cross-reactive markers in the processed dataset;
2) removing unavailable markers in the processed dataset; and/or
3) removing sex-specific markers from the processed dataset;
d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
e) selecting a training dataset from the pool of relevant and unique markers of (d), wherein the selection step comprises balancing the age distribution of samples from which the relevant and unique markers are obtained; wherein the system setup step (B) comprises
f) training a machine-learning algorithm comprising a Ridge regression machine learning algorithm with the training dataset of e), thereby generating a plurality of age-specific, unique and relevant methylation markers, wherein the methylation markers comprises the markers listed in Table 1; and
g) optionally validating the trained machine learning algorithm of (f) with a validation dataset; and wherein the analytical step (C) comprises
h) detecting the methylation status of age-specific, unique and relevant methylation markers identified in (e) or a gene linked to said methylation marker or locus thereto in the biological sample; and
i) determining the age of the biological sample based on the detected methylation status of the biological sample, wherein the markers in Table 1 are listed in descending order of relevance to the determined age of the biological sample.

9. A method for calculating an age of a biological sample, comprising detecting the methylation status of age-specific, unique and relevant methylation markers in the biological sample and determining the age of the biological sample based on the detected methylation status of the biological sample, wherein the age-specific, unique and relevant methylation markers are identified in a methylome dataset by employing (A) pre-analytical data processing, filtering, selection and balancing steps; and (B) setting-up step, wherein, the pre-analytical data processing, filtering, selection and balancing step (A) comprises:
a) receiving a plurality of methylome datasets from a plurality of heterogeneous samples of different age or age groups, wherein each dataset comprises a plurality of methylation markers;
b) processing to homogenize the plurality of methylome datasets and merging the homogenized dataset into a single data frame, thereby generating a processed dataset comprising a string of homogenized and merged methylation markers;
c) filtering confounding markers from the processed dataset of (b), wherein filtration step comprises:
1) removing cross-reactive markers in the processed dataset;
2) removing unavailable markers in the processed dataset; and/or
3) removing sex-specific markers from the processed dataset;
d) identifying relevant and unique markers from the filtered markers of (c), wherein the identification comprises carrying out a plurality of correlation or regression steps to classify each marker based on the association thereof to aging, combining the results of each regression step to identify relevant markers, and eliminating redundant markers, thereby generating a pool of relevant and unique markers;
e) selecting a training dataset from the pool of relevant and unique markers of (d), wherein the selection step comprises balancing the age distribution of samples from which the relevant and unique markers are obtained; and the setting up step (B) comprises
f) training a machine-learning algorithm comprising a Ridge regression machine learning algorithm with the training dataset of e), thereby generating a plurality of age-specific, unique and relevant methylation markers, wherein the methylation markers comprises the markers listed in Table 1, and wherein the markers in Table 1 are listed in descending order of relevance to the calculated age of a biological sample; and
g) optionally validating the trained machine learning algorithm of (f) with a validation dataset.

10. The method of claim 8, wherein the methylation markers comprise levels and/or activity of methylated genomic DNA (gDNA) in the samples.

11. The method of claim 8, wherein in step c), (i) the cross-reactive markers are identified by comparing the dataset of (b) with a standard, non-specific probe dataset; (ii) the unavailable markers comprise markers that are not included in the pool of markers which are assayable with the methylation assay instrument; and/or, (iii) the sex-specific markers comprise markers that are specific to a single sex.

12. The method of claim 8, wherein in step d), the correlation or regression comprises application of a regression analysis comprising glmnet-lasso, xgboost, and ranger; and/or in step e), the age balancing step comprises not having more than n samples per age window of y years, beginning with age z years, wherein n, y, and z are integers >0, and wherein n=5 or 6; y=7 years or 8 years; and z=16 years to 20 years.

13. The method of claim 12, wherein n=5, y=7 years and z=18 years.

14. The method of claim 8, wherein the age of the biological sample is determined using a regression model that predicts sample age based on a weighted average of the methylation marker levels plus an offset, preferably, the offset comprises an addition or subtraction of a delta age (δ), derived from a validation dataset of samples obtained from the subject, e.g., as provided in a hash table of Table 4.

15. The method of claim 8, wherein the methylation status comprises level and/or amount of methylation markers or pattern of methylation markers in the biological sample.

16. The method of claim 9, wherein in step c), (i) the cross-reactive markers are identified by comparing the dataset of (b) with a standard, non-specific probe dataset; (ii) the unavailable markers comprise markers that are not included in the pool of markers which are assayable with the methylation assay instrument; and/or, (iii) the sex-specific markers comprise markers that are specific to a single sex.

17. The method of claim 9, wherein in step d), the correlation or regression comprises application of a regression analysis comprising glmnet-lasso, xgboost, and ranger; and/or in step e), the age balancing step comprises not having more than n samples per age window of y years, beginning with age z years, wherein n, y, and z are integers >0, and wherein n=5 or 6; y=7 years or 8 years; and z=16 years to 20 years.

18. The method of claim 17, wherein n=5, y=7 years and z=18 years.

19. The method of claim 9, wherein the age of the biological sample is determined using a regression model that predicts sample age based on a weighted average of the methylation marker levels plus an offset, preferably, the offset comprises an addition or subtraction of a delta age (δ), derived from a validation dataset of samples obtained from the subject, e.g., as provided in a hash table of Table 4.

20. The method of claim 9, wherein the methylation status comprises level and/or amount of methylation markers or pattern of methylation markers in the biological sample.


SEQUENCE LISTING (300 sequences)

SEQ ID NO: 1 - DNA, 122 bp, Homo sapiens
gaggctcctccgggaaagctccttctgctccaggtgacagcggagagagatgccaccgcgcggcgaccggcagggccgcgtcccctctgcgtcctagcacagcgacgccccgcccgccaccc

SEQ ID NO: 2 - DNA, 122 bp, Homo sapiens
cccgctcgcctataaggagctgtccgccacccgggtgctgattccagctctcgcgcccgacgaggtggatttggctgtccaccgagctccggcgcctgtcgttctaattgggtttggatttg

SEQ ID NO: 3 - DNA, 122 bp, Homo sapiens
tcggtcttctcccgcccctccctcccttccccgcctctcccccaagctcctcagtggccgcggcccgtcaacactgtcgcgcagtcactggcgcaggttcccagctctcagctgggggtttc

SEQ ID NO: 4 - DNA, 122 bp, Homo sapiens
ctgcaccccggcgggcgcacagacggtccccagcggcggcctgggccagcggcgaagcagcggcagacggttctccggcccccgccgccccctcaccgctcccggggcaatctggcgctcag

SEQ ID NO: 5 - DNA, 122 bp, Homo sapiens
ccgtggtgctgaaagcttgaccggcgcgagctggagccgccaccggctgcctcggggtctcgccgggccttacctgctccgcgccctggaagcagatcttgcagatgggctggtggtgctgg

SEQ ID NO: 6 - DNA, 122 bp, Homo sapiens
ttgtctcggtcccaagttccgtggttcgctggtgcgggcgctgcagtgtcagggcgctggcgaggctccgcgtgccgcgatgcaaagaaatacatcaataaaaacagaagcagagtgggggt

SEQ ID NO: 7 - DNA, 122 bp, Homo sapiens
acagtcgcagcttaaccccgttgggggcgccgccccgctgaggtggttgcgtctccaagtcgtgagcctccaatagctgctcccgctttcgcgtcgcaaccccaggaccccgggaaattacc

SEQ ID NO: 8 - DNA, 122 bp, Homo sapiens
ttgcagcctggagctcagctccattggaatgctccgggcgctgtccaaggtgctggaatgcgccgcgcccgggggcagagctgcgggccgggggattatcgctgcccacggcttcgggctga

SEQ ID NO: 9 - DNA, 122 bp, Homo sapiens
tcctgtgctcccaggtctgggcgttaggattctctcagtcccggagccacgccggctgaccgcagggctcggggagcgcggctgggccccttttcccgggtccgggaagcgccgggccacgc

SEQ ID NO: 10 - DNA, 122 bp, Homo sapiens
cgcacgaaggtagctccgggcggggagcgaggcgctgtcctcggtgctgaaaggccgaggcgcgcggtgggcgcgacagccccggagacccgaggtctcgcggagggacagcggctacgggc

SEQ ID NO: 11 - DNA, 122 bp, Homo sapiens
agcctgcgaagtggtgccggctgctctcgggctgccctccctccccgaggcgtggagaaccgtacctgtcttcggaagacggaggccccctcacctggtcctcccggctctcagcgtgcgcc

SEQ ID NO: 12 - DNA, 122 bp, Homo sapiens
tcgcggtgcggtccgggactgcgcccctgcgcaccgctcgaggacgaatgtgaggcgccgcgtgagtcctgcgttcgaccccaccccgtcccagccggggaccccggcccctcctgagcgtc

SEQ ID NO: 13 - DNA, 122 bp, Homo sapiens
ggccgcagggagaactcgcctccccgccccggcacgggcactgtctgcggccacgtgccccggaggtcgcggcccaaccagccccgccgacttgttccgctttcgccccagcccccggcggg

SEQ ID NO: 14 - DNA, 122 bp, Homo sapiens
ccgccgctggtccttggcgcgcaaatagcgggcgaagtcaaagggtcccgtaggcgtgggcggcgccggtgtgtccccttcgtaggccggcggggctgcacccgcgtcgggtaactggaacg

SEQ ID NO: 15 - DNA, 122 bp, Homo sapiens
ccggtgcgccgggctctacctcaaggagctcagggccatcgtgctgaaccaacagaggctcgtccgcacccagcgccagagcatcgacgagctggagcggcggctgaacgagctgagcgcct

SEQ ID NO: 16 - DNA, 122 bp, Homo sapiens
gttgaagggtatctcgcagacttttgggaagcggtcccggtagcccatggcgttgcccagcgtcagctccgagaacttgagcagcgccgtctccgatgcgtctccaatcacgatgcgctggg

SEQ ID NO: 17 - DNA, 122 bp, Homo sapiens
tcacctagggcggaggcgcaagctctgctgggtgctctccgcccccttgatcgccgctctcggttttcagcaccaggatccggacagctccccacctggccctgaggggcctctttccttgc

SEQ ID NO: 18 - DNA, 122 bp, Homo sapiens
ggccgagatgcggcagcgcattgccgagctggagatccaggtgatcggcggggccggggtcggggggcgggggcgggggcagggcccggggcaggagcggggccggaccccaggcccagcat

SEQ ID NO: 19 - DNA, 122 bp, Homo sapiens
gttcatcgagaaggtgcatcagctggagacgcagaaccgcgcgttggaggccgagctggccgcgctgcgacagcgccacgctgagccgtcgcgcgtcggcgagctcttccagcgcgagctgc

SEQ ID NO: 20 - DNA, 122 bp, Homo sapiens
gccaggcgctggagcgtggctaaggcagggaccacgtcccagccgccctttcccgccctgcggcgcaggcccactctcttggctctcctggcccgcacactcagctcggccgccgcggctgc

SEQ ID NO: 21 - DNA, 122 bp, Homo sapiens
agtggaagggagggggaacgcaggggagggagaggaggggaggagccgcgcggcccgcgccgcttccgaaccggaaagttggtcttgccgaagtcctgccaccccggcgtgcgcactccgct

SEQ ID NO: 22 - DNA, 122 bp, Homo sapiens
ctcggaaggggcaggggaatgagcccagggaccccagcggggcgcaggtaggaggctgtgcgctcgccgggtgcgctccggccccgattcccagcgcagccagtaagtggcgctgggcctcg

SEQ ID NO: 23 - DNA, 122 bp, Homo sapiens
cactgaggtcgaaggtgggcaggtcgtcggcctccacgccgtggtactggatgtccatgtcgcggtagtagtcgggcccagtgtccacgttccagcggccgtgggccgcgttcagcacgtgc

SEQ ID NO: 24 - DNA, 122 bp, Homo sapiens
ctcgggaggcgctttgcctttgaggaagatggagaggagtcgggagaagcgcctagaaaccgcattgatttagacatcaatcctggccggctccctccgcctgccgagctgcggggccgcgc

SEQ ID NO: 25 - DNA, 122 bp, Homo sapiens
gctggacacgctcaggctggcgtccagctacatcgcccacttgaggcagatcctggctaacgacaaatacgagaacgggtacattcacccggtcaacctggtgagtgctcccggggctgcag

SEQ ID NO: 26 - DNA, 122 bp, Homo sapiens
gcagccccgggtgaccggcgtcgtcctcttccggcagcttgcgccccgcgccaagctcgacgccttcttcgccctggagggcttcccgaccgagccgaacagctccagccgcgccatccacg

SEQ ID NO: 27 - DNA, 122 bp, Homo sapiens
ccgcgcaggtgggggagacctggctggccggaactgggattcggggggagcattgcccttcggcgtaagcgctgctcaggtagagcccagcgctccgcttctccacagaacgtgctggcgcg

SEQ ID NO: 28 - DNA, 122 bp, Homo sapiens
gtaaatgagaaaagacgtgaggttccttttgttctttacctgtggcctccctgccctacacggggactctagggtggaatgtagcaaagcccatccaccagccatgtactaccccccaaccc

SEQ ID NO: 29 - DNA, 122 bp, Homo sapiens
gcgggggaggttgcgggggaggctcggcgtccccgctctccgccccgcgacaccgactgccgccgtggccgccctcaaagctcatggttgtgccgccgccgccctcctgccggcccggctgg

SEQ ID NO: 30 - DNA, 122 bp, Homo sapiens
tggtactagcacgtcacctagaaggaagaatcctggaatggcacgggtccaaactagaggcggcctctcagcatggacccgcttcaacctcatctgcatggcaggcgttttgcaaggcgtca

SEQ ID NO: 31 - DNA, 122 bp, Homo sapiens
ggctcccaaattcctgggagaccctctcccagggcctcctgatgcagctaccatactgagcgatccgtcgataacgcccttggcccaccgatcagtttaccttattagagagaaaagcactc

SEQ ID NO: 32 - DNA, 122 bp, Homo sapiens
aggttccttcttaggggtcctcgctctgctccgcagcccctcctggggatccgggctctgcggtccagcgcgacctgcctggggccacgtgttcaagcacgaagcccctgcgtggagtccac

SEQ ID NO: 33 - DNA, 122 bp, Homo sapiens
tgaccctagtttgatgggttttttcctttgtcctctctttcttggattgagtcctcacagcgcggcggactgcggcgtggtaggaactacaccacccagaatactgtgcgccgagcgtgccg

SEQ ID NO: 34 - DNA, 122 bp, Homo sapiens
gggaggttgcctccaggcgggcctgggataggggacccgaaggggtcaaggtctgcgctccggtgccttcgggggtacccctgccccatcctcttccgcttcacccctgcaggacccagaca

SEQ ID NO: 35 - DNA, 122 bp, Homo sapiens
ctggacgactgtggctgggatggcctcccggcagtaatcttgcgcaaacaccctgccacgcgcaaggacgccagctcagacacgcagcgccccgcgcatacaaaggaatgttccctctttaa

SEQ ID NO: 36 - DNA, 122 bp, Homo sapiens
ggcggcggccccattagcggagcctccgcctatgattggcttcgcccgggaagctggagacgggcgatgaataattgatgtgtgcggtgcggtagccggacggcggcggcggtggcgggcag

SEQ ID NO: 37 - DNA, 122 bp, Homo sapiens
agcgcctgaggagacagacagtgtagactttagggtacaattgcttcccctctgtcgcggcggggtggggagcgtgggaaggggacagccgcgcaaggggccagcctgctccaggtttgagc

SEQ ID NO: 38 - DNA, 122 bp, Homo sapiens
agagcgctacgtcgccggcgggcagcagcagcgcctacaaactggaggcggcggcgcaggcgcacggcaaggccaagccgctgagccgctctctcaaagagttcccgcgtgcgccgccagcc

SEQ ID NO: 39 - DNA, 122 bp, Homo sapiens
gatgggttttttcctttgtcctctctttcttggattgagtcctcacagcgcggcggactgcggcgtggtaggaactacaccacccagaatactgtgcgccgagcgtgccggggccttagacc

SEQ ID NO: 40 - DNA, 122 bp, Homo sapiens
gaatgaaaggggcccaagtagggaacaggagtgaggagagacagggttagcgggggcagtcgaaggagacaacggaaaggcagaaaacagaaaaataacgcaagagagagaaaaagtaaagg

SEQ ID NO: 41 - DNA, 122 bp, Homo sapiens
ggcgcgtggagggtgggaggatccggccgctgccgggcggatgggagctgcgcgaggagacgggcgcgcgtggagagggcgcgggagttggcattcggtggtcctggcagttagctgagcac

SEQ ID NO: 42 - DNA, 122 bp, Homo sapiens
gcgctgcgggctgccgggaactgttctccgctcggggtgctgaaagcggacgcgggagagcgcgcagagaaggcgaggagccgggtcggccaggctctcctgcaggcgcgggtcctgctcgc

SEQ ID NO: 43 - DNA, 122 bp, Homo sapiens
cgagccgtggccgctgctggacgacagggagccggggctggtggcggcgggcggcgagtgcgccacgggcatggacatggagcggctgtgttgcagcgcgccccctgccggcagcagcgcca

SEQ ID NO: 44 - DNA, 122 bp, Homo sapiens
agagcggggagcctcagacccagccgagccccacttctgggcttagagcttgacccaacacgttcgcaccgtagcgagcgaggtccacatttagccatgccgcaggcaaaagaaggattcgg

SEQ ID NO: 45 - DNA, 122 bp, Homo sapiens
gctggaagctccgccttctgtccccgtaagtcccacccccgtcccccgcttcggccaccgcgcttcggccacggcgacttggccaacaacagcggcagcagggtctccccattgagggaagc

SEQ ID NO: 46 - DNA, 122 bp, Homo sapiens
tataactgactgctcaggatatgccaggccttttgctatgtagtgtctgttaacctcatgcggtgctcccagccctgtgaggtacgcattatgctctgcatttttttcagatgagaaaacag

SEQ ID NO: 47 - DNA, 122 bp, Homo sapiens
cacagtcgcgggacaggtgcggagagagctgtggcaggcaggagctggatcgcagcgactcggcctcctcccgcctgcagggcaggctgcaccctgaggagcagagaccctgggctgacccc

SEQ ID NO: 48 - DNA, 122 bp, Homo sapiens
ctctcttcatgagagagtctaaggagggggtccccaaactccccaagcctggtcactgcccgcagccctccaccggatgccccccgcccggaaaagcgctgctgcaagggtttctgcatcga

SEQ ID NO: 49 - DNA, 122 bp, Homo sapiens
cggcgcgcgccgggctgtagctctgcgacgacagcgagcggttctgctgcgggtacgtggcgcacggccgcagcgcccccacggccggcgcgcacgcctcgtcccgcgcgcccgacgcctgc

SEQ ID NO: 50 - DNA, 122 bp, Homo sapiens
gcactggccgcccgctcggctactacgccgacccgtcgggctggggcgcccgcagtcccccgcagtactgcggcaccaagtcgggctcggtgctgccctgctggcccaacagcgccgcggcc

SEQ ID NO: 51 - DNA, 122 bp, Homo sapiens
accgggtgggctctgcttccccgggaccccactctgaccccatcccctaagccgctcccgcgagcacctcagctccgctcccgcgcgggtcagcaattcgaagtccgccccagacccctggg

SEQ ID NO: 52 - DNA, 122 bp, Homo sapiens
aatcatttttttttagcttgaaaccaaagcaaacaagcgcgcacagagaagcccattctccgcggccggcgcggcagcctggccgctgtgggtagctcagggacgcacagaggcccggctgt

SEQ ID NO: 53 - DNA, 122 bp, Homo sapiens
atgagaggagagaggcttgttgatcgcagccaatggctgcggcaggagaggaattagcagcggaaactccaggttcggttcaagaaagatgacacagagcctgtcgggcccgcgcactcttg

SEQ ID NO: 54 - DNA, 122 bp, Homo sapiens
attcattttatttccagaactctccgaccataaattattcaaagagtaagccaacccgagcggggcggccgcgcgccttccccacgcgcgccgggctggctctggccgctcagctcacccga

SEQ ID NO: 55 - DNA, 122 bp, Homo sapiens
tctgcgcagcaaggtttgtctccatggcaaccagactggcggcgcaagggggaggaaacgcgagccgctggctgggaccccggggcactagtaggcttggcacctaagaagccgaaatgcaa

SEQ ID NO: 56 - DNA, 122 bp, Homo sapiens
gtcccctcccccaatgcagagggacttcccgccaaagctcttccggttttcagtctggtccgcagaggttacccataaaagaaagctgccatcacaggcagcagacctttgttctctgacca

SEQ ID NO: 57 - DNA, 122 bp, Homo sapiens
ggcgagtctcctgggacgctgccgaggcacttgctggggagtgtggcccgcgcggggctgcggtctagatgccgagccccttccaggcgcaggcgtcgctgcggaggtgcgttgtcggggga

SEQ ID NO: 58 - DNA, 122 bp, Homo sapiens
gagagatcccgggtcgtcccacatggggctgtgctgcacctggaagcccggggtggtgggcgtcgggggcgaggagggcttgtagtaaaccgacccggagtgcggcatcatctcctcagact

SEQ ID NO: 59 - DNA, 122 bp, Homo sapiens
cctccaggcctgcagccacgcttggcgctgtccgctagggccaggtgctgaagtgttggccgcgagcggagctgctgcagcgctggcttccccgggccgctgcgggtggacttggacaacat

SEQ ID NO: 60 - DNA, 122 bp, Homo sapiens
gaacacctgcttcctctcgttgccttgtgtgaaagtcgcgttgtattttcctgcgcttggcgctgcgcccgcggagctcagggccgtgacccggtgctcgcagccccccgaccccgcagcgg

SEQ ID NO: 61 - DNA, 122 bp, Homo sapiens
gccctcggaggaggcatccttcataacgctgggggcggggagcgcaggccgggccagcggcgccacacgaacggccccgcgggacgctgccacccccgcctcggtcgccccggcgcgtcggc

SEQ ID NO: 62 - DNA, 122 bp, Homo sapiens
cgagggattcagacagtcaagcgccaaggcagcccgaggctccccaaagcctcgctcggccgcacgcgggcaggaatctgcgcttgcactcgggctcagctcctcatcttcctttggccaga

SEQ ID NO: 63 - DNA, 122 bp, Homo sapiens
ggcttccgttgcgctggatgctgacttgccagggccactcgccctcctgcgtgtcctgcccgcccaccattcggttcagcatcctggggcgaccacaggctgggggagcatggggagcgggt

SEQ ID NO: 64 - DNA, 122 bp, Homo sapiens
ggccgggcggcgatttgcaggtccagccggcgccggtttcgcgcggcggctcaacgtccacggagccccaggaatacccacccgctgcccagatcggcagccgctgctgcggggagaagcag

SEQ ID NO: 65 - DNA, 122 bp, Homo sapiens
tttcctccaggaaagataaagtaatcgatagggtcttttaaatagctccgcgtttcctgtcgggagaggagtatcagcgcgcgcaccaaatctgctctggtatgtcaccttatctctcgtcc

SEQ ID NO: 66 - DNA, 122 bp, Homo sapiens
tgcaaaagctgcctgcccgcgcgttatcagcggcgcgcaggcctgtggttttctcgctctcgcaaccctgctttaactgccggtttatttttcgacaaacaggatgcctccatctgaggctg

SEQ ID NO: 67 - DNA, 122 bp, Homo sapiens
gccgggatccgagaacccaaagccccgcaaactgcgcaggcccagtaggggctcgcaaaccgggggccccagggttctcactggccagcatacttgtgtagaactttgttttttctttttgg

SEQ ID NO: 68 - DNA, 122 bp, Homo sapiens
agttctccctcgcagcccgtttggatgcgtgcgtctacagcccagtcgcactttggtgaccggcctgggctgtgaagcaccctttagcgaacagcctccgcacttggggacactggcacaag

SEQ ID NO: 69 - DNA, 122 bp, Homo sapiens
gcctgccctgcaggaccctcctccctcccaagtccgcgtgcctgcccagccccatctaaacgcggggtacggagctcgcaggtctctcttaatctgaaacctgttcctatgaagtgtaagat

SEQ ID NO: 70 - DNA, 122 bp, Homo sapiens
acgtgggggaagaagggggttacgccatcaagtcctgaagcccgtcggaccacccatcgccgcctgcgcagacccaaatcttggtcccgccgtaaggtgccgcagtcccgaatgttccagaa

SEQ ID NO: 71 - DNA, 122 bp, Homo sapiens
atcccgtgctgcaggtgctaagagcccatagggcagagctgagtcggcagaaaaggtgaccgaccctccatccccagagtctatgacactgggccccggagacctctgagacccggttaggc

SEQ ID NO: 72 - DNA, 122 bp, Homo sapiens
ccggctaagtcatgtttaacagcctcagaaattatcttgtctccgcgttctttcttctgccggcgagccaggtaatggtaacagagcgaaactccccagtcggaacttctgggttgcagcag

SEQ ID NO: 73 - DNA, 122 bp, Homo sapiens
ctgcccgtggcccctgcggcctgcgaggccctcaacaagaatgagtcggtgctacgcgcacgagccatcgtggcctttcacggtggcaactaccgcgagctctatcatatcctggaaaacca

SEQ ID NO: 74 - DNA, 122 bp, Homo sapiens
gcagcccgggaaggggcattggtggcgcttggcagcaggtgtgacagacctcctccggggcgcctgatccgcggcgggggcggggcctgcccctagggcccctccagagaacccaccagagg

SEQ ID NO: 75 - DNA, 122 bp, Homo sapiens
caactgggcgagctgtgcatggggcgtggctaaggccgtggtttggttacgattggccagcgggacttaagtgttgtctctgaagagcatggacattagtctggagggtcctggaagagtga

SEQ ID NO: 76 - DNA, 122 bp, Homo sapiens
cggctaaacctttgccgcaggatcccggagccggcgtccttcaaggagcacagagggccccgtagcacgccccttgcccagcgccaccgacccttaagcagcgtcaaggaaggagtcccgat

SEQ ID NO: 77 - DNA, 122 bp, Homo sapiens
tggattccaccccagcccgccccctccccacgcacacagccacggcccctcgcgtcttcgcggcacgttaattaaatgcggaaaacagacagaggctgatgtcattgctctcacaagatcat

SEQ ID NO: 78 - DNA, 122 bp, Homo sapiens
aactgctaaagctctcgcagagtccccagaccccccgcgggacatgaggtcttgcctgttcgtatgcgaacatccttgtacccgcctagcagccctgcagactgcaaattttccctgggtgc

SEQ ID NO: 79 - DNA, 122 bp, Homo sapiens
gccgagcccgaaccccaagccgcggagccagcacctcctccagtcggggtcgtccgctcccggccgttgagccaccgccgccacccggtagtgtgtcccgctgccccaatccgcctcatcaa

SEQ ID NO: 80 - DNA, 122 bp, Homo sapiens
tctccacaaccaccagattatctcaccggcgagtgagactgcaaggtttgggggcccggccgtaccactccgcgctgcgcacggggggttcgtacccatctggccgcgaccgtccgtttccc

SEQ ID NO: 81 - DNA, 122 bp, Homo sapiens
acctccattcaaggtcaaaactttgcccagctcagccttgctcgaccctgggcagggaagcgcggacatcggcagagggagcccgaggctctccgtgcccttcgcgccggtgagttcccgac

SEQ ID NO: 82 - DNA, 122 bp, Homo sapiens
ggcgccgcctcccggcgtctgagctgacacctccttagcgctggccgcgggccgcctctgcggcagcgctagtcgccttctccgaatcggctccgcacaggtaagatcaggggacccggcgc

SEQ ID NO: 83 - DNA, 122 bp, Homo sapiens
cagtccttttccgagatgaggtgagacaagggtccaacttttcctggattcgcctcccagcggacgtgagcttccactgcggctgcagagacgcgagcaacctcttctcatcggctcttatg

SEQ ID NO: 84 - DNA, 122 bp, Homo sapiens
gcggttgctggggtccccgcgcgcgcgcctcggcctccccggcgtccagctcgccccatgcggcccgcagctccaagcacagctgcttccagggctggtggcgcaggccctgccacacgtcg

SEQ ID NO: 85 - DNA, 122 bp, Homo sapiens
cctgccttgttcctgtatgtgccgcttcaccggtatcacgtcctgggtctggtgggaccccggcctggctgccctaccggaagctaagaaaactcctcccccaggggtggccgtcgggcctc

SEQ ID NO: 86 - DNA, 122 bp, Homo sapiens
cactgcccagagatcaccgttccctcattctccccgccacctccccttcccattcctcagcgcctgtcaccacctcccaggcgcctcggagcaagtggcttctcctgtggtctcgcagccgg

SEQ ID NO: 87 - DNA, 122 bp, Homo sapiens
actcggcgctgggctctcccgggctccgggtccccggctgcccccggccgccagtcgggtcggccccgcacctgtttgtgctttgcaggctcccggccccctcgctgagcgaggaagctggt

SEQ ID NO: 88 - DNA, 122 bp, Homo sapiens
aacgtctggcagagctcacagacgtcgttttccactcggcaccaaatgttttacagtcttcgtgagcccatatagattctggcttctgcccagtcgtttgtttgaaactgtaggctctgaga

SEQ ID NO: 89 - DNA, 122 bp, Homo sapiens
aaaagaaaatcggaaaatagatccggaggctgtttaaaaatgtcttcttggagagacttccgtagggtcggccagcgcggagtcttcagttgcgcctggccaagttttttgcaaacgtcaaa

SEQ ID NO: 90 - DNA, 122 bp, Homo sapiens
cacggcctgaccccttttaagagagggacctcaagaggggagctgaattccttgagccctcgcctttcaatcaagttttcaaggcacgctttggccgggccctcccggactggctgtgctgc

SEQ ID NO: 91 - DNA, 122 bp, Homo sapiens
catgcccctctcgctgcaacgcggccaaccgcaggcgggtgctgacgacacctccacccccggctcgtaagctaatttgcgtcacatatggcgtaagagccctgtcggagcgggggacctac

SEQ ID NO: 92 - DNA, 122 bp, Homo sapiens
gccggggtaggagcgacggtcgaggtctggcgtcccgtgggctgggctcagctgggtcggcgcggctccgggcggctagctcgctccggcttcagcacgctggacagcgcccgcgcctccac

SEQ ID NO: 93 - DNA, 122 bp, Homo sapiens
ctcaaaaatcctaacattcagctgattgccggcaggcttagagtcaggcatctgctgcttcggtgggggcccaacgcgcatgctgggcgcccgggtgattgagatccaaagagaagggcact

SEQ ID NO: 94 - DNA, 122 bp, Homo sapiens
ggctgcgccagccgtcgggtagaagtcgggcgtcggtctgtctgcggggccgcctgtgtccgtctttccgtccgattgtcggcaggactcgctttcaggaggacctggctgcattcaggacg

SEQ ID NO: 95 - DNA, 122 bp, Homo sapiens
tgtccagtcctcaagggcagctacttatggctgtggcatctggcattcccgcggattctccgaatatacatatgcccctatttcttgagttatgaattttagatcttttgacttctttttta

SEQ ID NO: 96 - DNA, 122 bp, Homo sapiens
gagcgattggggagctgagcgaccacccaccgctccatggccgtccccttcgaaacacggcgcactggccatgactgactcgcccatcgccctggtttccgtccctctggtttcctggggtt

SEQ ID NO: 97 - DNA, 122 bp, Homo sapiens
acgcctcgcaacctctgaaccagagcataaccccgaggggtggacggagaaatacggcttcggagcagggagcgatgggccggggctggggcgccgccctgcctcgcgcaaagaagggggac

SEQ ID NO: 98 - DNA, 122 bp, Homo sapiens
tcctgcgcacctgcgggcgggcggggagcgggcagcgttagcaccgttagcacccctccgcggcgcctctgccgccagcccgcccctaacccgtcccagcacggcggctcgctcctgtaaac

SEQ ID NO: 99 - DNA, 122 bp, Homo sapiens
ggaagtgaatcatggggcgtgaactcgcaagcgcagtttcctgaagacccggaagccgatcgcgtggggagccggtcttggagcagcgggtgagtttccctttgtctagattagatccgctt

SEQ ID NO: 100 - DNA, 122 bp, Homo sapiens
ccggggatgcccaagttgcacttgcagaaagtttgagcctggcctgcgcgcgcagcgccccgctcttccttgacgcacctcgcggagcgcgcgccggcacgcgggcagagggcgcggggtgg

SEQ ID NO: 101 - DNA, 122 bp, Homo sapiens
tgaaccctccccaggagctcacctggggcacccacgagaaaactacggaagctgtgaagacggaggtgtgcatgtggccgggagaacccggggggggagccgcactggggacagaggggtgg

SEQ ID NO: 102 - DNA, 122 bp, Homo sapiens
caccgccatggacgtggtctacgcgctcaagcgccagggccgcaccctctatggcttcggcggctaaatggcattttgaagcccagtcattctctaaaaaggccctttttagggcccctaag

SEQ ID NO: 103 - DNA, 122 bp, Homo sapiens
ctgagccaagaatgatccctagagaagaatctgagaggccagaggattggaagaattaagcgaattttgaaataaccaagagttatgacaatagtagtaatgaatgacagtgaaccagaagc

SEQ ID NO: 104 - DNA, 122 bp, Homo sapiens
ccagcacagggcctagggcatggggactggccctcttggctgaaacgactccgaccctctcggaagatgcccgcgcggcctctgcccccggggagaggggactgtgcccgatgctcaggcgc

SEQ ID NO: 105 - DNA, 122 bp, Homo sapiens
cgcgaaccagggctgggaggctcggctggaggtgtgaccagggcagggactgacctggcccggaacagaagcgcgcagagtcccatcctgccacgccacgaggagagaagaaggaaagatac

SEQ ID NO: 106 - DNA, 122 bp, Homo sapiens
tagtgacttttggaaaaggctcaatacatcattttaatgagacgtgcaaactcatcattacgatatactaggagaaatgctttgacagacgaagtgggaacaactgggagagtgaatgatgg

SEQ ID NO: 107 - DNA, 122 bp, Homo sapiens
gcctgtttcccttttaggtcccctcccccaatgcagagggacttcccgccaaagctcttccggttttcagtctggtccgcagaggttacccataaaagaaagctgccatcacaggcagcaga

SEQ ID NO: 108 - DNA, 122 bp, Homo sapiens
ctgctcagggcttcctcaaggtgagctcaagacccgcagggcttccctatggcaagccgtcgaggctttctttggatgcaggtggccgcagagcgctcatgcggcgtcggtgctggcagcca

SEQ ID NO: 109 - DNA, 122 bp, Homo sapiens
tgaacgcccgcagcctcagtcccacccccggcccagccccagcgcccccagtcccacccccggccccagcttcagcctcagcgcccccaggcccagccccagtcccacccccagtcccaaca

SEQ ID NO: 110 - DNA, 122 bp, Homo sapiens
gggtcctgcgcaaggccccagtgccccggctaaacctttgccgcaggatcccggagccggcgtccttcaaggagcacagagggccccgtagcacgccccttgcccagcgccaccgaccctta

SEQ ID NO: 111 - DNA, 122 bp, Homo sapiens
cctggggctgcactccgaaacactccactgtaccattcacaaaggcatgggcttccctggcgtcggctgtctacaccgtcgcctggaagctagatgccctgggcagcgaagggcaggtgggg

SEQ ID NO: 112 - DNA, 122 bp, Homo sapiens
agctttcagaaagactgcaatgcagcggttaccaaagtccttgttaatatggaaacaactcgtggtgaagccttttgctccccttcacaactgctgactgttgcctgcagtcggaaggagga

SEQ ID NO: 113 - DNA, 122 bp, Homo sapiens
tgggccattggtcagtctagcctgagggcgggttgttgggcggaagagagagacttcttccggcctcactcgctgtcaccatagagattgcccatccaggcagcgaagcagcagggccaggc

SEQ ID NO: 114 - DNA, 122 bp, Homo sapiens
cttgctccggcttagctgtgcacgggcagaaccgtgaggctactggggctggcccacccccggcatctatcaagaccccatcctgcccctcccaagagtccacaccccttttaggtacaggc

SEQ ID NO: 115 - DNA, 122 bp, Homo sapiens
acttcatccaatccgagcatcgggtgcgtcgtgctcttttctaggagcgtggggtgccttcgcgaataaaatctgaaggcatctctgctctcgcggagcttgttctttcttattttcaagtg

SEQ ID NO: 116 - DNA, 122 bp, Homo sapiens
attgccctatagttttgtaggagagagtggagccagcccagacccgcttcgatctcctctcgcggctcctattcatcatctccgcattgtatatggcagcctcgcaggggcaggggccggcg

SEQ ID NO: 117 - DNA, 122 bp, Homo sapiens
cgcgcggcgcccaattccccgcggaggggagtagccaattaaggcacttgaaaagggagtcgggtggaagatcccccgcccaccagtatcctggatttacccaggtcgagttcagagagcct

SEQ ID NO: 118 - DNA, 122 bp, Homo sapiens
ggaaagaatggggaacgagtgacaccgggaccggagggcgagtcttccaggagcacgtctcggccttctttgcccggcccgaccggcccgacccgtgccgcagcgctcctccctccgctcct

SEQ ID NO: 119 - DNA, 122 bp, Homo sapiens
tttggatgttggcacaaggctgcctgcttgcattagaactcagccggcaaggaaagcaggcggctcaaagactgggtcagcctcagggactggatggggatggagctttcagaggagtggcc

SEQ ID NO: 120 - DNA, 122 bp, Homo sapiens
gatggtttcagagaaagatgaagtttcaactgtggtcctctcagatcaggcctctcggaccgattttcccagctctgcgggcgctctacgcgctggcgcgagccgcccctcaggaggccacc

SEQ ID NO: 121 - DNA, 122 bp, Homo sapiens
tcgcccagcccagaggagaggtccctgtttggccttggttccagcccggctcattcaattcgctgaatgtcgggtctcccggcccgccccgcgattctccgggaattggccttggccgcggg

SEQ ID NO: 122 - DNA, 122 bp, Homo sapiens
ccatgggctgcccattgccacctctgggcagccctccttgatggtgtggagtccgcggtccgcattggttaacttaactgtgcttcctcagatccagtctggaattaattattgaattgtat

SEQ ID NO: 123 - DNA, 122 bp, Homo sapiens
attgcctttgttctgttcgccgctggttttaaaccagcttgctgtgtgcatctcagacgtcggttggtacgtcctccgctgttcttcaggaaagcgatagcctcacctatttgaaacaagcc

SEQ ID NO: 124 - DNA, 122 bp, Homo sapiens
ccgtgcccgccccgggagttcgaagggtgctggggccgaggggaaggctctggtcggcggcgtcagcggcagctcccagacgacctaggactgcaaagggcccaggacggggggcggggcgg

SEQ ID NO: 125 - DNA, 122 bp, Homo sapiens
ctcggcaacgcgccctcggcccgcagcctcctgccccctgtgccccgcttcggcccccagcgcagctgcagaggggcccccctcgacgcatacactcaagagcccgaccgcgcggctgaaat

SEQ ID NO: 126 - DNA, 122 bp, Homo sapiens
tctttaggtccaaaatgaccctgaaggagagtccagaatgcccagtggccgcgtctgcaacggagtcttctttctccaattgccttctgccccatcaccatgggccccacctgcgccacctg

SEQ ID NO: 127 - DNA, 122 bp, Homo sapiens
gacacgcgggacttcggcagtcccagtaacttgctttgctgttctgagacctcagcggggcggtcagacctctgctgtctccgcagcgagttgcagtacttggcgcggggagaggaactcga

SEQ ID NO: 128 - DNA, 122 bp, Homo sapiens
gggccgaaaaccccatttccgtttgaggtaactaaagtacccagcgagcaaggtgacttgcgcgtgtgtctgtgtttgtgtgttttaatgattggcgccttgctttgggtttctcttctgtg

SEQ ID NO: 129 - DNA, 122 bp, Homo sapiens
ggatgatgtggtggctggtggtcaaccgtccgccgcagggggtggccatgaagatggagtcgccggtgcggggtgggtgctgcgggcgctgctgttccgatggtgtctttgatgttgggctg

SEQ ID NO: 130 - DNA, 122 bp, Homo sapiens
tgaactctgcattcctaacagtagaggggctcgtgttcttgtgcatagatcacacttcgacgggcaatgttctaggtagaattggagctcagtggaaaggcagatccctgacagcttgaaca

SEQ ID NO: 131 - DNA, 122 bp, Homo sapiens
atagaagaggtatttgcaagttcaatcgagccacacgtaggaccatacacggaagtgaaccgtgtgaggaatgtgtgtgggagagttcgcgtgaagtctgcgtgcacaaggcagcggcggcc

SEQ ID NO: 132 - DNA, 122 bp, Homo sapiens
tcgtaaggataaaattgctctttcaggttttactgggggagccagctggagccttgggcacgcgcgccctggggaacctttcctctttgccgcccctgcgtgtcgcccctttaaagccttct

SEQ ID NO: 133 - DNA, 122 bp, Homo sapiens
tggctcccgtggctggggctgtgcttctgggcggcagggaccgcggctgcccgaggtaagcgctgggcggagcgggcagctgggggcgagggcgcaggggcgccagcctgacggagcgggac

SEQ ID NO: 134 - DNA, 122 bp, Homo sapiens
ttactggctccccctcctgaggcctccgaggtgtacctggcgcctgcgcagtaaggctagcgccgccgcctgtgcggaggacccggggaggtggtgggctggggagagttagaaaggtctgg

SEQ ID NO: 135 - DNA, 122 bp, Homo sapiens
aactgcaaggcatcggccaatgggaactattgctgggctcgttcgaaagtaaacggtggacggcgcggcccgaggcaggtggcgggagtcagtttaaggctggcgcccagctttccgcgcct

SEQ ID NO: 136 - DNA, 122 bp, Homo sapiens
gcggttccccatcccagggccaccagggcccccgggcccccccgctgcaccggcgtcatccgccatttgctgggaaaagcgacaagaaggaactagtcagtgtggcctacgcatctggcagc

SEQ ID NO: 137 - DNA, 122 bp, Homo sapiens
gggcacttgaacattcttcatgagggctgaggcaggcaagctgagtggagcagtgagtcacggcgtgctgcggcagtggtgtcctgaaataacagcaagcagcagcagcagcagcagcagta

SEQ ID NO: 138 - DNA, 122 bp, Homo sapiens
gccgtgcctgccttccctgccgcctcgcgtcgcccaccgaagggacccggccgtgctgtccgcgcccagaggccgaaggcctgtcaccgggctctactcgctgcctttgtggcgggagcgag

SEQ ID NO: 139 - DNA, 122 bp, Homo sapiens
accaaatacataggttttggcagcacatagatttctgtggttttgctatgcttttagcagcggctgtaaaaagcattgcacactaagcattgctagattgccaaacaaacctaattacattt

SEQ ID NO: 140 - DNA, 122 bp, Homo sapiens
atcccagcctaatttttcttgtgcttttgtttgtatcaggggatgtggctctaaatcagccggacatgtgcgtctaccgaagagggaggaagaagagagtgccttacaccaaactgcagctt

SEQ ID NO: 141 - DNA, 122 bp, Homo sapiens
gggatgtgtccgcagttgccagagcaatgacaacactgcgggaccgcggaggcggctgggcggggctggagcctgtgaccgcgcccgctgcgcgcatgcccaaggccccagcgcttctgcag

SEQ ID NO: 142 - DNA, 122 bp, Homo sapiens
ttttaaactcccatggaagtcaggaaatgccggcaaaagcgatttctggtttacgaagctcggtttgacgatagcaatttccgccgaacgcgactttttcctcttgtggaccaagtcgggat

SEQ ID NO: 143 - DNA, 122 bp, Homo sapiens
tcgacgtgccaagaacctggacagctctcagccgagacccttcatctggtgacgaatggacgttgagtgagtgctcaagctcagacagctgcctaacaaggttctcgaagtccccgccacac

SEQ ID NO: 144 - DNA, 122 bp, Homo sapiens
cggcctggacggggtggggggcgccgcggaggccggcgggacttcccatgtctttctcctcgagctcggaaaaagttcccacccggggaatcccgaccctccaacttcgagaccgccggttc

SEQ ID NO: 145 - DNA, 122 bp, Homo sapiens
ggaagccccgaccctgcagtgctgagggagcggccccgttcctgcctccgccaaaactgtcgagtgttctgttactgacaaccgaacattcccagctaaaacaaagcttgtcctatgccgcc

SEQ ID NO: 146 - DNA, 122 bp, Homo sapiens
tgttaaaatatgtggtctgaagttccctatcactctcgatttgcccaccagccgggtctgcggtgcccgtgcaaacgctgcagctaggatataggggggaggaggggcgggagaatgacaaa

SEQ ID NO: 147 - DNA, 122 bp, Homo sapiens
cgtccgatccaagcgccaaattcaaatttgcggccatcttgagcgggcggaattcagtcgcgcgcggtgcagtcgggaggtggaggcaccggctgcattgttttcgggatcgaggggtgagg

SEQ ID NO: 148 - DNA, 122 bp, Homo sapiens
accgtgctgtgggggcgggaatccccgggcgcccgtggggtgctgtcagtgttcgccctccgcccccgtggtcgacaccgcctccctgtgttgtgaaaccttcctacccctctctggagtct

SEQ ID NO: 149 - DNA, 122 bp, Homo sapiens
cggagccacttccctgaaagccagtgaacctatttaccattgtcatagtaacacacaattcgggcccacgtagacttaatcccgagaggcaattgttcccttgcttgggcggctacgctccc

SEQ ID NO: 150 - DNA, 122 bp, Homo sapiens
ctcccacccacctggaggcaggtctctgtctggctgggccgggtggggggcccaagagggcggggtggggagcggaaaggggcgtggccgaggggcggggtctcccgggccgaggggcggga

SEQ ID NO: 151 - DNA, 122 bp, Homo sapiens
ctgctggaggcgagtcagggacccgaagtctctaaacactcgcctctacccgccgccccgcgaaccccacacactgcagacgcgacactcgcaagtttcggggatggcggccggcgagggcc

SEQ ID NO: 152 - DNA, 122 bp, Homo sapiens
ccgctaacgccctttctggtgagtttggggtcctggccggggggtggggggccatcaccccgggctcgggcccagttggctttggggcacctgagcctcagcagacagcagggcttgaggag

SEQ ID NO: 153 - DNA, 122 bp, Homo sapiens
actttcaacaagcctgcgggccatagaggaccacaagtgagtcgggattgagagggacaccgacctcagactaaatcagagtcagcctcagaactcctaagcaccagccccaccctgaccta

SEQ ID NO: 154 - DNA, 122 bp, Homo sapiens
ctgacctcaccacccaccagggaggtgggtcttattctgggcatcgtgccaagttcttagcggggccctctagaatctctaaagcaaatcaggctgaagaggggaaaaccagcagggggagg

SEQ ID NO: 155 - DNA, 122 bp, Homo sapiens
gctttgccaaagccatcctgttaatagttgatcacatgttgatgagaaccttttcttctacgagaggattacccattaccggtgatatgcacttctgacttatttctctccccccaacccca

SEQ ID NO: 156 - DNA, 122 bp, Homo sapiens
gcaccggagcccgcgggggcggcagagacccgccccggcccgcaggacaccccctcggaacgcgcggccccccggctaagtcatgtttaacagcctcagaaattatcttgtctccgcgttct

SEQ ID NO: 157 - DNA, 122 bp, Homo sapiens
cagggagggtgggatgcatggcaaagtgaggctgcttgctgttcatggacatcatcgtggcggcttggcatgtatatccacaaacactccgaaagtccgcgggaaagtgcgtacgccggctc

SEQ ID NO: 158 - DNA, 122 bp, Homo sapiens
ccttgaaccactgttggcaaagggacagataacgagcccagggcagtgtgggggactttgcgttttgaagtctgggtcagccagatagtaagcatcttttgcttttcctgctataacagata

SEQ ID NO: 159 - DNA, 122 bp, Homo sapiens
ggtggcatgcggaactgcggacggctgcgcaggagcggacagcggagaggcggtactgaccggtgcgaggcggtgctgaccggtgcgggccggtgcgggccagtgcaggccaggcccggccg

SEQ ID NO: 160 - DNA, 122 bp, Homo sapiens
ttatttgaagcctgtcttgcatggccatttggaactgacatttctgctgcaattccaaagcgcgaactccgggggctgaagtccacctacgctccacttaaccccatatactcagaatgcgc

SEQ ID NO: 161 - DNA, 122 bp, Homo sapiens
accaatcccttagcccttttattttttttttgcctaattttaagtcctcgtcctggcattcgcatccctgcttggcctgacccttgcccacatttcgcaccataccccgtccctcacctgct

SEQ ID NO: 162 - DNA, 122 bp, Homo sapiens
taaggcgcccaggttcctcccccttatccctgcagggctggtgccttgcggcaccgcccacgctcggattggtccgaggtgagattcgcccttgtgccctcgtaggccttcggaacagcgga

SEQ ID NO: 163 - DNA, 122 bp, Homo sapiens
ttctggaatacacactacccactgcaaacctctggctgcaggggtcggctcagttgctagcgataccgttgctaactactcgcctgaaagtgacacctgtgatctaaccctggctgctagat

SEQ ID NO: 164 - DNA, 122 bp, Homo sapiens
ggaggaaaagagagggaggaaaggcagggagagaggaataaaggcggggagcaggcgagacgagagcagctccgagaagcagtgtgcgcgccgctttcccaaatcttgcagcccagcgagcc

SEQ ID NO: 165 - DNA, 122 bp, Homo sapiens
ccaggccctgcgcccgcgtgccgcggtgttttcagcggctggcaggagctccttctcaaccgttagcacccaaagagaatcccaacagcacacttccagcgcggattaaaacaaacaaacaa

SEQ ID NO: 166 - DNA, 122 bp, Homo sapiens
cgcgttcagaagctccagctgggaaactggagttggcctgaaagcagctccaggatctcccggcggcggagaggtggctggaacgtctgtctgtcgctgtccattttactttgccgctcccg

SEQ ID NO: 167 - DNA, 122 bp, Homo sapiens
ttccccgagcgggtggccctgtttttctctccctttctcgctcctactcctgttctggcacgggccccccggctcacctggaaggagtggaagaggtaccagaaggcccaggcgttgatgac

SEQ ID NO: 168 - DNA, 122 bp, Homo sapiens
aaggatattagctctttcattctctcaagggtcagatgtaatcttccaacatctgactttcgcgtcacccatttaggaagagacgcggtccctttaaggccctggaaagggtctaagtgttg

SEQ ID NO: 169 - DNA, 122 bp, Homo sapiens
tgcaaactctaaatctgaggcagccgtgaagtcccatgccctgaatcatctcatccttagcgtcatcagcaagaagggaggacactgagaatcaaaggttttatttattgaactcgagcatg

SEQ ID NO: 170 - DNA, 122 bp, Homo sapiens
tgaggacaccgccccaaaccccatgactctacccagaatgcaagcaagatggtgccagggcgcactaaatccccagcatgcactgcgaccgcccttagtagcaagcgtaaactacaatcccc

SEQ ID NO: 171 - DNA, 122 bp, Homo sapiens
gggctgcggcagaggtcgaaggagtgggactcaatgcgcaagcgcggtccggctcttattcgcgccgcagcacccggatgaagaaggcggggtttcgggtgcaccaaggaagacactcaagg

SEQ ID NO: 172 - DNA, 122 bp, Homo sapiens
actcatccccgagacctaccaggccaacaattgccagggcgtgtgcggctggcctcagtccgaccgcaacccgcgctacggcaaccacgtggtgctgctgctgaagatgcaggtccgtgggg

SEQ ID NO: 173 - DNA, 122 bp, Homo sapiens
tttggattccttccaacttttgccactgccatctgctagaaactggttaaaactggcaaccggccaagagagatacatccactcttaaaacccatgcccggaagtgatgcacattatttaca

SEQ ID NO: 174 - DNA, 122 bp, Homo sapiens
tcttcttttttattataaacaatgctaacctgtgagagtgggctgaccctgtaaatccaacggaggagtcttcggaccgaacggcgaaccgccttcaaaccccaattcttacagccaagccg

SEQ ID NO: 175 - DNA, 122 bp, Homo sapiens
atacctgcatcctagaggacagtgccccaacccccgcagggtgtcgtccctaacaggaaccgtaggtaagcctttaataagccacttttatcaggccagctgtttctgggtgctgtgctata

SEQ ID NO: 176 - DNA, 122 bp, Homo sapiens
ctccattttatcaggagtcattctgccactgcagtggatttccttcctgtgatggtgcaccggctcccaggtagagggtttgcccctttctcttcctcatcctcctcttcttgccagtctgc

SEQ ID NO: 177 - DNA, 122 bp, Homo sapiens
tggctcctataggtggcgctgtgacaaggtgcggtggccgggagaggcggctgggggactcgaagactgcgggaaattttctgcgactccgacgctaacccgctgctcccagcctccgcttc

SEQ ID NO: 178 - DNA, 122 bp, Homo sapiens
tcctgctatgacaaccaaaaacgtctttaaatgttgccaaatgtacccggtgagcaaaaacgtgcctagtagagaaccactgctctaatgtgaccaagctgtcctcactcctgatttgtagg

SEQ ID NO: 179 - DNA, 122 bp, Homo sapiens
ttgggaagtgggcaggagacagcccagggtcggggaggcggaggctgtcctgagcaggggcgcagagtccgggctcctgggggccatgccactggctgggctgtctgaacagcagagtggac

SEQ ID NO: 180 - DNA, 122 bp, Homo sapiens
aggaacagactggcaggaagcacaccggggttaacactggttgacttgaataggattattcgatttttaaaaatacttttccatgttttctgagtgctctatgataaatcagttgcatctgt

SEQ ID NO: 181 - DNA, 122 bp, Homo sapiens
tcgacccgctgccctgagtgctcaccacgtgaggaactggagtggccgagttcgccaaggcgccggggacacctgagcagatgagaactggagcctccagctgcttccagcgaatctacaca

SEQ ID NO: 182 - DNA, 122 bp, Homo sapiens
aaaggcaaaggaaaaaaacatgtggatgttttccaaaatattaaccccatcacaatgtctcgctgtcactatccttttacagattaggaaaagaagttacagggagttaattaccctcagat

SEQ ID NO: 183 - DNA, 122 bp, Homo sapiens
tgggtgggaacagaacagccttggtcgtggctgaggagaaatcccacagatgtcactggacgagggtgacgggtggggccgggctttcccctgggtacaggcacaaccgtgctcttccctcg

SEQ ID NO: 184 - DNA, 122 bp, Homo sapiens
tgtctcgtgttgctatgaggtttgcatctgtgtggctggaatagcttgtttgtgggggcccgcgcgtgacctgtgtgtgcgttactgtgtgtgtctcaggcaggatagtgacgggccgtgtg

SEQ ID NO: 185 - DNA, 122 bp, Homo sapiens
tgaggtaggtggtggggcttggggacacgcggctggactggccggagaagtcctcctggccggaggggagccaagtgttcctgttccaggactgcagaactggcccagacctctgtattgga

SEQ ID NO: 186 - DNA, 122 bp, Homo sapiens
aaaaaaaaatcataaggagcccattagttttaaggcagtcacacaaaatgtattaaataccgaatgcaaagaaccccctgccaggctcttctactgctttagaattctttcctctgctcctt

SEQ ID NO: 187 - DNA, 122 bp, Homo sapiens
aacgcacccttgaagtcatcgggttggtcaaagcgcagcctgatctggtcccggaagcggcgggtgctctggcacacgctggtgatgccaaagcagaagcagggcaggcaggcggcgctgtg

SEQ ID NO: 188 - DNA, 122 bp, Homo sapiens
tcctggagctcagcaagggaggggccagcgccagcccgcgtgtgggtggctgggtgggggcgtgggtgggggtccgcctataattatctggggaaatgcatccgcgctctgcttttcgctgc

SEQ ID NO: 189 - DNA, 122 bp, Homo sapiens
gtgtttacttaagaccgatagcaggtgaactcgaagcccacaatcctcgtctgaatcatccgaggcaaagagaaaagccacggaccgttgcacaaaaaggaaagtttgggaagggatgggag

SEQ ID NO: 190 - DNA, 122 bp, Homo sapiens
cgcggggcgcgagggctgaggctctgggcgtggcatcactctcggtccctctgctgggggcggcgaggagagtgcagtgtgtggaaagggatgctgggatgaagggtgtgcgctgagagggg

SEQ ID NO: 191 - DNA, 122 bp, Homo sapiens
tgcagggggcagagcccgaagctgtacccaatcaggggcaccggggaggagctctgcgatcggtccaatcaggcgcgccgtcggggacgcagctgcagacgttcaaccttctcgcgggattt

SEQ ID NO: 192 - DNA, 122 bp, Homo sapiens
tctatgaaatgtacccttttctctggtgacattggcccatccttatgagcataataaaatcgcagaatcaaagcgctgcaagagatcttaaaaccacctaagtctaccactgagagcccaag

SEQ ID NO: 193 - DNA, 122 bp, Homo sapiens
ccaaggtcaccaactagaaagtggcaaggcgggaaaaatgtcttcagagagttcggactccgagctttcaaccaccaagccactaactttgaccctgttggcccactgatggtttaactggc

SEQ ID NO: 194 - DNA, 122 bp, Homo sapiens
ggaatgctgccttcggtgattttaatttcacttttctacttctctcaataacaaaatccgcgtttcaaactccagggaaaagaaaacggaattggctccaggaggatctgcaatcaccaccg

SEQ ID NO: 195 - DNA, 122 bp, Homo sapiens
agtatgtacttgctgacccaattcctgaatttttgcaggataattaagtagcattttcaccgggagtgtagtcaaatatgatttgtactggaggtccttattctgccaggtgcgtgcagaga

SEQ ID NO: 196 - DNA, 122 bp, Homo sapiens
gccaggggcggtgcaagccccgcccggccctacccagggcggctcctccctccgcagcgccgagacttttagtttcgctttcgctaaaggggccccagacccttgctgcggagcgacggaga

SEQ ID NO: 197 - DNA, 122 bp, Homo sapiens
ttacgtggcacagtgttggcctgggcctcgccgtccctggcacgacccatgggatgaggccgcgcctccccccccagcggggccgccgggcagaggtgatgtgggatgctcagtgacttttt

SEQ ID NO: 198 - DNA, 122 bp, Homo sapiens
aacgtggatatacaggcttttctgtaatcaccctgatgacgattcattgactgtgagcctcgttgcatgttgggacggagaggggcggaaggcttagggacagcgcggtgccttctgggatg

SEQ ID NO: 199 - DNA, 122 bp, Homo sapiens
actttccaaagcagccttggcctccttcatgtccagcaacctgagataaggccacgccaccggctaagagttccgccaggggcccagctctcaggaggcctcttcggtgccgccagcctccc

SEQ ID NO: 200 - DNA, 122 bp, Homo sapiens
gggcacaggcgcttgcgcagtagggtggccgctcccggccgcgtgcagcgcgaacgtcggcgcaggcgccaaggctctggcagttggccagcacaccactacgcatgtgtgtcaactctagg

SEQ ID NO: 201 - DNA, 122 bp, Homo sapiens
cactcagagccatcctcttcccaaagctctggccggtagcatactctcccctcctcccgccgacgacaccgttctagatgagaatgccaagtgcaggtcctccgccccattaatgaccccag

SEQ ID NO: 202 - DNA, 122 bp, Homo sapiens
ggcagctgttgaggctcagcagcgccaggctgagggtgtgcaggatgtcgagcgtggaggcggcgcgacaccggtctccgttgtcttcccccccagccacctagggcgccagcagcaggtgg

SEQ ID NO: 203 - DNA, 122 bp, Homo sapiens
gatggggtttcaccatgttggccaggcggactcaaactactgacctcgttattcacccggcgcggcctcccaaagtgctgggattatagtcatgagcccggccctctttttttttttcgttt

SEQ ID NO: 204 - DNA, 122 bp, Homo sapiens
ccagcgtgttaagcgccgtgctgatggccagcaggtcggtgcctgagaaggcgggcagcgcggcgggaaaggcccccacgccagccagcccggcggggcccagcaggccggaggcggcggcg

SEQ ID NO: 205 - DNA, 122 bp, Homo sapiens
cggctcctgaagaccggccctagtcctggccggtttccccaccgcactggtccgccggtccggattttagaagtttggggccgcacgtttttcagttacctttaagccaattcacaaacatt

SEQ ID NO: 206 - DNA, 122 bp, Homo sapiens
ggcggggcctcagtcaggggtatagctggggagagtgaggaggctgcccagtcacagggccgggctgagattggccaaggggactttgatgatctgtctttgcagatgtcagtgcagctgcc

SEQ ID NO: 207 - DNA, 122 bp, Homo sapiens
ggtacctgtgggtgggacagcatgagagattgtacacacttggtgcaggggtcctcaggacgataaggacaattcagtaactgccctccctcatgaccttgatgactgccccctgctcggct

SEQ ID NO: 208 - DNA, 122 bp, Homo sapiens
ggtcagctctggggctctggccccaactgctctccctggggacttgtttaaaaagcagctcgtgacctcggcactttggctggggttttccctttgaggaatgtgggctagacctgggagat

SEQ ID NO: 209 - DNA, 122 bp, Homo sapiens
cagctcgcctggcggaattgcacgcggcggcgggagctggaatagcagaaggaaccacctcgtggagtcgggccggagccctgcagtggctcagacggttgcagggaccgccaggtcggtgc

SEQ ID NO: 210 - DNA, 122 bp, Homo sapiens
ctgggactacgaacttcttctcctaggctggcgtgaggaggggaattcaaccatcgcaagcgttagcgcgaagcggggcctcctgacttcttcccttcgcggggcaggctggggcatgtagt

SEQ ID NO: 211 - DNA, 122 bp, Homo sapiens
aatgctttgaaaactaaagaaaatcacgttatattagaagccttaccctggtttcactttcgctgaagatatcactgtttgccacacaggcaatcagggagctaaaactgtagttaaagttt

SEQ ID NO: 212 - DNA, 122 bp, Homo sapiens
cagcagcgctggggtggagacgaagatcagctggagggcccacagccggatgtgggacaccgggaaaaagtggtcatagcacacatttttgcatcccggttgcagtgtgttgcagacgaagt

SEQ ID NO: 213 - DNA, 122 bp, Homo sapiens
acaagcaggagagaggggccagaaggaagaaataaagacccagcctcagtgggccagtggcgacgtgagatcccagcaagggcgacatcagggagagaccccagcaagggctacgtcagggt

SEQ ID NO: 214 - DNA, 122 bp, Homo sapiens
acctcaggtgatccacccacttcggcctcccagagtgctgggattacaggcgtgagccaccgcgcccggcccattaatactgttaattcgagcagaatgttcttggccccgccccaacagcc

SEQ ID NO: 215 - DNA, 122 bp, Homo sapiens
gcagccggtggtaaaaccgctggagctcaggctcgggcttcgggggctccatcatagagccggcggccgccaccgtccaggaacagaaagccgaggggttactaaggcaaccaggagcccga

SEQ ID NO: 216 - DNA, 122 bp, Homo sapiens
cagttttgtgctgagtaaagaacacggctgttactgacagatggacttgggtcagaatcccgatttcacccttcctttgctgtattaccttgcttgacaggagggctgctggtcacatacag

SEQ ID NO: 217 - DNA, 122 bp, Homo sapiens
cagaacaaagacgccgtgcggaggacgctggagcagatggacgtggtccaccgcatgtgccggatgtacccggagaccttcctgtatgtcaccagcagtgcaggtggggtcctgacctgggt

SEQ ID NO: 218 - DNA, 122 bp, Homo sapiens
gtcttattcgcctcttgtgacacagctatgatgtgacgtcctgcattttactgatgtggacgctgaggtccaaagacaagcagcctcccagggacacacggagctggagtcccccgagtctc

SEQ ID NO: 219 - DNA, 122 bp, Homo sapiens
gggcagaggccgttgctgacgggccggccgctgctgcacagtcagcttgggtgcggagcgcgatcctggaggatgagagaccacttgaccccaaggatgcactgtctcctgctgggaatgct

SEQ ID NO: 220 - DNA, 122 bp, Homo sapiens
tccgtgaagttatcgccataggccggccagggggcgcgagaggcaccggggtgatttccgcgggaatcgataaccaatcggattcccaggccgaacggagcacacccgcccgccctcgctct

SEQ ID NO: 221 - DNA, 122 bp, Homo sapiens
ctagggcctaaggcacaactgccttgccctgggctgaattctaccctagggcagagttttcggtggcctcggtgtactcttagtagtatttctactaaaaagccaacatagagggcatagac

SEQ ID NO: 222 - DNA, 122 bp, Homo sapiens
gttctctcaagagaacaaggaatcaggtcttactacataagggctttctctatggtgacacgtcacatctcaaaacaaaacagaaagtaagacaaaccaagctgtgatgcaggaaaacagag

SEQ ID NO: 223 - DNA, 122 bp, Homo sapiens
ggcggggacggggggaacccatttgaaataaatacttgtgagtctctgacagactccagacgggccgtcgacgccgcctggcaatgtctgggacctgtcacactctgtgatcggtcttttta

SEQ ID NO: 224 - DNA, 122 bp, Homo sapiens
tgatgtgttcccataaaacgccacttaaaagatttaaactttagatggtccaaaaggaaccgttgatgtcaggacaaccataaaccaaattttatctcatggggaaatatgagattggatga

SEQ ID NO: 225 - DNA, 122 bp, Homo sapiens
gagtcagaatgtcagcaccattaaaggaccagagcgccaagtttcttaatacgggtatctcgacaaacacttcaaagtcactgcagaggaagtgtgaatggcttattcctgaatggtttatt

SEQ ID NO: 226 - DNA, 122 bp, Homo sapiens
gacaggggactggagagcaggaagacaggagaacaaggagatttctcctccttcagcagccgcagcagcaacggcgtgtcctccacagttaactggaagaaaaagcctgagtcctggtctcc

SEQ ID NO: 227 - DNA, 122 bp, Homo sapiens
tgcccggctaattcctgtattttcatacttagttgtatttcctattagggccttggatcccgagtataattttgtactcaaatataatttataaataaggccttagcctcccaacaaggtca

SEQ ID NO: 228 - DNA, 122 bp, Homo sapiens
aacggaggtgccgggtgaccttgggagggaccggggctgccaccgggatggggaggggtccggcctcccttcaaacctgcgcccacctcaagcagagtgggttctacatgcttttagacaaa

SEQ ID NO: 229 - DNA, 122 bp, Homo sapiens
gcaaccggggcgtggccaggtgggggcgtggccagtgggagcggcaggtggggcggggctcgtcggtcggggcggagccaggtgaaggcggggccagttaggggcgtggctagtgtgcgcgg

SEQ ID NO: 230 - DNA, 122 bp, Homo sapiens
atgtgcacgacagtggaacggaggcctctccaagaggcgggggcagtgctgtgggcttcacgcctgctgtggcacgagatcctccctgcacgtccacccgtgacagagcagatgatgctcca

SEQ ID NO: 231 - DNA, 122 bp, Homo sapiens
acacttgctgagctataaccttatgaaaaaaagaaagaaaaaaagtgtttatacttcacacgatacaatgtggtgggtacgccaataactaagtgaacggttacatataatggtctatacaa

SEQ ID NO: 232 - DNA, 122 bp, Homo sapiens
ttcgcagggtcccgtcccgggccgcagagagcagccacctccggtcctggctccagcacacggcattcactgccccgtcgtgacctaacaggaatgaccacagaaggttactatttctacta

SEQ ID NO: 233 - DNA, 122 bp, Homo sapiens
tctccgcctgggtggggtggcggcggggggtctctgatctcccttggtccacacagaccccgccggggggttcgcggaaaatggaggaggcgccgcttggaaagcgggtcccgcaggggcct

SEQ ID NO: 234 - DNA, 122 bp, Homo sapiens
tttattatctataaatgtttaatcaaactgtggcattttaaagtcttgtttcaaattcctcgccttcagttggccggtattcttacagctttttcttgagtgcaaggcagcactgcaactgc

SEQ ID NO: 235 - DNA, 122 bp, Homo sapiens
ctgctcggtgttttaaagtttaaagcacaccactgcggaaaggataccccaccactcactcggagcagcttagacgcccctgtcttctagaactaggcgctgcctgggtgccacgaagatca

SEQ ID NO: 236 - DNA, 122 bp, Homo sapiens
ccaggcccaacggcctctttggagcgcagcccggtcttggtcaccagaggtgcccccagtcgctcgtgtctctgccctttggccgggcaatgaggtgcagctcaggacttgccaggcggcgg

SEQ ID NO: 237 - DNA, 122 bp, Homo sapiens
accctctagtttacttgctcgggagaagaaactgactcgttttatttagtgcctatttagcgagcccagagtaacgtacatttgtgctgttttcaattttgtgctatcgcaaatcacaaaaa

SEQ ID NO: 238 - DNA, 122 bp, Homo sapiens
tatccccctcccggtcctggaaaagtagagaggcagccgggagcctgccttctgtgttctcggtgcaggggtattctgagaacggcccctgctcacacgggtttaaaaggaactcagtgacc

SEQ ID NO: 239 - DNA, 122 bp, Homo sapiens
gaccgggtggggacaaggagtactcgtagttgtggggcctgaggaaagtgacagattagacgaaagtatgctaaattagaggactggaggttttgctaaggaagaacttgtatgctgggagg

SEQ ID NO: 240 - DNA, 122 bp, Homo sapiens
ggcaggagggtagctgagatgaccgcgagccagttagaggaatttcgctgcctccagccccgcagcccgccgcagtgccaaataacagacggcagagggcgctcctacctaacctttcccat

SEQ ID NO: 241 - DNA, 122 bp, Homo sapiens
tagctgggcctttctgatacaggatgcttagaaatctgtaacaagcccttttttcagcagcgatttgaaatcctcttacactggaaatcccaactcataatatcaggaattttgcctatgtg

SEQ ID NO: 242 - DNA, 122 bp, Homo sapiens
tttcttgttcttgccgcccatgttgcagctgtggcagaagatccttcgcggcccaggccccgacggtaccactgcacagccgagagctcttcacattccccggctccggggctgccaccctg

SEQ ID NO: 243 - DNA, 122 bp, Homo sapiens
ctgctccgccggcggccactgccgctacacataccaacaagaagcgatctgagtggctggcgcccactggggctaaaggttaaaggctgccctgcgctacggggcgggatcagcggggccaa

SEQ ID NO: 244 - DNA, 122 bp, Homo sapiens
cattagctgagtcaggcttcattatgttcttctcatacagacttggcagcggctgacgtgcgtgcgcagctcccctgccttcaaggtggacggcgtaggcttcctaaaacacgacacagaga

SEQ ID NO: 245 - DNA, 122 bp, Homo sapiens
aggggcaagctttcaggaggtgccagtgcagggtcagctcctccttaacaattctgcacccggccctgacaccaagtctaaagggtcatgaacctctgagtgaaaacaccaagtgcaggatc

SEQ ID NO: 246 - DNA, 122 bp, Homo sapiens
gttccattgcaatctgtcaggacctgggagcctcttcttcttccgccctggcagggtctccgcagaagatttgttgccgtcatgtcggctgcgattgcagctctggccgcttcctatggttc

SEQ ID NO: 247 - DNA, 122 bp, Homo sapiens
gttcttttcacgttggcgcaaatgagcaatgcgcacgaagctgctccatctcctctgctgcgatttcgctgccgaagagccgaggaaggttaggatgcaattaacagagcggagtgacctgc

SEQ ID NO: 248 - DNA, 122 bp, Homo sapiens
cacgtggttcaaccagaagatccgcagaatcaaggcccggcaagccaaagggcgctgcatcgccccgcgcccggagagtcgggacccatctggcccattgtgctgtgccctgctgtgcgtta

SEQ ID NO: 249 - DNA, 122 bp, Homo sapiens
aactgtctttttaggcaagaaactgagcccactaaatagattcagttttcactcttttcccgcttgatggttttattcattcaccatttgcatctctttcagatagactgggtggtattgat

SEQ ID NO: 250 - DNA, 122 bp, Homo sapiens
ccaccttgcgcccagtgtggccagagcttcggccagaaggagctcagtgcgccgcaccagcgcgtgcatcgtggcccccggcctttcgctggtgctcagtgtcccaagagcttcacgcagcg

SEQ ID NO: 251 - DNA, 122 bp, Homo sapiens
cccccggcccctcgggcacccgcatgcgcagttggaagtaggcaaaggtgtcaggctgggcggtccagaccacacggaggcgccctgtctcatctctgcccagcaccctcaactctcccagc

SEQ ID NO: 252 - DNA, 122 bp, Homo sapiens
tcccccaaacctgctgcctctgaaggcatctccacacattgacagccaatgccttcagtgcgttcctagggcaggtgtcctggcttgagtgactgtcctccaataatcagagctcaaactaa

SEQ ID NO: 253 - DNA, 122 bp, Homo sapiens
acaggcacgtgggtgacccgaggcttctctgaacactagaaagcgctgtgagtgagctcacgcccggcacagctcacttttcaatggtggaattgaaagttgtgctttttagaaaagtggcc

SEQ ID NO: 254 - DNA, 122 bp, Homo sapiens
tcagtctccccatatttacaataaaaggggagcgaggtgggatggcgctgaggatccctacgtccgatcctaatctccagctcaggcaggctcggccgccactagcatcctggagcgacaac

SEQ ID NO: 255 - DNA, 122 bp, Homo sapiens
ggggacacgtgggcctttccagttccctgcagccacctttggtctgtaggaaggcagtggcgcagggagcggtgggagcccgggtctgcagggctcaaggtggcgacggcgaagcggtctgc

SEQ ID NO: 256 - DNA, 122 bp, Homo sapiens
attcggggcgcttctccgtgcgcagcgcgaagcagcagcgcctgcacacgccagttagtacggatggaaggtgtgcccccaagggaggcctgaactctagaatttgccctgcctccccaggc

SEQ ID NO: 257 - DNA, 122 bp, Homo sapiens
caagcccgtaaactttctgtggacacccctcaagttgcgcatagtgttgtcccttcactccggtctcagccagggcagaaagtagggtggggagagtgagtcacaagctctatcccgtcctg

SEQ ID NO: 258 - DNA, 122 bp, Homo sapiens
gatggggcactaaggaagcaccaagcaagctccaggagggaaagcaggcaaggctggagccgcagggaaagtaggctgcaaagggatgtgatcttggcctttaggatgtcattttactgtca

SEQ ID NO: 259 - DNA, 122 bp, Homo sapiens
aggctcaagggagggtgacactgactaaggctgcacagcagggctatgaacctgctctaccgactcctgtggcctgtggggcatggtgtgggagcatcttcctgaggctgctgttaagaaca

SEQ ID NO: 260 - DNA, 122 bp, Homo sapiens
ccttctttctttctcgtgtgctgggatccatatagaaggagatgggctccaccgtctggccggagaaagacctgcagtccaccaattaggctagttgctatagtgacacagccttgtcattt

SEQ ID NO: 261 - DNA, 122 bp, Homo sapiens
ctgcactccagcctgggcgacagagtaagactctgtctcaaaaaaaaaaaaaaacattatcgaagtgtgaattcaaatatgtgcagtctatggtatgtcaatgatagctcaacaaaaattat

SEQ ID NO: 262 - DNA, 122 bp, Homo sapiens
gaacgcctagagagtcggactcccctcccttcccaggctctacggggcgccgcggatccgcgaacagccgtgcccggctagcgggcggcccagcaagtgtcaagacccttcggaacgacact

SEQ ID NO: 263 - DNA, 122 bp, Homo sapiens
aaatctggagtaaattgctaagagggattttatctgacttaggtttgcaatatctttgagcgtattgtgttatcaccctattgcatatttggtggtaaggcaacagaacaccaacaaaatta

SEQ ID NO: 264 - DNA, 122 bp, Homo sapiens
ataatacaagacaccaggtacatggtgatgagcaaaaactggcccttctctgtaattattcgcaatataatattaaacccaacttacaataaaagaaattcaaaataaaatggtgccaggga

SEQ ID NO: 265 - DNA, 122 bp, Homo sapiens
ttatgaaataaagtctacattaagagtatgtggggagcaggagaggagggaacaaaatgccgaagacagagacaagagagcaaacggaattaagtgcttttcgatatagttggaaagcagag

SEQ ID NO: 266 - DNA, 122 bp, Homo sapiens
ggagctgctggggctcccctagggggtgggcggcgggcgggtcagcagagcgcattggaacgccagcctagacctctggcctggccccgcctcccctaactcaccaggccgcagcgtgaccc

SEQ ID NO: 267 - DNA, 122 bp, Homo sapiens
cagcctgaccgtccaaggaaagcagcacgtggtgtctgtggaagaagctctcctggcaaccggccagtggaagagcatcaccctgtttgtgcaggaagacagggcccagctgtacatcgact

SEQ ID NO: 268 - DNA, 122 bp, Homo sapiens
tggagtaggagaaagaggaagcgtcttgggctgggtctgcttgagcaactggtgaaactccgcgcctcacgccccgggtgtgtccttgtccaggggcgacgagcattctgggcgaagtccgc

SEQ ID NO: 269 - DNA, 122 bp, Homo sapiens
cctggcggagatgagaacaggagagaaacccacaggcagctgcactgcccacagctgcagcgaagccaatctctaggtctgcaatcacccttaggggccagaaacccagccccgcaccagcg

SEQ ID NO: 270 - DNA, 122 bp, Homo sapiens
agtactaagagtgtttcagatatactagtttgtattgtctcttgggaaactaggattgggcgcgcagatacatcgccatctgctggtcagtttatctgtggtgaaactgcagctttcttgag

SEQ ID NO: 271 - DNA, 122 bp, Homo sapiens
gaagatagggatggggaccccgaacttgaaccactctacgacatagggtgggggctgtcccgtcactgggtggatcacgtcgcatcgcaggaccacgctctccccagctcttgccgtcacaa

SEQ ID NO: 272 - DNA, 122 bp, Homo sapiens
aagcttgtgggagacacagagaggcaaaagctgagctgggaaaatggcaaggcagggaggcgccagagggagcactgcttaacacgtccgtggggctccaaggcttttaataaagggatcct

SEQ ID NO: 273 - DNA, 122 bp, Homo sapiens
tgacattgtatataacgccagtgcagtgatcaaacacagggcactcgcactgggataatgcgattagctaatctacagcacttaccacatttcattaattgcccctctaagggtccttttct

SEQ ID NO: 274 - DNA, 122 bp, Homo sapiens
ggggtttccgcttccgggagaggctgaccgtttccgcttccgtccacttggcgagtgagacgctgatgggaggatggacgtactggtgtctgagtgctccgcgcggctgctgcagcaggttt

SEQ ID NO: 275 - DNA, 122 bp, Homo sapiens
ccattcacgagaggggcttccttccttttgaccttgggaggggtccagagacccgggggacgatctgggagcagaagctggtcgttctgagttttccatccaaatggtttgcttatgaaatt

SEQ ID NO: 276 - DNA, 122 bp, Homo sapiens
acatggaagtcacaagcctggcaccggattcggggcatggccgggagccagggcagagctcgtcgttgccaaactcagagtcagcccatcccccgccacccagagcgcgtcggcgctaggac

SEQ ID NO: 277 - DNA, 122 bp, Homo sapiens
gcgggccgagacttgggttccccaggtccttggtggggaggtttccaggaggctcgggcgcgcccccgtccacggccccggaagctgacgtcgccgaagcgtacgccgctgcccagcctgcg

SEQ ID NO: 278 - DNA, 122 bp, Homo sapiens
ggggttgagcatggccttgcggagcagtgttatggtaggggcggggctgggatccggagccgttacaaaggaggaaggcggggccgcgcagagcagggtcagggtaggagggcgctcagggt

SEQ ID NO: 279 - DNA, 122 bp, Homo sapiens
ccagttttcccgcgaaaacgctgccgcgcagggggtcagaccatctggaccaaggggggccgagcgaggcctacttctggtttacgcacgggcgctgaaagaagcggcactgtccccccctg

SEQ ID NO: 280 - DNA, 122 bp, Homo sapiens
tgaactcagtggctgctgttttctgagcacctgaaccctgtgggggacgacagagttgcccgaggcggcaggatgtccccacactcgcggtcccccgcacatcttcctgttgctttgggact

SEQ ID NO: 281 - DNA, 122 bp, Homo sapiens
caggagacacggaatgtgaaggcccagtcacagactgaccgagtggacctggggaccctgcgcggctactacaaccagagcgaggccggtgagtgaccccggccgggggcgcaggtcaggac

SEQ ID NO: 282 - DNA, 122 bp, Homo sapiens
ctggcgccggacctaaggggagacaaaacaggagacaggtcaggtcgaggcctctggagtcgggtcgttccccagtgactccagggcagcgcaccccgcgaatgcccacttcggcgatactc

SEQ ID NO: 283 - DNA, 122 bp, Homo sapiens
gagaacagcgattagggccttaaacctcacacccgaacaaattcggccggagttactgagcggcaggctctctgatggagatgggtgctttcagacttaagacgtgaaaacaaagatcagcc

SEQ ID NO: 284 - DNA, 122 bp, Homo sapiens
ctgtctgtctcgtactcttatctcttcccttttctgtggccggcacccccacgacggcctcgcccccgcatccgggccccttcgcgattccggaggaatcccccagagccgcctgaccccgc

SEQ ID NO: 285 - DNA, 122 bp, Homo sapiens
tcggcgtgcgggcgccgggctgcccagctgacttacggatcgggttggtcccgcccccggcgcggccgttttgaaaatcctggtccgcccttggcgattttggtggaagcctgtccctcaga

SEQ ID NO: 286 - DNA, 122 bp, Homo sapiens
ttctcacactccgcgaaggccagccactcgagtcgccagagtagtcgtcccggtcgccgccgctgcttcaaaggcagccttagcctcgctgcagccccgatttcctcacacacacacaccga

SEQ ID NO: 287 - DNA, 122 bp, Homo sapiens
gccaagcacgaagagaaagccccgcctgaaactgcctggaggccccccggctgtcactctcgccacattccgtggagtatgtggttgcaacttctgtcactcaaggtctgatggcggggaga

SEQ ID NO: 288 - DNA, 122 bp, Homo sapiens
gtgtatcctctttttctcaatgtttctatttcctttccaggtccacctcccccaggaatgcgtccaccaagaccttagcatactgttgatccatctcagtcactttttcccctgcaatgcgt

SEQ ID NO: 289 - DNA, 122 bp, Homo sapiens
atttattattaattgtaggtgaatactcgtttttgtccacttttctgtctaaaatgagctcgatgaggacaagaaccttctctgtattgctcactgtgtcttcctaatgattagtagagtgc

SEQ ID NO: 290 - DNA, 122 bp, Homo sapiens
ccgcaccgtgagctttgtgactgatccgaggcggcgagcgggggcactgcactgctgtggcggggaagtcacggctgacaagaactgccagggacgaagccacgtgcattaattcattaaaa

SEQ ID NO: 291 - DNA, 122 bp, Homo sapiens
cccacattttgcagacaaggatatttagttccagagtggctgagtgagtagcccgggtcacgaggcagcccaaaagagagtgtcttgtccacattctgaggatgggcatcaacagatgggga

SEQ ID NO: 292 - DNA, 122 bp, Homo sapiens
cggcgagccgccgactggctggtcccctccatccacctcaccctccccgcccctccctcccggcagccccagccccggcgagcacccagctagccgcctcctgcaggggctcgggagagcaa

SEQ ID NO: 293 - DNA, 122 bp, Homo sapiens
tgtgtggcatcaggtgtgacttctgagaagaaacaatcttggcgcgcgccgcttggatgccggagaaaatggttcttgggtgcgctgatcatcccaggggaggggaggaccttgcttgggcc

SEQ ID NO: 294 - DNA, 122 bp, Homo sapiens
tcctgccagatgagggagccccggcggaggccaggagggcttgcgttgcacaatctggagcggatccccgggggcggctgagggcctgggaccccagtctccctcgaggtcttcactcaccc

SEQ ID NO: 295 - DNA, 122 bp, Homo sapiens
tggcagatcagaggcaggcgggccaggggctctggtttacacaccaaacctccagggcttcggctccaggggccagcagctgggtccaccctgagggagagtccccaggtgagcgagaagct

SEQ ID NO: 296 - DNA, 122 bp, Homo sapiens
cccacccccagggcagcacgtgcggggcggggctgtggcccgagcccggagctgattgggcgcgggcctggtgggcggggccgggccgcagctgtcagagccgcggcggcgaacgaggcgca

SEQ ID NO: 297 - DNA, 122 bp, Homo sapiens
cgctctcggagggacaccgggggcgggaggcgagactgcagcgcaggggccagaacgctgcgactttaagagccgaggatcccggaccatgtgctcggcgtgagacaaaagcaacaacaaag

SEQ ID NO: 298 - DNA, 122 bp, Homo sapiens
ggaaactcgcgggtctcccctgcccctccctgaaggcggcccttcagcgccgcgcgcttccgcccccacactcgggttgaggagcaaggagagaaaagagcgtctttctctcttgctcaaag

SEQ ID NO: 299 - DNA, 122 bp, Homo sapiens
gggcggggcgggggcgggggcggggcgctcctctgggcaccgcccccggcccgccccccgcgctcgcagtcccgctcgcacactggctcccacccgccgcccgcccaggcactgcccgcggg

SEQ ID NO: 300 - DNA, 122 bp, Homo sapiens
cgagccggaggctgggacgcagctggacgcagctgggcgcggaagcttggggcggaggcgcgtgcccgccttcccagctcagccccggcagggctcccggctccagcccactgggagctcgc